SEQUENCE LISTING (266 sequences)

SEQ ID NO: 1 - protein, 5 aa, synthetic construct
  REGION 1..5: note = Description of Artificial Sequence: Synthetic peptide
DYNMD

SEQ ID NO: 2 - protein, 5 aa, synthetic construct
  REGION 1..5: note = Description of Artificial Sequence: Synthetic peptide
SYWIH

SEQ ID NO: 3 - protein, 7 aa, synthetic construct
  REGION 1..7: note = Description of Artificial Sequence: Synthetic peptide
TYGMGVT

SEQ ID NO: 4 - protein, 7 aa, synthetic construct
  REGION 1..7: note = Description of Artificial Sequence: Synthetic peptide
TYGMGVS

SEQ ID NO: 5 - protein, 7 aa, synthetic construct
  REGION 1..7: note = Description of Artificial Sequence: Synthetic peptide
TYGMGVG

SEQ ID NO: 6 - protein, 7 aa, synthetic construct
  REGION 1..7: note = Description of Artificial Sequence: Synthetic peptide
TSGMGVS

SEQ ID NO: 7 - protein, 17 aa, synthetic construct
  REGION 1..17: note = Description of Artificial Sequence: Synthetic peptide
QINPNNGGIFFNQKFKG

SEQ ID NO: 8 - protein, 17 aa, synthetic construct
  REGION 1..17: note = Description of Artificial Sequence: Synthetic peptide
DINPSNGRSKYNEKFKN

SEQ ID NO: 9 - protein, 16 aa, synthetic construct
  REGION 1..16: note = Description of Artificial Sequence: Synthetic peptide
HIYWDDDKRYNPSLKS

SEQ ID NO: 10 - protein, 17 aa, synthetic construct
  REGION 1..17: note = Description of Artificial Sequence: Synthetic peptide
EINPNNGGTFYNQKFKG

SEQ ID NO: 11 - protein, 16 aa, synthetic construct
  REGION 1..16: note = Description of Artificial Sequence: Synthetic peptide
DIWWDDDKYYNPSLKS

SEQ ID NO: 12 - protein, 16 aa, synthetic construct
  REGION 1..16: note = Description of Artificial Sequence: Synthetic peptide
HNDWDDDKRYKSSLKS

SEQ ID NO: 13 - protein, 17 aa, synthetic construct
  REGION 1..17: note = Description of Artificial Sequence: Synthetic peptide
QINPNNGGIFFNQKFQG

SEQ ID NO: 14 - protein, 16 aa, synthetic construct
  REGION 1..16: note = Description of Artificial Sequence: Synthetic peptide
HIYWDDDKRYNPSLKT

SEQ ID NO: 15 - protein, 11 aa, synthetic construct
  REGION 1..11: note = Description of Artificial Sequence: Synthetic peptide
EAITTVGAMDY

SEQ ID NO: 16 - protein, 9 aa, synthetic construct
  REGION 1..9: note = Description of Artificial Sequence: Synthetic peptide
EVLDGAMDY

SEQ ID NO: 17 - protein, 9 aa, synthetic construct
  REGION 1..9: note = Description of Artificial Sequence: Synthetic peptide
TGYSNLFAY

SEQ ID NO: 18 - protein, 9 aa, synthetic construct
  REGION 1..9: note = Description of Artificial Sequence: Synthetic peptide
RGYDDYWGY

SEQ ID NO: 19 - protein, 9 aa, synthetic construct
  REGION 1..9: note = Description of Artificial Sequence: Synthetic peptide
RGHYSAMDY

SEQ ID NO: 20 - protein, 11 aa, synthetic construct
  REGION 1..11: note = Description of Artificial Sequence: Synthetic peptide
RVGGLEGYFDY

SEQ ID NO: 21 - protein, 11 aa, synthetic construct
  REGION 1..11: note = Description of Artificial Sequence: Synthetic peptide
RTSENLHNYLA

SEQ ID NO: 22 - protein, 15 aa, synthetic construct
  REGION 1..15: note = Description of Artificial Sequence: Synthetic peptide
RASESVDNYGISFMN

SEQ ID NO: 23 - protein, 11 aa, synthetic construct
  REGION 1..11: note = Description of Artificial Sequence: Synthetic peptide
KASQNVGTNVA

SEQ ID NO: 24 - protein, 11 aa, synthetic construct
  REGION 1..11: note = Description of Artificial Sequence: Synthetic peptide
RASGNIHNYLA

SEQ ID NO: 25 - protein, 15 aa, synthetic construct
  REGION 1..15: note = Description of Artificial Sequence: Synthetic peptide
RASQSVSTSRFSYMH

SEQ ID NO: 26 - protein, 7 aa, synthetic construct
  REGION 1..7: note = Description of Artificial Sequence: Synthetic peptide
DAKTLAD

SEQ ID NO: 27 - protein, 7 aa, synthetic construct
  REGION 1..7: note = Description of Artificial Sequence: Synthetic peptide
AASNQGS

SEQ ID NO: 28 - protein, 7 aa, synthetic construct
  REGION 1..7: note = Description of Artificial Sequence: Synthetic peptide
SASYRYS

SEQ ID NO: 29 - protein, 7 aa, synthetic construct
  REGION 1..7: note = Description of Artificial Sequence: Synthetic peptide
NAKTLAD

SEQ ID NO: 30 - protein, 7 aa, synthetic construct
  REGION 1..7: note = Description of Artificial Sequence: Synthetic peptide
SPSYRYS

SEQ ID NO: 31 - protein, 7 aa, synthetic construct
  REGION 1..7: note = Description of Artificial Sequence: Synthetic peptide
YASNLES

SEQ ID NO: 32 - protein, 9 aa, synthetic construct
  REGION 1..9: note = Description of Artificial Sequence: Synthetic peptide
QHFWSSPYT

SEQ ID NO: 33 - protein, 9 aa, synthetic construct
  REGION 1..9: note = Description of Artificial Sequence: Synthetic peptide
QQSKEVPWT

SEQ ID NO: 34 - protein, 9 aa, synthetic construct
  REGION 1..9: note = Description of Artificial Sequence: Synthetic peptide
QQYNSYPYT

SEQ ID NO: 35 - protein, 9 aa, synthetic construct
  REGION 1..9: note = Description of Artificial Sequence: Synthetic peptide
QQYNNYPLT

SEQ ID NO: 36 - protein, 9 aa, synthetic construct
  REGION 1..9: note = Description of Artificial Sequence: Synthetic peptide
QQYNSYPHT

SEQ ID NO: 37 - protein, 9 aa, synthetic construct
  REGION 1..9: note = Description of Artificial Sequence: Synthetic peptide
QHSWEIPYT

SEQ ID NO: 38 - protein, 7 aa, synthetic construct
  REGION 1..7: note = Description of Artificial Sequence: Synthetic peptide
GYTFTDY

SEQ ID NO: 39 - DNA, 360 bp, synthetic construct
  misc_feature 1..360: note = Description of Artificial Sequence: Synthetic polynucleotide
  source 1..360: mol_type = other DNA
gaggtcctgctgcaacagtctggacctgagctggtgaagcctggggcttcagtgaagataccctgcaaggcttctggatacacattcactgactacaacatggactgggtgaagcagagccatggaaagagccttgagtggattggacaaattaatcctaacaatggtggtattttcttcaaccagaagttcaagggcaaggccacattgactgtagacaagtcctccaatacagccttcatggaggtccgcagcctgacatctgaggacactgcagtctattactgtgcaagagaggcaattactacggtaggcgctatggactactggggtcaaggaacctcagtcaccgtctcctca

SEQ ID NO: 40 - protein, 120 aa, synthetic construct
  REGION 1..120: note = Description of Artificial Sequence: Synthetic polypeptide
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSS

SEQ ID NO: 41 - DNA, 354 bp, synthetic construct
  misc_feature 1..354: note = Description of Artificial Sequence: Synthetic polynucleotide
  source 1..354: mol_type = other DNA
caggtccaactgcagcagcctggggctgaactggtgaagcctggggcttcagtgaagctgtcctgcaaggcttctggctacaccttcaccagctactggattcactgggtgaaccagaggcctggacaaggccttgagtggattggagacattaatcctagcaacggccgtagtaagtataatgagaagttcaagaacaaggccacaatgactgcagacaaatcctccaacacagcctacatgcaactcagcagcctgacatctgaggactctgcggtctattactgtgcaagagaggttctggatggtgctatggactactggggtcaaggaacctcagtcaccgtctcctca

SEQ ID NO: 42 - protein, 118 aa, synthetic construct
  REGION 1..118: note = Description of Artificial Sequence: Synthetic polypeptide
QVQLQQPGAELVKPGASVKLSCKASGYTFTSYWIHWVNQRPGQGLEWIGDINPSNGRSKYNEKFKNKATMTADKSSNTAYMQLSSLTSEDSAVYYCAREVLDGAMDYWGQGTSVTVSS

SEQ ID NO: 43 - DNA, 357 bp, synthetic construct
  misc_feature 1..357: note = Description of Artificial Sequence: Synthetic polynucleotide
  source 1..357: mol_type = other DNA
caggttactctgaaagagtctggccctgggatattgcagccctcccagaccctcagtctgacttgttctttctctgggttttcactgagcacttatggtatgggtgtgacctggattcgtcagccttcaggaaagggtctggagtggctggcacacatttactgggatgatgacaagcgctataacccatccctgaagagccggctcacaatctccaaggatacctccaacaaccaggtattcctcaagatcaccagtgtggacactgcagatactgccacatactactgtgctcaaacggggtatagtaacttgtttgcttactggggccaagggactctggtcactgtctctgca

SEQ ID NO: 44 - protein, 119 aa, synthetic construct
  REGION 1..119: note = Description of Artificial Sequence: Synthetic polypeptide
QVTLKESGPGILQPSQTLSLTCSFSGFSLSTYGMGVTWIRQPSGKGLEWLAHIYWDDDKRYNPSLKSRLTISKDTSNNQVFLKITSVDTADTATYYCAQTGYSNLFAYWGQGTLVTVSA

SEQ ID NO: 45 - DNA, 357 bp, synthetic construct
  misc_feature 1..357: note = Description of Artificial Sequence: Synthetic polynucleotide
  source 1..357: mol_type = other DNA
caggttactctgaaagagtctggccctgggatattgcagccctcccagaccctcagtctgacttgttctttctctgggttttcactgaacacttatggtatgggtgtgagctggattcgtcagccttcaggaaagggtctggagtggctggcacacatttactgggatgatgacaagcgctataacccatccctgaagagccggctcacaatctccaaggatgcctccaacaaccgggtcttcctcaagatcaccagtgtggacactgcagatactgccacatactactgtgctcaaagaggttatgatgattactggggttactggggccaagggactctggtcactatctctgca

SEQ ID NO: 46 - protein, 119 aa, synthetic construct
  REGION 1..119: note = Description of Artificial Sequence: Synthetic polypeptide
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISA

SEQ ID NO: 47 - DNA, 360 bp, synthetic construct
  misc_feature 1..360: note = Description of Artificial Sequence: Synthetic polynucleotide
  source 1..360: mol_type = other DNA
gaggtcctgctgcaacagtctggacctgaggtggtgaagcctggggcttcagtgaagataccctgcaaggcttctggatacacattcactgactacaacatggactgggtgaagcagagccatggaaagagccttgagtggattggagagattaatcctaacaatggtggtactttctacaaccagaagttcaagggcaaggccacattgactgtagacaagtcctccagcacagcctacatggagctccgcagcctgacatctgaggacactgcagtctattactgtgcaagagaggcaattactacggtaggcgctatggactactggggtcaaggaacctcagtcaccgtctcctca

SEQ ID NO: 48 - protein, 120 aa, synthetic construct
  REGION 1..120: note = Description of Artificial Sequence: Synthetic polypeptide
EVLLQQSGPEVVKPGASVKIPCKASGYTFTDYNMDWVKQSHGKSLEWIGEINPNNGGTFYNQKFKGKATLTVDKSSSTAYMELRSLTSEDTAVYYCAREAITTVGAMDYWGQGTSVTVSS

SEQ ID NO: 49 - DNA, 357 bp, synthetic construct
  misc_feature 1..357: note = Description of Artificial Sequence: Synthetic polynucleotide
  source 1..357: mol_type = other DNA
caggttactctgaaagagtctggccctggaatattgcagccctcccagaccctcagtctgacttgttctttctctgggttttcactgagcacttatggtatgggtgtaggctggattcgtcagccttcaggaaagggtctagagtggctggcagacatttggtgggatgacgataagtactataacccatccctgaagagccggctcacaatctccaaggatacctccagcaatgaggtattcctcaagatcgccattgtggacactgcagatactgccacttactactgtgctcgaagaggtcactactctgctatggactactggggtcaaggaacctcagtcaccgtctcctca

SEQ ID NO: 50 - protein, 119 aa, synthetic construct
  REGION 1..119: note = Description of Artificial Sequence: Synthetic polypeptide
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSS

SEQ ID NO: 51 - DNA, 363 bp, synthetic construct
  misc_feature 1..363: note = Description of Artificial Sequence: Synthetic polynucleotide
  source 1..363: mol_type = other DNA
caggttactctgaaagagtctggccctgggatattgcagccctcccagaccctcagtctgacttgttctttctctgggttttcactgagcacttctggtatgggtgtgagttggattcgtcagccttcaggaaagggtctggagtggctggcacacaatgactgggatgatgacaagcgctataagtcatccctgaagagccggctcacaatatccaaggatacctccagaaaccaggtattcctcaagatcaccagtgtggacactgcagatactgccacatactactgtgctcgaagagttgggggattagagggctattttgattactggggccaaggcaccactctcacagtctcctca

SEQ ID NO: 52 - protein, 121 aa, synthetic construct
  REGION 1..121: note = Description of Artificial Sequence: Synthetic polypeptide
QVTLKESGPGILQPSQTLSLTCSFSGFSLSTSGMGVSWIRQPSGKGLEWLAHNDWDDDKRYKSSLKSRLTISKDTSRNQVFLKITSVDTADTATYYCARRVGGLEGYFDYWGQGTTLTVSS

SEQ ID NO: 53 - DNA, 360 bp, synthetic construct
  misc_feature 1..360: note = Description of Artificial Sequence: Synthetic polynucleotide
  source 1..360: mol_type = other DNA
caagtgcaacttgtgcagtcgggtgcggaagtcaaaaagccgggagcgtcggtgaaagtatcgtgtaaagcgtcgggatatacgtttacggactataacatggactgggtacgacaggcaccggggaaatcgttggaatggatcggacagattaatccgaacaatgggggaattttctttaatcagaaattcaaaggacgggcgacgttgacggtcgatacatcgacgaatacggcgtatatggaattgaggtcgcttcgctcggacgatacggcggtctattactgcgccagggaggcgatcacgacggtaggggcgatggattattggggacaggggacgcttgtgacggtatcgtcg

SEQ ID NO: 54 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGKSLEWIGQINPNNGGIFFNQKFKGRATLTVDTSTNTAYMELRSLRSDDTAVYYCAREAITTVGAMDYWGQGTLVTVSS

SEQ ID NO: 55 - DNA, 360 bp, synthetic construct
  misc_feature 1..360: note = Description of Artificial Sequence: Synthetic polynucleotide
  source 1..360: mol_type = other DNA
caagtccagcttgtccagtcgggagcggaagtgaagaaaccggggtcgtcggtcaaagtatcgtgtaaagcgtcgggatatacgtttacggactataacatggattgggtacgacaggctccgggaaaatcattggaatggattggacagattaatccgaataatgggggtatcttctttaatcaaaagtttaaagggagggcgacgttgacggtggacaaatcgacaaatacggcgtatatggaattgtcgtcgcttcggtcggaggacacggcggtgtattactgcgcgagggaggcgatcacgacggtcggggcgatggattattggggacagggaacgcttgtgacggtatcgtcg

SEQ ID NO: 56 - protein, 120 aa, synthetic construct
  REGION 1..120: note = Description of Artificial Sequence: Synthetic polypeptide
QVQLVQSGAEVKKPGSSVKVSCKASGYTFTDYNMDWVRQAPGKSLEWIGQINPNNGGIFFNQKFKGRATLTVDKSTNTAYMELSSLRSEDTAVYYCAREAITTVGAMDYWGQGTLVTVSS

SEQ ID NO: 57 - DNA, 360 bp, synthetic construct
  misc_feature 1..360: note = Description of Artificial Sequence: Synthetic polynucleotide
  source 1..360: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagggtcttgaatggatggggcagattaatccgaataatggagggatcttctttaatcagaaattcaaaggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcg

SEQ ID NO: 58 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGQGLEWMGQINPNNGGIFFNQKFKGRVTLTTDTSTSTAYMELRSLRSDDTAVYYCAREAITTVGAMDYWGQGTLVTVSS

SEQ ID NO: 59 - DNA, 360 bp, synthetic construct
  misc_feature 1..360: note = Description of Artificial Sequence: Synthetic polynucleotide
  source 1..360: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgaataatggagggatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcg

SEQ ID NO: 60 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGQSLEWMGQINPNNGGIFFNQKFQGRVTLTTDTSTSTAYMELRSLRSDDTAVYYCAREAITTVGAMDYWGQGTLVTVSS

SEQ ID NO: 61 - DNA, 360 bp, synthetic construct
  misc_feature 1..360: note = Description of Artificial Sequence: Synthetic polynucleotide
  source 1..360: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagggtcttgaatggatggggcagattaatccgaataatggagggatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcg

SEQ ID NO: 62 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGQGLEWMGQINPNNGGIFFNQKFQGRVTLTTDTSTSTAYMELRSLRSDDTAVYYCAREAITTVGAMDYWGQGTLVTVSS

SEQ ID NO: 63 - DNA, 360 bp, synthetic construct
  misc_feature 1..360: note = Description of Artificial Sequence: Synthetic polynucleotide
  source 1..360: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggggaatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcg

SEQ ID NO: 64 - protein, 120 aa, synthetic construct
  REGION 1..120: note = Description of Artificial Sequence: Synthetic polypeptide
QVQLVQSGAEVKKPGSSVKVSCKASGYTFSDYNMDWVRQAPGQGLEWMGQINPNNGGIFFNQKFKGRVTLTADKSTSTAYMELSSLRSEDTAVYYCAREAITTVGAMDYWGQGTLVTVSS

SEQ ID NO: 65 - DNA, 360 bp, synthetic construct
  misc_feature 1..360: note = Description of Artificial Sequence: Synthetic polynucleotide
  source 1..360: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggggaatctttttcaatcagaagtttcaggggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcg

SEQ ID NO: 66 - protein, 120 aa, synthetic construct
  REGION 1..120: note = Description of Artificial Sequence: Synthetic polypeptide
QVQLVQSGAEVKKPGSSVKVSCKASGYTFSDYNMDWVRQAPGQGLEWMGQINPNNGGIFFNQKFQGRVTLTADKSTSTAYMELSSLRSEDTAVYYCAREAITTVGAMDYWGQGTLVTVSS

SEQ ID NO: 67 - DNA, 357 bp, synthetic construct
  misc_feature 1..357: note = Description of Artificial Sequence: Synthetic polynucleotide
  source 1..357: mol_type = other DNA
caggtgactttgaaagaatccggtcccgcattggtaaagccaacccagacacttacgctcacatgtacattttccggattcagcttgaacacttacgggatgggagtgtcgtggattcggcaacctccggggaaggctctggagtggctggcgcacatctactgggatgatgacaaaaggtataacccctcacttaaaacgagactgacgatctcgaaggacacaagcaagaatcaggtcgtcctcacgattacgaatgtagacccggtggatactgccgtctattactgcgcgcaacgcgggtatgatgactactggggatattggggtcagggcaccctcgtgaccatctcgtca

SEQ ID NO: 68 - protein, 119 aa, synthetic construct
  REGION 1..119: note = Description of Artificial Sequence: Synthetic polypeptide
QVTLKESGPALVKPTQTLTLTCTFSGFSLNTYGMGVSWIRQPPGKALEWLAHIYWDDDKRYNPSLKTRLTISKDTSKNQVVLTITNVDPVDTAVYYCAQRGYDDYWGYWGQGTLVTISS

SEQ ID NO: 69 - DNA, 357 bp, synthetic construct
  misc_feature 1..357: note = Description of Artificial Sequence: Synthetic polynucleotide
  source 1..357: mol_type = other DNA
caagtaacgctcaaggagtccggacccaccttggtgaagccgacgcagaccttgactcttacgtgcactttctcggggttttcactgaatacgtacgggatgggtgtctcatggatcaggcaacctccggggaaaggattggaatggctggcgcacatctactgggatgacgataagagatataacccaagcctcaagtcgcggctcaccattacaaaagatacatcgaaaaatcaggtcgtacttactatcacgaacatggaccccgtggacacagcaacatattactgtgcccagcgcggctatgacgattattggggttactggggacagggaacactggtcacggtgtccagc

SEQ ID NO: 70 - protein, 119 aa, synthetic construct
  REGION 1..119: note = Description of Artificial Sequence: Synthetic polypeptide
QVTLKESGPTLVKPTQTLTLTCTFSGFSLNTYGMGVSWIRQPPGKGLEWLAHIYWDDDKRYNPSLKSRLTITKDTSKNQVVLTITNMDPVDTATYYCAQRGYDDYWGYWGQGTLVTVSS

SEQ ID NO: 71 - DNA, 357 bp, synthetic construct
  misc_feature 1..357: note = Description of Artificial Sequence: Synthetic polynucleotide
  source 1..357: mol_type = other DNA
cagatcactttgaaagaaagcggaccgaccttggtcaagcccacacaaaccctcacgctcacgtgtacattttcggggttctcgctttcaacttacgggatgggagtagggtggattcgccagccgcctggtaaagcgttggagtggcttgcagacatctggtgggacgacgataagtactataatccctcgctcaagtccagactgaccatcacgaaagatacgagcaagaaccaggtcgtgctgacaatgactaacatggacccagtggatacggctacatattactgcgccaggcggggtcactactcagcgatggattattggggccagggaacactggtaacggtgtcgtcc

SEQ ID NO: 72 - protein, 119 aa, synthetic construct
  REGION 1..119: note = Description of Artificial Sequence: Synthetic polypeptide
QITLKESGPTLVKPTQTLTLTCTFSGFSLSTYGMGVGWIRQPPGKALEWLADIWWDDDKYYNPSLKSRLTITKDTSKNQVVLTMTNMDPVDTATYYCARRGHYSAMDYWGQGTLVTVSS

SEQ ID NO: 73 - DNA, 357 bp, synthetic construct
  misc_feature 1..357: note = Description of Artificial Sequence: Synthetic polynucleotide
  source 1..357: mol_type = other DNA
caagtgactctcaaggagtccggacccgccctggtcaaaccaacgcagacactgacgctcacatgcaccttcagcggattttcgttgtcaacgtacggcatgggtgtggggtggattcgccagcctccggggaaagcccttgaatggttggcggacatctggtgggatgatgacaagtactataatccctcacttaagtcacggttgacgatctcgaaagacaccagcaagaaccaggtagtgctgacaatgactaacatggacccggtcgatacagcggtctactattgtgctagaaggggacactactccgcaatggattattggggtcaggggacgctcgtaaccgtgtcgtcg

SEQ ID NO: 74 - protein, 119 aa, synthetic construct
  REGION 1..119: note = Description of Artificial Sequence: Synthetic polypeptide
QVTLKESGPALVKPTQTLTLTCTFSGFSLSTYGMGVGWIRQPPGKALEWLADIWWDDDKYYNPSLKSRLTISKDTSKNQVVLTMTNMDPVDTAVYYCARRGHYSAMDYWGQGTLVTVSS

SEQ ID NO: 75 - DNA, 321 bp, synthetic construct
  misc_feature 1..321: note = Description of Artificial Sequence: Synthetic polynucleotide
  source 1..321: mol_type = other DNA
gacatccagatgactcagtctccagcctccctatctgcatctgtgggagaaactgtcaccatcacatgtcgaacaagtgagaatcttcacaattatttagcatggtatcagcagaaacagggaaaatctcctcagctcctggtctatgatgcaaaaaccttagcagatggtgtgccatcaaggttcagtggcagtggatcaggaacacaatattctctcaagatcaacagcctgcagcctgaagattttgggagttattactgtcaacatttttggagtagtccttacacgttcggaggggggaccaagctggaaataaaa

SEQ ID NO: 76 - protein, 107 aa, synthetic construct
  REGION 1..107: note = Description of Artificial Sequence: Synthetic polypeptide
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIK

SEQ ID NO: 77 - DNA, 333 bp, synthetic construct
  misc_feature 1..333: note = Description of Artificial Sequence: Synthetic polynucleotide
  source 1..333: mol_type = other DNA
gacattgtgttgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggctccggggtccctgccaggtttagtggcagtgggtctgggacagacttcagcctcaacatccatcctatggaggaggatgatactgcaatgtatttctgtcagcaaagtaaggaggttccgtggacgttcggtggaggctccaagctggaaatcaaa

SEQ ID NO: 78 - protein, 111 aa, synthetic construct
  REGION 1..111: note = Description of Artificial Sequence: Synthetic polypeptide
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGSKLEIK

SEQ ID NO: 79 - DNA, 321 bp, synthetic construct
  misc_feature 1..321: note = Description of Artificial Sequence: Synthetic polynucleotide
  source 1..321: mol_type = other DNA
gacattgtgatgacccagtctcaaaaattcatgtccacatcagtaggagacagggtcagcgtcacctgcaaggccagtcagaatgtgggtactaatgtagcctggtatcaacagaaattaggacaatctcctaaaacactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaa

SEQ ID NO: 80 - protein, 107 aa, synthetic construct
  REGION 1..107: note = Description of Artificial Sequence: Synthetic polypeptide
DIVMTQSQKFMSTSVGDRVSVTCKASQNVGTNVAWYQQKLGQSPKTLIYSASYRYSGVPDRFTGSGSGTDFTLTISNVQSEDLAEYFCQQYNSYPYTFGGGTKLEIK

SEQ ID NO: 81 - DNA, 321 bp, synthetic construct
  misc_feature 1..321: note = Description of Artificial Sequence: Synthetic polynucleotide
  source 1..321: mol_type = other DNA
gacattgtgatgacccagtctcaaaaattcatgtccacatcagtaggagacagggtcagcgtcacctgcaaggccagtcagaatgtgggtactaatgtagcctggtttcaacagaaaccaggtcaatctcctaaagcactgatttactcggcatcttaccggtacagtggagtccctgatcgcttcacaggcagtggatctgggacagatttcattctcaccatcagcaatgtgcagtctgaagacctggcagagtatttctgtcagcaatataacaactatcctctcacgttcggtgctgggaccaagctggagctgaaa

SEQ ID NO: 82 - protein, 107 aa, synthetic construct
  REGION 1..107: note = Description of Artificial Sequence: Synthetic polypeptide
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELK

SEQ ID NO: 83 - DNA, 321 bp, synthetic construct
  misc_feature 1..321: note = Description of Artificial Sequence: Synthetic polynucleotide
  source 1..321: mol_type = other DNA
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagttctccttacacgttcggaggggggaccaagctggaaataaaa

SEQ ID NO: 84 - protein, 107 aa, synthetic construct
  REGION 1..107: note = Description of Artificial Sequence: Synthetic polypeptide
DIQMTQSPASLSASVGETVTITCRASGNIHNYLAWYQQKQGKSPQLLVYNAKTLADGVPSRFSGSGSGTQYSLKINSLQPEDFGSYYCQHFWSSPYTFGGGTKLEIK

SEQ ID NO: 85 - DNA, 321 bp, synthetic construct
  misc_feature 1..321: note = Description of Artificial Sequence: Synthetic polynucleotide
  source 1..321: mol_type = other DNA
gacattgtaatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcgccatcctaccggtacagtggagtccctgatcgcttcacaggcagtggatctgggacagatttcactctcaccatcagcaatgtgcagtctgaagacttggcagaatatttctgtcagcaatataacagctatcctcacacgttcggaggggggaccaagctggaaatgaaa

SEQ ID NO: 86 - protein, 107 aa, synthetic construct
  REGION 1..107: note = Description of Artificial Sequence: Synthetic polypeptide
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMK

SEQ ID NO: 87 - DNA, 333 bp, synthetic construct
  misc_feature 1..333: note = Description of Artificial Sequence: Synthetic polynucleotide
  source 1..333: mol_type = other DNA
gacattgtgctgacacagtctcctgcttccttagctgtatctctggggcagagggccaccatctcatgcagggccagccaaagtgtcagtacatctaggtttagttatatgcactggttccaacagaaaccaggacaggcacccaaactcctcatcaagtatgcatccaacctagaatctggggtccctgccaggttcagtggcagtgggtctgggacagacttcaccctcaacatccatcctgtggagggggaggatactgcaacatattactgtcagcacagttgggagattccgtacacgttcggaggggggaccaagctggaaataaaa

SEQ ID NO: 88 - protein, 111 aa, synthetic construct
  REGION 1..111: note = Description of Artificial Sequence: Synthetic polypeptide
DIVLTQSPASLAVSLGQRATISCRASQSVSTSRFSYMHWFQQKPGQAPKLLIKYASNLESGVPARFSGSGSGTDFTLNIHPVEGEDTATYYCQHSWEIPYTFGGGTKLEIK

SEQ ID NO: 89 - DNA, 321 bp, synthetic construct
  misc_feature 1..321: note = Description of Artificial Sequence: Synthetic polynucleotide
  source 1..321: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttgtctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaag

SEQ ID NO: 90 - protein, 107 aa, synthetic construct
  REGION 1..107: note = Description of Artificial Sequence: Synthetic polypeptide
DIQMTQSPSSLSASVGDRVTITCRTSENLHNYLAWYQQKPGKSPKLLVYDAKTLADGVPSRFSGSGSGTDYTLTISSLQPEDFATYYCQHFWSSPYTFGQGTKLEIK

SEQ ID NO: 91 - DNA, 321 bp, synthetic construct
  misc_feature 1..321: note = Description of Artificial Sequence: Synthetic polynucleotide
  source 1..321: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaaggccccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaag

SEQ ID NO: 92 - protein, 107 aa, synthetic construct
  REGION 1..107: note = Description of Artificial Sequence: Synthetic polypeptide
DIQMTQSPSSLSASVGDRVTITCRTSENLHNYLAWYQQKPGKAPKLLIYDAKTLADGVPSRFSGSGSGTDYTLTISSLQPEDFATYYCQHFWSSPYTFGQGTKLEIK

SEQ ID NO: 93 - DNA, 321 bp, synthetic construct
  misc_feature 1..321: note = Description of Artificial Sequence: Synthetic polynucleotide
  source 1..321: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaag

SEQ ID NO: 94 - protein, 107 aa, synthetic construct
  REGION 1..107: note = Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSSPYTFGQGTKLEIK

SEQ ID NO: 95 - DNA, 321 bp, synthetic construct
  misc_feature 1..321: note = Description of Artificial Sequence: Synthetic polynucleotide
  source 1..321: mol_type = other DNA
gacatccaaatgacccaatcgccctcctccctctccgcatcagtaggggaccgcgtcacaattacttgcaaagcgtcgcagaacgtcggaacgaatgtggcgtggtttcagcagaagcccggaaaagctccgaagagcttgatctactcggcctcatataggtattcgggtgtgccgagccggtttagcgggtcggggtcaggtactgatttcacgctcacaatttcatcgttgcagccagaagatttcgccacatattactgtcagcagtacaacaattaccctctgacgttcggccagggaaccaaacttgagatcaag

SEQ ID NO: 96 - protein, 107 aa, synthetic construct
  REGION 1..107: note = Description of Artificial Sequence: Synthetic polypeptide
DIQMTQSPSSLSASVGDRVTITCKASQNVGTNVAWFQQKPGKAPKSLIYSASYRYSGVPSRFSGSGSGTDFTLTISSLQPEDFATYYCQQYNNYPLTFGQGTKLEIK

SEQ ID NO: 97 - DNA, 321 bp, synthetic construct
  misc_feature 1..321: note = Description of Artificial Sequence: Synthetic polynucleotide
  source 1..321: mol_type = other DNA
gatatccagatgacacagtcaccctcgtcgctctcagcttccgtaggcgacagggtcactattacgtgtaaagcatcacagaacgtcggaacgaatgtggcgtggtttcagcagaagcccgggaagagccccaaagcgcttatctactccccgtcgtatcggtattccggtgtgccaagcagattttcggggtcaggttcgggaactgactttaccctgaccatctcgtccctccaaccggaagatttcgccacgtacttctgccagcagtacaacagctatcctcacacattcggacaagggacaaagttggagattaaa

SEQ ID NO: 98 - protein, 107 aa, synthetic construct
  REGION 1..107: note = Description of Artificial Sequence: Synthetic polypeptide
DIQMTQSPSSLSASVGDRVTITCKASQNVGTNVAWFQQKPGKSPKALIYSPSYRYSGVPSRFSGSGSGTDFTLTISSLQPEDFATYFCQQYNSYPHTFGQGTKLEIK

SEQ ID NO: 99 - DNA, 1332 bp, synthetic construct
  misc_feature 1..1332: note = Description of Artificial Sequence: Synthetic polynucleotide
  source 1..1332: mol_type = other DNA
gaggtcctgctgcaacagtctggacctgagctggtgaagcctggggcttcagtgaagataccctgcaaggcttctggatacacattcactgactacaacatggactgggtgaagcagagccatggaaagagccttgagtggattggacaaattaatcctaacaatggtggtattttcttcaaccagaagttcaagggcaaggccacattgactgtagacaagtcctccaatacagccttcatggaggtccgcagcctgacatctgaggacactgcagtctattactgtgcaagagaggcaattactacggtaggcgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 100 - protein, 444 aa, synthetic construct
  REGION 1..444: note = Description of Artificial Sequence: Synthetic polypeptide
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 101 - DNA, 642 bp, synthetic construct
  misc_feature 1..642: note = Description of Artificial Sequence: Synthetic polynucleotide
  source 1..642: mol_type = other DNA
gacatccagatgactcagtctccagcctccctatctgcatctgtgggagaaactgtcaccatcacatgtcgaacaagtgagaatcttcacaattatttagcatggtatcagcagaaacagggaaaatctcctcagctcctggtctatgatgcaaaaaccttagcagatggtgtgccatcaaggttcagtggcagtggatcaggaacacaatattctctcaagatcaacagcctgcagcctgaagattttgggagttattactgtcaacatttttggagtagtccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 102 - protein, 214 aa, synthetic construct
  REGION 1..214: note = Description of Artificial Sequence: Synthetic polypeptide
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 103 - DNA, 1326 bp, synthetic construct
  misc_feature 1..1326: note = Description of Artificial Sequence: Synthetic polynucleotide
  source 1..1326: mol_type = other DNA
caggtccaactgcagcagcctggggctgaactggtgaagcctggggcttcagtgaagctgtcctgcaaggcttctggctacaccttcaccagctactggattcactgggtgaaccagaggcctggacaaggccttgagtggattggagacattaatcctagcaacggccgtagtaagtataatgagaagttcaagaacaaggccacaatgactgcagacaaatcctccaacacagcctacatgcaactcagcagcctgacatctgaggactctgcggtctattactgtgcaagagaggttctggatggtgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 104 - protein, 442 aa, synthetic construct
  REGION 1..442: note = Description of Artificial Sequence: Synthetic polypeptide
QVQLQQPGAELVKPGASVKLSCKASGYTFTSYWIHWVNQRPGQGLEWIGDINPSNGRSKYNEKFKNKATMTADKSSNTAYMQLSSLTSEDSAVYYCAREVLDGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 105 - DNA, 654 bp, synthetic construct
  misc_feature 1..654: note = Description of Artificial Sequence: Synthetic polynucleotide
  source 1..654: mol_type = other DNA
gacattgtgttgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggctccggggtccctgccaggtttagtggcagtgggtctgggacagacttcagcctcaacatccatcctatggaggaggatgatactgcaatgtatttctgtcagcaaagtaaggaggttccgtggacgttcggtggaggctccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 106 - protein, 218 aa, synthetic construct
  REGION 1..218: note = Description of Artificial Sequence: Synthetic polypeptide
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGSKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 107 - DNA, 1329 bp, synthetic construct
  misc_feature 1..1329: note = Description of Artificial Sequence: Synthetic polynucleotide
  source 1..1329: mol_type = other DNA
caggttactctgaaagagtctggccctgggatattgcagccctcccagaccctcagtctgacttgttctttctctgggttttcactgagcacttatggtatgggtgtgacctggattcgtcagccttcaggaaagggtctggagtggctggcacacatttactgggatgatgacaagcgctataacccatccctgaagagccggctcacaatctccaaggatacctccaacaaccaggtattcctcaagatcaccagtgtggacactgcagatactgccacatactactgtgctcaaacggggtatagtaacttgtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 108 - protein, 443 aa, synthetic construct
  REGION 1..443: note = Description of Artificial Sequence: Synthetic polypeptide
QVTLKESGPGILQPSQTLSLTCSFSGFSLSTYGMGVTWIRQPSGKGLEWLAHIYWDDDKRYNPSLKSRLTISKDTSNNQVFLKITSVDTADTATYYCAQTGYSNLFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 109 - DNA, 642 bp, synthetic construct
  misc_feature 1..642: note = Description of Artificial Sequence: Synthetic polynucleotide
  source 1..642: mol_type = other DNA
gacattgtgatgacccagtctcaaaaattcatgtccacatcagtaggagacagggtcagcgtcacctgcaaggccagtcagaatgtgggtactaatgtagcctggtatcaacagaaattaggacaatctcctaaaacactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 110 - protein, 214 aa, synthetic construct
  REGION 1..214: note = Description of Artificial Sequence: Synthetic polypeptide
DIVMTQSQKFMSTSVGDRVSVTCKASQNVGTNVAWYQQKLGQSPKTLIYSASYRYSGVPDRFTGSGSGTDFTLTISNVQSEDLAEYFCQQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 111 - DNA, 1329 bp, synthetic construct
  misc_feature 1..1329: note = Description of Artificial Sequence: Synthetic polynucleotide
  source 1..1329: mol_type = other DNA
caggttactctgaaagagtctggccctgggatattgcagccctcccagaccctcagtctgacttgttctttctctgggttttcactgaacacttatggtatgggtgtgagctggattcgtcagccttcaggaaagggtctggagtggctggcacacatttactgggatgatgacaagcgctataacccatccctgaagagccggctcacaatctccaaggatgcctccaacaaccgggtcttcctcaagatcaccagtgtggacactgcagatactgccacatactactgtgctcaaagaggttatgatgattactggggttactggggccaagggactctggtcactatctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 112 - protein, 443 aa, synthetic construct
  REGION 1..443: note = Description of Artificial Sequence: Synthetic polypeptide
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 113 - DNA, 642 bp, synthetic construct
  misc_feature 1..642: note = Description of Artificial Sequence: Synthetic polynucleotide
  source 1..642: mol_type = other DNA
gacattgtgatgacccagtctcaaaaattcatgtccacatcagtaggagacagggtcagcgtcacctgcaaggccagtcagaatgtgggtactaatgtagcctggtttcaacagaaaccaggtcaatctcctaaagcactgatttactcggcatcttaccggtacagtggagtccctgatcgcttcacaggcagtggatctgggacagatttcattctcaccatcagcaatgtgcagtctgaagacctggcagagtatttctgtcagcaatataacaactatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 114 - protein, 214 aa, synthetic construct
  REGION 1..214: note = Description of Artificial Sequence: Synthetic polypeptide
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 115 - DNA, 1368 bp, synthetic construct
  misc_feature 1..1368: note = Description of Artificial Sequence: Synthetic polynucleotide
  source 1..1368: mol_type = other DNA
gaggtcctgctgcaacagtctggacctgaggtggtgaagcctggggcttcagtgaagataccctgcaaggcttctggatacacattcactgactacaacatggactgggtgaagcagagccatggaaagagccttgagtggattggagagattaatcctaacaatggtggtactttctacaaccagaagttcaagggcaaggccacattgactgtagacaagtcctccagcacagcctacatggagctccgcagcctgacatctgaggacactgcagtctattactgtgcaagagaggcaattactacggtaggcgctatggactactggggtcaaggaacctcagtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctagactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 116 - protein, 456 aa, synthetic construct
  REGION 1..456: note = Description of Artificial Sequence: Synthetic polypeptide
EVLLQQSGPEVVKPGASVKIPCKASGYTFTDYNMDWVKQSHGKSLEWIGEINPNNGGTFYNQKFKGKATLTVDKSSSTAYMELRSLTSEDTAVYYCAREAITTVGAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 117 - DNA, 642 bp, synthetic construct
  misc_feature 1..642: note = Description of Artificial Sequence: Synthetic polynucleotide
  source 1..642: mol_type = other DNA
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagttctccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 118 - protein, 214 aa, synthetic construct
  REGION 1..214: note = Description of Artificial Sequence: Synthetic polypeptide
DIQMTQSPASLSASVGETVTITCRASGNIHNYLAWYQQKQGKSPQLLVYNAKTLADGVPSRFSGSGSGTQYSLKINSLQPEDFGSYYCQHFWSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 119 - DNA, 1329 bp, synthetic construct
  misc_feature 1..1329: note = Description of Artificial Sequence: Synthetic polynucleotide
  source 1..1329: mol_type = other DNA
caggttactctgaaagagtctggccctggaatattgcagccctcccagaccctcagtctgacttgttctttctctgggttttcactgagcacttatggtatgggtgtaggctggattcgtcagccttcaggaaagggtctagagtggctggcagacatttggtgggatgacgataagtactataacccatccctgaagagccggctcacaatctccaaggatacctccagcaatgaggtattcctcaagatcgccattgtggacactgcagatactgccacttactactgtgctcgaagaggtcactactctgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 120 - protein, 443 aa, synthetic construct
  REGION 1..443: note = Description of Artificial Sequence: Synthetic polypeptide
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 121 - DNA, 642 bp, synthetic construct
  misc_feature 1..642: note = Description of Artificial Sequence: Synthetic polynucleotide
  source 1..642: mol_type = other DNA
gacattgtaatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcgccatcctaccggtacagtggagtccctgatcgcttcacaggcagtggatctgggacagatttcactctcaccatcagcaatgtgcagtctgaagacttggcagaatatttctgtcagcaatataacagctatcctcacacgttcggaggggggaccaagctggaaatgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 122 - protein, 214 aa, synthetic construct
  REGION 1..214: note = Description of Artificial Sequence: Synthetic polypeptide
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 123 - DNA, 1335 bp, synthetic construct
  misc_feature 1..1335: note = Description of Artificial Sequence: Synthetic polynucleotide
  source 1..1335: mol_type = other DNA
caggttactctgaaagagtctggccctgggatattgcagccctcccagaccctcagtctgacttgttctttctctgggttttcactgagcacttctggtatgggtgtgagttggattcgtcagccttcaggaaagggtctggagtggctggcacacaatgactgggatgatgacaagcgctataagtcatccctgaagagccggctcacaatatccaaggatacctccagaaaccaggtattcctcaagatcaccagtgtggacactgcagatactgccacatactactgtgctcgaagagttgggggattagagggctattttgattactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 124 - protein, 445 aa, synthetic construct
  REGION 1..445: note = Description of Artificial Sequence: Synthetic polypeptide
QVTLKESGPGILQPSQTLSLTCSFSGFSLSTSGMGVSWIRQPSGKGLEWLAHNDWDDDKRYKSSLKSRLTISKDTSRNQVFLKITSVDTADTATYYCARRVGGLEGYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 125 - DNA, 654 bp, synthetic construct
  misc_feature 1..654: note = Description of Artificial Sequence: Synthetic polynucleotide
  source 1..654: mol_type = other DNA
gacattgtgctgacacagtctcctgcttccttagctgtatctctggggcagagggccaccatctcatgcagggccagccaaagtgtcagtacatctaggtttagttatatgcactggttccaacagaaaccaggacaggcacccaaactcctcatcaagtatgcatccaacctagaatctggggtccctgccaggttcagtggcagtgggtctgggacagacttcaccctcaacatccatcctgtggagggggaggatactgcaacatattactgtcagcacagttgggagattccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 126 - protein, 218 aa, synthetic construct
  REGION 1..218: note = Description of Artificial Sequence: Synthetic polypeptide
DIVLTQSPASLAVSLGQRATISCRASQSVSTSRFSYMHWFQQKPGQAPKLLIKYASNLESGVPARFSGSGSGTDFTLNIHPVEGEDTATYYCQHSWEIPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 127 - DNA, 360 bp, synthetic construct
  misc_feature 1..360: note = Description of Artificial Sequence: Synthetic polynucleotide
  source 1..360: mol_type = other DNA
gaagtgttgttgcagcagtcagggccggagttggtaaaaccgggagcgtcggtgaaaatcccgtgcaaagcgtcggggtatacgtttacggactataacatggattgggtgaaacagtcgcatgggaaatcgcttgaatggattggtcagatcaatccgaataatggaggaatcttctttaatcagaagtttaaaggaaaagcgacgcttacagtcgataagtcgtcgaacacggcgttcatggaagtacggtcgcttacgtcggaagatacggcggtctattactgtgcgagggaggcgattacgacggtgggagcgatggactattggggacaagggacgtcggtcacggtatcgtcg

SEQ ID NO: 128 - protein, 7 aa, synthetic construct
  REGION 1..7: note = Description of Artificial Sequence: Synthetic peptide
GYTFTSY

SEQ ID NO: 129 - DNA, 357 bp, synthetic construct
  misc_feature 1..357: note = Description of Artificial Sequence: Synthetic polynucleotide
  source 1..357: mol_type = other DNA
caggtgacactcaaagaatcaggacccggaatccttcagcccagccagaccttgtcgctgacttgttcgttctccggtttcagcctgaatacttatgggatgggtgtgtcatggatcaggcaaccgtccgggaaaggattggagtggctcgcgcacatctactgggacgatgacaaacgctacaatccttcgctgaagagccgattgacgatttccaaggatgcctcgaacaaccgggtatttcttaagatcacgtcggtcgatacggcagacacggcgacctattactgcgcccaaagagggtacgatgactattggggatattggggccaggggacactcgtcacaatttcagct

SEQ ID NO: 130 - protein, 9 aa, synthetic construct
  REGION 1..9: note = Description of Artificial Sequence: Synthetic peptide
GFSLSTYGM

SEQ ID NO: 131 - DNA, 357 bp, synthetic construct
  misc_feature 1..357: note = Description of Artificial Sequence: Synthetic polynucleotide
  source 1..357: mol_type = other DNA
caggtcacgctgaaagagtcaggtcccggaatccttcaaccttcgcagacattgtcactcacatgttccttctccgggttctcgctctcgacttatggcatgggtgtaggatggattcggcagcccagcgggaaggggcttgagtggttggcggatatctggtgggacgacgacaaatactacaatccgagcctgaagtcccgcctcaccatttcgaaagatacgtcatcaaacgaagtctttttgaagatcgccatcgtggacacggcggatacagcgacgtattactgcgccagaaggggacactacagcgcaatggattattggggacaggggacctcggtgactgtgtcgtcc

SEQ ID NO: 132 - protein, 9 aa, synthetic construct
  REGION 1..9: note = Description of Artificial Sequence: Synthetic peptide
GFSLNTYGM

SEQ ID NO: 133 - DNA, 321 bp, synthetic construct
  misc_feature 1..321: note = Description of Artificial Sequence: Synthetic polynucleotide
  source 1..321: mol_type = other DNA
gacatccaaatgacccagtcacccgcgagcctttcggcgtcggtcggagaaacggtcacgatcacgtgccggacatcagagaatctccataactacctcgcgtggtatcaacagaagcaggggaagtcgccccagttgcttgtatacgatgcgaaaacgttggcggatggggtgccgtccagattctcgggatcgggctcggggacgcagtactcgctcaagatcaattcgctgcagccggaggactttgggtcgtactattgtcagcatttttggtcatcaccgtatacatttggaggtggaacgaaacttgagattaag

SEQ ID NO: 134 - protein, 9 aa, synthetic construct
  REGION 1..9: note = Description of Artificial Sequence: Synthetic peptide
GFSLSTSGM

SEQ ID NO: 135 - DNA, 321 bp, synthetic construct
  misc_feature 1..321: note = Description of Artificial Sequence: Synthetic polynucleotide
  source 1..321: mol_type = other DNA
gatatcgtcatgacccagtcccagaagttcatgtcaacttcagtgggagacagagtgtccgtcacatgtaaagcctcgcaaaatgtgggaaccaacgtagcgtggttccagcagaaacctggccaatcaccgaaggcactgatctactcggccagctataggtactcgggagtaccagatcggtttacggggtcggggagcgggacggactttatcctcactatttccaatgtccagtcggaggaccttgcggaatacttctgccagcagtataacaactatcccctcacgtttggtgctggtacaaaattggagttgaag

SEQ ID NO: 136 - protein, 8 aa, synthetic construct
  REGION 1..8: note = Description of Artificial Sequence: Synthetic peptide
GYTFTDYN

SEQ ID NO: 137 - DNA, 321 bp, synthetic construct
  misc_feature 1..321: note = Description of Artificial Sequence: Synthetic polynucleotide
  source 1..321: mol_type = other DNA
gacatcgtgatgacacagtcacagaaattcatgtccacatccgtcggtgatagagtatccgtcacgtgtaaggcctcgcaaaacgtaggaactaatgtggcgtggtatcaacagaagccaggacagtcacccaaagcactcatctacagcccctcatatcggtacagcggggtgccggacaggttcacgggatcggggagcgggaccgattttacactgaccatttcgaatgtccagtcggaggaccttgcggaatacttctgccagcagtataactcgtaccctcacacgtttggaggtggcactaagttggagatgaaa

SEQ ID NO: 138 - protein, 8 aa, synthetic construct
  REGION 1..8: note = Description of Artificial Sequence: Synthetic peptide
GYTFTSYW

SEQ ID NO: 139 - protein, 5 aa, synthetic construct
  REGION 1..5: note = Description of Artificial Sequence: Synthetic peptide
GGGGS

SEQ ID NO: 140 - protein, 10 aa, synthetic construct
  REGION 1..10: note = Description of Artificial Sequence: Synthetic peptide
GFSLSTYGMG

SEQ ID NO: 141 - protein, 10 aa, synthetic construct
  REGION 1..10: note = Description of Artificial Sequence: Synthetic peptide
GFSLNTYGMG

SEQ ID NO: 142 - protein, 10 aa, synthetic construct
  REGION 1..10: note = Description of Artificial Sequence: Synthetic peptide
GFSLSTSGMG

SEQ ID NO: 143 - protein, 6 aa, synthetic construct
  REGION 1..6: note = Description of Artificial Sequence: Synthetic peptide
NPNNGG

SEQ ID NO: 144 - protein, 6 aa, synthetic construct
  REGION 1..6: note = Description of Artificial Sequence: Synthetic peptide
NPSNGR

SEQ ID NO: 145 - protein, 5 aa, synthetic construct
  REGION 1..5: note = Description of Artificial Sequence: Synthetic peptide
YWDDD

SEQ ID NO: 146 - protein, 5 aa, synthetic construct
  REGION 1..5: note = Description of Artificial Sequence: Synthetic peptide
WWDDD

SEQ ID NO: 147 - protein, 5 aa, synthetic construct
  REGION 1..5: note = Description of Artificial Sequence: Synthetic peptide
DWDDD

SEQ ID NO: 148 - protein, 8 aa, synthetic construct
  REGION 1..8: note = Description of Artificial Sequence: Synthetic peptide
INPNNGGI

SEQ ID NO: 149 - protein, 8 aa, synthetic construct
  REGION 1..8: note = Description of Artificial Sequence: Synthetic peptide
INPSNGRS

SEQ ID NO: 150 - protein, 7 aa, synthetic construct
  REGION 1..7: note = Description of Artificial Sequence: Synthetic peptide
IYWDDDK

SEQ ID NO: 151 - protein, 8 aa, synthetic construct
  REGION 1..8: note = Description of Artificial Sequence: Synthetic peptide
INPNNGGT

SEQ ID NO: 152 - protein, 7 aa, synthetic construct
  REGION 1..7: note = Description of Artificial Sequence: Synthetic peptide
IWWDDDK

SEQ ID NO: 153 - protein, 7 aa, synthetic construct
  REGION 1..7: note = Description of Artificial Sequence: Synthetic peptide
NDWDDDK

SEQ ID NO: 154 - protein, 13 aa, synthetic construct
  REGION 1..13: note = Description of Artificial Sequence: Synthetic peptide
AREAITTVGAMDY

SEQ ID NO: 155 - protein, 11 aa, synthetic construct
  REGION 1..11: note = Description of Artificial Sequence: Synthetic peptide
AREVLDGAMDY

SEQ ID NO: 156 - protein, 11 aa, synthetic construct
  REGION 1..11: note = Description of Artificial Sequence: Synthetic peptide
AQTGYSNLFAY

SEQ ID NO: 157 - protein, 11 aa, synthetic construct
  REGION 1..11: note = Description of Artificial Sequence: Synthetic peptide
AQRGYDDYWGY

SEQ ID NO: 158 - protein, 11 aa, synthetic construct
  REGION 1..11: note = Description of Artificial Sequence: Synthetic peptide
ARRGHYSAMDY

SEQ ID NO: 159 - protein, 13 aa, synthetic construct
  REGION 1..13: note = Description of Artificial Sequence: Synthetic peptide
ARRVGGLEGYFDY

SEQ ID NO: 160 - protein, 6 aa, synthetic construct
  REGION 1..6: note = Description of Artificial Sequence: Synthetic peptide
ENLHNY

SEQ ID NO: 161 - protein, 10 aa, synthetic construct
  REGION 1..10: note = Description of Artificial Sequence: Synthetic peptide
ESVDNYGISF

SEQ ID NO: 162 - protein, 6 aa, synthetic construct
  REGION 1..6: note = Description of Artificial Sequence: Synthetic peptide
QNVGTN

SEQ ID NO: 163 - protein, 6 aa, synthetic construct
  REGION 1..6: note = Description of Artificial Sequence: Synthetic peptide
GNIHNY

SEQ ID NO: 164 - protein, 10 aa, synthetic construct
  REGION 1..10: note = Description of Artificial Sequence: Synthetic peptide
QSVSTSRFSY

SEQ ID NO: 165 - DNA, 972 bp, Mus sp.
  source 1..972: mol_type = genomic DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 166 - protein, 324 aa, Mus sp.
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 167 - DNA, 1008 bp, Mus sp.
  source 1..1008: mol_type = genomic DNA
gccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctagactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 168 - protein, 336 aa, Mus sp.
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 169 - DNA, 321 bp, Mus sp.
  source 1..321: mol_type = genomic DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 170 - protein, 107 aa, Mus sp.
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 171 - DNA, 990 bp, Homo sapiens
  source 1..990: mol_type = genomic DNA
gcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 172 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - DNA, 321 bp, Homo sapiens
  source 1..321: mol_type = genomic DNA
cgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 174 - protein, 107 aa, Homo sapiens [sequence omitted]

SEQ ID NO: 175 - DNA, 1350 bp, synthetic construct
  misc_feature 1..1350: note = Description of Artificial Sequence: Synthetic polynucleotide
  source 1..1350: mol_type = other DNA
gaagtgttgttgcagcagtcagggccggagttggtaaaaccgggagcgtcggtgaaaatcccgtgcaaagcgtcggggtatacgtttacggactataacatggattgggtgaaacagtcgcatgggaaatcgcttgaatggattggtcagatcaatccgaataatggaggaatcttctttaatcagaagtttaaaggaaaagcgacgcttacagtcgataagtcgtcgaacacggcgttcatggaagtacggtcgcttacgtcggaagatacggcggtctattactgtgcgagggaggcgattacgacggtgggagcgatggactattggggacaagggacgtcggtcacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 176 - protein, 450 aa, synthetic construct
  REGION 1..450: note = Description of Artificial Sequence: Synthetic polypeptide
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - DNA, 1350 bp, synthetic construct
  misc_feature 1..1350: note = Description of Artificial Sequence: Synthetic polynucleotide
  source 1..1350: mol_type = other DNA
caagtgcaacttgtgcagtcgggtgcggaagtcaaaaagccgggagcgtcggtgaaagtatcgtgtaaagcgtcgggatatacgtttacggactataacatggactgggtacgacaggcaccggggaaatcgttggaatggatcggacagattaatccgaacaatgggggaattttctttaatcagaaattcaaaggacgggcgacgttgacggtcgatacatcgacgaatacggcgtatatggaattgaggtcgcttcgctcggacgatacggcggtctattactgcgccagggaggcgatcacgacggtaggggcgatggattattggggacaggggacgcttgtgacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 178 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGKSLEWIGQINPNNGGIFFNQKFKGRATLTVDTSTNTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - DNA, 1350 bp, synthetic construct
  misc_feature 1..1350: note = Description of Artificial Sequence: Synthetic polynucleotide
  source 1..1350: mol_type = other DNA
caagtccagcttgtccagtcgggagcggaagtgaagaaaccggggtcgtcggtcaaagtatcgtgtaaagcgtcgggatatacgtttacggactataacatggattgggtacgacaggctccgggaaaatcattggaatggattggacagattaatccgaataatgggggtatcttctttaatcaaaagtttaaagggagggcgacgttgacggtggacaaatcgacaaatacggcgtatatggaattgtcgtcgcttcggtcggaggacacggcggtgtattactgcgcgagggaggcgatcacgacggtcggggcgatggattattggggacagggaacgcttgtgacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 180 - protein, 450 aa, synthetic construct
  REGION 1..450: note = Description of Artificial Sequence: Synthetic polypeptide
QVQLVQSGAEVKKPGSSVKVSCKASGYTFTDYNMDWVRQAPGKSLEWIGQINPNNGGIFFNQKFKGRATLTVDKSTNTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1350 bp, synthetic construct
  misc_feature 1..1350: note = Description of Artificial Sequence: Synthetic polynucleotide
  source 1..1350: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagggtcttgaatggatggggcagattaatccgaataatggagggatcttctttaatcagaaattcaaaggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 182 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGQGLEWMGQINPNNGGIFFNQKFKGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - DNA, 1350 bp, synthetic construct
  misc_feature 1..1350: note = Description of Artificial Sequence: Synthetic polynucleotide
  source 1..1350: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgaataatggagggatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 184 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGQSLEWMGQINPNNGGIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - DNA, 1350 bp, synthetic construct
  misc_feature 1..1350: note = Description of Artificial Sequence: Synthetic polynucleotide
  source 1..1350: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagggtcttgaatggatggggcagattaatccgaataatggagggatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 186 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGQGLEWMGQINPNNGGIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - DNA, 1350 bp, synthetic construct
  misc_feature 1..1350: note = Description of Artificial Sequence: Synthetic polynucleotide
  source 1..1350: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggggaatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 188 - protein, 450 aa, synthetic construct
  REGION 1..450: note = Description of Artificial Sequence: Synthetic polypeptide
QVQLVQSGAEVKKPGSSVKVSCKASGYTFSDYNMDWVRQAPGQGLEWMGQINPNNGGIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 1350 bp, synthetic construct
  misc_feature 1..1350: note = Description of Artificial Sequence: Synthetic polynucleotide
  source 1..1350: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggggaatctttttcaatcagaagtttcaggggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 190 - protein, 450 aa, synthetic construct
  REGION 1..450: note = Description of Artificial Sequence: Synthetic polypeptide
QVQLVQSGAEVKKPGSSVKVSCKASGYTFSDYNMDWVRQAPGQGLEWMGQINPNNGGIFFNQKFQGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - DNA, 1347 bp, synthetic construct
  misc_feature 1..1347: note = Description of Artificial Sequence: Synthetic polynucleotide
  source 1..1347: mol_type = other DNA
caggtgacactcaaagaatcaggacccggaatccttcagcccagccagaccttgtcgctgacttgttcgttctccggtttcagcctgaatacttatgggatgggtgtgtcatggatcaggcaaccgtccgggaaaggattggagtggctcgcgcacatctactgggacgatgacaaacgctacaatccttcgctgaagagccgattgacgatttccaaggatgcctcgaacaaccgggtatttcttaagatcacgtcggtcgatacggcagacacggcgacctattactgcgcccaaagagggtacgatgactattggggatattggggccaggggacactcgtcacaatttcagctgcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 192 - protein, 449 aa, synthetic construct
  REGION 1..449: note = Description of Artificial Sequence: Synthetic polypeptide
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - DNA, 1347 bp, synthetic construct
  misc_feature 1..1347: note = Description of Artificial Sequence: Synthetic polynucleotide
  source 1..1347: mol_type = other DNA
caggtgactttgaaagaatccggtcccgcattggtaaagccaacccagacacttacgctcacatgtacattttccggattcagcttgaacacttacgggatgggagtgtcgtggattcggcaacctccggggaaggctctggagtggctggcgcacatctactgggatgatgacaaaaggtataacccctcacttaaaacgagactgacgatctcgaaggacacaagcaagaatcaggtcgtcctcacgattacgaatgtagacccggtggatactgccgtctattactgcgcgcaacgcgggtatgatgactactggggatattggggtcagggcaccctcgtgaccatctcgtcagcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 194 - protein, 449 aa, synthetic construct
  REGION 1..449: note = Description of Artificial Sequence: Synthetic polypeptide
QVTLKESGPALVKPTQTLTLTCTFSGFSLNTYGMGVSWIRQPPGKALEWLAHIYWDDDKRYNPSLKTRLTISKDTSKNQVVLTITNVDPVDTAVYYCAQRGYDDYWGYWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1347 bp, synthetic construct
  misc_feature 1..1347: note = Description of Artificial Sequence: Synthetic polynucleotide
  source 1..1347: mol_type = other DNA
caagtaacgctcaaggagtccggacccaccttggtgaagccgacgcagaccttgactcttacgtgcactttctcggggttttcactgaatacgtacgggatgggtgtctcatggatcaggcaacctccggggaaaggattggaatggctggcgcacatctactgggatgacgataagagatataacccaagcctcaagtcgcggctcaccattacaaaagatacatcgaaaaatcaggtcgtacttactatcacgaacatggaccccgtggacacagcaacatattactgtgcccagcgcggctatgacgattattggggttactggggacagggaacactggtcacggtgtccagcgcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 196 - protein, 449 aa, synthetic construct
  REGION 1..449: note = Description of Artificial Sequence: Synthetic polypeptide
QVTLKESGPTLVKPTQTLTLTCTFSGFSLNTYGMGVSWIRQPPGKGLEWLAHIYWDDDKRYNPSLKSRLTITKDTSKNQVVLTITNMDPVDTATYYCAQRGYDDYWGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - DNA, 1347 bp, synthetic construct
  misc_feature 1..1347: note = Description of Artificial Sequence: Synthetic polynucleotide
  source 1..1347: mol_type = other DNA
caggtcacgctgaaagagtcaggtcccggaatccttcaaccttcgcagacattgtcactcacatgttccttctccgggttctcgctctcgacttatggcatgggtgtaggatggattcggcagcccagcgggaaggggcttgagtggttggcggatatctggtgggacgacgacaaatactacaatccgagcctgaagtcccgcctcaccatttcgaaagatacgtcatcaaacgaagtctttttgaagatcgccatcgtggacacggcggatacagcgacgtattactgcgccagaaggggacactacagcgcaatggattattggggacaggggacctcggtgactgtgtcgtccgcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 198 - protein, 449 aa, synthetic construct
  REGION 1..449: note = Description of Artificial Sequence: Synthetic polypeptide
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - DNA, 1347 bp, synthetic construct
  misc_feature 1..1347: note = Description of Artificial Sequence: Synthetic polynucleotide
  source 1..1347: mol_type = other DNA
cagatcactttgaaagaaagcggaccgaccttggtcaagcccacacaaaccctcacgctcacgtgtacattttcggggttctcgctttcaacttacgggatgggagtagggtggattcgccagccgcctggtaaagcgttggagtggcttgcagacatctggtgggacgacgataagtactataatccctcgctcaagtccagactgaccatcacgaaagatacgagcaagaaccaggtcgtgctgacaatgactaacatggacccagtggatacggctacatattactgcgccaggcggggtcactactcagcgatggattattggggccagggaacactggtaacggtgtcgtccgcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 200 - protein, 449 aa, synthetic construct
  REGION 1..449: note = Description of Artificial Sequence: Synthetic polypeptide
QITLKESGPTLVKPTQTLTLTCTFSGFSLSTYGMGVGWIRQPPGKALEWLADIWWDDDKYYNPSLKSRLTITKDTSKNQVVLTMTNMDPVDTATYYCARRGHYSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - DNA, 1347 bp, synthetic construct
  misc_feature 1..1347: note = Description of Artificial Sequence: Synthetic polynucleotide
  source 1..1347: mol_type = other DNA
caagtgactctcaaggagtccggacccgccctggtcaaaccaacgcagacactgacgctcacatgcaccttcagcggattttcgttgtcaacgtacggcatgggtgtggggtggattcgccagcctccggggaaagcccttgaatggttggcggacatctggtgggatgatgacaagtactataatccctcacttaagtcacggttgacgatctcgaaagacaccagcaagaaccaggtagtgctgacaatgactaacatggacccggtcgatacagcggtctactattgtgctagaaggggacactactccgcaatggattattggggtcaggggacgctcgtaaccgtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 202 - protein, 449 aa, synthetic construct
  REGION 1..449: note = Description of Artificial Sequence: Synthetic polypeptide
QVTLKESGPALVKPTQTLTLTCTFSGFSLSTYGMGVGWIRQPPGKALEWLADIWWDDDKYYNPSLKSRLTISKDTSKNQVVLTMTNMDPVDTAVYYCARRGHYSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - DNA, 642 bp, synthetic construct
  misc_feature 1..642: note = Description of Artificial Sequence: Synthetic polynucleotide
  source 1..642: mol_type = other DNA
gacatccaaatgacccagtcacccgcgagcctttcggcgtcggtcggagaaacggtcacgatcacgtgccggacatcagagaatctccataactacctcgcgtggtatcaacagaagcaggggaagtcgccccagttgcttgtatacgatgcgaaaacgttggcggatggggtgccgtccagattctcgggatcgggctcggggacgcagtactcgctcaagatcaattcgctgcagccggaggactttgggtcgtactattgtcagcatttttggtcatcaccgtatacatttggaggtggaacgaaacttgagattaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 204 - protein, 214 aa, synthetic construct
  REGION 1..214: note = Description of Artificial Sequence: Synthetic polypeptide
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - DNA, 642 bp, synthetic construct
  misc_feature 1..642: note = Description of Artificial Sequence: Synthetic polynucleotide
  source 1..642: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttgtctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 206 - protein, 214 aa, synthetic construct
  REGION 1..214: note = Description of Artificial Sequence: Synthetic polypeptide
DIQMTQSPSSLSASVGDRVTITCRTSENLHNYLAWYQQKPGKSPKLLVYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 642 bp, synthetic construct
  misc_feature 1..642: note = Description of Artificial Sequence: Synthetic polynucleotide
  source 1..642: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaaggccccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 208 - protein, 214 aa, synthetic construct
  REGION 1..214: note = Description of Artificial Sequence: Synthetic polypeptide
DIQMTQSPSSLSASVGDRVTITCRTSENLHNYLAWYQQKPGKAPKLLIYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 642 bp, synthetic construct
  misc_feature 1..642: note = Description of Artificial Sequence: Synthetic polynucleotide
  source 1..642: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcgtcgccctacacatttgggcaggggaccaagttggaaatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 210 - protein, 214 aa, synthetic construct
  REGION 1..214: note = Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - DNA, 642 bp, synthetic construct
  misc_feature 1..642: note = Description of Artificial Sequence: Synthetic polynucleotide
  source 1..642: mol_type = other DNA
gatatcgtcatgacccagtcccagaagttcatgtcaacttcagtgggagacagagtgtccgtcacatgtaaagcctcgcaaaatgtgggaaccaacgtagcgtggttccagcagaaacctggccaatcaccgaaggcactgatctactcggccagctataggtactcgggagtaccagatcggtttacggggtcggggagcgggacggactttatcctcactatttccaatgtccagtcggaggaccttgcggaatacttctgccagcagtataacaactatcccctcacgtttggtgctggtacaaaattggagttgaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 212 - protein, 214 aa, synthetic construct
  REGION 1..214: note = Description of Artificial Sequence: Synthetic polypeptide
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - DNA, 642 bp, synthetic construct
  misc_feature 1..642: note = Description of Artificial Sequence: Synthetic polynucleotide
  source 1..642: mol_type = other DNA
gacatccaaatgacccaatcgccctcctccctctccgcatcagtaggggaccgcgtcacaattacttgcaaagcgtcgcagaacgtcggaacgaatgtggcgtggtttcagcagaagcccggaaaagctccgaagagcttgatctactcggcctcatataggtattcgggtgtgccgagccggtttagcgggtcggggtcaggtactgatttcacgctcacaatttcatcgttgcagccagaagatttcgccacatattactgtcagcagtacaacaattaccctctgacgttcggccagggaaccaaacttgagatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 214 - protein, 214 aa, synthetic construct
  REGION 1..214: note = Description of Artificial Sequence: Synthetic polypeptide
DIQMTQSPSSLSASVGDRVTITCKASQNVGTNVAWFQQKPGKAPKSLIYSASYRYSGVPSRFSGSGSGTDFTLTISSLQPEDFATYYCQQYNNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - DNA, 642 bp, synthetic construct
  misc_feature 1..642: note = Description of Artificial Sequence: Synthetic polynucleotide
  source 1..642: mol_type = other DNA
gacatcgtgatgacacagtcacagaaattcatgtccacatccgtcggtgatagagtatccgtcacgtgtaaggcctcgcaaaacgtaggaactaatgtggcgtggtatcaacagaagccaggacagtcacccaaagcactcatctacagcccctcatatcggtacagcggggtgccggacaggttcacgggatcggggagcgggaccgattttacactgaccatttcgaatgtccagtcggaggaccttgcggaatacttctgccagcagtataactcgtaccctcacacgtttggaggtggcactaagttggagatgaaacgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 216 - protein, 214 aa, synthetic construct
  REGION 1..214: note = Description of Artificial Sequence: Synthetic polypeptide
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - DNA, 642 bp, synthetic construct
  misc_feature 1..642: note = Description of Artificial Sequence: Synthetic polynucleotide
  source 1..642: mol_type = other DNA
gatatccagatgacacagtcaccctcgtcgctctcagcttccgtaggcgacagggtcactattacgtgtaaagcatcacagaacgtcggaacgaatgtggcgtggtttcagcagaagcccgggaagagccccaaagcgcttatctactccccgtcgtatcggtattccggtgtgccaagcagattttcggggtcaggttcgggaactgactttaccctgaccatctcgtccctccaaccggaagatttcgccacgtacttctgccagcagtacaacagctatcctcacacattcggacaagggacaaagttggagattaaacgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 218 - protein, 214 aa, synthetic construct
  REGION 1..214: note = Description of Artificial Sequence: Synthetic polypeptide
DIQMTQSPSSLSASVGDRVTITCKASQNVGTNVAWFQQKPGKSPKALIYSPSYRYSGVPSRFSGSGSGTDFTLTISSLQPEDFATYFCQQYNSYPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - DNA, 1035 bp, synthetic construct
  misc_feature 1..1035: note = Description of Artificial Sequence: Synthetic polynucleotide
  source 1..1035: mol_type = other DNA
gggtgtaaaccctgcatctgcacggtgccggaggtgtcctccgtctttatcttccctcccaaacccaaggatgtgctgacaatcactttgactccaaaagtcacatgcgtagtcgtggacatctcgaaagacgacccggaagtgcagttctcgtggtttgttgatgatgtagaagtgcataccgctcaaacccagccgagggaagaacagtttaacagcacgtttaggagtgtgtcggaactgcccattatgcaccaggattggcttaatgggaaggagttcaaatgtcgcgtgaatagtgcggcgttcccagcccctattgaaaagactatttccaaaacgaagggtcggcccaaagctccccaagtatacacaatccctccgccgaaagaacaaatggcaaaagacaaagtgagtttgacgtgcatgatcacggactttttcccggaggatatcaccgtcgaatggcaatggaatgggcaacctgccgaaaactacaagaatacacaacccattatggataccgatggatcgtatttcgtctactcaaagttgaacgtacagaagtcaaattgggaggcagggaatacgttcacttgcagtgttttgcacgaaggcctccataaccaccatacggaaaagtcactgtcgcactccccgggaaaaatcgagggcagaatggatggtggaggagggtcggcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 220 - protein, 345 aa, synthetic construct
  REGION 1..345: note = Description of Artificial Sequence: Synthetic polypeptide
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 221 - DNA, 1026 bp, synthetic construct
  misc_feature 1..1026: note = Description of Artificial Sequence: Synthetic polynucleotide
  source 1..1026: mol_type = other DNA
tcgaaacccacttgccctcctccggagctgttgggcggaccctccgtgtttatctttcccccgaagccgaaagatacccttatgatctcacggacgccggaggtcacttgcgtagtagtggatgtgtcggaggatgaccccgaagtccagttcacctggtatatcaataacgagcaagtgaggacagcgaggcccccacttagggagcagcagttcaactccacaattcgggtcgtcagcactttgcccatcgctcatgaggactggctccgcggaaaagagttcaagtgtaaggtgcataacaaggcattgccagcgcctattgaaaagacaatctcgaaggcgcgagggcagccgctcgagcccaaagtgtatacgatgggacccccgagggaagaattgtcgtcgcgctcagtaagccttacgtgcatgattaacggtttctaccctagcgacatcagcgtagagtgggaaaagaatggaaaggcggaggataactacaagacgactcccgcggtgctggattcggatgggtcgtactttctgtatagcaaattgtcagtcccgacctcagaatggcagaggggtgacgtgttcacgtgctccgtgatgcacgaagcacttcacaatcactacacccagaaatcaatctcgcggtccccaggcaaaggtggaggagggtcggctcacgcccaccctcgcgattcgtgtccgctggggcctggtagatgctgtcatctcgagacagtccaggccacgctggaggacctcgggtggtcagactgggtcctgtccccacgacaactgcagctttcgatgtgcgtgggggaatgtccgcacttgtacagatcggcgaatacccacgctcagattaaggcacgactccatggtttgcagccagataaagtccccgcaccttgctgtgtccccagctcatatactcctgtcgtactcatgcatcggacagacagcggcgtgtcgcttcaaacgtatgacgacctcgtagcgagaggatgtcattgcgcc

SEQ ID NO: 222 - protein, 342 aa, synthetic construct
  REGION 1..342: note = Description of Artificial Sequence: Synthetic polypeptide
SKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKGGGGSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 223 - protein, 350 aa, synthetic construct
  REGION 1..350: note = Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 224 - protein, 344 aa, synthetic construct
  REGION 1..344: note = Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 225 - DNA, 22 bp, synthetic construct
  misc_feature 1..22: note = Description of Artificial Sequence: Synthetic primer
  source 1..22: mol_type = other DNA
ctaatacgactcactatagggc

SEQ ID NO: 226 - DNA, 21 bp, synthetic construct
  misc_feature 1..21: note = Description of Artificial Sequence: Synthetic primer
  source 1..21: mol_type = other DNA
tatgcaaggcttacaaccaca

SEQ ID NO: 227 - DNA, 21 bp, synthetic construct
  misc_feature 1..21: note = Description of Artificial Sequence: Synthetic primer
  source 1..21: mol_type = other DNA
aggacaggggttgattgttga

SEQ ID NO: 228 - DNA, 27 bp, synthetic construct
  misc_feature 1..27: note = Description of Artificial Sequence: Synthetic primer
  source 1..27: mol_type = other DNA
ctcattcctgttgaagctcttgacaat

SEQ ID NO: 229 - DNA, 23 bp, synthetic construct
  misc_feature 1..23: note = Description of Artificial Sequence: Synthetic primer
  source 1..23: mol_type = other DNA
aagcagtggtatcaacgcagagt

SEQ ID NO: 230 - DNA, 23 bp, synthetic construct
  misc_feature 1..23: note = Description of Artificial Sequence: Synthetic primer
  source 1..23: mol_type = other DNA
cgactgaggcacctccagatgtt

SEQ ID NO: 231 - DNA, 17 bp, synthetic construct
  misc_feature 1..17: note = Description of Artificial Sequence: Synthetic primer
  source 1..17: mol_type = other DNA
gtaaaacgacggccagt

SEQ ID NO: 232 - DNA, 18 bp, synthetic construct
  misc_feature 1..18: note = Description of Artificial Sequence: Synthetic primer
  source 1..18: mol_type = other DNA
caggaaacagctatgacc

SEQ ID NO: 233 - DNA, 45 bp, synthetic construct
  misc_feature 1..45: note = Description of Artificial Sequence: Synthetic primer
  source 1..45: mol_type = other DNA
ctaatacgactcactatagggcaagcagtggtatcaacgcagagt

SEQ ID NO: 234 - protein, 7 aa, synthetic construct
  REGION 1..7: note = Description of Artificial Sequence: Synthetic peptide
GYTFSDY

SEQ ID NO: 235 - protein, 8 aa, synthetic construct
  REGION 1..8: note = Description of Artificial Sequence: Synthetic peptide
GYTFSDYN

SEQ ID NO: 236 - protein, 17 aa, synthetic construct
  REGION 1..17: note = Description of Artificial Sequence: Synthetic peptide
QINPYNHLIFFNQKFQG

SEQ ID NO: 237 - protein, 17 aa, synthetic construct
  REGION 1..17: note = Description of Artificial Sequence: Synthetic peptide
QINPNNGLIFFNQKFQG

SEQ ID NO: 238 - protein, 17 aa, synthetic construct
  REGION 1..17: note = Description of Artificial Sequence: Synthetic peptide
QINPNNGLIFFNQKFKG

SEQ ID NO: 239 - protein, 17 aa, synthetic construct
  REGION 1..17: note = Description of Artificial Sequence: Synthetic peptide
QINPYNHLIFFNQKFKG

SEQ ID NO: 240 - protein, 6 aa, synthetic construct
  REGION 1..6: note = Description of Artificial Sequence: Synthetic peptide
NPYNHL

SEQ ID NO: 241 - protein, 6 aa, synthetic construct
  REGION 1..6: note = Description of Artificial Sequence: Synthetic peptide
NPNNGL

SEQ ID NO: 242 - protein, 8 aa, synthetic construct
  REGION 1..8: note = Description of Artificial Sequence: Synthetic peptide
INPYNHLI

SEQ ID NO: 243 - protein, 8 aa, synthetic construct
  REGION 1..8: note = Description of Artificial Sequence: Synthetic peptide
INPNNGLI

SEQ ID NO: 244 - protein, 9 aa, synthetic construct
  REGION 1..9: note = Description of Artificial Sequence: Synthetic peptide
QHFWSDPYT

SEQ ID NO: 245 - DNA, 360 bp, synthetic construct
  misc_feature 1..360: note = Description of Artificial Sequence: Synthetic polynucleotide
  source 1..360: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgtacaatcacctgatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcg

SEQ ID NO: 246 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGQSLEWMGQINPYNHLIFFNQKFQGRVTLTTDTSTSTAYMELRSLRSDDTAVYYCAREAITTVGAMDYWGQGTLVTVSS

SEQ ID NO: 247 - DNA, 360 bp, synthetic construct
  misc_feature 1..360: note = Description of Artificial Sequence: Synthetic polynucleotide
  source 1..360: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgaataatggactgatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcg

SEQ ID NO: 248 - protein, 120 aa, synthetic construct
  REGION 1..120: note = Description of Artificial Sequence: Synthetic polypeptide
QVQLVQSGAEVKKPGASVKVSCKASGYTFTDYNMDWVRQAPGQSLEWMGQINPNNGLIFFNQKFQGRVTLTTDTSTSTAYMELRSLRSDDTAVYYCAREAITTVGAMDYWGQGTLVTVSS

SEQ ID NO: 249 - DNA, 360 bp, synthetic construct
  misc_feature 1..360: note = Description of Artificial Sequence: Synthetic polynucleotide
  source 1..360: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggctgatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcg

SEQ ID NO: 250 - protein, 120 aa, synthetic construct
  REGION 1..120: note = Description of Artificial Sequence: Synthetic polypeptide
QVQLVQSGAEVKKPGSSVKVSCKASGYTFSDYNMDWVRQAPGQGLEWMGQINPNNGLIFFNQKFKGRVTLTADKSTSTAYMELSSLRSEDTAVYYCAREAITTVGAMDYWGQGTLVTVSS

SEQ ID NO: 251 - DNA, 360 bp, synthetic construct
  misc_feature 1..360: note = Description of Artificial Sequence: Synthetic polynucleotide
  source 1..360: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgtacaatcacctgatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcg

SEQ ID NO: 252 - protein, 120 aa, synthetic construct
  REGION 1..120: note = Description of Artificial Sequence: Synthetic polypeptide
QVQLVQSGAEVKKPGSSVKVSCKASGYTFSDYNMDWVRQAPGQGLEWMGQINPYNHLIFFNQKFKGRVTLTADKSTSTAYMELSSLRSEDTAVYYCAREAITTVGAMDYWGQGTLVTVSS

SEQ ID NO: 253 - DNA, 321 bp, synthetic construct
  misc_feature 1..321: note = Description of Artificial Sequence: Synthetic polynucleotide
  source 1..321: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcggacccctacacatttgggcaggggaccaagttggaaatcaag

SEQ ID NO: 254 - protein, 107 aa, synthetic construct
  REGION 1..107: note = Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSDPYTFGQGTKLEIK

SEQ ID NO: 255 - DNA, 1350 bp, synthetic construct
  misc_feature 1..1350: note = Description of Artificial Sequence: Synthetic polynucleotide
  source 1..1350: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgtacaatcacctgatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 256 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGQSLEWMGQINPYNHLIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - DNA, 1350 bp, synthetic construct
  misc_feature 1..1350: note = Description of Artificial Sequence: Synthetic polynucleotide
  source 1..1350: mol_type = other DNA
caggtccagcttgtgcaatcgggagcggaagtgaagaaaccgggagcgtcggtaaaagtctcgtgcaaagcgtcggggtatacgtttacggactataacatggactgggtgcgccaagcgcctggacagagccttgaatggatggggcagattaatccgaataatggactgatcttctttaatcagaaattccagggaagggtaacgctgacgacagacacgtcaacatcgacggcctatatggaattgcggtcgttgcgatcagatgatacggcggtctactattgtgcgagggaggcgattacgacggtgggagcgatggattattggggacaggggacgttggtaacggtatcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 258 - protein, 450 aa, synthetic construct
  REGION 1..450: note = Description of Artificial Sequence: Synthetic polypeptide
QVQLVQSGAEVKKPGASVKVSCKASGYTFTDYNMDWVRQAPGQSLEWMGQINPNNGLIFFNQKFQGRVTLTTDTSTSTAYMELRSLRSDDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - DNA, 1350 bp, synthetic construct
  misc_feature 1..1350: note = Description of Artificial Sequence: Synthetic polynucleotide
  source 1..1350: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgaataatgggctgatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 260 - protein, 450 aa, synthetic construct
  REGION 1..450: note = Description of Artificial Sequence: Synthetic polypeptide
QVQLVQSGAEVKKPGSSVKVSCKASGYTFSDYNMDWVRQAPGQGLEWMGQINPNNGLIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - DNA, 1350 bp, synthetic construct
  misc_feature 1..1350: note = Description of Artificial Sequence: Synthetic polynucleotide
  source 1..1350: mol_type = other DNA
caagtacagcttgtacagtcgggagcggaagtcaagaaaccgggatcgtcggtcaaagtgtcgtgtaaagcgtcgggatatacgtttagcgactataacatggattgggtgcgacaagcgcctgggcagggacttgaatggatgggtcagatcaatccgtacaatcacctgatctttttcaatcagaagtttaaagggagggtaacgctgacggcggataaaagcacgtcaacggcgtatatggagttgtcgtcgttgcggtcggaggacacggcggtctattactgcgcgagggaagcgattacgacggtgggagcgatggattattgggggcagggaacgcttgtaacggtgtcatcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 262 - protein, 450 aa, synthetic construct
  REGION 1..450: note = Description of Artificial Sequence: Synthetic polypeptide
QVQLVQSGAEVKKPGSSVKVSCKASGYTFSDYNMDWVRQAPGQGLEWMGQINPYNHLIFFNQKFKGRVTLTADKSTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - DNA, 642 bp, synthetic construct
  misc_feature 1..642: note = Description of Artificial Sequence: Synthetic polynucleotide
  source 1..642: mol_type = other DNA
gacatccaaatgacccagtcgccgtcgtcgctttcagcgtcggtaggggatcgggtcacaattacgtgccgaacgtcagagaatttgcataactacctcgcgtggtatcagcagaagcccgggaagtcaccgaaactccttatctacgatgcgaaaacgctggcggatggagtgccgtcgagattctcgggaagcggatccggtacggactatacgcttacgatctcatcgctccagcccgaggactttgcgacgtactattgtcagcatttttggtcggacccctacacatttgggcaggggaccaagttggaaatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 264 - protein, 214 aa, synthetic construct
  REGION 1..214: note = Description of Artificial Sequence: Synthetic polypeptide
DIQMTQSPSSLSASVGDRVTITCRTSENLHNYLAWYQQKPGKSPKLLIYDAKTLADGVPSRFSGSGSGTDYTLTISSLQPEDFATYYCQHFWSDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 4 aa, synthetic construct
  REGION 1..4: note = Description of Artificial Sequence: Synthetic peptide
GGGG

SEQ ID NO: 266 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0166734 A1
(43) Pub. Date: May 23, 2024

(54) ANTI-GDF-15 ANTIBODIES

(71) Applicant: AVEO Pharmaceuticals, Inc., Cambridge, MA (US)

(72) Inventors: Lorena LERNER, Newton Centre, MA (US); Sandra ABBOTT, Boston, MA (US); Ailin BAI, Newton, MA (US); Ting CHEN, Acton, MA (US); Maria Isabel CHIU, Newton Centre, MA (US); Qing LIU, Acton, MA (US); Laura POLING, Boston, MA (US); Nianjun TAO, Brighton, MA (US); Solly WEILER, Newton, MA (US); Zhigang WENG, Brookline, MA (US); William M. WINSTON, JR., Marlborough, MA (US); Jeno GYURIS, Lincoln, MA (US)

(21) Appl. No.: 18/336,713

(22) Filed: Jun. 16, 2023

Related U.S. Application Data
(63) Continuation of application No. 16/824,034, filed on Mar. 19, 2020, now Pat. No. 11,725,047.
Application No. 16/824,034 is a continuation of application No. 15/655,263, filed on Jul. 20, 2017, now Pat. No. 10,597,444.
Application No. 15/655,263 is a continuation of application No. 14/863,870, filed on Sep. 24, 2015, now Pat. No. 9,725,505.
Application No. 14/863,870 is a division of application No. 14/137,415, filed on Dec. 20, 2013, now Pat. No. 9,175,076.
(60) Provisional application No. 61/827,325, filed on May 24, 2013.
(60) Provisional application No. 61/745,508, filed on Dec. 21, 2012.

(51) Int. Cl. C07K 16/22; A61K 39/395; A61K 45/06; A61P 3/02

(52) U.S. Cl. CPC C07K 16/22; A61K 39/3955; A61K 45/06; A61P 3/02; A61K 2039/505

(57) ABSTRACT

Monoclonal antibodies that bind and inhibit the activity of human GDF15 are disclosed. The antibodies can be used to treat body weight loss, including cachexia, associated with the over-expression of human GDF15.

### CROSS-REFERENCE TO RELATED APPLICATIONS

This application is a continuation of U.S. patent application Ser. No. 16/824,034, filed Mar. 19, 2020, which is a continuation of U.S. patent application Ser. No. 15/655,263, filed Jul. 20, 2017, issued as U.S. Pat. No. 10,597,444 on Mar. 24, 2020, which is a continuation of U.S. patent application Ser. No. 14/863,870, filed Sep. 24, 2015, issued as U.S. Pat. No. 9,725,505 on Aug. 8, 2017, which is a divisional of U.S. patent application Ser. No. 14/137,415, filed Dec. 20, 2013, issued as U.S. Pat. No. 9,175,076 on Nov. 3, 2015, which claims the benefit of and priority to U.S. Provisional Patent Application No. 61/827,325, filed May 24, 2013, and U.S. Provisional Patent Application No. 61/745,508, filed Dec. 21, 2012, the entire disclosures of each of which are incorporated by reference herein in their entireties.

### SEQUENCE LISTING

The instant application contains a Sequence Listing which has been submitted electronically in ST.26 XML format and is hereby incorporated by reference in its entirety. Said ST.26 XML copy, created on Jun. 16, 2023, is named 406767_029USD1C3_SEQUENCE_LISTING_ST.26.XML and is 425,984 bytes in size.

### FIELD OF THE INVENTION

The field of the invention is molecular biology, immunology, cachexia and cachexia-like disorders, and oncology. More particularly, the field is therapeutic antibodies.

### BACKGROUND

Involuntary weight loss can be categorized into three primary etiologies that include, cachexia, sarcopenia and starvation. Cachexia is a debilitating metabolic syndrome associated with numerous diseases, including cancer, AIDS, chronic heart failure (also known as congestive heart failure), chronic obstructive pulmonary disease (COPD), chronic kidney disease, tuberculosis, sepsis and other forms of systemic inflammation. Cachexia varies in its manifestations, but generally involves involuntary loss of skeletal muscle mass and some form of underlying illness (Evans et al. (2008) CLIN. NUTR. 27:793-799). Cachexia is a wasting disorder involving involuntary weight loss and may be associated with systemic inflammation and/or an acute inflammatory response. Thomas (2007) CLIN. NUTRITION 26:389-399. Loss of fat mass as well as fat-free mass, such as muscle mass, often is a prominent clinical feature of cachexia. In many but not all cases, cachexia progresses through stages that have been designated precachexia, cachexia and refractory cachexia (Fearon et al. (2011) LANCET ONC. 12:489-495). Two different, but sometimes overlapping, processes appear to drive the development and progression of cachexia: (a) metabolic processes that act directly on muscle, reducing its mass and function; and (b) reduced food intake, which leads to loss of both fat and muscle (Tsai et al. (2012) J. CACHEXIA SARCOPENIA MUSCLE 3:239-243).

Although cachexia is a complex and incompletely understood syndrome, it is clear that GDF15 (also known as MIC-1, PLAB, PDF and NAG-1), a member of the TGF-β superfamily, is an important mediator of cachexia in various diseases (Tsai et al., supra). At least some tumors over-express and secrete GDF15, and elevated serum GDF15 levels have been associated with various cancers (Johnen et al. (2007) NAT. MED. 13:1333-1340; Bauskin et al. (2006) CANCER RES. 66:4983-4986). Monoclonal antibodies against GDF15 have been recognized as potential anti-cachexia therapeutic agents. See, e.g., U.S. Pat. No. 8,192,735.

Weight loss resulting from cachexia is associated with poor prognosis in various diseases (Evans et al., supra), and cachexia and its consequences are considered to be the direct cause of death in about 20% of cancer deaths (Tisdale (2002) NAT. REV. CANCER 2:862-871). Cachexia is infrequently reversed by nutritional intervention, and currently this syndrome is seldom treated with drug therapy (Evans et al., supra).

Sarcopenia is a clinical condition related to cachexia that is characterized by loss of skeletal muscle mass and muscle strength. The decrease in muscle mass can lead to functional impairment, with loss of strength, increased likelihood of falls, and loss of autonomy. Respiratory function may also be impaired with a reduced vital capacity. During metabolic stress, muscle protein is rapidly mobilized in order to provide the immune system, liver and gut with amino acids, particularly glutamine. Sarcopenia often is a disease of the elderly; however, its development may also be associated with muscle disuse and malnutrition, and may coincide with cachexia. Sarcopenia can be diagnosed based upon functional observations such as low muscle weight and low gait speed. See, e.g., Muscaritoli et al. (2010) CLIN. NUTRITION 29:154-159.

Starvation typically results in a loss of body fat and non-fat mass due to inadequate diet and/or nutritional uptake (Thomas (2007) supra). The effects of starvation often are reversed by improving diet and nutritional, for example, protein, uptake.

Naturally occurring antibodies are multimeric proteins that contain four polypeptide chains (FIG. 1). Two of the polypeptide chains are called heavy chains (H chains), and two of the polypeptide chains are called light chains (L chains). The immunoglobulin heavy and light chains are connected by an interchain disulfide bond. The immunoglobulin heavy chains are connected by interchain disulfide bonds. A light chain consists of one variable region (V_{L} in FIG. 1) and one constant region (C_{L} in FIG. 1). The heavy chain consists of one variable region (V_{H} in FIG. 1) and at least three constant regions (C_{H1}, C_{H2} and C_{H3} in FIG. 1). The variable regions determine the specificity of the antibody. Each variable region comprises three hypervariable regions also known as complementarity determining regions (CDRs) flanked by four relatively conserved framework regions (FRs). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

There is a significant unmet need for effective therapeutic agents for treating cachexia and sarcopenia, including monoclonal antibodies targeting GDF15. Such therapeutic agents have the potential to play an important role in the treatment of various cancers and other life-threatening diseases.

### SUMMARY

The invention is based, in part, upon the discovery of a family of antibodies that specifically bind human GDF15 (hGDF15). The antibodies contain hGDF15 binding sites based on the CDRs of the antibodies. The antibodies can be used as therapeutic agents. When used as therapeutic agents, the antibodies are engineered, e.g., humanized, to reduce or eliminate an immune response when administered to a human patient.

The disclosed antibodies prevent or inhibit the activity of (i.e., neutralize) hGDF15. When administered to a mammal, the antibodies can inhibit the loss of muscle mass, for example, the loss of muscle mass associated with an underlying disease. The underlying disease may be selected from the group consisting of cancer, chronic heart failure, chronic kidney disease, COPD, AIDS, multiple sclerosis, rheumatoid arthritis, sepsis, and tuberculosis. In some embodiments, the loss of muscle mass may be accompanied by a loss of fat mass. The disclosed antibodies can also be used to inhibit involuntary weight loss in a mammal. In some embodiments, the disclosed antibodies may also be used to inhibit the loss of organ mass. Further, a method of treating cachexia and/or sarcopenia in a mammal comprising administering an effective amount of one of at least one of the disclosed antibodies to a mammal in need thereof is disclosed.

Also disclosed is a method for establishing a steady-state level of mature recombinant human GDF15 (rhGDF15) in plasma or serum in a mammal comprising administering a rhGDF15-immunoglobulin Fc (Fc-rhGDF15) fusion protein to the mammal. The Fc-rhGDF15 can be a mouse Fc mature recombinant human GDF15 (mFc-rhGDF15). In some embodiments, the mammal is a rodent, e.g., a mouse.

In another aspect, a method of treating obesity in a mammal, for example, a human, comprising administering a therapeutically effective amount of Fc-rhGDF15, e.g., a human Fc mature recombinant human GDF15 (hFc-rhGDF15), to the mammal in need thereof, is disclosed. Pharmaceutical compositions comprising an Fc-rhGDF15 fusion protein and a pharmaceutically acceptable carrier are also disclosed.

These and other aspects and advantages of the invention will become apparent upon consideration of the following figures, detailed description, and claims. As used herein, “including” means without limitation, and examples cited are non-limiting. As used herein, “antibody 01G06, 03G05, 04F08, 06C11, 08G01, 14F11, or 17B11” means antibody 01G06, 03G05, 04F08, 06C11, 08G01, 14F11, or 17B11, or humanized variants thereof.

### DESCRIPTION OF THE DRAWINGS

The invention can be more completely understood with reference to the following drawings.

FIG. 1 (prior art) is a schematic representation of atypical naturally-occurring antibody.

FIG. 2 is a graph representing results from an experiment to measure hGDF15 serum levels in naïve mice or mice bearing human xenograft tumors (Chago, RPMI7951, PC3, TOV21G, HT-1080, K-562, LS1034), as determined by ELISA.

FIG. 3 is a plot representing results from an experiment to determine the plasma pharmacokinetics (PK) of cleaved rhGDF15 administered by subcutaneous injection (1 μg/g) in naïve ICR-SCID mice, as determined by ELISA.

FIG. 4 is a graph summarizing results from an experiment to measure cachectic activity of cleaved rhGDF15 protein (▪) and negative control (PBS (•)) to induce body weight loss in immune-incompetent mice, ICR-SCID. Arrows indicate subcutaneous doses of 1 μg/g of rhGDF15.

FIGS. 5A and 5B are graphs summarizing results from an experiment to measure cachectic activity of mFc-rhGDF15 (a mouse Fc fused to the amino terminus of a mature recombinant human GDF15; ▪), rFc-rmGDF15 (a rabbit Fc fused to the amino terminus of a mature recombinant mouse GDF15; ▴), and negative control (PBS; •) to induce body weight loss in immune-competent Balb/C mice (FIG. 5A) and immune-incompetent CB17-SCID mice (FIG. 5B). Arrows indicate subcutaneous doses of 1 μg/g of recombinant protein.

FIGS. 6A-6E are graphs summarizing results from an experiment to demonstrate cachectic activity of mFc-rhGDF15 (▪) and negative control (PBS; •) to induce body weight loss in immune-incompetent ICR-SCID mice (FIG. 6A; arrows indicate subcutaneous doses of 1 μg/g of mFc-rhGDF15); to induce loss of adipose tissue or gonadal fat mass (FIG. 6B); to induce loss of muscle mass of gastrocnemius muscle (FIG. 6C; Gastroc Mass); and to increase mRNA expression of muscle degradation molecular markers (mMuRF1 (FIG. 6D) and mAtrogin (FIG. 6E)).

FIG. 7 is a graph summarizing results from an experiment to measure cachectic activity of mFc-rhGDF15 (▪) and negative control (PBS; •) to induce body weight loss in immune-incompetent Balb/C nude mice. Arrows indicate subcutaneous doses of 1.33 μg/g of mFc-rhGDF15.

FIG. 8 is a graph summarizing results from an experiment to measure serum levels of mFc-rhGDF15 in mice dosed with the recombinant protein. The presence of mFc-rhGDF15 was determined by Western Blot. Two positive bands corresponding to mFc-rhGDF15 and rhGDF15 (according to the appropriate molecular size) were quantified by Licor. The percentage of released-rhGDF15 versus mFc-rhGDF15 was calculated.

FIG. 9A is a graph summarizing results from an experiment to measure cachectic activity of mFc-rhGDF15 (0.1 μg/g (▪), 0.01 μg/g (Δ)) and negative control (mIgG 0.1 μg/g (•)) to induce body weight loss in immune-incompetent ICR-SCID mice. Arrows indicate the intraperitoneal dose of the recombinant protein. FIG. 9B is a graph representing the total level of rhGDF15 in the plasma of mice dosed with mFc-rhGDF15 (0.1 μg/g (□), 0.01 μg/g (▪)) five days post dose, as determined by ELISA.

FIG. 10 is a sequence alignment showing the amino acid sequence of the complete immunoglobulin heavy chain variable region of antibodies 01G06, 03G05, 04F08, 06C11, 08G01, 14F11, and 17B11. The amino acid sequences for each antibody are aligned against one another, and CDR₁, CDR₂, and CDR₃, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

FIG. 11 is a sequence alignment showing the CDR₁, CDR₂, and CDR₃ sequences for each of the immunoglobulin heavy chain variable region sequences in FIG. 10.

FIG. 12 is a sequence alignment showing the amino acid sequence of the complete immunoglobulin light chain variable region of antibodies 01G06, 03G05, 04F08, 06C11, 08G01, 14F11, and 17B11. The amino acid sequences for each antibody are aligned against one another, and CDR₁, CDR₂, and CDR₃, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

FIG. 13 is a sequence alignment showing the CDR₁, CDR₂, and CDR₃ sequences for each of the immunoglobulin light chain variable region sequences in FIG. 12.

FIG. 14 is a graph summarizing results from an experiment to measure cachectic inhibitory activity of anti-GDF15 antibodies 01G06 (▪), 03G05 (▴), 04F08 (▾), 06C11 (⋄), 14F11 ( [symbol] ), and 17B11 (□), and a murine IgG control (•; mIgG) dosed at 10 mg/kg in an mFc-rhGDF15 cachectic model in ICR-SCID mice. The arrow indicates intra-peritoneal injection of antibody.

FIG. 15 is a graph summarizing results from an experiment to measure cachectic inhibitory activity of anti-GDF15 antibodies 01G06 (▴), 03G05 (⋄), 04F08 (∇), 06C11 (□), 08G01 (▪), 14F11 (♦), and 17B11 ( [symbol] ), and a murine IgG control (•; mIgG), dosed at 10 [symbol] mg/kg in an HT-1080 fibrosarcoma tumor xenograft model in ICR-SCID mice. The arrows indicate intra-peritoneal injection of antibody every three days.

FIGS. 16A-16E are graphs summarizing results from an experiment to demonstrate anti-cachectic activity of anti-GDF15 antibody 01G06 (▪), dosed at 10 mg/kg, in immune-incompetent mice (ICR-SCID) bearing an HT-1080 fibrosarcoma tumor xenograft model. Treatment with antibody 01G06 reversed body weight loss (FIG. 16A); induced a significant increase in food consumption for up to three days post dose (FIG. 16B); induced a gain of gonadal fat mass (FIG. 16C); induced a gain of muscle mass of gastrocnemius muscle (FIG. 16D); and decreased mRNA expression of muscle degradation molecular markers (mMuRF1 and mAtrogin (FIG. 16E)) compared to negative control (murine IgG (•)). In FIG. 16A, the arrow indicates intra-peritoneal injection of antibody.

FIGS. 17A-17B are graphs summarizing results from an experiment to demonstrate anti-cachectic activity of anti-GDF15 antibody 01G06 (▪), dosed at 2 mg/kg, in immune-incompetent mice (ICR-SCID) bearing an HT-1080 fibrosarcoma tumor xenograft model. Treatment with antibody 01G06 reversed body weight loss compared to murine IgG (•) (FIG. 17A); and induced a gain of organ mass (liver, heart, spleen, kidney) and induced a gain of tissue mass (gonadal and gastrocnemius) (FIG. 17B) compared to negative control (murine IgG) and baseline (day 1). The arrows in FIG. 17A indicate intra-peritoneal injection of antibody.

FIG. 18 is a graph summarizing results from an experiment to measure cachectic inhibitory activity of anti-GDF15 antibodies 01G06 (▪), 03G05 (▴), 04F08 (♦), 06C11 (δ), 08G01 (∘), 14F11 (□), and 17B11 (Δ), and a murine IgG control (•) dosed at 10 mg/kg in a K-562 leukemia tumor xenograft model in immune-incompetent (CB17SCRFMF) mice. The arrows indicate intra-peritoneal injection of antibody.

FIG. 19 is a sequence alignment showing the amino acid sequence of the complete immunoglobulin heavy chain variable region of chimeric 01G06 variable region denoted as Ch01G06 Chimeric; humanized 01G06 heavy chain variable regions denoted as Hu01G06 IGHV1-18, Hu01G06 IGHV1-69, Sh01G06 IGHV1-18 M69L, Sh01G06 IGHV1-18 M69L K64Q G44S, Sh01G06 IGHV1-18 M69L K64Q, Sh01G06 IGHV1-69 T30S I69L, Sh01G06 IGHV1-69 T30S K64Q I69L, Hu01G06 IGHV1-18 F1, Hu01G06 IGHV1-18 F2, Hu01G06 IGHV1-69 F1, and Hu01G06 IGHV1-69 F2; chimeric 06C11 denoted as Ch06C11 Chimeric; humanized 06C11 heavy chain variable regions denoted as HE LM 06C11 IGHV2-70, and Hu06C11 IGHV2-5; chimeric 14F11 denoted as Ch14F11 Chimeric; and humanized 14F11 heavy chain variable regions denoted as Sh14F11 IGHV2-5 and Sh14F11 IGHV2-70. The amino acid sequences for each antibody are aligned against one another, and CDR₁, CDR₂, and CDR₃, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

FIG. 20 is a sequence alignment showing the CDR₁, CDR₂, and CDR₃ sequences for each of the immunoglobulin heavy chain variable region sequences in FIG. 19.

FIG. 21 is a sequence alignment showing the amino acid sequence of the complete immunoglobulin light chain variable region of chimeric 01G06 denoted as Ch01G06 Chimeric; humanized 01G06 light chain variable regions denoted as Hu01G06 IGKV1-39, Hu01G06 IGKV1-39 S43A V48I, Hu01G06 IGKV1-39 V48I, Hu01G06 IGKV1-39 F1, and Hu01G06 IGKV1-39 F2; chimeric 06C11 denoted as Ch06C11 Chimeric; humanized 06C11 light chain variable region denoted as Sh06C11 IGKV1-16; chimeric 14F11 denoted as Ch14F11 Chimeric; and humanized 14F11 light chain variable region denoted as Hu14F11 IGKV1-16. The amino acid sequences for each antibody are aligned against one another, and CDR₁, CDR₂, and CDR₃, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

FIG. 22 is a sequence alignment showing the CDR₁, CDR₂, and CDR₃ sequences for each of the immunoglobulin light chain variable region sequences in FIG. 21.

FIG. 23 is a graph summarizing results from an experiment to measure cachectic inhibitory activity of anti-GDF15 antibodies 01G06 (▪), Hu01G06-46 (▴), and Hu01G06-52 (*), and a murine IgG control (•) dosed at 2 mg/kg in an HT-1080 fibrosarcoma tumor xenograft model in ICR-SCID mice. The arrow indicates intra-peritoneal injection of antibody.

FIG. 24 is a graph summarizing results from an experiment to measure cachectic inhibitory activity of anti-GDF15 antibodies 06C11 (♦), Hu06C11-27 (□), and Hu06C11-30 (▴), and a murine IgG control (•) dosed at 2 mg/kg in an HT-1080 fibrosarcoma tumor xenograft model in ICR-SCID mice. The arrow indicates intra-peritoneal injection of antibody.

FIG. 25 is a graph summarizing results from an experiment to measure cachectic inhibitory activity of anti-GDF15 antibodies 14F11 (▴), Hu14F11-39 (□), and Hu14F11-47 (♦), and a murine IgG control (•) dosed at 2 mg/kg in an HT-1080 fibrosarcoma tumor xenograft model in ICR-SCID mice. The arrow indicates intra-peritoneal injection of antibody.

FIG. 26 is a graph summarizing results from an experiment to measure cachectic inhibitory activity of anti-GDF15 antibodies Hu01G06-122 (▾), Hu01G06-127 (□), Hu01G06-135 (⋄), Hu01G06-138 (▪), and Hu01G06-146 (*), and a human IgG control (•) dosed at 2 mg/kg in an HT-1080 fibrosarcoma tumor xenograft model in ICR-SCID mice. The arrow indicates intra-peritoneal injection of antibody.

FIG. 27 is a graph summarizing results from an experiment to measure cachectic inhibitory activity of anti-GDF15 antibodies Hu01G06-122 (▾), Hu01G06-127 (□), Hu01G06-135 (⋄), Hu01G06-138 (▪), and Hu01G06-146 (*), and a human IgG control (•) dosed at 2 mg/kg in an mFc-rhGDF15 cachectic model in ICR-SCID mice. The arrow indicates intra-peritoneal injection of antibody.

FIG. 28 is a graph summarizing results from an experiment to measure cachectic dose response inhibitory activity of anti-GDF15 antibodies Hu01G06-127 dosed at 20 mg/kg (□), 2 mg/kg (Δ), and 0.2 mg/kg (∇); Hu01G06-135 at 20 mg/kg (▪), 2 mg/kg (▴), and 0.2 mg/kg (▾), and a human IgG control at 20 mg/kg (•) in an HT-1080 fibrosarcoma tumor xenograft model in ICR-SCID mice. The arrow indicates intravenous injection of antibody.

FIGS. 29A-29C are graphs summarizing results from an experiment to demonstrate anti-cachectic activity of anti-GDF15 antibodies Hu01G06-127 (▪), dosed at 10 mg/kg, in immune-incompetent mice (ICR-SCID) bearing an HT-1080 fibrosarcoma tumor xenograft model. Treatment with antibody Hu01G06-127 reversed body weight loss (FIG. 29A); induced a gain of gonadal fat mass (FIG. 29B); and induced a gain of muscle mass of gastrocnemius muscle (FIG. 29 C) compared to negative control (hIgG (•); FIG. 29A) similar to levels found in non tumor bearing mice (SHAM (▴); FIG. 29A). The arrows in FIG. 29A indicate intra-peritoneal injection of antibody.

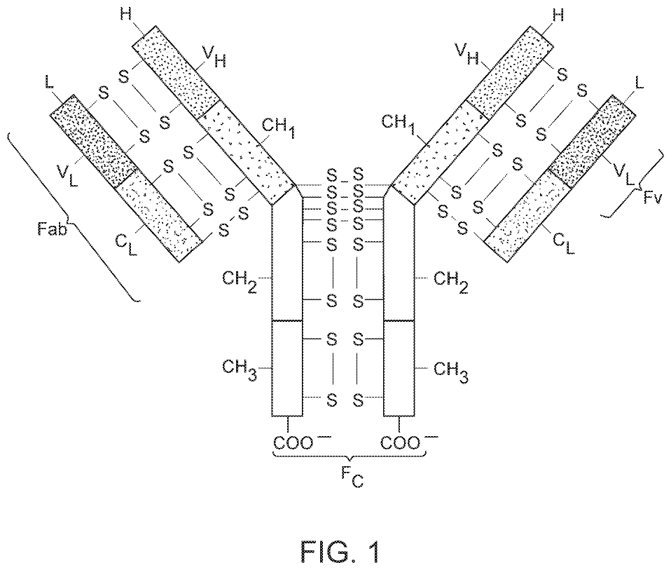

### DETAILED DESCRIPTION

The anti-GDF15 antibodies disclosed herein are based on the antigen binding sites of certain monoclonal antibodies that have been selected on the basis of binding and neutralization of human GDF15 (hGDF15). The antibodies contain immunoglobulin variable region CDR sequences that define a binding site for hGDF15.

By virtue of the neutralizing activity of these antibodies, they are useful for treating cachexia and/or sarcopenia. For use as therapeutic agents, the antibodies can be engineered to minimize or eliminate an immune response when administered to a human patient. Various features and aspects of the invention are discussed in more detail below.

As used herein, “cachexia” means a metabolic syndrome associated with underlying disease and characterized by involuntary loss of muscle mass. Cachexia is often accompanied by involuntary weight loss, loss of fat mass, anorexia, inflammation, insulin resistance, fatigue, weakness, significant loss of appetite, and/or increased muscle protein breakdown. Cachexia is distinct from starvation, age-related loss of muscle mass, malabsorption, and hyperthyroidism. Underlying diseases associated with cachexia include cancer, chronic heart failure, chronic kidney disease, COPD, AIDS, multiple sclerosis, rheumatoid arthritis, sepsis, and tuberculosis.

As used herein, “sarcopenia” is understood to be a condition characterized primarily by loss of skeletal muscle mass and muscle strength. Sarcopenia is frequently associated with aging. See, Ruegg and Glass (2011) ANNUAL REV. PHARMACOL. TOXICOL. 51:373-395. In one approach, sarcopenia can be identified in a subject if a value of the appendicular skeletal muscle mass of a subject divided by the height of the subject in meters is more than two standard deviations below the young normal mean. (Thomas (2007) supra; see also Baumgartner et al. (1999) MECH. AGEING DEV. 147:755-763).

As used herein, unless otherwise indicated, “antibody” means an intact antibody (e.g., an intact monoclonal antibody) or antigen-binding fragment of an antibody, including an intact antibody or antigen-binding fragment that has been modified or engineered, or that is a human antibody. Examples of antibodies that have been modified or engineered are chimeric antibodies, humanized antibodies, and multispecific antibodies (e.g., bispecific antibodies). Examples of antigen-binding fragments include Fab, Fab′, F(ab′)₂, Fv, single chain antibodies (e.g., scFv), minibodies and diabodies.

I. Antibodies that Bind GDF15

The antibodies disclosed herein comprise: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding hGDF15 protein.

In some embodiments, the antibody comprises: (a) an immunoglobulin heavy chain variable region comprising the structure CDR_{H1}-CDR_{H2}-CDR_{H3} and (b) an immunoglobulin light chain variable region, wherein the heavy chain variable region and the light chain variable region together define a single binding site for binding hGDF15. A CDR_{H1} comprises an amino acid sequence selected from the group consisting of SEQ ID NO:1 (01G06, 08G01, Ch01G06 Chimeric, Hu01G06 IGHV1-18, Hu01G06 IGHV1-69, Sh01G06 IGHV1-18 M69L, Sh01G06 IGHV1-18 M69L K64Q G44S, Sh01G06 IGHV1-18 M69L K64Q, Sh01G06 IGHV1-69 T30S I69L, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), SEQ ID NO:2 (03G05), SEQ ID NO:3 (04F08), SEQ ID NO:4 (06C11, Ch06C11 Chimeric, HE LM 06C11 IGHV2-70, Hu06C11 IGHV2-5), SEQ ID NO:5 (14F11, Ch14F11 Chimeric, Sh14F11 IGHV2-5, Sh14F11 IGHV2-70), and SEQ ID NO:6 (17B11); a CDR_{H2} comprises an amino acid sequence selected from the group consisting of SEQ ID NO:7 (01G06, Ch01G06 Chimeric, Hu01G06 IGHV1-18, Hu01G06 IGHV1-69, Sh01G06 IGHV1-18 M69L, Sh01G06 IGHV1-69 T30S I69L), SEQ ID NO:8 (03G05), SEQ ID NO:9 (04F08, 06C11, Ch06C11 Chimeric, Hu06C11 IGHV2-5), SEQ ID NO: 10 (08G01), SEQ ID NO: 11 (14F11, Ch14F11 Chimeric, Sh14F11 IGHV2-5, Sh14F11 IGHV2-70), SEQ ID NO: 12 (17B11), SEQ ID NO: 13 (Sh01G06 IGHV1-18 M69L K64Q G44S, Sh01G06 IGHV1-18 M69L K64Q, Sh01G06 IGHV1-69 T30S K64Q I69L), SEQ ID NO:236 (Hu01G06 IGHV1-18 F1), SEQ ID NO:237 (Hu01G06 IGHV1-18 F2), SEQ ID NO:238 (Hu01G06 IGHV1-69 F1), SEQ ID NO:239 (Hu01G06 IGHV1-69 F2), and SEQ ID NO: 14 (HE LM 06C11 IGHV2-70); and a CDR_{H3} comprises an amino acid sequence selected from the group consisting of SEQ ID NO: 15 (01G06, 08G01, Ch01G06 Chimeric, Hu01G06 IGHV1-18, Hu01G06 IGHV1-69, Sh01G06 IGHV1-18 M69L, Sh01G06 IGHV1-18 M69L K64Q G44S, Sh01G06 IGHV1-18 M69L K64Q, Sh01G06 IGHV1-69 T30S I69L, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), SEQ ID NO:16 (03G05), SEQ ID NO:17 (04F08), SEQ ID NO: 18 (06C11, Ch06C11 Chimeric, HE LM 06C11 IGHV2-70, Hu06C11 IGHV2-5), SEQ ID NO: 19 (14F11, Ch14F11 Chimeric, Sh14F11 IGHV2-5, Sh14F11 IGHV2-70), and SEQ ID NO:20 (17B11). Throughout this specification, a particular SEQ ID NO. is followed in parentheses by the antibody that was the origin of that sequence. For example, “SEQ ID NO:2 (03G05)” means that SEQ ID NO:2 comes from antibody 03G05.

In some embodiments, the antibody comprises an immunoglobulin heavy chain variable region comprising a CDR_{H1} comprising the amino acid sequence of SEQ ID NO:1 (01G06, Ch01G06 Chimeric, Hu01G06 IGHV1-18, Hu01G06 IGHV1-69, Sh01G06 IGHV1-69 T30S I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO:7 (01G06, Ch01G06 Chimeric, Hu01G06 IGHV1-18, Hu01G06 IGHV1-69, Sh01G06 IGHV1-69 T30S I69L), and a CDR_{H3} comprising the amino acid sequence of SEQ ID NO:15 (01G06, Ch01G06 Chimeric, Hu01G06 IGHV1-18, Hu01G06 IGHV1-69, Sh01G06 IGHV1-69 T30S I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:2 (03G05), a CDR_{H2} comprising the amino acid sequence of SEQ ID NO:8 (03G05), and a CDR_{H3} comprising the amino acid sequence of SEQ ID NO:16 (03G05).

In some embodiments, the antibody comprises an immunoglobulin heavy chain variable region comprising a CDR_{H1} comprising the amino acid sequence of SEQ ID NO:3 (04F08), a CDR_{H2} comprising the amino acid sequence of SEQ ID NO:9 (04F08), and a CDR_{H3} comprising the amino acid sequence of SEQ ID NO:17 (04F08).

In some embodiments, the antibody comprises an immunoglobulin heavy chain variable region comprising a CDR_{H1} comprising the amino acid sequence of SEQ ID NO:4 (06C11, Ch06C11 Chimeric, Hu06C11 IGHV2-5), a CDR_{H2} comprising the amino acid sequence of SEQ ID NO:9 (06C11, Ch06C11 Chimeric, Hu06C11 IGHV2-5), and a CDR_{H3} comprising the amino acid sequence of SEQ ID NO:18 (06C11, Ch06C11 Chimeric, Hu06C11 IGHV2-5).

In some embodiments, the antibody comprises an immunoglobulin heavy chain variable region comprising a CDR_{H1} comprising the amino acid sequence of SEQ ID NO:1 (08G01), a CDR_{H2} comprising the amino acid sequence of SEQ ID NO: 10 (08G01), and a CDR_{H3} comprising the amino acid sequence of SEQ ID NO:15 (08G01).

In some embodiments, the antibody comprises an immunoglobulin heavy chain variable region comprising a CDR_{H1} comprising the amino acid sequence of SEQ ID NO:5 (14F11, Ch14F11 Chimeric, Sh14F11 IGHV2-5, Sh14F11 IGHV2-70), a CDR_{H2} comprising the amino acid sequence of SEQ ID NO: 11 (14F11, Ch14F11 Chimeric, Sh14F11 IGHV2-5, Sh14F11 IGHV2-70), and a CDR_{H3} comprising the amino acid sequence of SEQ ID NO: 19 (14F11, Ch14F11 Chimeric, Sh14F11 IGHV2-5, Sh14F11 IGHV2-70).

In some embodiments, the antibody comprises an immunoglobulin heavy chain variable region comprising a CDR_{H1} comprising the amino acid sequence of SEQ ID NO:6 (17B11), a CDR_{H2} comprising the amino acid sequence of SEQ ID NO: 12 (17B11), and a CDR_{H3} comprising the amino acid sequence of SEQ ID NO:20 (17B11).

In some embodiments, the antibody comprises an immunoglobulin heavy chain variable region comprising a CDR_{H1} comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO: 13 (Sh01G06 IGHV1-18 M69L K64Q G44S, Sh01G06 IGHV1-18 M69L K64Q, Sh01G06 IGHV1-69 T30S K64Q I69L), and a CDR_{H3} comprising the amino acid sequence of SEQ ID NO: 15 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO:236 (Hu01G06 IGHV1-18 F1), and a CDR_{H3} comprising the amino acid sequence of SEQ ID NO: 15 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO:237 (Hu01G06 IGHV1-18 F2), and a CDR_{H3} comprising the amino acid sequence of SEQ ID NO: 15 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO:238 (Hu01G06 IGHV1-69 F1), and a CDR_{H3} comprising the amino acid sequence of SEQ ID NO: 15 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), a CDR_{H2} comprising the amino acid sequence of SEQ ID NO:239 (Hu01G06 IGHV1-69 F2), and a CDR_{H3} comprising the amino acid sequence of SEQ ID NO: 15 (Sh01G06 IGHV1-18 M69L K64Q G44S, Sh01G06 IGHV1-18 M69L K64Q, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2).

In some embodiments, the antibody comprises an immunoglobulin heavy chain variable region comprising a CDR_{H1} comprising the amino acid sequence of SEQ ID NO:4 (HE LM 06C11 IGHV2-70), a CDR_{H2} comprising the amino acid sequence of SEQ ID NO: 14 (HE LM 06C11 IGHV2-70), and a CDR_{H3} comprising the amino acid sequence of SEQ ID NO:18 (HE LM 06C11 IGHV2-70).

Preferably, the CDR_{H}1, CDR_{H2}, and CDR_{H3} sequences are interposed between fully human or humanized immunoglobulin FR sequences.

In some embodiments, the antibody comprises (a) an immunoglobulin light chain variable region comprising the structure CDR_{L1}-CDR_{L2}-CDR_{L3}, and (b) an immunoglobulin heavy chain variable region, wherein the immunoglobulin light chain variable region and the immunoglobulin heavy chain variable region together define a single binding site for binding hGDF15. A CDR_{L1} comprises an amino acid sequence selected from the group consisting of SEQ ID NO:21 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), SEQ ID NO:22 (03G05), SEQ ID NO:23 (04F08, 06C11, Ch06C11 Chimeric, Sh06C11 IGKV1-16, 14F11, Ch14F11 Chimeric, Hu14F11 IGKV1-16), SEQ ID NO:24 (08G01), and SEQ ID NO:25 (17B11); a CDR_{L2} comprises an amino acid sequence selected from the group consisting of SEQ ID NO:26 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), SEQ ID NO:27 (03G05), SEQ ID NO:28 (04F08, 06C11, Ch06C11 Chimeric, Sh06C11 IGKV1-16), SEQ ID NO:29 (08G01), SEQ ID NO:30 (14F11, Ch14F11 Chimeric, Hu14F11 IGKV1-16), and SEQ ID NO:31 (17B11); and a CDR_{L3} comprises an amino acid sequence selected from the group consisting of SEQ ID NO:32 (01G06, Ch01G06 Chimeric, Hu01G06 IGKV1-39, Hu01G06 IGKV1-39 S43A V48I, Hu01G06 IGKV1-39 V48I, 08G01, Hu01G06 IGKV1-39 F1), SEQ ID NO:244 (Hu01G06 IGKV1-39 F2), SEQ ID NO:33 (03G05), SEQ ID NO:34 (04F08), SEQ ID NO:35 (06C11, Ch06C11 Chimeric, Sh06C11 IGKV1-16), SEQ ID NO:36 (14F11, Ch14F11 Chimeric, Hu14F11 IGKV1-16), and SEQ ID NO:37 (17B11).

In some embodiments, the antibody comprises an immunoglobulin light chain variable region comprising a CDR_{L1} comprising the amino acid sequence of SEQ ID NO:21 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), a CDR_{L2} comprising the amino acid sequence of SEQ ID NO:26 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), and a CDR_{L3} comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1).

In some embodiments, the antibody comprises an immunoglobulin light chain variable region comprising a CDR_{L1} comprising the amino acid sequence of SEQ ID NO:21 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), a CDR_{L2} comprising the amino acid sequence of SEQ ID NO:26 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), and a CDR_{L3} comprising the amino acid sequence of SEQ ID NO:244 (Hu01G06 IGKV1-39 F2).

In some embodiments, the antibody comprises an immunoglobulin light chain variable region comprising a CDR_{L1} comprising the amino acid sequence of SEQ ID NO:22 (03G05), a CDR_{L2} comprising the amino acid sequence of SEQ ID NO:27 (03G05), and a CDR_{L3} comprising the amino acid sequence of SEQ ID NO:33 (03G05).

In some embodiments, the antibody comprises an immunoglobulin light chain variable region comprising a CDR_{L1} comprising the amino acid sequence of SEQ ID NO:23 (04F08), a CDR_{L2} comprising the amino acid sequence of SEQ ID NO:28 (04F08), and a CDR_{L3} comprising the amino acid sequence of SEQ ID NO:34 (04F08).

In some embodiments, the antibody comprises an immunoglobulin light chain variable region comprising a CDR_{L1} comprising the amino acid sequence of SEQ ID NO:23 (06C11, Ch06C11 Chimeric, Sh06C11 IGKV1-16), a CDR_{L2} comprising the amino acid sequence of SEQ ID NO:28 (06C11, Ch06C11 Chimeric, Sh06C11 IGKV1-16), and a CDR_{L3} comprising the amino acid sequence of SEQ ID NO:35 (06C11, Ch06C11 Chimeric, Sh06C11 IGKV1-16).

In some embodiments, the antibody comprises an immunoglobulin light chain variable region comprising a CDR_{L1} comprising the amino acid sequence of SEQ ID NO:24 (08G01), a CDR_{L2} comprising the amino acid sequence of SEQ ID NO:29 (08G01), and a CDR_{L3} comprising the amino acid sequence of SEQ ID NO:32 (08G01).

In some embodiments, the antibody comprises an immunoglobulin light chain variable region comprising a CDR_{L1} comprising the amino acid sequence of SEQ ID NO:23 (14F11, Ch14F11 Chimeric, Hu14F11 IGKV1-16), a CDR_{L2} comprising the amino acid sequence of SEQ ID NO:30 (14F11, Ch14F11 Chimeric, Hu14F11 IGKV1-16), and a CDR_{L3} comprising the amino acid sequence of SEQ ID NO:36 (14F11, Ch14F11 Chimeric, Hu14F11 IGKV1-16).

In some embodiments, the antibody comprises an immunoglobulin light chain variable region comprising a CDR_{L1} comprising the amino acid sequence of SEQ ID NO:25 (17B11), a CDR_{L2} comprising the amino acid sequence of SEQ ID NO:31 (17B11), and a CDR_{L3} comprising the amino acid sequence of SEQ ID NO:37 (17B11).

Preferably, the CDR_{L1}, CDR_{L2}, and CDR_{L3} sequences are interposed between fully human or humanized immunoglobulin FR sequences.

In some embodiments, the antibody comprises: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding hGDF15. The CDR_{H1} is an amino acid sequence selected from the group consisting of SEQ ID NO:1 (01G06, 08G01, Ch01G06 Chimeric, Hu01G06 IGHV1-18, Hu01G06 IGHV1-69, Sh01G06 IGHV1-18 M69L, Sh01G06 IGHV1-18 M69L K64Q G44S, Sh01G06 IGHV1-18 M69L K64Q, Sh01G06 IGHV1-69 T30S I69L, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), SEQ ID NO:2 (03G05), SEQ ID NO:3 (04F08), SEQ ID NO:4 (06C11, Ch06C11 Chimeric, HE LM 06C11 IGHV2-70, Hu06C11 IGHV2-5), SEQ ID NO:5 (14F11, Ch14F11 Chimeric, Sh14F11 IGHV2-5, Sh14F11 IGHV2-70), and SEQ ID NO:6 (17B11); the CDR_{H2} is an amino acid sequence selected from the group consisting of SEQ ID NO:7 (01G06, Ch01G06 Chimeric, Hu01G06 IGHV1-18, Hu01G06 IGHV1-69, Sh01G06 IGHV1-18 M69L, Sh01G06 IGHV1-69 T30S I69L), SEQ ID NO:8 (03G05), SEQ ID NO:9 (04F08, 06C11, Ch06C11 Chimeric, Hu06C11 IGHV2-5), SEQ ID NO:10 (08G01), SEQ ID NO:11 (14F11, Ch14F11 Chimeric, Sh14F11 IGHV2-5, Sh14F11 IGHV2-70), SEQ ID NO: 12 (17B11), SEQ ID NO: 13 (Sh01G06 IGHV1-18 M69L K64Q G44S, Sh01G06 IGHV1-18 M69L K64Q, Sh01G06 IGHV1-69 T30S K64Q 169L), SEQ ID NO:236 (Hu01G06 IGHV1-18 F1), SEQ ID NO:237 (Hu01G06 IGHV1-18 F2), SEQ ID NO:238 (Hu01G06 IGHV1-69 F1), SEQ ID NO:239 (Hu01G06 IGHV1-69 F2), and SEQ ID NO: 14 (HE LM 06C11 IGHV2-70); and the CDR_{H3} is an amino acid sequence selected from the group consisting of SEQ ID NO: 15 (01G06, 08G01, Ch01G06 Chimeric, Hu01G06 IGHV1-18, Hu01G06 IGHV1-69, Sh01G06 IGHV1-18 M69L, Sh01G06 IGHV1-18 M69L K64Q G44S, Sh01G06 IGHV1-18 M69L K64Q, Sh01G06 IGHV1-69 T30S I69L, Sh01G06 IGHV1-69 T30S K64Q I69L, Hu01G06 IGHV1-18 F1, Hu01G06 IGHV1-18 F2, Hu01G06 IGHV1-69 F1, Hu01G06 IGHV1-69 F2), SEQ ID NO:16 (03G05), SEQ ID NO:17 (04F08), SEQ ID NO: 18 (06C11, Ch06C11 Chimeric, HE LM 06C11 IGHV2-70, Hu06C11 IGHV2-5), SEQ ID NO: 19 (14F11, Ch14F11 Chimeric, Sh14F11 IGHV2-5, Sh14F11 IGHV2-70), and SEQ ID NO:20 (17B11). The CDR_{L1} is an amino acid sequence selected from the group consisting of SEQ ID NO:21 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), SEQ ID NO:22 (03G05), SEQ ID NO:23 (04F08, 06C11, Ch06C11 Chimeric, Sh06C11 IGKV1-16, 14F11, Ch14F11 Chimeric, Hu14F11 IGKV1-16), SEQ ID NO:24 (08G01), and SEQ ID NO:25 (17B11); the CDR_{L2} is an amino acid sequence selected from the group consisting of SEQ ID NO:26 (01G06, Ch01G06 Chimeric, Hu01G06 IGKV1-39, Hu01G06 IGKV1-39 S43A V48I, Hu01G06 IGKV1-39 V48I, Hu01G06 IGKV1-39 F1, Hu01G06 IGKV1-39 F2), SEQ ID NO:27 (03G05), SEQ ID NO:28 (04F08, 06C11, Ch06C11 Chimeric, Sh06C11 IGKV1-16), SEQ ID NO:29 (08G01), SEQ ID NO:30 (14F11, Ch14F11 Chimeric, Hu14F11 IGKV1-16), and SEQ ID NO:31 (17B11); and the CDR_{L3} is an amino acid sequence selected from the group consisting of SEQ ID NO:32 (01G06, Ch01G06 Chimeric, Hu01G06 IGKV1-39, Hu01G06 IGKV1-39 S43A V48I, Hu01G06 IGKV1-39 V48I, 08G01, Hu01G06 IGKV1-39 F1), SEQ ID NO:244 (Hu01G06 IGKV1-39 F2), SEQ ID NO:33 (03G05), SEQ ID NO:34 (04F08), SEQ ID NO:35 (06C11, Ch06C11 Chimeric, Sh06C11 IGKV1-16), SEQ ID NO:36 (14F11, Ch14F11 Chimeric, Hu14F11 IGKV1-16), and SEQ ID NO:37 (17B11).

In some embodiments, the antibody comprises an immunoglobulin heavy chain variable region comprising a CDR_{H1} comprising an amino acid sequence selected from the group consisting of SEQ ID NO:1 and SEQ ID NO:38 (Hu01G06 IGHV1-18 F1), a CDR_{H2} comprising an amino acid sequence selected from the group consisting of SEQ ID NO:236 and SEQ ID NO:240 (Hu01G06 IGHV1-18 F1), and a CDR_{H3} comprising the amino acid sequence of SEQ ID NO:15 (Hu01G06 IGHV1-18 F1); and an immunoglobulin light chain variable region comprising a CDR_{L1} comprising the amino acid sequence of SEQ ID NO:21 (Hu01G06 IGKV1-39 F1), a CDR_{L2} comprising the amino acid sequence of SEQ ID NO:26 (Hu01G06 IGKV1-39 F1), and a CDR_{L3} comprising the amino acid sequence of SEQ ID NO:32 (Hu01G06 IGKV1-39 F1).

In some embodiments, the antibody comprises an immunoglobulin heavy chain variable region comprising a CDR_{H1} comprising an amino acid sequence selected from the group consisting of SEQ ID NO:1 and SEQ ID NO:38 (Hu01G06 IGHV1-18 F2), a CDR_{H2} comprising an amino acid sequence selected from the group consisting of SEQ ID NO:237 and SEQ ID NO:241 (Hu01G06 IGHV1-18 F2), and a CDR_{H3} comprising the amino acid sequence of SEQ ID NO:15 (Hu01G06 IGHV1-18 F2); and an immunoglobulin light chain variable region comprising a CDR_{L1} comprising the amino acid sequence of SEQ ID NO:21 (Hu01G06 IGKV1-39 F2), a CDR_{L2} comprising the amino acid sequence of SEQ ID NO:26 (Hu01G06 IGKV1-39 F2), and a CDR_{L3} comprising the amino acid sequence of SEQ ID NO:244 (Hu01G06 IGKV1-39 F2).

In some embodiments, the antibody comprises an immunoglobulin heavy chain variable region comprising a CDR_{H1} comprising an amino acid sequence selected from the group consisting of SEQ ID NO:1 and SEQ ID NO:234 (Hu01G06 IGHV1-69 F1), a CDR_{H2} comprising an amino acid sequence selected from the group consisting of SEQ ID NO:238 and SEQ ID NO:241 (Hu01G06 IGHV1-69 F1), and a CDR_{H3} comprising the amino acid sequence of SEQ ID NO:15 (Hu01G06 IGHV1-69 F1); and an immunoglobulin light chain variable region comprising a CDR_{L1} comprising the amino acid sequence of SEQ ID NO:21 (Hu01G06 IGKV1-39 F1), a CDR_{L2} comprising the amino acid sequence of SEQ ID NO:26 (Hu01G06 IGKV1-39 F1), and a CDR_{L3} comprising the amino acid sequence of SEQ ID NO:32 (Hu01G06 IGKV1-39 F1).

In some embodiments, the antibody comprises an immunoglobulin heavy chain variable region comprising a CDR_{H1} comprising an amino acid sequence selected from the group consisting of SEQ ID NO:1 and SEQ ID NO:234 (Hu01G06 IGHV1-69 F2), a CDR_{H2} comprising an amino acid sequence selected from the group consisting of SEQ ID NO:239 and SEQ ID NO:240 (Hu01G06 IGHV1-69 F2), and a CDR_{H3} comprising the amino acid sequence of SEQ ID NO:15 (Hu01G06 IGHV1-69 F2); and an immunoglobulin light chain variable region comprising a CDR_{L1} comprising the amino acid sequence of SEQ ID NO:21 (Hu01G06 IGKV1-39 F1), a CDR_{L2} comprising the amino acid sequence of SEQ ID NO:26 (Hu01G06 IGKV1-39 F1), and a CDR_{L3} comprising the amino acid sequence of SEQ ID NO:32 (Hu01G06 IGKV1-39 F1).

In some embodiments, the antibody comprises an immunoglobulin heavy chain variable region comprising a CDR_{H1} comprising an amino acid sequence selected from the group consisting of SEQ ID NO:1 and SEQ ID NO:234 (Hu01G06 IGHV1-69 F2), a CDR_{H2} comprising an amino acid sequence selected from the group consisting of SEQ ID NO:239 and SEQ ID NO:240 (Hu01G06 IGHV1-69 F2), and a CDR_{H3} comprising the amino acid sequence of SEQ ID NO:15 (Hu01G06 IGHV1-69 F2); and an immunoglobulin light chain variable region comprising a CDR_{L1} comprising the amino acid sequence of SEQ ID NO:21 (Hu01G06 IGKV1-39 F2), a CDR_{L2} comprising the amino acid sequence of SEQ ID NO:26 (Hu01G06 IGKV1-39 F2), and a CDR_{L3} comprising the amino acid sequence of SEQ ID NO:244 (Hu01G06 IGKV1-39 F2).

The antibodies disclosed herein comprise an immunoglobulin heavy chain variable region and an immunoglobulin light chain variable region. In some embodiments, the antibody comprises an immunoglobulin heavy chain variable region selected from the group consisting of SEQ ID NO:40 (01G06, Ch01G06 Chimeric), SEQ ID NO:42 (03G05), SEQ ID NO:44 (04F08), SEQ ID NO:46 (06C11, Ch06C11 Chimeric), SEQ ID NO:48 (08G01), SEQ ID NO:50 (14F11, Ch14F11 Chimeric), SEQ ID NO:52 (17B11), SEQ ID NO:54 (Hu01G06 IGHV1-18), SEQ ID NO:56 (Hu01G06 IGHV1-69), SEQ ID NO:58 (Sh01G06 IGHV1-18 M69L), SEQ ID NO:60 (Sh01G06 IGHV1-18 M69L K64Q G44S), SEQ ID NO:62 (Sh01G06 IGHV1-18 M69L K64Q), SEQ ID NO:64 (Sh01G06 IGHV1-69 T30S I69L), SEQ ID NO:66 (Sh01G06 IGHV1-69 T30S K64Q I69L), SEQ ID NO:246 (Hu01G06 IGHV1-18 F1), SEQ ID NO:248 (Hu01G06 IGHV1-18 F2), SEQ ID NO:250 (Hu01G06 IGHV1-69 F1), SEQ ID NO:252 (Hu01G06 IGHV1-69 F2), SEQ ID NO:68 (HE LM 06C11 IGHV2-70), SEQ ID NO:70 (Hu06C11 IGHV2-5), SEQ ID NO:72 (Sh14F11 IGHV2-5), and SEQ ID NO:74 (Sh14F11 IGHV2-70); and an immunoglobulin light chain variable region.

In other embodiments, the antibody comprises an immunoglobulin light chain variable region selected from the group consisting of SEQ ID NO:76 (01G06, Ch01G06 Chimeric), SEQ ID NO:78 (03G05), SEQ ID NO:80 (04F08), SEQ ID NO:82 (06C11, Ch06C11 Chimeric), SEQ ID NO:84 (08G01), SEQ ID NO:86 (14F11, Ch14F11 Chimeric), SEQ ID NO:88 (17B11), SEQ ID NO:90 (Hu01G06 IGKV1-39), SEQ ID NO:92 (Hu01G06 IGKV1-39 S43A V48I or Hu01G06 IGKV1-39 F1), SEQ ID NO:94 (Hu01G06 IGKV1-39 V48I), SEQ ID NO:96 (Sh06C11 IGKV1-16), SEQ ID NO:254 (Hu01G06 IGKV1-39 F2), and SEQ ID NO:98 (Hu14F11 IGKV1-16), and an immunoglobulin heavy chain variable region.

In some embodiments, the antibody comprises an immunoglobulin heavy chain variable region selected from the group consisting of SEQ ID NO:40 (01G06, Ch01G06 Chimeric), SEQ ID NO:42 (03G05), SEQ ID NO:44 (04F08), SEQ ID NO:46 (06C11, Ch06C11 Chimeric), SEQ ID NO:48 (08G01), SEQ ID NO:50 (14F11, Ch14F11 Chimeric), SEQ ID NO:52 (17B11), SEQ ID NO:54 (Hu01G06 IGHV1-18), SEQ ID NO:56 (Hu01G06 IGHV1-69), SEQ ID NO:58 (Sh01G06 IGHV1-18 M69L), SEQ ID NO:60 (Sh01G06 IGHV1-18 M69L K64Q G44S), SEQ ID NO:62 (Sh01G06 IGHV1-18 M69L K64Q), SEQ ID NO:64 (Sh01G06 IGHV1-69 T30S I69L), SEQ ID NO:66 (Sh01G06 IGHV1-69 T30S K64Q I69L), SEQ ID NO:246 (Hu01G06 IGHV1-18 F1), SEQ ID NO:248 (Hu01G06 IGHV1-18 F2), SEQ ID NO:250 (Hu01G06 IGHV1-69 F1), SEQ ID NO:252 (Hu01G06 IGHV1-69 F2), SEQ ID NO:68 (HE LM 06C11 IGHV2-70), SEQ ID NO:70 (Hu06C11 IGHV2-5), SEQ ID NO:72 (Sh14F11 IGHV2-5), and SEQ ID NO:74 (Sh14F11 IGHV2-70), and an immunoglobulin light chain variable region selected from the group consisting of SEQ ID NO:76 (01G06, Ch01G06 Chimeric), SEQ ID NO:78 (03G05), SEQ ID NO:80 (04F08), SEQ ID NO:82 (06C11, Ch06C11 Chimeric), SEQ ID NO:84 (08G01), SEQ ID NO:86 (14F11, Ch14F11 Chimeric), SEQ ID NO:88 (17B11), SEQ ID NO:90 (Hu01G06 IGKV1-39), SEQ ID NO:92 (Hu01G06 IGKV1-39 S43A V48I or Hu01G06 IGKV1-39 F1), SEQ ID NO:94 (Hu01G06 IGKV1-39 V48I), SEQ ID NO:96 (Sh06C11 IGKV1-16), SEQ ID NO:254 (Hu01G06 IGKV1-39 F2), and SEQ ID NO:98 (Hu14F11 IGKV1-16).

In some embodiments, the antibody comprises an immunoglobulin heavy chain variable region comprising the amino acid sequence of SEQ ID NO:40 (01G06, Ch01G06 Chimeric), and an immunoglobulin light chain variable region comprising the amino acid sequence of SEQ ID NO:76 (01G06, Ch01G06 Chimeric).

In some embodiments, the antibody comprises an immunoglobulin heavy chain variable region comprising the amino acid sequence of SEQ ID NO:42 (03G05), and an immunoglobulin light chain variable region comprising the amino acid sequence of SEQ ID NO:78 (03G05).

In some embodiments, the antibody comprises an immunoglobulin heavy chain variable region comprising the amino acid sequence of SEQ ID NO:44 (04F08), and an immunoglobulin light chain variable region comprising the amino acid sequence of SEQ ID NO:80 (04F08).

In some embodiments, the antibody comprises an immunoglobulin heavy chain variable region comprising the amino acid sequence of SEQ ID NO:46 (06C11), and an immunoglobulin light chain variable region comprising the amino acid sequence of SEQ ID NO:82 (06C11).

In some embodiments, the antibody comprises an immunoglobulin heavy chain variable region comprising the amino acid sequence of SEQ ID NO:48 (08G01), and an immunoglobulin light chain variable region comprising the amino acid sequence of SEQ ID NO:84 (08G01).

In some embodiments, the antibody comprises an immunoglobulin heavy chain variable region comprising the amino acid sequence of SEQ ID NO:50 (14F11), and an immunoglobulin light chain variable region comprising the amino acid sequence of SEQ ID NO:86 (14F11).

In some embodiments, the antibody comprises an immunoglobulin heavy chain variable region comprising the amino acid sequence of SEQ ID NO:52 (17B11), and an immunoglobulin light chain variable region comprising the amino acid sequence of SEQ ID NO:88 (17B11).

In some embodiments, the antibody comprises an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:40 (Ch01G06 Chimeric), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:90 (Hu01G06 IGKV1-39).

In some embodiments, the antibody comprises an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:90 (Hu01G06 IGKV1-39).

In some embodiments, the antibody comprises an immuoglobulin heavy chain variable region comprising the amino acid sequence of SEQ ID NO:40 (Ch01G06 Chimeric), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:92 (Hu01G06 IGKV1-39 S43A V48I).

In some embodiments, the antibody comprises an immunoglobulin heavy chain variable region comprising the amino acid sequence of SEQ ID NO:40 (Ch01G06 Chimeric), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:66 (Sh01G06 IGHV1-69 T30S K64Q 169L), and an immunoglobulin light chain variable region comprising the amino acid sequence of SEQ ID NO:94 (Hu01G06 IGKV1-39 V48I).

In some embodiments, the antibody comprises an immunoglobulin heavy chain variable region comprising the amino acid sequence of SEQ ID NO:246 (Hu01G06 IGHV1-18 F1), and an immunoglobulin light chain variable region comprising the amino acid sequence of SEQ ID NO:92 (Hu01G06 IGKV1-39 F1).

In some embodiments, the antibody comprises an immunoglobulin heavy chain variable region comprising the amino acid sequence of SEQ ID NO:248 (Hu01G06 IGHV1-18 F2), and an immunoglobulin light chain variable region comprising the amino acid sequence of SEQ ID NO:254 (Hu01G06 IGKV1-39 F2).

In some embodiments, the antibody comprises an immunoglobulin heavy chain variable region comprising the amino acid sequence of SEQ ID NO:250 (Hu01G06 IGHV1-69 F1), and an immunoglobulin light chain variable region comprising the amino acid sequence of SEQ ID NO:92 (Hu01G06 IGKV1-39 F1).

In some embodiments, the antibody comprises an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:92 (Hu01G06 IGKV1-39 F1).

In some embodiments, the antibody comprises an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:254 (Hu01G06 IGKV1-39 F2).

In some embodiments, the antibody comprises an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:82 (Ch06C11 Chimeric).

In some embodiments, the antibody comprises an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:82 (Ch06C11 Chimeric).

In some embodiments, the antibody comprises an immunoglobulin heavy chain variable region comprising the amino acid sequence of SEQ ID NO:46 (Ch06C11 Chimeric), and an immunoglobulin light chain variable region comprising the amino acid sequence of SEQ ID NO:96 (Sh06C11 IGKV1-16).

In some embodiments, the antibody comprises an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:96 (Sh06C11 IGKV1-16).

In some embodiments, the antibody comprises an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:96 (Sh06C11 IGKV1-16).

In some embodiments, the antibody comprises an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:86 (Ch14F11 Chimeric).

In some embodiments, the antibody comprises an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:86 (Ch14F11 Chimeric).

In some embodiments, the antibody comprises an immunoglobulin heavy chain variable region comprising the amino acid sequence of SEQ ID NO:50 (Ch14F11 Chimeric), and an immunoglobulin light chain variable region comprising the amino acid sequence of SEQ ID NO:98 (Hu14F11 IGKV1-16).

In some embodiments, the antibody comprises an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:98 (Hu14F11 IGKV1-16).

In some embodiments, the antibody comprises an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:98 (Hu14F11 IGKV1-16).

In some embodiments, the antibody comprises an immunoglobulin heavy chain variable region comprising the amino acid sequence of SEQ ID NO:46 (Ch06C11 Chimeric), and an immunoglobulin light chain variable region comprising the amino acid sequence of SEQ ID NO:80 (04F08).

In some embodiments, the antibody comprises an immunoglobulin heavy chain variable region comprising the amino acid sequence of SEQ ID NO:50 (Ch14F11 Chimeric), and an immunoglobulin light chain variable region comprising the amino acid sequence of SEQ ID NO:80 (04F08).

In some embodiments, the antibody comprises an immunoglobulin heavy chain variable region comprising the amino acid sequence of SEQ ID NO:44 (04F08), and an immunoglobulin light chain variable region comprising the amino acid sequence of SEQ ID NO:82 (Ch06C11 Chimeric).

In some embodiments, the antibody comprises an immunoglobulin heavy chain variable region comprising the amino acid sequence of SEQ ID NO:50 (Ch14F11 Chimeric), and an immunoglobulin light chain variable region comprising the amino acid sequence of SEQ ID NO:82 (Ch06C11 Chimeric).

In some embodiments, the antibody comprises an immunoglobulin heavy chain variable region comprising the amino acid sequence of SEQ ID NO:44 (04F08), and an immunoglobulin light chain variable region comprising the amino acid sequence of SEQ ID NO:86 (Ch14F11 Chimeric).

In some embodiments, the antibody comprises an immunoglobulin heavy chain variable region comprising the amino acid sequence of SEQ ID NO:46 (Ch06C11 Chimeric), and an immunoglobulin light chain variable region comprising the amino acid sequence of SEQ ID NO:86 (Ch14F11 Chimeric).

In some embodiments, the antibody comprises an immunoglobulin heavy chain variable region comprising the amino acid sequence of SEQ ID NO:48 (08G01), and an immunoglobulin light chain variable region comprising the amino acid sequence of SEQ ID NO:76 (Ch01G06 Chimeric).

In some embodiments, the antibody comprises an immunoglobulin heavy chain variable region comprising the amino acid sequence of SEQ ID NO:40 (Ch01G06 Chimeric), and an immunoglobulin light chain variable region comprising the amino acid sequence of SEQ ID NO:84 (08G01).

In certain embodiments, the antibodies disclosed herein comprise an immunoglobulin heavy chain and an immunoglobulin light chain. In some embodiments, the antibody comprises an immunoglobulin heavy chain selected from the group consisting of SEQ ID NO:100 (01G06), SEQ ID NO:104 (03G05), SEQ ID NO:108 (04F08), SEQ ID NO:112 (06C11), SEQ ID NO:116 (08G01), SEQ ID NO:120 (14F11), SEQ ID NO:124 (17B11), SEQ ID NO:176 (Ch01G06 Chimeric), SEQ ID NO:178 (Hu01G06 IGHV1-18), SEQ ID NO:180 (Hu01G06 IGHV1-69), SEQ ID NO:182 (Sh01G06 IGHV1-18 M69L), SEQ ID NO:184 (Sh01G06 IGHV1-18 M69L K64Q G44S), SEQ ID NO:186 (Sh01G06 IGHV1-18 M69L K64Q), SEQ ID NO:188 (Sh01G06 IGHV1-69 T30S I69L), SEQ ID NO:190 (Sh01G06 IGHV1-69 T30S K64Q I69L), SEQ ID NO:256 (Hu01G06 IGHV1-18 F1), SEQ ID NO:258 (Hu01G06 IGHV1-18 F2), SEQ ID NO:260 (Hu01G06 IGHV1-69 F1), SEQ ID NO:262 (Hu01G06 IGHV1-69 F2), SEQ ID NO:192 (Ch06C11 Chimeric), SEQ ID NO:194 (HE LM 06C11 IGHV2-70), SEQ ID NO:196 (Hu06C11 IGHV2-5), SEQ ID NO:198 (Ch14F11 Chimeric), SEQ ID NO:200 (Sh14F11 IGHV2-5), and SEQ ID NO:202 (Sh14F11 IGHV2-70); and an immunoglobulin light chain.

In other embodiments, the antibody comprises an immunoglobulin light chain selected from the group consisting of SEQ ID NO: 102 (01G06), SEQ ID NO:106 (03G05), SEQ ID NO:110 (04F08), SEQ ID NO:114 (06C11), SEQ ID NO:118 (08G01), SEQ ID NO:122 (14F11), SEQ ID NO:126 (17B11), SEQ ID NO:204 (Ch01G06 Chimeric), SEQ ID NO:206 (Hu01G06 IGKV1-39), SEQ ID NO:208 (Hu01G06 IGKV1-39 S43A V48I or Hu01G06 IGKV1-39 F1), SEQ ID NO:210 (Hu01G06 IGKV1-39 V48I), SEQ ID NO:264 (Hu01G06 IGKV1-39 F2), SEQ ID NO:212 (Ch06C11 Chimeric), SEQ ID NO:214 (Sh06C11 IGKV1-16), SEQ ID NO:216 (Ch14F11 Chimeric), and SEQ ID NO:218 (Hu14F11 IGKV1-16), and an immunoglobulin heavy chain.

In some embodiments, the antibody comprises (i) an immunoglobulin heavy chain selected from the group consisting of SEQ ID NO: 100 (01G06), SEQ ID NO: 104 (03G05), SEQ ID NO: 108 (04F08), SEQ ID NO: 112 (06C11), SEQ ID NO: 116 (08G01), SEQ ID NO: 120 (14F11), SEQ ID NO: 124 (17B11), SEQ ID NO: 176 (Ch01G06 Chimeric), SEQ ID NO:178 (Hu01G06 IGHV1-18), SEQ ID NO:180 (Hu01G06 IGHV1-69), SEQ ID NO:182 (Sh01G06 IGHV1-18 M69L), SEQ ID NO: 184 (Sh01G06 IGHV1-18 M69L K64Q G44S), SEQ ID NO: 186 (Sh01G06 IGHV1-18 M69L K64Q), SEQ ID NO: 188 (Sh01G06 IGHV1-69 T30S I69L), SEQ ID NO: 190 (Sh01G06 IGHV1-69 T30S K64Q I69L), SEQ ID NO:256 (Hu01G06 IGHV1-18 F1), SEQ ID NO:258 (Hu01G06 IGHV1-18 F2), SEQ ID NO:260 (Hu01G06 IGHV1-69 F1), SEQ ID NO:262 (Hu01G06 IGHV1-69 F2), SEQ ID NO: 192 (Ch06C11 Chimeric), SEQ ID NO: 194 (HE LM 06C11 IGHV2-70), SEQ ID NO: 196 (Hu06C11 IGHV2-5), SEQ ID NO:198 (Ch14F11 Chimeric), SEQ ID NO:200 (Sh14F11 IGHV2-5), and SEQ ID NO:202 (Sh14F11 IGHV2-70), and (ii) an immunoglobulin light chain selected from the group consisting of SEQ ID NO: 102 (01G06), SEQ ID NO: 106 (03G05), SEQ ID NO:110 (04F08), SEQ ID NO:114 (06C11), SEQ ID NO:118 (08G01), SEQ ID NO: 122 (14F11), SEQ ID NO: 126 (17B11), SEQ ID NO:204 (Ch01G06 Chimeric), SEQ ID NO:206 (Hu01G06 IGKV1-39), SEQ ID NO:208 (Hu01G06 IGKV1-39 S43A V48I or Hu01G06 IGKV1-39 F1), SEQ ID NO:210 (Hu01G06 IGKV1-39 V48I), SEQ ID NO:264 (Hu01G06 IGKV1-39 F2), SEQ ID NO:212 (Ch06C11 Chimeric), SEQ ID NO:214 (Sh06C11 IGKV1-16), SEQ ID NO:216 (Ch14F11 Chimeric), and SEQ ID NO:218 (Hu14F11 IGKV1-16).

In some embodiments, the antibody comprises an immunoglobulin heavy chain comprising the amino acid sequence of SEQ ID NO: 176 (Ch01G06 Chimeric), and an immunoglobulin light chain comprising the amino acid sequence of SEQ ID NO:204 (Ch01G06 Chimeric).

In some embodiments, the antibody comprises an immunoglobulin heavy chain comprising the amino acid sequence of SEQ ID NO: 192 (Ch06C11 Chimeric), and an immunoglobulin light chain comprising the amino acid sequence of SEQ ID NO:212 (Ch06C11 Chimeric).

In some embodiments, the antibody comprises an immunoglobulin heavy chain comprising the amino acid sequence of SEQ ID NO:198 (Ch14F11 Chimeric), and an immunoglobulin light chain comprising the amino acid sequence of SEQ ID NO:216 (Ch14F11 Chimeric).

In some embodiments, the antibody comprises an immunoglobulin heavy chain comprising the amino acid sequence of SEQ ID NO:178 (Hu01G06 IGHV1-18), and an immunoglobulin light chain comprising the amino acid sequence of SEQ ID NO:206 (Hu01G06 IGKV1-39).

In some embodiments, the antibody comprises an immunoglobulin heavy chain comprising the amino acid sequence of SEQ ID NO:180 (Hu01G06 IGHV1-69), and an immunoglobulin light chain comprising the amino acid sequence of SEQ ID NO:206 (Hu01G06 IGKV1-39).

In some embodiments, the antibody comprises an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:210 (Hu01G06 IGKV1-39 V48I).

In some embodiments, the antibody comprises an immunoglobulin heavy chain comprising the amino acid sequence of SEQ ID NO:188 (Sh01G06 IGHV1-69 T30S I69L), and an immunoglobulin light chain comprising the amino acid sequence of SEQ ID NO:210 (Hu01G06 IGKV1-39 V48I).

In some embodiments, the antibody comprises an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:208 (Hu01G06 IGKV1-39 S43A V48I).

In some embodiments, the antibody comprises an immunoglobulin heavy chain comprising the amino acid sequence of SEQ ID NO:188 (Sh01G06 IGHV1-69 T30S I69L), and an immunoglobulin light chain comprising the amino acid sequence of SEQ ID NO:208 (Hu01G06 IGKV1-39 S43A V48I).

In some embodiments, the antibody comprises an immunoglobulin heavy chain comprising the amino acid sequence of SEQ ID NO:256 (Hu01G06 IGHV1-18 F1), and an immunoglobulin light chain comprising the amino acid sequence of SEQ ID NO:208 (Hu01G06 IGKV1-39 F1).

In some embodiments, the antibody comprises an immunoglobulin heavy chain comprising the amino acid sequence of SEQ ID NO:258 (Hu01G06 IGHV1-18 F2), and an immunoglobulin light chain comprising the amino acid sequence of SEQ ID NO:264 (Hu01G06 IGKV1-39 F2).

In some embodiments, the antibody comprises an immunoglobulin heavy chain comprising the amino acid sequence of SEQ ID NO:260 (Hu01G06 IGHV1-69 F1), and an immunoglobulin light chain comprising the amino acid sequence of SEQ ID NO:208 (Hu01G06 IGKV1-39 F1).

In some embodiments, the antibody comprises an immunoglobulin heavy chain comprising the amino acid sequence of SEQ ID NO:262 (Hu01G06 IGHV1-69 F2), and an immunoglobulin light chain comprising the amino acid sequence of SEQ ID NO:208 (Hu01G06 IGKV1-39 F1).

In some embodiments, the antibody comprises an immunoglobulin heavy chain comprising the amino acid sequence of SEQ ID NO:262 (Hu01G06 IGHV1-69 F2), and an immunoglobulin light chain comprising the amino acid sequence of SEQ ID NO:264 (Hu01G06 IGKV1-39 F2).

In some embodiments, the antibody comprises an immunoglobulin heavy chain comprising the amino acid sequence of SEQ ID NO:194 (HE LM 06C11 IGHV2-70), and an immunoglobulin light chain comprising the amino acid sequence of SEQ ID NO:214 (Sh06C11 IGKV1-16).

In some embodiments, the antibody comprises an immunoglobulin heavy chain comprising the amino acid sequence of SEQ ID NO:196 (Hu06C11 IGHV2-5), and an immunoglobulin light chain comprising the amino acid sequence of SEQ ID NO:214 (Sh06C11 IGKV1-16).

In some embodiments, the antibody comprises an immunoglobulin heavy chain comprising the amino acid sequence of SEQ ID NO:200 (Sh14F11 IGHV2-5), and an immunoglobulin light chain comprising the amino acid sequence of SEQ ID NO:218 (Hu14F11 IGKV1-16)

In some embodiments, the antibody comprises an immunoglobulin heavy chain comprising the amino acid sequence of SEQ ID NO:202 (Sh14F11 IGHV2-70), and an immunoglobulin light chain comprising the amino acid sequence of SEQ ID NO:218 (Hu14F11 IGKV1-16).

In certain embodiments, an isolated antibody that binds hGDF15 comprises an immunoglobulin heavy chain variable region comprising an amino acid sequence that is at least 70%, 75%, 80%, 85%, 90%, 95%, 98%, or 99% identical to the entire variable region or the FR sequence of SEQ ID NO:40 (01G06, Ch01G06 Chimeric), SEQ ID NO:42 (03G05), SEQ ID NO:44 (04F08), SEQ ID NO:46 (06C11, Ch06C11 Chimeric), SEQ ID NO:48 (08G01), SEQ ID NO:50 (14F11, Ch14F11 Chimeric), SEQ ID NO:52 (17B11), SEQ ID NO:54 (Hu01G06 IGHV1-18), SEQ ID NO:56 (Hu01G06 IGHV1-69), SEQ ID NO:58 (Sh01G06 IGHV1-18 M69L), SEQ ID NO:60 (Sh01G06 IGHV1-18 M69L K64Q G44S), SEQ ID NO:62 (Sh01G06 IGHV1-18 M69L K64Q), SEQ ID NO:64 (Sh01G06 IGHV1-69 T30S I69L), SEQ ID NO:66 (Sh01G06 IGHV1-69 T30S K64Q I69L), SEQ ID NO:246 (Hu01G06 IGHV1-18 F1), SEQ ID NO:248 (Hu01G06 IGHV1-18 F2), SEQ ID NO:250 (Hu01G06 IGHV1-69 F1), SEQ ID NO:252 (Hu01G06 IGHV1-69 F2), SEQ ID NO:68 (HE LM 06C11 IGHV2-70), SEQ ID NO:70 (Hu06C11 IGHV2-5), SEQ ID NO:72 (Sh14F11 IGHV2-5), and SEQ ID NO:74 (Sh14F11 IGHV2-70).

In certain embodiments, an isolated antibody that binds hGDF15 comprises an immunoglobulin light chain variable region comprising an amino acid sequence that is at least 70%, 75%, 80%, 85%, 90%, 95%, 98%, or 99% identical to the entire variable region or the FR sequence of SEQ ID NO:76 (01G06, Ch01G06 Chimeric), SEQ ID NO:78 (03G05), SEQ ID NO:80 (04F08), SEQ ID NO:82 (06C11, Ch06C11 Chimeric), SEQ ID NO:84 (08G01), SEQ ID NO:86 (14F11, Ch14F11 Chimeric), SEQ ID NO:88 (17B11), SEQ ID NO:90 (Hu01G06 IGKV1-39), SEQ ID NO:92 (Hu01G06 IGKV1-39 S43A V48I or Hu01G06 IGKV1-39 F1), SEQ ID NO:94 (Hu01G06 IGKV1-39 V48I), SEQ ID NO:254 (Hu01G06 IGKV1-39 F2), SEQ ID NO:96 (Sh06C11 IGKV1-16), and SEQ ID NO:98 (Hu14F11 IGKV1-16).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

In each of the foregoing embodiments, it is contemplated herein that immunoglobulin heavy chain variable region sequences and/or light chain variable region sequences that together bind human GDF15 may contain amino acid alterations (e.g., at least 1, 2, 3, 4, 5, or 10 amino acid substitutions, deletions, or additions) in the framework regions of the heavy and/or light chain variable regions.

In some embodiments, the antibody binds hGDF15 with a K_{D} of about 300 pM, 250 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, or 10 pM, or lower. Unless otherwise specified, K_{D} values are determined by surface plasmon resonance methods or biolayer interferometry under the conditions described in Examples 8, 14, and 15.

In some embodiments, a monoclonal antibody binds to the same epitope on hGDF15 (e.g., mature hGDF15 or cleaved rhGDF15) bound by one or more of the antibodies disclosed herein (e.g., antibodies 01G06, 03G05, 04F08, 06C11, 08G01, 14F11, or 17B11). In some embodiments, a monoclonal antibody competes for binding to hGDF15 with one or more of the antibodies disclosed herein (e.g., antibody 01G06, 03G05, 04F08, 06C11, 08G01, 14F11, or 17B11).

Competition assays for determining whether an antibody binds to the same epitope as, or competes for binding with, an anti-GDF15 antibody disclosed herein are known in the art. Exemplary competition assays include immunoassays (e.g., ELISA assays, RIA assays), surface plasmon resonance analysis (e.g., using a BIAcore™ instrument), biolayer interferometry and flow cytometry.

Typically, a competition assay involves the use of an antigen (e.g., a hGDF15 protein or fragment thereof) bound to a solid surface or expressed on a cell surface, a test anti-GDF15-binding antibody and a reference antibody (e.g., antibody 01G06, 03G05, 04F08, 06C11, 08G01, 14F11, or 17B11). The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

In an exemplary competition assay, a reference anti-GDF15 antibody (e.g., antibody 01G06, 03G05, 04F08, 06C11, 08G01, 14F11, or 17B11) is biotinylated using commercially available reagents. The biotinylated reference antibody is mixed with serial dilutions of the test antibody or unlabeled reference antibody (self-competition control) resulting in a mixture of various molar ratios (e.g., 1×, 5×, 10×, 20× or 100×) of test antibody (or unlabeled reference antibody) to labeled reference antibody. The antibody mixture is added to a hGDF15 polypeptide coated-ELISA plate. The plate is then washed, and horseradish peroxidase (HRP)-strepavidin is added to the plate as the detection reagent. The amount of labeled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are known in the art. Optical density readings (OD units) are measured using a SpectraMax® M2 spectrometer (Molecular Devices). OD units corresponding to zero percent inhibition are determined from wells without any competing antibody. OD units corresponding to 100% inhibition, i.e., the assay background are determined from wells without any labeled reference antibody or test antibody. Percent inhibition of labeled reference antibody to GDF15 by the test antibody (or the unlabeled reference antibody) at each concentration is calculated as follows: % inhibition=(1−(OD units−100% inhibition)/(0% inhibition−100% inhibition))*100. Persons skilled in the art will appreciate that the competition assay can be performed using various detection systems known in the art.

A competition assay may be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99%, as measured in a competitive binding assay.

Two antibodies bind to the same epitope if essentially all amino acid mutations in the antigen that reduce or eliminate binding of one antibody reduce or eliminate binding of the other. Two antibodies bind to overlapping epitopes if only a subset of the amino acid mutations that reduce or eliminate binding of one antibody reduce or eliminate binding of the other.

### II. Production of Antibodies

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukayotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

A monoclonal antibody that binds hGDF15, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

### III. Antibody Modifications

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an anti-GDF15 antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains are said to be produced. See, e.g., U.S. Pat. No. 6,706,477 (Zauderer); U.S. Pat. No. 6,800,442 (Zauderer); and 6,872,518 (Zauderer).

Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. No. 5,766,886 (Studnicka); U.S. Pat. No. 5,770,196 (Studnicka); U.S. Pat. No. 5,821,123 (Studnicka); and 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human mAbs can also be prepared and optimized from phage display libraries by techniques referenced in, e.g., Knappik et al., J. MOL. BIOL. 296:57-86, 2000; and Krebs et al., J. Immunol. Meth. 254:67-84 2001).

### IV. Therapeutic Uses

The antibodies disclosed herein can be used to treat a variety of disorders, for example, cachexia and/or sarcopenia. In some embodiments, the antibodies disclosed herein (e.g., 01G06, 03G05, 04F08, 06C11, 08G01, 14F11, or 17B11) are used to inhibit the loss of muscle mass, for example, the loss of muscle mass associated with an underlying disease. Underlying diseases associated with cachexia include, but are not limited to, cancer, chronic heart failure, chronic kidney disease, COPD, AIDS, multiple sclerosis, rheumatoid arthritis, sepsis, and tuberculosis. In some embodiments, the disclosed antibodies inhibit loss of muscle mass by at least 40%, 50%, 60%, 70%, 80%, 90%, 95%, 98%, 99%, or 100%.

In some embodiments, a loss of muscle mass is accompanied by a loss of fat mass. The antibodies disclosed herein (e.g., 01G06, 03G05, 04F08, 06C11, 08G01, 14F11, or 17B11) may inhibit loss of fat mass by at least 40%, 50%, 60%, 70%, 80%, 90%, 95%, 98%, 99%, or 100%.

In other embodiments, the antibodies disclosed herein (e.g., 01G06, 03G05, 04F08, 06C11, 08G01, 14F11, or 17B11) are used to treat one or more features accompanying cachexia and/or sarcopenia, e.g., involuntary body weight loss. In some embodiments, the antibodies revert involuntary body weight loss by at least 2%, 5%, 10%, 15%, 20%, 25%, 30% or 35%.

In another embodiment, the antibodies disclosed herein (e.g., 01G06, 03G05, 04F08, 06C11, 08G01, 14F11, or 17B11) are used to inhibit loss of organ mass, for example, loss of organ mass associated with an underlying disease. Underlying diseases associated with cachexia include, but are not limited to, cancer, chronic heart failure, chronic kidney disease, COPD, AIDS, multiple sclerosis, rheumatoid arthritis, sepsis, and tuberculosis. In some embodiments, the disclosed antibodies inhibit loss of organ mass by at least 40%, 50%, 60%, 70%, 80%, 90%, 95%, 98%, 99%, or 100%. In some embodiments, loss of organ mass is observed in heart, liver, kidney, and/or spleen. In some embodiments, the loss of organ mass in accompanied by a loss of muscle mass, a loss of fat mass and/or involuntary weight loss.

Antibody 01G06, 03G05, 04F08, 06C11, 08G01, 14F11, or 17B11 can be used in therapy. For example, antibody 01G06, 03G05, 04F08, 06C11, 08G01, 14F11, or 17B11 can be used to treat cachexia and/or sarcopenia. Use of antibody 01G06, 03G05, 04F08, 06C11, 08G01, 14F11, or 17B11 to treat cachexia and/or sarcopenia in a mammal comprises administering to the mammal a therapeutically effective amount of the antibody.

Sarcopenia, muscle wasting disorders and significant muscle weight loss may occur in the absence of cachexia, decreased appetite or body weight loss. In certain embodiments, therefore, one or more of the anti-GDF antibodies of the invention (for example, antibody 01G06, 03G05, 04F08, 06C11, 08G01, 14F11, or 17B11) can be used to treat a subject suffering from, or who has been diagnosed with, sarcopenia, a muscle wasting disorder and/or significant muscle weight loss, whether or not the subject has, or has been diagnosed with, cachexia or decreased appetite. Such a method comprises administering a therapeutically effective amount of one or more antibodies of the invention to the subject in need thereof.

The Fc-rhGDF15 fusion proteins disclosed herein can be used to treat obesity. In some embodiments, the hFc-rhGDF15 fusion proteins disclosed herein are used to inhibit weight gain or to reduce body weight by at least 5%, 10%, 15%, 20%, 25%, 30%, 35%, 40%, 45% or 50%. Use of an hFc-hGDF15 fusion protein to treat obesity in a mammal comprises administering to the mammal a therapeutically effective amount of the fusion protein.

As used herein, “treat,” “treating” and “treatment” mean the treatment of a disease in a mammal, e.g., in a human. This includes: (a) inhibiting the disease, i.e., arresting its development; and (b) relieving the disease, i.e., causing regression of the disease state.

Generally, a therapeutically effective amount of an active component (e.g., an antibody or a fusion protein) is in the range of 0.1 mg/kg to 100 mg/kg, e.g., 1 mg/kg to 100 mg/kg, e.g., 1 mg/kg to 10 mg/kg, e.g., 2.0 mg/kg to 10 mg/kg. The amount administered will depend on variables such as the type and extent of disease or indication to be treated, the overall health of the patient, the in vivo potency of the antibody or fusion protein, the pharmaceutical formulation, the serum half-life of the antibody or fusion protein, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody or fusion protein, and the disease being treated. Exemplary dosing frequencies are once per day, once per week and once every two weeks. In some embodiments, dosing is once every two weeks. A preferred route of administration is parenteral, e.g., intravenous infusion. Formulation of monoclonal antibody-based drugs and fusion protein-based drugs are within ordinary skill in the art. In some embodiments, the antibody or fusion protein is lyophilized, and then reconstituted in buffered saline, at the time of administration. The effective amount of a second active agent, for example, an anti-cancer agent or the other agents discussed below, will also follow the principles discussed hereinabove and will be chosen so as to elicit the required therapeutic benefit in the patient.

For therapeutic use, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies or fusion proteins, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

In addition to the GDF15 (i.e., MIC-1/PLAB/PDF/NAG-1) pathway, other cytokines implicated in cachexia include Activin A and IL-6. Increased activin levels have been associated with cancer-associated cachexia and gonadal tumors. See, e.g., Marino et al. (2013) CYTOKINE & GROWTH FACTOR REV. 24:477-484. Activin A is a member of the TGF-beta family, and is a ligand of the activin type 2 receptor, ActRIIB. See, e.g., Zhou et al. (2010) CELL 142:531-543. Circulating levels of IL-6 have been shown to correlate with weight loss in cancer patients, as well as with reduced survival. See, e.g., Fearon et al. (2012) CELL METABOLISM 16:153-166.

Accordingly, in certain embodiments of the present invention, one or more inhibitors of Activin-A or the Activin-A receptor, ActRIIB, IL-6 or the IL-6 receptor (IL-6R), may be administered in combination with (for example, administered at the same time as, administered before, or administered after) an antibody of the present invention that inhibits GDF-15 activity. Exemplary inhibitors of Activin A or ActRIIB, include, for example, an anti-Activin-A antibody or an antigen binding fragment thereof, an anti-ActRIIB antibody or an antigen binding fragment thereof, a small molecule inhibitor of Activin-A, a small molecule inhibitor of ActRIIB, and a ‘decoy’ receptor of ActRIIB, such as a soluble ActRIIB receptor and a fusion of the soluble ActRIIB receptor with an Fc molecule (ActRIIB-Fc). See, for example, Zhou et al. (2010), supra. Suitable inhibitors of IL-6 or IL-6R, include an anti-IL-6 antibody or an antigen binding fragment thereof, an anti-IL-6R antibody or an antigen binding fragment thereof, a small molecule inhibitor of IL-6, a small molecule inhibitor of IL-6R, and a ‘decoy’ receptor of IL-6R, such as a soluble IL-6 receptor and a fusion of the soluble IL-6 receptor with an Fc molecule (IL6R-Fc). See, e.g., Enomoto et al. (2004) BIOCHEM. AND BIOPHYS. RES. COMM. 323:1096-1102; Argiles et al. (2011) EUR. J. PHARMACOL. 668:S81-S86; Tuca et al. (2013) ONCOLOGY/HEMATOLOGY 88:625-636. Suitable inhibitors of IL-6 or IL-6R may include, for example, Tocilizumab (Actemra®, Hoffmann-LaRoche), a humanized anti-IL-6R monoclonal antibody approved for treatment of rheumatoid arthritis, and Sarilumab/REGN88 (Regeneron), a humanized anti-IL6R antibody in clinical development for treatment of rheumatoid arthritis; and Selumetinib/AZD6244 (AstraZeneca), an allosteric inhibitor of MEK, which has been shown to inhibit IL-6 production. Prado et al. (2012) BRITISH J. CANCER 106:1583-1586.

TNFα and IL-1 are cytokines known to be involved in mediation of the proinflammatory response, which are also implicated in muscle depletion, anorexia and cachexia. Increased circulating levels of TNFα appear to inhibit myogenesis. TNFα, also known as “cachectin,” stimulates interleukin-1 secretion and is implicated in the induction of cachexia. IL-1 is a potent trigger of the acute-phase inflammatory response, and it has been shown that infusion of IL-1 can lead to marked weight loss and appetite loss. IL-1 has been shown to contribute to the initiation of cancer cachexia in mice bearing a murine colon-26 adenocarcinoma (Strassmann et al. (1993) J. IMMUNOL. 150:2341). See also, Mathys and Billiau (1997) NUTRITION 13:763-770; Fong et al. (1989) AM. J. PHYSIOL.—REGULATORY, INTEGRATIVE AND COMPARATIVE PHYSIOL., 256:R659-R665. Thus, TNFα inhibitors and IL-1 inhibitors that are used in the treatment of rheumatoid arthritis may also be useful in the treatment of cachexia.

Accordingly, in certain embodiments of the present invention, one or more inhibitors of TNFα or IL-1 may be administered in combination with (for example, administered at the same time as, administered before, or administered after) an antibody of the present invention that inhibits GDF-15 activity. Suitable inhibitors of TNFα or IL-1 include an anti-TNFα antibody or an antigen binding fragment thereof, an anti-IL-1 antibody or an antigen binding fragment thereof, a small molecule inhibitor of TNFα or IL-1, and a ‘decoy’ receptor of TNFα or IL-1, such as a soluble TNFα or IL-1 receptor and a fusion of the soluble form of TNFα or IL-1 with an Fc molecule. Suitable inhibitors of TNFα include for example, etanercept (Enbrel®, Pfizer/Amgen), infliximab (Remicade®, Janssen Biotech), adalimumab (Humira®, Abbvie), golimumab (Simponi®, Johnson and Johnson/Merck), and certolizumab pegol (Cimzia®, UCB). Suitable IL-1 inhibitors include, for example, Xilonix® antibody that targets IL-1α (XBiotech), anikinra (Kinaret®, Amgen), canakinumab (Ilaris®, Novartis), and rilonacept (Arcalyst®, Regeneron). In certain embodiments, the TNFα inhibitor or IL-1 inhibitor, which is typically administered systemically for the treatment of rheumatoid arthritis may be administered locally and directly to the tumor site.

Myostatin, also known as GDF-8, is a member of the TGF-0 family of peptides that is a negative regulator of muscle mass, as shown by increased muscle mass in myostatin deficient mammals. Myostatin is a ligand of the activin type 2 receptor, ActRIIB. Accordingly, in certain embodiments of the present invention, one or more inhibitors of myostatin or its receptor may be administered in combination with (for example, administered at the same time as, administered before, or administered after) an antibody of the invention that inhibits GDF-15 activity. Suitable inhibitors of myostatin or ActRIIB, include an anti-myostatin antibody or an antigen binding fragment thereof, an anti-ActRIIB antibody or an antigen binding fragment thereof, a small molecule inhibitor of myostatin, a small molecule inhibitor of ActRIIB, and a ‘decoy’ receptor of GDF-8, such as a soluble ActRIIB and a fusion of the soluble form of ActRIIB with an Fc molecule. See, e.g., Lokireddy et al. (2012) BIOCHEM. J. 446(1):23-26. Myostatin inhibitors that may be suitable for the present invention include REGN1033 (Regeneron); see Bauerlein et al. (2013) J. CACHEXIA SARCOPENIA MUSCLE: Abstracts of the 7^{th} Cachexia Conference, Kobe/Osaka, Japan, Dec. 9-11, 2013, Abstract 4-06; LY2495655 (Lilly), a humanized anti-myostatin antibody in clinical development by Eli Lilly; see also “A PHASE 2 STUDY OF LY2495655 IN PARTICIPANTS WITH PANCREATIC CANCER,” available on the world wide web at clinicaltrials.gov/ct2/NCT01505530; NML identifier: NCT01505530; ACE-031 (Acceleron Pharma); and stamulumab (Pfizer).

Agents such as Ghrelin or ghrelin mimetics, or other growth hormone secretagogues (GHS) which are able to activate the GHS receptor (GHS-R1a), also known as the ghrelin receptor, may be useful for increasing food intake and body weight in humans. See Guillory et al. (2013) in VITAMINS AND HORMONES vol. 92, chap.3; and Steinman and DeBoer (2013) VITAMINS AND HORMONES vol. 92, chap. 8. Suitable ghrelin mimetics include anamorelin (Helsinn, Lugano, CH); See Temel et al. (2013) J. CACHEXIA SARCOPENIA MUSCLE: Abstracts of the 7^{th} Cachexia Conference, Kobe/Osaka, Japan, Dec. 9-11, 2013, Abstract 5-01. Other suitable GHS molecules can be identified, for example, using the growth hormone secretagogue receptor Ghrelin competition assay described in PCT Publication Nos. WO2011/117254 and WO2012/113103.

Agonists of the androgen receptor, including small molecules and other selective androgen receptor modulators (SARMs) may be useful in treating cachexia and/or sarcopenia. See, e.g., Mohler et al. (2009) J. MED. CHEM. 52:3597-3617; Nagata et al. (2011) BIOORGANIC AND MED. CHEM. LETTERS 21:1744-1747; and Chen et al. (2005) MOL. INTERV. 5:173-188. Ideally, SARMs should act as full agonists, like testosterone, in anabolic target tissues, such as muscle and bone, but should demonstrate only partial or pure androgen receptor antagonistic activities on prostate tissue. See, e.g., Bovee et al. (2010) J. STEROID BIOCHEM. & MOL. BIOL. 118:85-92. Suitable SARMs can be identified, for example, by use of the methods and assays described in Zhang et al. (2006) BIOORG. MED. CHEM. LETT. 16:5763-5766; and Zhang et al. (2007) BIOORG. MED. CHEM. LETT. 17:439-443. Suitable SARMs include, for example, GTx-024 (enobosarm, Ostarine®, GTx, Inc.), a SARM in phase II clinical development by GTx, Inc. See also, Dalton et al. (2011) J. CACHEXIA SARCOPENIA MUSCLE 2:153-161. Other suitable SARMs include 2-(2,2,2)-trifluoroethyl-benzimidazoles (Ng et al. (2007) BIOORG. MED. CHEM. LETT. 17:1784-1787) and JNJ-26146900 (Allan et al. (2007) J. STEROID BIOCHEM. & MOL. BIOL. 103:76-83).

β-adrenergic receptor blockers, or beta-blockers, have been studied for their effect on body weight in cachexic subjects, and have been associated with partial reversal of cachexia in patients with congestive heart failure. See, e.g., Hryniewicz et al. (2003) J. CARDIAC FAILURE 9:464-468. Beta-blocker MT-102 (PsiOxus Therapeutics, Ltd.) has been evaluated in a phase 2 clinical trial for subjects with cancer cachexia. See Coats et al. (2011) J. CACHEXIA SARCOPENIA MUSCLE 2:201-207.

Melanocortin receptor-knockout mice with a genetic defect in melanocortin signaling exhibit a phenotype opposite that of cachexia: increased appetite, increased lean body mass, and decreased metabolism. Thus, melanocortin antagonism has emerged as a potential treatment for cachexia associated with chronic disease (DeBoer and Marks (2006) TRENDS IN ENDOCRINOLOGY AND METABOLISM 17:199-204). Accordingly, in certain embodiments of the present invention, one or more inhibitors of a melanocortin peptide or a melanocortin receptor may be administered in combination (for example, administered at the same time as, administered before, or administered after) with an antibody of the invention that inhibits GDF-15 activity. Suitable inhibitors of melanocortins or melanocortin receptors include an anti-melanocortin peptide antibody or an antigen binding fragment thereof, an anti-melanocortin receptor antibody or an antigen binding fragment thereof, a small molecule inhibitor of a melanocortin peptide, a small molecule inhibitor of a melanocortin receptor, and a ‘decoy’ receptor of a melanocortin receptor, such as soluble melanocortin receptor and a fusion of a soluble melanocortin receptor with an Fc molecule. Suitable melacortin receptor inhibitors include, for example, the melanocortin receptor antagonist agouri-related peptide (AgRP(83-132)), which has been demonstrated to prevent cachexia-related symptoms in a mouse model of cancer-related cachexia (Joppa et al. (2007) PEPTIDES 28:636-642).

Anti-cancer agents, especially those that can cause cachexia and elevate GDF-15 levels, such as cisplatin, may be used in methods of the present invention in combination with (for example, administered at the same time as, administered before, or administered after) an anti-GDF-15 antibody of the invention. Many cancer patients are weakened by harsh courses of radio- and/or chemotherapy, which can limit the ability of the patient to tolerate such therapies, and hence restrict the dosage regimen. Certain cancer agents themselves, such as fluorouracil, Adriamycin, methotrexate and cisplatin, may contribute to cachexia, for example by inducing severe gastrointestinal complications. See, e.g., Inui (2002) CANCER J. FOR CLINICIANS 52:72-91. By the methods of the present invention, in which an anti-cancer agent is administered in combination with an anti-GDF-15 antibody of the invention, it is possible to decrease the incidence and/or severity of cachexia, and ultimately increase the maximum tolerated dose of such an anti-cancer agent. Accordingly, efficacy of treatment with anti-cancer agents that may cause cachexia can be improved by reducing the incidence of cachexia as a dose-limiting adverse effect, and by allowing administration of higher doses of a given anti-cancer agent.

Thus, the present invention includes pharmaceutical compositions comprising an anti-GDF-15 antibody of the present invention in combination with an agent selected from the group consisting of an inhibitor of Activin-A, an inhibitor of ActRIIB, an inhibitor of IL-6 or an inhibitor of IL-6R, a ghrelin, a ghrelin mimetic or a GHS-R1a agonist, a SARM, a TNFα inhibitor, an IL-1α inhibitor, a myostatin inhibitor, a beta-blocker, a melanocortin peptide inhibitor, a melanocortin receptor inhibitor, and an anti-cancer agent. The present invention also includes methods of treating, preventing or minimizing cachexia and/or sarcopenia in a mammal comprising administering to a mammal in need thereof a pharmaceutical composition or compositions comprising an effective amount of an anti-GDF-15 antibody of the invention in combination with an effective amount of an inhibitor of Activin-A, an inhibitor of ActRIIB, an inhibitor of IL-6 or an inhibitor of IL-6R, a ghrelin, a ghrelin mimetic or a GHS-R1a agonist, a SARM, a TNFα inhibitor, an IL-1α inhibitor, a myostatin inhibitor, a beta-blocker, a melanocortin peptide inhibitor, or a melanocortin receptor inhibitor.

In another embodiment, the invention comprises a method of inhibiting loss of muscle mass associated with an underlying disease comprising administering to a mammal in need thereof a pharmaceutical composition or compositions comprising an effective amount of an anti-GDF-15 antibody of the invention in combination with an effective amount of an inhibitor of Activin-A, an inhibitor of ActRIIB, an inhibitor of IL-6 or an inhibitor of IL-6R, a ghrelin, a ghrelin mimetic or a GHS-R1a agonist, a SARM, a TNFα inhibitor, an IL-1α inhibitor, a myostatin inhibitor, a beta-blocker, a melanocortin peptide inhibitor, or a melanocortin receptor inhibitor to prevent or reduce loss of muscle mass. The underlying disease may be selected from the group consisting of cancer, chronic heart failure, chronic kidney disease, COPD, AIDS, multiple sclerosis, rheumatoid arthritis, sepsis, and tuberculosis. Additionally, in certain embodiments, the loss of muscle mass is accompanied by a loss of fat mass.

In yet further embodiments, the present invention comprises a method of inhibiting or reducing involuntary weight loss in a mammal comprising administering to a mammal in need thereof a pharmaceutical composition or pharmaceutical compositions comprising an effective amount of an anti-GDF-15 antibody of the invention in combination with an effective amount of an inhibitor of Activin-A, an inhibitor of ActRIIB, an inhibitor of IL-6 or an inhibitor of IL-6R, a ghrelin, a ghrelin mimetic or a GHS-R1a agonist, a SARM, a TNFα inhibitor, a IL-1α inhibitor, a myostatin inhibitor, a beta-blocker, a melanocortin peptide inhibitor, or a melanocortin receptor inhibitor.

Certain anti-cancer agents, such as cisplatin, have one or more undesirable adverse effects that involve causing or increasing one or more syndromes such as cachexia, sarcopenia, muscle wasting, bone wasting or involuntary body weight loss. Accordingly, in certain embodiments, the present invention comprises a method of treating cancer, while preventing, minimizing or reducing the occurrence, frequency or severity of cachexia, sarcopenia, or muscle wasting, bone wasting or involuntary loss of body weight in a mammal, comprising administering to a mammal in need thereof a pharmaceutical composition comprising an effective amount of an anti-GDF-15 antibody of the present invention in combination with with one or more anti-cancer agents. In particular embodiments, the invention comprises a method of treating cancer, while preventing, minimizing or reducing the occurrence, frequency or severity of cachexia, sarcopenia or muscle wasting, bone wasting or involuntary loss of body weight in a mammal, comprising administering to a mammal in need thereof a pharmaceutical composition comprising an effective amount of an anti-GDF-15 antibody of the invention in combination with one or more anti-cancer agents known to cause or increase the occurrence, frequency or severity of cachexia, sarcopenia, or muscle wasting, bone wasting or involuntary loss of body weight in a mammal.

### EXAMPLES

The following Examples are merely illustrative and are not intended to limit the scope or content of the invention in any way.

### Example 1: Human GDF15 Serum Levels in Mouse Xenograft Tumor Models

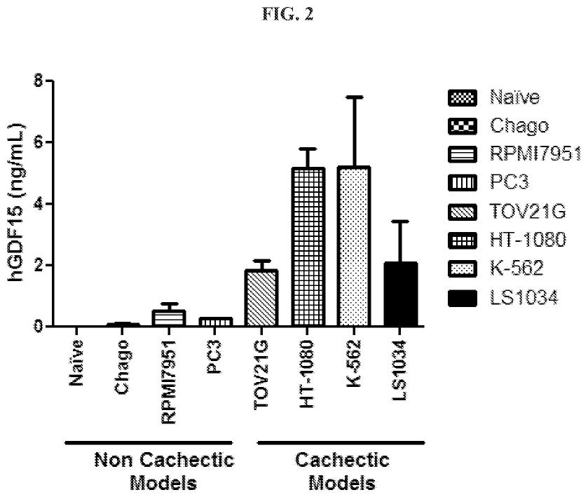

In this example, the amount of hGDF15 in the serum of mice bearing various xenograft tumors was measured. Serum was collected from three mice for each of the following tumor xenograft models: Chago, RPMI7951, PC3, TOV21G, HT-1080, K-562, and LS1034. Serum was also collected from three naïve mice as a control. Human GDF15 serum levels were determined by ELISA (R&D Systems, Cat. No. DY957E). Mice bearing human xenograft tumors that induce cachexia had serum levels of hGDF15 above 2 ng/mL, while mice bearing human xenograft tumors that do not induce cachexia had serum levels of hGDF15 below 1 ng/mL (FIG. 2). Naïve mice had no detectable hGDF15 (control). These results indicate that a serum level of approximately 2 ng/mL GDF15 is a threshold for inducing cachexia in this mouse model. Similar levels of hGDF15 were also observed in plasma when determined by ELISA.

### Example 2: Non-Tumor Bearing Mouse Model of Cachexia

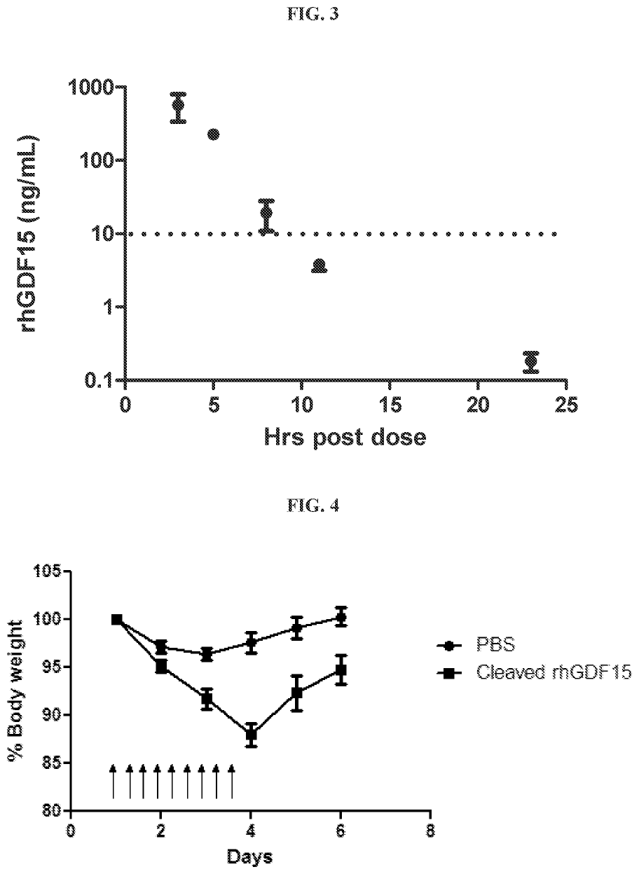

An existing non-tumor bearing mouse model of cachexia is based on the injection of mature rhGDF15 into a mouse (Johnen et al. (1997) NAT. MED. 13:1333-1340). Mature rhGDF15 corresponds to amino acids 197 to 308 of hGDF15 protein. Mature rhGDF15 can be produced in the yeast Pichia pastoris as described in Fairlie et al. (2000) GENE 254:67-76). Cleaved-rhGDF15 corresponds to amino acids 197 to 308 of hGDF15 protein released from an Fc-rhGDF15 fusion protein. In FIGS. 3-4 described below, cleaved-rhGDF15 was produced by enzymatic digestion of mFc-rhGDF15 fusion protein with Factor Xa, and subsequent purification, prior to injection in mice.

To investigate the half-life of cleaved-rhGDF15, plasma was collected from a group of three mice after single dose of cleaved-rhGDF15 (1 μg/g) at different time points (2, 5, 8, 11, and 23 hours). Human GDF15 plasma levels were determined by ELISA (R&D Systems, Cat. No. DY957E). As shown in FIG. 3, cleaved-rhGDF15 was rapidly cleared from the plasma following injection. Eleven hours post-injection, the amount of cleaved-rhGDF15 in the plasma was below 10 ng/mL, and, within 23-hours, cleaved-rhGDF15 was almost completely cleared from the plasma.

The rapid clearance of cleaved-rhGDF15 in non-tumor bearing mice was further investigated. Eight-week old female ICR-SCID mice were randomized into two groups of ten mice each. Mice were dosed subcutaneously into the flank every eight hours for three days (a total of nine doses) with one of the following treatments: PBS (control) or cleaved-rhGDF15 at 1 μg/g. Body weight was measured daily. Statistical analyses were performed using a two-way ANOVA.

As shown in FIG. 4, cleaved-rhGDF15 induced body weight loss. After nine doses over a three day period, percent body weight dropped to 88% at day 4 (p<0.001), but approximately 24 hours after the last dose the mice began to gain weight. On day 6, the last day of the experiment, percent body weight increased to 94.8 percent (p<0.001). These results indicate that weight loss induced by cleaved-rhGDF15 is not sustained over long periods of time. The activity observed with cleaved-rhGDF15 described herein was similar to that observed with mature rhGDF15 in the existing mouse model (Johnen et al., supra).

The existing non-tumor bearing mouse model for cachexia relies on the injection of large amounts of mature rhGDF15 delivered in multiple doses per day to induce muscle loss and body weight loss (Johnen et al., supra). It appears that if mature rhGDF15 or cleaved-rhGDF15 is used, the mice do not sustain muscle weight loss or body weight loss for long periods of time without continuous dosing. This limits the usefulness of such models. Moreover, repeated dosing requires frequent handling of these mice which introduces stress that can compromise the reliability of body weight loss measurements. For example, as shown in FIG. 4, mice treated with multiple doses of PBS demonstrated a body weight drop due to the stress of repeated dosing and handling.

### Example 3: GDF15 Fusion Proteins

In view of the large amounts of mature rhGDF15 (or cleaved-rhGDF15) and the labor intensity required to induce non-tumor bearing cachexia mouse models (as well as the resulting limitations of these models), we investigated alternate forms of rhGDF15 to induce a cachetic phenotype in mice. This Example describes the construction and production of two fusion proteins consisting of GDF15 and an immunoglobulin Fc fragment, designated mFc-rhGDF15 (mouse IgG1 Fc fused to the amino terminus of mature human GDF15) and rFc-rmGDF15 (rabbit IgG1 Fc fused to the amino terminus of mature mouse GDF15). The GDF15 fusion proteins were designed using methods known in the art. The mFc-rhGDF15 DNA sequences were constructed from fragments using overlap extension PCR to include (in the following order): 5′ HindIII restriction site, Kozak consensus sequence, amino terminal signal sequence, mouse IgG1 Fc, Factor Xa cleavage site, a polypeptide linker (GGGGS) (SEQ ID NO: 139), mature hGDF15, stop codon, and a 3′ EcoRI restriction site. The rFc-rmGDF15 amino acid sequences were converted to codon-optimized DNA sequences and synthesized to include (in the following order): 5′ HindIII restriction site, Kozak consensus sequence, amino terminal signal sequence, rabbit IgG1 Fc, a polypeptide linker (GGGG) (SEQ ID NO: 265), mature mouse GDF15, stop codon, and a 3′ EcoRI restriction site.

The GDF15 fusion proteins were subcloned into the mammalian expression vector pEE14.4 (Lonza, Basel, Switzerland) via HindIII and EcoRI sites using In-Fusion™ PCR cloning (Clontech, Mountain View, CA). GDF15 fusion proteins were stably expressed in CHOK1SV cells using the GS System™ (Lonza Biologics) in order to produce large quantities of purified protein. Each expression vector was linearized and transfected into CHOK1SV cells. Stable clones were selected in the presence of methionine sulfoximine. Secreted proteins produced by CHOK1SV stably transfected cell lines were purified by Protein A and size exclusion chromatography.

The nucleic acid sequence and the encoded protein sequence defining the mouse IgG1 Fc-mature human GDF15 fusion protein (mFc-rhGDF15) are shown below. mFc-rhGDF15 contains mouse IgG1 Fc from amino acids 1-222, Factor Xa cleavage site from amino acids 223-228, an artificial linker sequence from amino acids 229-233, and mature hGDF15 from amino acids 234-345.

Nucleic Acid Sequence Encoding the Mouse IgG1 Fc—Mature Human GDF15 Fusion Protein (mFc-rhGDF15) (SEQ ID NO:219)

| | |
|---|---|
| 1 | gggtgtaaac cctgcatctg cacggtgccg gaggtgtcct |
| | ccgtctttat cttccctccc |
| 61 | aaacccaagg atgtgctgac aatcactttg actccaaaag |
| | tcacatgcgt agtcgtggac |
| 121 | atctcgaaag acgacccgga agtgcagttc tcgtggtttg |
| | ttgatgatgt agaagtgcat |
| 181 | accgctcaaa cccagccgag ggaagaacag tttaacagca |
| | cgtttaggag tgtgtcggaa |
| 241 | ctgcccatta tgcaccagga ttggcttaat gggaaggagt |
| | tcaaatgtcg cgtgaatagt |
| 301 | gcggcgttcc cagcccctat tgaaaagact atttccaaaa |
| | cgaagggtcg gcccaaagct |
| 361 | ccccaagtat acacaatccc tccgccgaaa gaacaaatgg |
| | caaaagacaa agtgagtttg |
| 421 | acgtgcatga tcacggactt tttcccggag gatatcaccg |
| | tcgaatggca atggaatggg |
| 481 | caacctgccg aaaactacaa gaatacacaa cccattatgg |
| | ataccgatgg atcgtatttc |
| 541 | gtctactcaa agttgaacgt acagaagtca aattgggagg |
| | cagggaatac gttcacttgc |
| 601 | agtgttttgc acgaaggcct ccataaccac catacggaaa |
| | agtcactgtc gcactccccg |
| 661 | ggaaaaatcg agggcagaat ggatggtgga ggagggtcgg |
| | cgcgcaacgg ggaccactgt |
| 721 | ccgctcgggc ccgggcgttg ctgccgtctg cacacggtcc |
| | gcgcgtcgct ggaagacctg |
| 781 | ggctgggccg attgggtgct gtcgccacgg gaggtgcaag |
| | tgaccatgtg catcggcgcg |
| 841 | tgcccgagcc agttccgggc ggcaaacatg cacgcgcaga |
| | tcaagacgag cctgcaccgc |
| 901 | ctgaagcccg acacggtgcc agcgccctgc tgcgtgcccg |
| | ccagctacaa tcccatggtg |
| 961 | ctcattcaaa agaccgacac cggggtgtcg ctccagacct |
| | atgatgactt gttagccaaa |
| 1021 | gactgccact gcata |

Protein Sequence Defining the Mouse IgG1 Fc—Mature Human GDF15 Fusion Protein (mFc-rhGDF15) (SEQ ID NO:220)

| | |
|---|---|
| 1 | gckpcictvp evssvfifpp kpkdvltitl tpkvtcvvvd iskddpevqf swfvddvevh |
| 61 | taqtqpreeq fnstfrsvse lpimhqdwln gkefkcrvns aafpapiekt isktkgrpka |
| 121 | pqvytipppk eqmakdkvsl tcmitdffpe ditvewqwng qpaenykntq pimdtdgsyf |
| 181 | vysklnvqks nweagntftc svlheglhnh htekslshsp gkiegrmdgg ggsarngdhc |
| 241 | plgpgrccrl htvrasledl gwadwvlspr evqvtmciga cpsqfraanm haqiktslhr |
| 301 | lkpdtvpapc cvpasynpmv liqktdtgvs lqtyddllak dchci |

The nucleic acid sequence and the encoded protein sequence defining the rabbit IgG1 Fc-mature mouse GDF15 fusion protein (rFc-rmGDF15) are shown below. rFc-BUSINESS 30261614.1 rmGDF15 contains rabbit IgG1 Fc from amino acids 1-223, an artificial linker sequence from amino acids 224-227, and mature mouse GDF15 from amino acids 228-342.

Nucleic Acid Sequence Encoding the Rabbit IgG1 Fc—Mature Mouse GDF15 Fusion Protein (rFc-rmGDF15) (SEQ ID NO:221)

| | |
|---|---|
| 1 | tcgaaaccca cttgccctcc tccggagctg ttgggcggac |
| | cctccgtgtt tatctttccc |
| 61 | ccgaagccga aagataccct tatgatctca cggacgccgg |
| | aggtcacttg cgtagtagtg |
| 121 | gatgtgtcgg aggatgaccc cgaagtccag ttcacctggt |
| | atatcaataa cgagcaagtg |
| 181 | aggacagcga ggcccccact tagggagcag cagttcaact |
| | ccacaattcg ggtcgtcagc |
| 241 | actttgccca tcgctcatga ggactggctc cgcggaaaag |
| | agttcaagtg taaggtgcat |
| 301 | aacaaggcat tgccagcgcc tattgaaaag acaatctcga |
| | aggcgcgagg gcagccgctc |
| 361 | gagcccaaag tgtatacgat gggacccccg agggaagaat |
| | tgtcgtcgcg ctcagtaagc |
| 421 | cttacgtgca tgattaacgg tttctaccct agcgacatca |
| | gcgtagagtg ggaaaagaat |
| 481 | ggaaaggcgg aggataacta caagacgact cccgcggtgc |
| | tggattcgga tgggtcgtac |
| 541 | tttctgtata gcaaattgtc agtcccgacc tcagaatggc |
| | agaggggtga cgtgttcacg |
| 601 | tgctccgtga tgcacgaagc acttcacaat cactacaccc |
| | agaaatcaat ctcgcggtcc |
| 661 | ccaggcaaag gtggaggagg gtcggctcac gcccaccctc |
| | gcgattcgtg tccgctgggg |
| 721 | cctggtagat gctgtcatct cgagacagtc caggccacgc |
| | tggaggacct cgggtggtca |
| 781 | gactgggtcc tgtccccacg acaactgcag ctttcgatgt |
| | gcgtggggga atgtccgcac |
| 841 | ttgtacagat cggcgaatac ccacgctcag attaaggcac |
| | gactccatgg tttgcagcca |
| 901 | gataaagtcc ccgcaccttg ctgtgtcccc agctcatata |
| | ctcctgtcgt actcatgcat |
| 961 | cggacagaca gcggcgtgtc gcttcaaacg tatgacgacc |
| | tcgtagcgag aggatgtcat |
| 1021 | tgcgcc |

Protein Sequence Defining the Rabbit IgG1 Fc—Mature Mouse GDF15 Fusion Protein (rFc-rmGDF15) (SEQ ID NO:222)

| | |
|---|---|
| 1 | skptcpppel lggpsvfifp pkpkdtlmis rtpevtcvvv dvseddpevq ftwyinneqv |
| 61 | rtarpplreq qfnstirvvs tlpiahedwl rgkefkckvh nkalpapiek tiskargqpl |
| 121 | epkvytmgpp reelssrsvs ltcmingfyp sdisvewekn gkaednyktt pavldsdgsy |
| 181 | flysklsvpt sewqrgdvft csvmhealhn hytqksisrs pgkggggsah ahprdscplg |
| 241 | pgrcchletv qatledlgws dwvlsprqlq lsmcvgecph lyrsanthaq ikarlhglqp |
| 301 | dkvpapccvp ssytpvvlmh rtdsgvslqt yddlvargch ca |

The following sequences represent exemplary protein sequences for human IgG1 Fc-mature human GDF15 fusion proteins (hFc-rhGDF15). hFc-rhGDF15 Xa consists of human IgG1 Fc from amino acids 1-227, Factor Xa cleavage site from amino acids 228-233, an artificial linker sequence from amino acids 234-238, and mature hGDF15 from amino acides 239-350. hFc-rhGDF15 consists of human IgG1 Fc from amino acids 1-227, an artificial linker sequence from amino acids 228-232, and mature hGDF15 from amino acids 233-344.

Protein Sequence Defining the Human IgG1 Fc—Mature Human GDF15 Fusion Protein with Xa Cleavage Site (hFc-hGDF15 Xa) (SEQ ID NO:223)

| | |
|---|---|
| 1 | dkthtcppcp apellggpsv flfppkpkdt lmisrtpevt |
| | cvvvdvshed pevkfnwyvd |
| 61 | gvevhnaktk preeqynsty rvvsvltvlh qdwlngkeyk |
| | ckvsnkalpa piektiskak |
| 121 | gqprepqvyt lppsreemtk nqvsltclvk gfypsdiave |
| | wesngqpenn ykttppvlds |
| 181 | dgsfflyskl tvdksrwqqg nvfscsvmhe alhnhytqks |
| | lslspgkieg rmdggggsar |
| 241 | ngdhcplgpg rccrlhtvra sledlgwadw vlsprevqvt |
| | mcigacpsqf raanmhaqik |
| 301 | tslhrlkpdt vpapccvpas ynpmvliqkt dtgvslqtyd |
| | dllakdchci |

Protein Sequence Defining the Human IgG1 Fe—Mature Human GDF15 Fusion Protein with (hFc-hGDF15) (SEQ ID NO:224)

| | |
|---|---|
| 1 | dkthtcppcp apellggpsv flfppkpkdt lmisrtpevt cvvvdvshed pevkfnwyvd |
| 61 | gvevhnaktk preeqynsty rvvsvltvlh qdwlngkeyk ckvsnkalpa piektiskak |
| 121 | gqprepqvyt lppsreemtk nqvsltclvk gfypsdiave wesngqpenn ykttppvlds |
| 181 | dgsfflyskl tvdksrwqqg nvfscsvmhe alhnhytqks lslspgkggg gsarngdhcp |
| 241 | lgpgrccrlh tvrasledlg wadwvlspre vqvtmcigac psqfraanmh aqiktslhrl |
| 301 | kpdtvpapcc vpasynpmvl iqktdtgvsl qtyddllakd chci |

### Example 4: Fc-rhGDF15 Induced Cachexia Model

This Example describes the generation of an Fc-GDF15-induced cachexia model in mice. Immune-competent (Balb/C) and immune-incompetent (CB17-Scid) mice were randomized into three groups of ten mice each. Each group received one of the following treatments: PBS (control), mFc-rhGDF15 (as described in Example 3), or rFc-rmGDF15 (as described in Example 3) at 1p g/g. Eight-week old female mice were dosed subcutaneously into the flank for three days (Balb/C) or once (CB17-Scid). Body weight was measured daily.

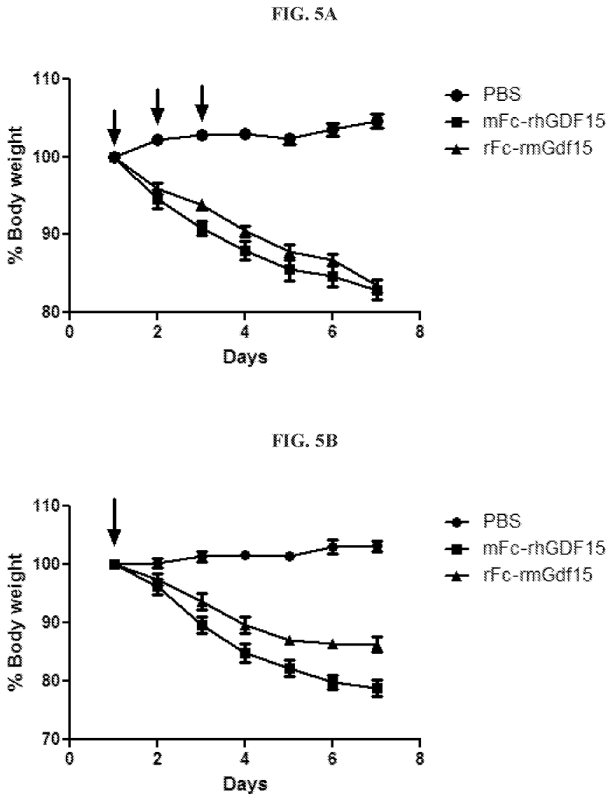

As shown in FIG. 5A and FIG. 5B, administration of mFc-rhGDF15 or rFc-rmGDF15 induced body weight loss in immune-competent mice (FIG. 5A) and immune-incompetent mice (FIG. 5B). These results indicate that a steady-state level of active rhGDF15 was achieved, because regardless of dose (one vs. three doses), both mFc-rhGDF15 and rFc-rmGDF15 induced sustained weight loss over the measured time course (7 days).

The fusion proteins, mFc-rhGDF15 and rFc-rmGDF15, were further tested in additional immune-competent (C57BL6, Swiss Webster) and immune-incompetent (ICR-SCID) mouse strains. In each tested mouse strain, the administration of mFc-rhGDF15 or rFc-rmGDF15 induced cachexia, as measured by body weight loss. Similar results were obtained regardless of whether mFc-rhGDF15 was dosed subcutaneously or intraperitoneally.

It was also investigated whether mFc-rhGDF15 induced weight loss regardless of the age of the mice treated with fusion protein. Swiss Webster (immune-competent) female mice of different ages (7, 13 and 25 weeks old) were divided into two groups of ten and treated with three doses per day of mFc-rhGDF15 or PBS (0.8 μg/g, 7 week old mice; 0.6 μg/g, 13 week old mice; or 0.4 μg/g, 25 week old mice). mFc-rhGDF15-induced weight loss was observed in all three mice age populations. In each age population, the mice lost approximately 10% of their body weight following treatment with mFc-rhGDF15 measured at ten days post treatment.

In another experiment, mFc-rhGDF15 induction of cachexia was investigated by measuring the loss of body weight, the loss of muscle mass, the loss of fat mass, and the expression levels of two molecular markers indicative of muscle degradation (i.e., mMuRF1 and mAtrogin). MuRF1 and Atrogin are E3-ubquitin ligases that are upregulated in multiple models of muscle atrophy and cachexia (Glass, D. (2010) CURR. OPIN. CLIN. NUTR. MET. CARE 13:225-229).

Eight-week old female ICR-SCID mice were randomly divided into ten groups of ten mice each. Five groups (ten mice each) were dosed subcutaneously in the flank with PBS (control) and five groups (ten mice each) were dosed subcutaneously in the flank with mFc-rhGDF15 at 1.6 μg/g on day one. Body weight was measured daily for up to 17 days. One control group and one treatment group were sacrificed at different time points (0, 1, 3, 7 and 16 days post dose). Gonadal fat and gastrocnemius muscles were removed surgically from each group of mice at the indicated sacrifice time, and weighed. Tissues were snap frozen in liquid nitrogen, and RNA was isolated from the gastrocnemius muscle samples. Levels of mMuRF1 and mAtrogin mRNA were measured by qRT-PCR in samples corresponding to groups collected after 1, 7, and 16 days post dose. Statistical analyses were performed using a two-way ANOVA.

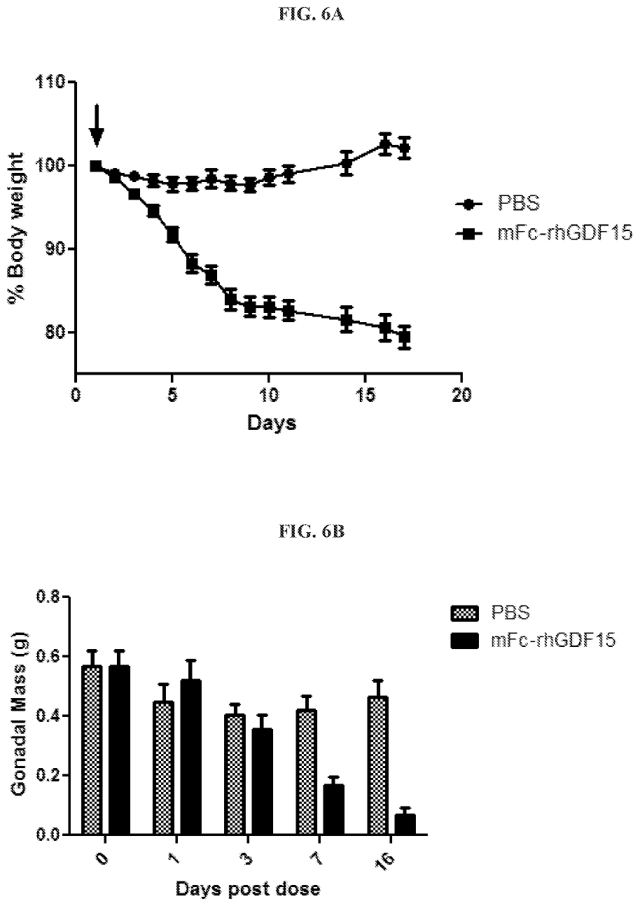

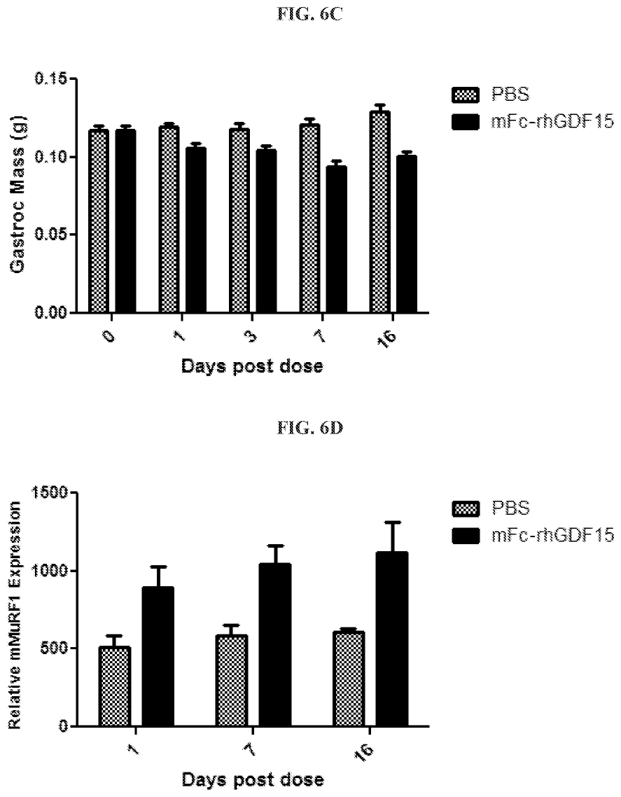

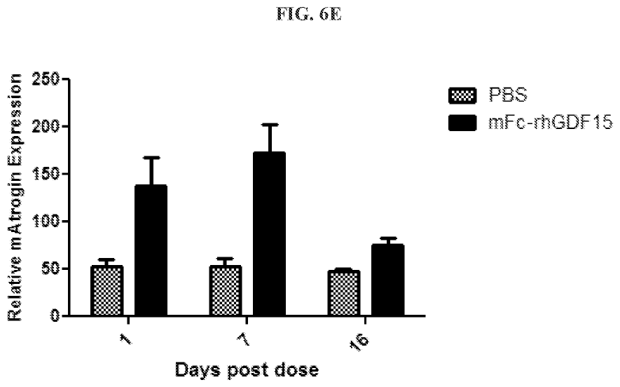

As shown in FIG. 6A, mFc-rhGDF15 induced body weight loss in ICR-SCID mice. Percent body weight was 79.4 percent when measured after 16 days following one dose of mFc-rhGDF15 (p<0.001). mFc-rhGDF15 also induced loss of fat (adipose tissue), as observed by the loss of gonadal fat (FIG. 6B; p<0.01 at day 7 and p<0.001 at day 16) and loss of muscle, as observed by the loss of gastrocnemius muscle (FIG. 6C; p<0.05 at days 1 and 3, and p<0.0001 at days 7 and 16). Administration of mFc-rhGDF15 also elevated gene expression of two enzymes associated with muscle degradation and cachexia, mMuRF1 (FIG. 6D; 6<0.001 at days 1, 7, and 16) and mAtrogin (FIG. 6E; p<0.001 at days 1 and 7, and p<0.01 at day 16).

These results indicated that mFc-rhGDF15 induces cachexia in mice.

### Example 5: mFc-rhGDF15 Induces Cachexia with a Longer GDF15 Half-Life in Serum

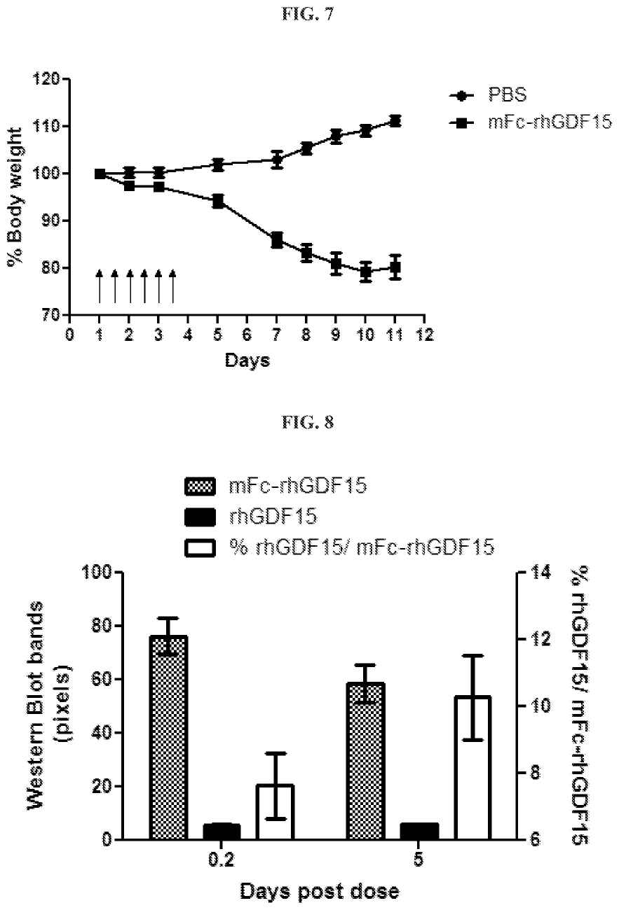

In this Example, the serum hGDF15 levels were measured following administration of mFc-rhGDF15, to determine the half-life of rhGDF15 in this model. Eight-week old female Balb/C nude mice were randomly divided into two groups of twelve mice each. Mice were dosed subcutaneously in the flank every twelve hours for three days (a total of six doses) with one of the following treatments: PBS (control) or mFc-rhGDF15 at 1.33 μg/g. Body weight was measured daily. As shown in FIG. 7, mFc-rhGDF15 induced sustained body weight loss for at least one week after the final injection.

In this experiment, hGDF15 serum levels were measured 0.2, 5, and 8 days after the last dose of mFc-rhGDF15. Mice were sacrificed at the indicated time, and sera were collected. Human GDF15 serum levels were determined by ELISA (R&D Systems, Cat. No. DY957E). Table 1 provides the serum levels (pg/mL) for each mouse in the study.

TABLE 1

| Days post | Mouse | Treatment | | Serum GDF15 |
|---|---|---|---|---|
| last dose | # | Agent | μg/g | (μg/mL); ELISA |
| 0.2 | 1 | mFc-rhGDF15 | 1.33 | 10.02 |
| 0.2 | 2 | mFc-rhGDF15 | 1.33 | 9.54 |
| 0.2 | 3 | mFc-rhGDF15 | 1.33 | 9.36 |
| 5 | 4 | mFc-rhGDF15 | 1.33 | 8.24 |
| 5 | 5 | mFc-rhGDF15 | 1.33 | 8.01 |
| 5 | 6 | mFc-rhGDF15 | 1.33 | 6.59 |
| 8 | 7 | mFc-rhGDF15 | 1.33 | 5.60 |
| 8 | 8 | mFc-rhGDF15 | 1.33 | 5.52 |
| 8 | 9 | mFc-rhGDF15 | 1.33 | 5.57 |

The results in Table 1 reveal that strong, sustained levels of hGDF15 are present in the serum at least eight days after the last dose of mFc-rhGDF15.

Serum samples from day 0.2 and day 5 after the last dose were also analyzed by Western blot (reducing gel; blot with an antibody against hGDF15 (R&D Systems, Cat. No AF957)) and quantified by Licor to determine the stability of mFc-rhGDF15 in the serum. Unexpectedly, two bands were observed. The upper band was approximately 40 kDa, and appeared to be mFc-rhGDF15. The lower band was approximately 15 kDa, and appeared to be cleaved mature rhGDF15. This indicated that mature rhGDF15 was released from mFc-rhGDF15 in the serum. Quantification of the two bands showed that about 90% of the rhGDF15 present in the serum was in the form of mFc-rhGDF15, with about 10% of the total rhGDF15 in the serum being present as the cleaved mature form (FIG. 8). Quantification showed a slight decrease in mFc-rhGDF15 in the serum samples collected five days after the last dose, but, surprisingly, a constant level of mature rhGDF15 remained in the serum. The ratio of mature rhGDF15 to mFc-rhGDF15 slightly increased over time, as a result of a decrease in mFc-rhGDF15 in the serum. Similar results were observed when rFc-rmGDF15 was injected into mice.

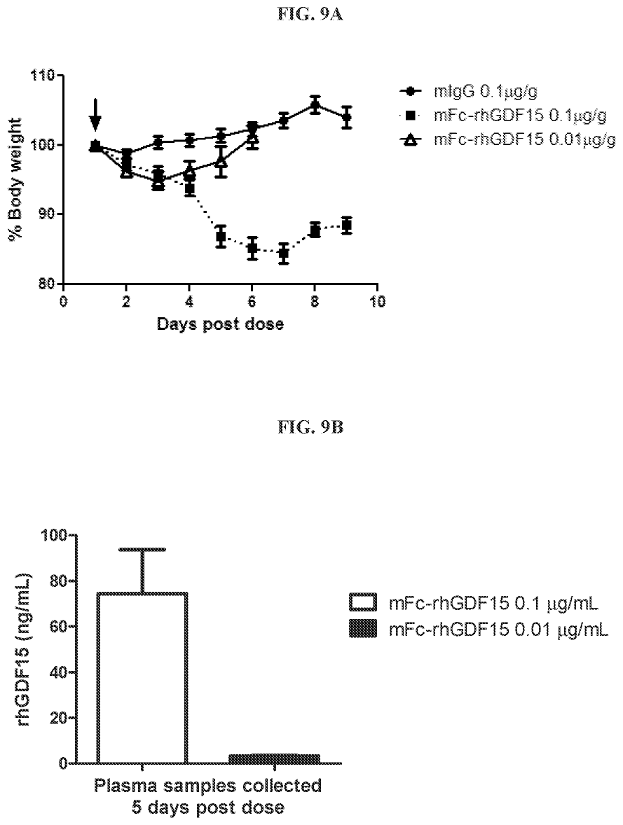

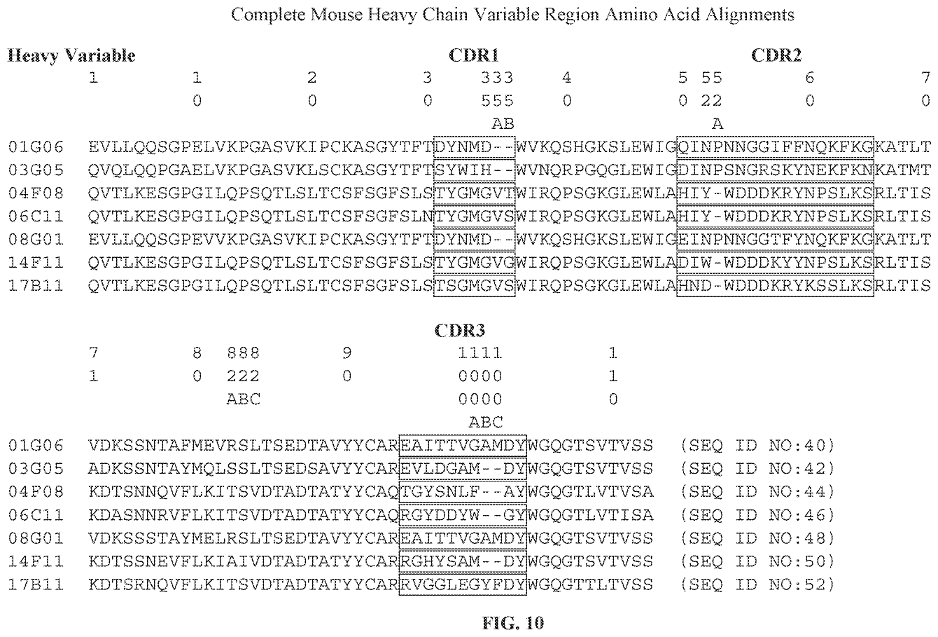

The results presented in FIGS. 7-8 and Table 1 were unexpected. The expectation was that very little, if any, mature rhGDF15 would be cleaved (released) from the mFc-rhGDF15 by day 0.2, and that any cleaved rhGDF15 would be rapidly cleared from the serum, as had been previously observed. For example, in FIG. 4, a series of nine doses at 1 μg/g per dose (for a total of 9 μg/g) of cleaved-rhGDF15 was required to induce significant body weight loss in mice. These mice gained weight, almost immediately when dosing stopped. In contrast, a single dose of mFc-rhGDF15 at 0.1 μg/g was sufficient to induce significant body weight loss for at least eight days (FIG. 9A; ten ICR-SCID mice dosed intrapertioneally with 0.1 μg/g on day 1). The data in Table 1 revealed that rhGDF15 serum levels were stable for at least eight days, when rhGDF15 was administered as an mFc-rhGDF15 fusion protein.

To determine the source of activity resulting in sustained body weight loss, we investigated whether the observed rhGDF15 activity was attributable to the mFc-rhGDF15 fusion protein, the released mature rhGDF15 form, or both. As shown in FIG. 9A, a low dose of mFc-rhGDF15 (0.1 μg/g) resulted in body weight loss continuing for at least eight days. A lower dose of mFc-rhGDF15 (0.01 μg/g) also induced body weight loss, but the effect was not sustained for longer than 3 days post dose.

In this experiment, plasma was collected from three mice each dosed with 0.1 μg/g or 0.01 μg/g at 5 days post dose. Total rhGDF15 was measured by ELISA as described above. Total rhGDF15 plasma levels in the mice dosed with 0.1 μg/g were above 70 ng/mL, consistent with the observation that these mice had significant weight loss (FIG. 9B). Total rhGDF15 plasma levels in mice dosed with 0.01 μg/g were approximately 3.3 ng/mL, but it was observed that these mice were gaining weight (FIG. 9A and FIG. 9B). As described in FIG. 2, the threshold for hGDF15 to induce cachexia in tumor bearing mice is approximately 2 ng/mL. Thus, if both forms of rhGDF15 were active (i.e., mFc-rhGDF15 and released mature rhGDF15), then these mice should be losing weight, not gaining weight (i.e., 3.3 ng/mL total rhGDF15 is above the threshold of approximately 2 ng/mL hGDF15).

To determine which form was the active form (i.e., either mFc-rhGDF15 or released mature rhGDF15), we considered the data from FIG. 8 which showed that approximately 90% of the total rhGDF15 in serum was in the mFc-rhGDF15 form, and the remaining 10% was the released mature form. Based on this extrapolation, approximately 3.0 ng/mL of rhGDF15 in the plasma was in the mFc-rhGDF15 form (i.e., 90% of 3.3 ng/mL). Once again, if mFc-rhGDF15 were active, these mice would be losing weight, not gaining weight because 3.3 ng/mL mFc-rhGDF15 is above the threshold of approximately 2 ng/mL hGDF15. The mice dosed with 0.1 μg/g mFc-rhGDF15 served as an internal control, because these mice had sustained body weight loss indicating that at least one of the two forms must be active. A calculation of 10% of 70 ng/mL total rhGDF15 in these mice is 7 ng/mL released mature rhGDF15. This amount is consistent with inducing the observed body weight loss and the threshold observed in FIG. 2. Thus, the data indicate that the mFc-rhGDF15 is not an active form of the protein, and only the mature rhGDF15 is active. These results were unexpected, because: (a) there was no reason to predict that the Fc fusion protein (mFc-rhGDF15) would be inactive; and (b) there was no reason to predict that the Fc fusion protein would release mature rhGDF15 at the observed rate.

These results indicate that mFc-rhGDF15 sustains a cachetic phenotype by slowly releasing mature rhGDF15 into the serum. These results further indicate that a steady state level of mature rhGDF15 in the plasma or serum can be achieved in a non-tumor bearing mouse by administering mFc-rhGDF15 to the mouse. Therefore, administration of mFc-rhGDF15 to non-tumor bearing mice is particularly useful as a mouse model of cachexia with a robust and sustained loss of muscle mass, loss of fat mass, and body weight loss (see FIGS. 6A-C).

### Example 6: Anti-GDF15 Antibodies

This Example describes the production of anti-GDF15 monoclonal antibodies. Immunizations, fusions, and primary screens were conducted using conventional methods following the Repetitive Immunization Multiple Sites (RIMMS) protocol. Five AJ mice and five Balb/c mice were immunized with 6×His (SEQ ID NO: 266) tagged recombinant human GDF15 (His-rhGDF15) (R&D Systems, Inc., Minneapolis, MN). Two Balb/c mice with sera displaying the highest anti-GDF15 activity by Enzyme Linked Immunosorbent Assay (ELISA) were chosen for subsequent fusion. Spleens and lymph nodes from the appropriate mice were harvested. B-cells were harvested and fused with a myeloma line. Fusion products were serially diluted onto forty 96-well plates to near clonality. Two AJ mice with sera displaying the highest anti-GDF15 activity by ELISA were chosen for subsequent fusion. Spleens and lymph nodes from the appropriate mice were harvested. B-cells were harvested and fused with a myeloma line. Fusion products were serially diluted onto forty 96-well plates to near clonality.

Approximately 3,840 supernatants from the cell fusions were screened by ELISA for binding to rhGDF15. A total of 172 supernatants containing antibodies against GDF15 were further characterized in vitro. A panel of hybridomas was selected, subcloned and expanded. Antibodies were expressed and subsequently purified by affinity chromatography on Protein G resin, under standard conditions.

### Example 7: Antibody Sequence Analysis

The light chain isotype and heavy chain isotype of each monoclonal antibody in Example 6 was determined using the IsoStrip™ Mouse Monoclonal Antibody Isotyping Kit according the kit vendor's instructions (Roche Applied Science, Indianapolis, IN). All antibodies were found to be kappa light chain, and IgG1 or IgG2b heavy chain.

The heavy and light chain variable regions of the mouse monoclonal antibodies were sequenced using 5′ RACE (Rapid Amplification of cDNA Ends). Total RNA was extracted from each monoclonal hybridoma cell line using the RNeasy® Miniprep kit according to the kit vendor's instructions (Qiagen, Valencia, CA). Full-length first strand cDNA containing 5′ ends was generated using the SMARTer™ RACE cDNA Amplification Kit (Clontech, Mountain View, CA) according to the kit vendor's instructions for 5′ RACE.

The variable regions of the light (kappa) and heavy (IgGlor IgG2b) chains were amplified by PCR using KOD Hot Start Polymerase (EMD Chemicals, Gibbstown, NJ) according to the kit vendor's instructions. For amplification of 5′ cDNA ends in conjunction with the SMARTer M RACE cDNA Amplification Kit, the Universal Primer Mix A primer (Clontech), a mix of: 5′ CTAATACGACTCACTATAGGGCAAGCAGTGGTATCAACGCAGAGT 3′ (SEQ ID NO:233) and 5′ CTAATACGACTCACTATAGGGC 3′ (SEQ ID NO:225), was used as a 5′ primer. Heavy chain variable regions were amplified using the above 5′ primers and a 3′ IgG1 constant region specific primer, 5′ TATGCAAGGCTTACAACCACA 3′ (SEQ ID NO:226), or a 3′ IgG2b constant region specific primer, 5′ AGGACAGGGGTTGATTGTTGA 3′ (SEQ ID NO:227). Kappa chain variable regions were first amplified with the above 5′ primers and a 3′ kappa constant region specific primer, 5′ CTCATTCCTGTTGAAGCTCTTGACAAT 3′ (SEQ ID NO:228). The light chains were subjected to a second, nested, round of PCR using the Nested Universal Primer A (Clontech) 5′ primer, 5′ AAGCAGTGGTATCAACGCAGAGT 3′ (SEQ ID NO:229) and a nested 3′ kappa constant region specific primer, 5′ CGACTGAGGCACCTCCAGATGTT 3′ (SEQ ID NO:230). Individual PCR products were either purified using the Qiaquick® PCR Purification kit or isolated by agarose gel electrophoresis and purified using the Qiaquick® Gel Purification kit according to the kit vendor's instructions (Qiagen). The PCR products were subsequently cloned into the pCR®4Blunt plasmid using the Zero Blunt® TOPO® PCR Cloning according to the kit vendor's instructions (Invitrogen) and transformed into DH5-α bacteria (Invitrogen) through standard molecular biology techniques. Plasmid DNA isolated from transformed bacterial clones was sequenced using M13 Forward (5′ GTAAAACGACGGCCAGT 3′) (SEQ ID NO:231) and M13 Reverse primers (5′ CAGGAAACAGCTATGACC 3′) (SEQ ID NO:232) by Beckman Genomics (Danvers, MA), using standard dideoxy DNA sequencing methods to identify the sequence of the variable region sequences. The sequences were analyzed using Vector NTI software (Invitrogen) and the IMGT/V-Quest web server (imgt.cines.fr) to identify and confirm variable region sequences.

The nucleic acid sequences encoding and the protein sequences defining variable regions of the murine monoclonal antibodies are shown below (amino terminal signal peptide sequences are not shown). CDR sequences (Kabat definition) are indicated by bold font and underlining in the amino acid sequences.

Nucleic Acid Sequence Encoding the Heavy Chain Variable Region of the 01G06 Antibody (SEQ ID NO:39)

| | |
|---|---|
| 1 | gaggtcctgc tgcaacagtc tggacctgag ctggtgaagc ctggggcttc agtgaagata |
| 61 | ccctgcaagg cttctggata cacattcact gactacaaca tggactgggt gaagcagagc |
| 121 | catggaaaga gccttgagtg gattggacaa attaatccta acaatggtgg tattttcttc |
| 181 | aaccagaagt tcaagggcaa ggccacattg actgtagaca agtcctccaa tacagccttc |
| 241 | atggaggtcc gcagcctgac atctgaggac actgcagtct attactgtgc aagagaggca |
| 301 | attactacgg taggcgctat ggactactgg ggtcaaggaa cctcagtcac cgtctcctca |

Protein Sequence Defining the Heavy Chain Variable Region of the 01G06 Antibody (SEQ ID NO:40)

| | |
|---|---|
| 1 | evllqqsgpe lvkpgasvki pckasgytft dynmdwvkqs hgkslewigq inpnnggiff |
| 61 | nqkfkgkatl tvdkssntaf mevrsltsed tavyycarea ittvgamdyw gqgtsvtvss |

Nucleic Acid Sequence Encoding the Kappa Chain Variable Region of the 01G06 Antibody (SEQ ID NO:75)

| | |
|---|---|
| 1 | gacatccaga tgactcagtc tccagcctcc ctatctgcat ctgtgggaga aactgtcacc |
| 61 | atcacatgtc gaacaagtga gaatcttcac aattatttag catggtatca gcagaaacag |
| 121 | ggaaaatctc ctcagctcct ggtctatgat gcaaaaacct tagcagatgg tgtgccatca |
| 181 | aggttcagtg gcagtggatc aggaacacaa tattctctca agatcaacag cctgcagcct |
| 241 | gaagattttg ggagttatta ctgtcaacat ttttggagta gtccttacac gttcggaggg |
| 301 | gggaccaagc tggaaataaa a |

Protein Sequence Defining the Kappa Chain Variable Region of the 01G06 Antibody (SEQ ID NO:76)

| | |
|---|---|
| 1 | diqmtqspas lsasvgetvt itcrtsenlh nylawyqqkq gkspqllvyd aktladgvps |
| 61 | rfsgsgsgtq yslkinslqp edfgsyycqh fwsspytfgg gtkleik |

Nucleic Acid Sequence Encoding the Heavy Chain Variable Region of the 03G05 Antibody (SEQ ID NO:41)

| | |
|---|---|
| 1 | caggtccaac tgcagcagcc tggggctgaa ctggtgaagc ctggggcttc agtgaagctg |
| 61 | tcctgcaagg cttctggcta caccttcacc agctactgga ttcactgggt gaaccagagg |
| 121 | cctggacaag gccttgagtg gattggagac attaatccta gcaacggccg tagtaagtat |
| 181 | aatgagaagt tcaagaacaa ggccacaatg actgcagaca aatcctccaa cacagcctac |
| 241 | atgcaactca gcagcctgac atctgaggac tctgcggtct attactgtgc aagagaggtt |
| 301 | ctggatggtg ctatggacta ctggggtcaa ggaacctcag tcaccgtctc ctca |

Protein Sequence Defining the Heavy Chain Variable Region of the 03G05 Antibody (SEQ ID NO:42)

| | |
|---|---|
| 1 | qvqlqqpgae lvkpgasvkl sckasgytft sywihwvnqr pgqglewigd inpsngrsky |
| 61 | nekfknkatm tadkssntay mqlssltsed savyycarev ldgamdywgq gtsvtvss |

Nucleic Acid Sequence Encoding the Kappa Chain Variable Region of the 03G05 Antibody (SEQ ID NO:77)

| | |
|---|---|
| 1 | gacattgtgt tgacccaatc tccagcttct ttggctgtgt ctctagggca gagggccacc |
| 61 | atctcctgca gagccagcga aagtgttgat aattatggca ttagttttat gaactggttc |
| 121 | caacagaaac caggacagcc acccaaactc ctcatctatg ctgcatccaa ccaaggctcc |
| 181 | ggggtccctg ccaggtttag tggcagtggg tctgggacag acttcagcct caacatccat |
| 241 | cctatggagg aggatgatac tgcaatgtat ttctgtcagc aaagtaagga ggttccgtgg |
| 301 | acgttcggtg gaggctccaa gctggaaatc aaa |

Protein Sequence Defining the Kappa Chain Variable Region of the 03G05 Antibody (SEQ ID NO:78)

| | |
|---|---|
| 1 | divltqspas lavslgqrat iscrasesvd nygisfmnwf qqkpgqppkl liyaasnqgs |
| 61 | gvparfsgsg sgtdfslnih pmeeddtamy fcqqskevpw tfgggsklei k |

Nucleic Acid Sequence Encoding the Heavy Chain Variable Region of the 04F08 Antibody (SEQ ID NO:43)

| | |
|---|---|
| 1 | caggttactc tgaaagagtc tggccctggg atattgcagc cctcccagac cctcagtctg |
| 61 | acttgttctt tctctgggtt ttcactgagc acttatggta tgggtgtgac ctggattcgt |
| 121 | cagccttcag gaaagggtct ggagtggctg gcacacattt actgggatga tgacaagcgc |
| 181 | tataacccat ccctgaagag ccggctcaca atctccaagg atacctccaa caaccaggta |
| 241 | ttcctcaaga tcaccagtgt ggacactgca gatactgcca catactactg tgctcaaacg |
| 301 | gggtatagta acttgtttgc ttactggggc caagggactc tggtcactgt ctctgca |

Protein Sequence Defining the Heavy Chain Variable Region of the 04F08 Antibody (SEQ ID NO:44)

| | |
|---|---|
| 1 | qvtlkesgpg ilqpsqtlsl tcsfsgfsls tygmgvtwir qpsgkglewl ahiywdddkr |
| 61 | ynpslksrlt iskdtsnnqv flkitsvdta dtatyycaqt gysnlfaywg qgtlvtvsa |

Nucleic Acid Sequence Encoding the Kappa Chain Variable Region of the 04F08 Antibody (SEQ ID NO:79)

| | |
|---|---|
| 1 | gacattgtga tgacccagtc tcaaaaattc atgtccacat cagtaggaga cagggtcagc |
| 61 | gtcacctgca aggccagtca gaatgtgggt actaatgtag cctggtatca acagaaatta |
| 121 | ggacaatctc ctaaaacact gatttactcg gcatcctacc ggtacagtgg agtccctgat |
| 181 | cgcttcacag gcagtggatc tgggacagat ttcactctca ccatcagcaa tgtgcagtct |
| 241 | gaagacttgg cagagtattt ctgtcagcaa tataacagct atccgtacac gttcggaggg |
| 301 | gggaccaagc tggaaataaa a |

Protein Sequence Defining the Kappa Chain Variable Region of the 04F08 Antibody (SEQ ID NO:80)

| | |
|---|---|
| 1 | divmtqsqkf mstsvgdrvs vtckasqnvg tnvawyqqkl gqspktliys asyrysgvpd |
| 61 | rftgsgsgtd ftltisnvqs edlaeyfcqq ynsypytfgg gtkleik |

Nucleic Acid Sequence Encoding the Heavy Chain Variable Region of the 06C11 Antibody (SEQ ID NO:45)

| | |
|---|---|
| 1 | caggttactc tgaaagagtc tggccctggg atattgcagc cctcccagac cctcagtctg |
| 61 | acttgttctt tctctgggtt ttcactgaac acttatggta tgggtgtgag ctggattcgt |
| 121 | cagccttcag gaaagggtct ggagtggctg gcacacattt actgggatga tgacaagcgc |
| 181 | tataacccat ccctgaagag ccggctcaca atctccaagg atgcctccaa caaccgggtc |
| 241 | ttcctcaaga tcaccagtgt ggacactgca gatactgcca catactactg tgctcaaaga |
| 301 | ggttatgatg attactgggg ttactggggc caagggactc tggtcactat ctctgca |

Protein Sequence Defining the Heavy Chain Variable Region of the 06C11 Antibody (SEQ ID NO:46)

| | |
|---|---|
| 1 | qvtlkesgpg ilqpsqtlsl tcsfsgfsln tygmgvswir qpsgkglewl ahiywdddkr |
| 61 | ynpslksrlt iskdasnnrv flkitsvdta dtatyycaqr gyddywgywg qgtlvtisa |

Nucleic Acid Sequence Encoding the Kappa Chain Variable Region of the 06C11 Antibody (SEQ ID NO:81)

| | |
|---|---|
| 1 | gacattgtga tgacccagtc tcaaaaattc atgtccacat cagtaggaga cagggtcagc |
| 61 | gtcacctgca aggccagtca gaatgtgggt actaatgtag cctggtttca acagaaacca |
| 121 | ggtcaatctc ctaaagcact gatttactcg gcatcttacc ggtacagtgg agtccctgat |
| 181 | cgcttcacag gcagtggatc tgggacagat ttcattctca ccatcagcaa tgtgcagtct |
| 241 | gaagacctgg cagagtattt ctgtcagcaa tataacaact atcctctcac gttcggtgct |
| 301 | gggaccaagc tggagctgaa a |

Protein Sequence Defining the Kappa Chain Variable Region of the 06C11 Antibody (SEQ ID NO:82)

| | |
|---|---|
| 1 | divmtqsqkf mstsvgdrvs vtckasqnvg tnvawfqqkp gqspkaliys asyrysgvpd |
| 61 | rftgsgsgtd filtisnvqs edlaeyfcqq ynnypltfga gtklelk |

Nucleic Acid Sequence Encoding the Heavy Chain Variable Region of the 08G01 Antibody (SEQ ID NO:47)

| | |
|---|---|
| 1 | gaggtcctgc tgcaacagtc tggacctgag gtggtgaagc ctggggcttc agtgaagata |
| 61 | ccctgcaagg cttctggata cacattcact gactacaaca tggactgggt gaagcagagc |
| 121 | catggaaaga gccttgagtg gattggagag attaatccta acaatggtgg tactttctac |
| 181 | aaccagaagt tcaagggcaa ggccacattg actgtagaca agtcctccag cacagcctac |
| 241 | atggagctcc gcagcctgac atctgaggac actgcagtct attactgtgc aagagaggca |
| 301 | attactacgg taggcgctat ggactactgg ggtcaaggaa cctcagtcac cgtctcctca |

241 atggagctcc gcagcctgac atctgaggac actgcagtct attactgtgc aagagaggca
301 attactacgg taggcgctat ggactactgg ggtcaaggaa cctcagtcac cgtctcctca

Protein Sequence Defining the Heavy Chain Variable Region of the 08G01 Antibody (SEQ ID NO:48)

| | |
|---|---|
| 1 | evllqqsgpe vvkpgasvki pckasgytft dynmdwvkqs hgkslewige inpnnggtfy |
| 61 | nqkfkgkatl tvdkssstay melrsltsed tavyycarea ittvgamdyw gqgtsvtvss |

Nucleic Acid Sequence Encoding the Kappa Chain Variable Region of the 08G01 Antibody (SEQ ID NO:83)

| | |
|---|---|
| 1 | gacatccaga tgactcagtc tccagcctcc ctatctgcat ctgtgggaga aactgtcacc |
| 61 | atcacatgtc gagcaagtgg gaatattcac aattatttag catggtatca gcagaaacag |
| 121 | ggaaaatctc ctcagctcct ggtctataat gcaaaaacct tagcagatgg tgtgccatca |
| 181 | aggttcagtg gcagtggatc aggaacacaa tattctctca agatcaacag cctgcagcct |
| 241 | gaagattttg ggagttatta ctgtcaacat ttttggagtt ctccttacac gttcggaggg |
| 301 | gggaccaagc tggaaataaa a |

Protein Sequence Defining the Kappa Chain Variable Region of the 08G01 Antibody (SEQ ID NO:84)

| | |
|---|---|
| 1 | diqmtqspas lsasvgetvt itcrasgnih nylawyqqkq gkspqllvyn aktladgvps |
| 61 | rfsgsgsgtq yslkinslqp edfgsyycqh fwsspytfgg gtkleik |

Nucleic Acid Sequence Encoding the Heavy Chain Variable Region of the 14F11 Antibody (SEQ ID NO:49)

| | |
|---|---|
| 1 | caggttactc tgaaagagtc tggccctgga atattgcagc cctcccagac cctcagtctg |
| 61 | acttgttctt tctctgggtt ttcactgagc acttatggta tgggtgtagg ctggattcgt |
| 121 | cagccttcag gaaagggtct agagtggctg gcagacattt ggtgggatga cgataagtac |
| 181 | tataacccat ccctgaagag ccggctcaca atctccaagg atacctccag caatgaggta |
| 241 | ttcctcaaga tcgccattgt ggacactgca gatactgcca cttactactg tgctcgaaga |
| 301 | ggtcactact ctgctatgga ctactggggt caaggaacct cagtcaccgt ctcctca |

Protein Sequence Defining the Heavy Chain Variable Region of the 14F11 Antibody (SEQ ID NO:50)

| | |
|---|---|
| 1 | qvtlkesgpg ilqpsqtlsl tcsfsgfsls tygmgvgwir qpsgkglewl adiwwdddky |
| 61 | ynpslksrlt iskdtssnev flkiaivdta dtatyycarr ghysamdywg qgtsvtvss |

Nucleic Acid Sequence Encoding the Kappa Chain Variable Region of the 14F11 Antibody (SEQ ID NO:85)

| | |
|---|---|
| 1 | gacattgtaa tgacccagtc tcaaaaattc atgtccacat cagtaggaga cagggtcagc |
| 61 | gtcacctgca aggccagtca gaatgtgggt actaatgtag cctggtatca acagaaacca |
| 121 | gggcaatctc ctaaagcact gatttactcg ccatcctacc ggtacagtgg agtccctgat |
| 181 | cgcttcacag gcagtggatc tgggacagat ttcactctca ccatcagcaa tgtgcagtct |
| 241 | gaagacttgg cagaatattt ctgtcagcaa tataacagct atcctcacac gttcggaggg |
| 301 | gggaccaagc tggaaatgaa a |

Protein Sequence Defining the Kappa Chain Variable Region of the 14F11 Antibody (SEQ ID NO:86)

| | |
|---|---|
| 1 | divmtqsqkf mstsvgdrvs vtckasqnvg tnvawyqqkp gqspkaliys psyrysgvpd |
| 61 | rftgsgsgtd ftltisnvqs edlaeyfcqq ynsyphtfgg gtklemk |

Nucleic Acid Sequence Encoding the Heavy Chain Variable Region of the 17B11 Antibody (SEQ ID NO:51)

| | |
|---|---|
| 1 | caggttactc tgaaagagtc tggccctggg atattgcagc cctcccagac cctcagtctg |
| 61 | acttgttctt tctctgggtt ttcactgagc acttctggta tgggtgtgag ttggattcgt |
| 121 | cagccttcag gaaagggtct ggagtggctg gcacacaatg actgggatga tgacaagcgc |
| 181 | tataagtcat ccctgaagag ccggctcaca atatccaagg atacctccag aaaccaggta |
| 241 | ttcctcaaga tcaccagtgt ggacactgca gatactgcca catactactg tgctcgaaga |
| 301 | gttgggggat tagagggcta ttttgattac tggggccaag gcaccactct cacagtctcc |
| 361 | tca |

Protein Sequence Defining the Heavy Chain Variable Region of the 17B11 Antibody (SEQ ID NO:52)

| | |
|---|---|
| 1 | qvtlkesgpg ilqpsqtlsl tcsfsgfsls tsgmgvswir qpsgkglewl ahndwdddkr |
| 61 | yksslksrlt iskdtsrnqv flkitsvdta dtatyycarr vgglegyfdy wgqgttltvs |
| 121 | s |

Nucleic Acid Sequence Encoding the Kappa Chain Variable Region of the 17B11 Antibody (SEQ ID NO:87)

| | |
|---|---|
| 1 | gacattgtgc tgacacagtc tcctgcttcc ttagctgtat ctctggggca gagggccacc |
| 61 | atctcatgca gggccagcca aagtgtcagt acatctaggt ttagttatat gcactggttc |
| 121 | caacagaaac caggacaggc acccaaactc ctcatcaagt atgcatccaa cctagaatct |
| 181 | ggggtccctg ccaggttcag tggcagtggg tctgggacag acttcaccct caacatccat |
| 241 | cctgtggagg gggaggatac tgcaacatat tactgtcagc acagttggga gattccgtac |
| 301 | acgttcggag gggggaccaa gctggaaata aaa |

Protein Sequence Defining the Kappa Chain Variable Region of the 17B11 Antibody (SEQ ID NO:88)

| | |
|---|---|
| 1 | divltqspas layslgqrat iscrasqsvs tsrfsymhwf qqkpgqapkl likyasnles |
| 61 | gvparfsgsg sgtdftlnih pvegedtaty ycqhsweipy tfgggtklei k |

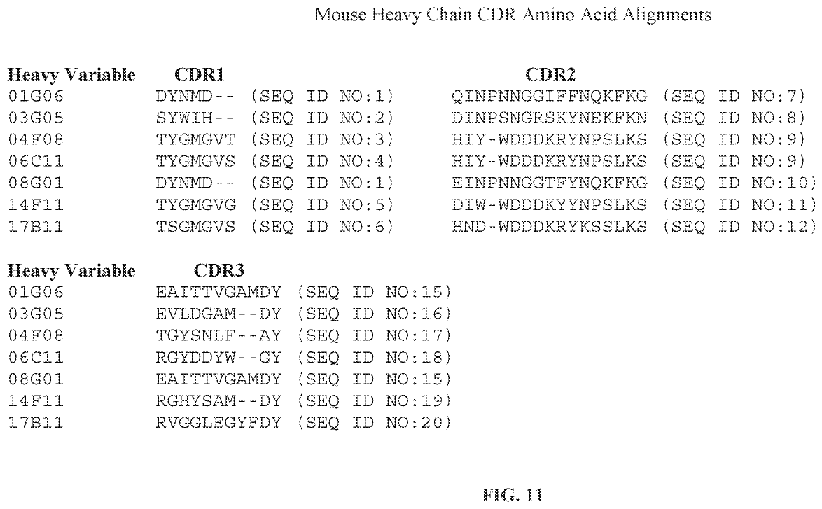

The amino acid sequences defining the immunoglobulin heavy chain variable regions for the antibodies produced in Example 6 are aligned in FIG. 10. Amino terminal signal peptide sequences (for expression/secretion) are not shown. CDR₁, CDR₂, and CDR₃ (Kabat definition) are identified by boxes. FIG. 11 shows an alignment of the separate CDR₁, CDR₂, and CDR₃ sequences for each antibody.

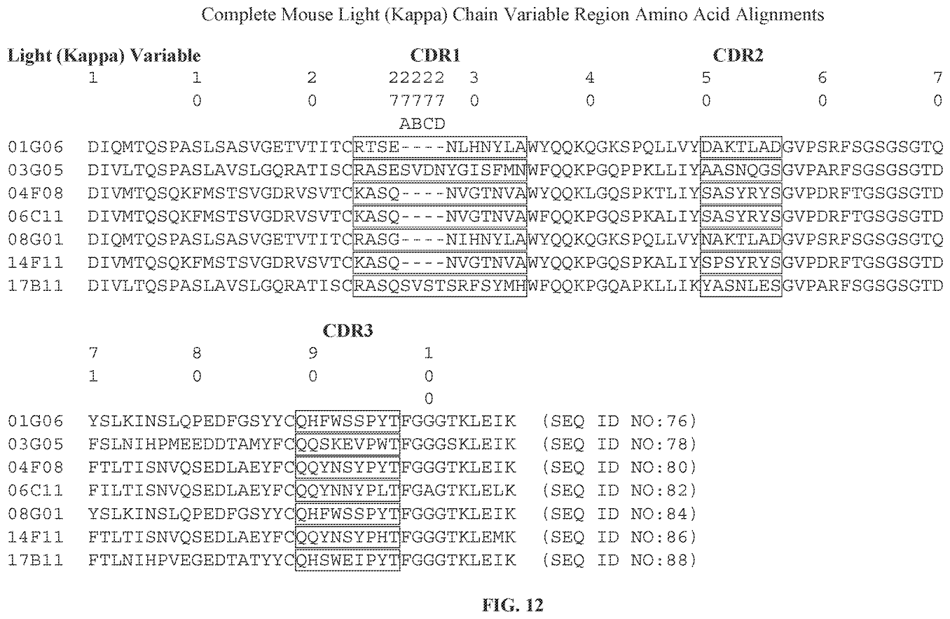

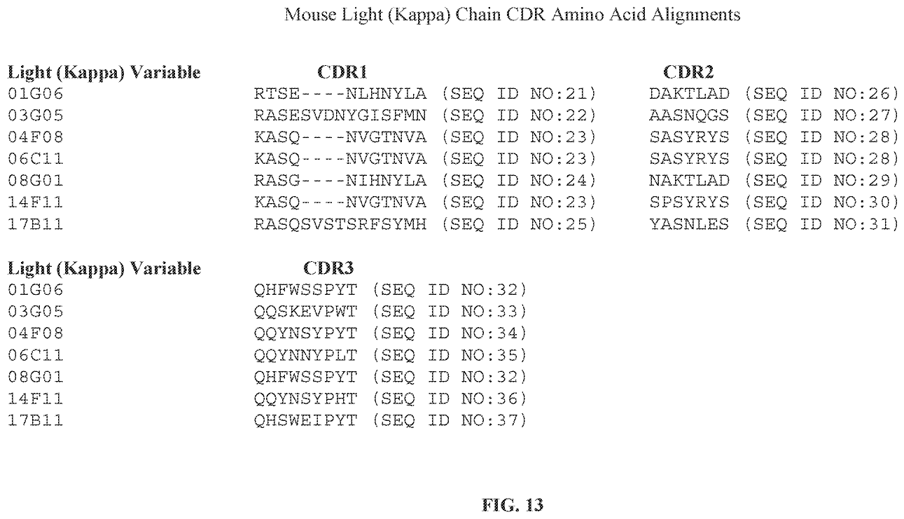

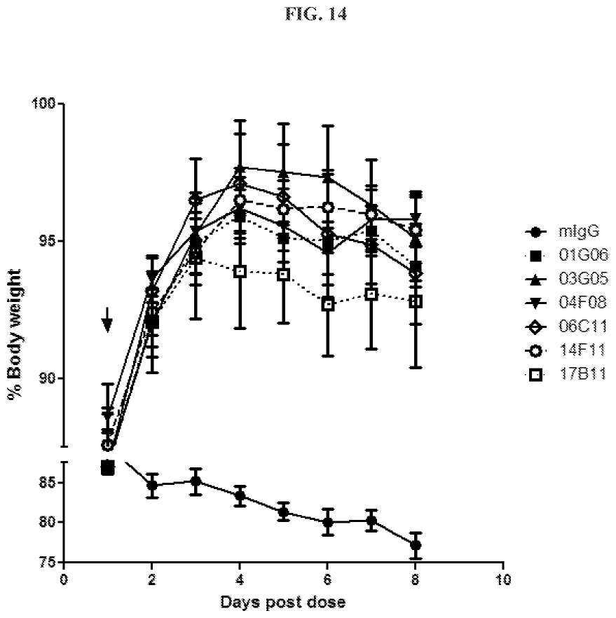

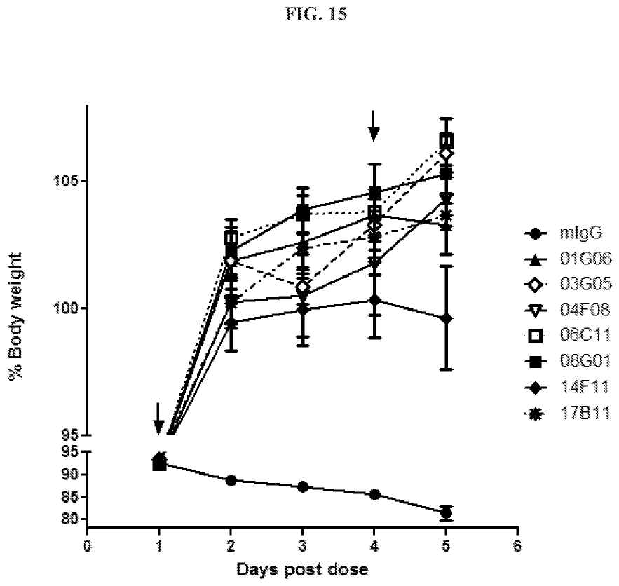

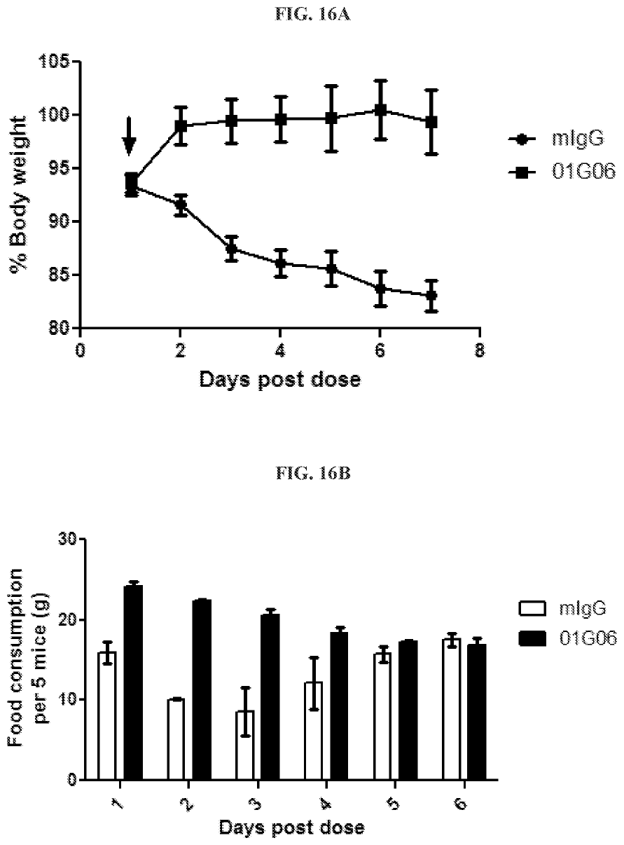

The amino acid sequences defining the immunoglobulin light chain variable regions of the antibodies in Example 6 are aligned in FIG. 12. Amino terminal signal peptide sequences (for expression/secretion) are not shown. CDR₁, CDR₂ and CDR₃ are identified by boxes. FIG. 13 shows an alignment of the separate CDR₁, CDR₂, and CDR₃ sequences for each antibody.

Table 2 shows the SEQ ID NO. of each sequence discussed in this Example.

TABLE 2

| SEQ. ID NO. | Nucleic Acid or Protein |
|---|---|
| 39 | 01G06 Heavy Chain Variable Region-nucleic acid |
| 40 | 01G06 Heavy Chain Variable Region-protein |
| 75 | 01G06 Light (kappa) Chain Variable Region-nucleic acid |
| 76 | 01G06 Light (kappa) Chain Variable Region-protein |
| 1 | 01G06 Heavy Chain CDR₁ |
| 7 | 01G06 Heavy Chain CDR₂ |
| 15 | 01G06 Heavy Chain CDR₃ |
| 21 | 01G06 Light (kappa) Chain CDR₁ |
| 26 | 01G06 Light (kappa) Chain CDR₂ |
| 32 | 01G06 Light (kappa) Chain CDR₃ |
| 41 | 03G05 Heavy Chain Variable Region-nucleic acid |
| 42 | 03G05 Heavy Chain Variable Region-protein |
| 77 | 03G05 Light (kappa) Chain Variable Region-nucleic acid |
| 78 | 03G05 Light (kappa) Chain Variable Region-protein |
| 2 | 03G05 Heavy Chain CDR₁ |
| 8 | 03G05 Heavy Chain CDR₂ |
| 16 | 03G05 Heavy Chain CDR₃ |
| 22 | 03G05 Light (kappa) Chain CDR₁ |
| 27 | 03G05 Light (kappa) Chain CDR₂ |
| 33 | 03G05 Light (kappa) Chain CDR₃ |
| 43 | 04F08 Heavy Chain Variable Region-nucleic acid |
| 44 | 04F08 Heavy Chain Variable Region-protein |
| 79 | 04F08 Light (kappa) Chain Variable Region-nucleic acid |
| 80 | 04F08 Light (kappa) Chain Variable Region-protein |
| 3 | 04F08 Heavy Chain CDR₁ |
| 9 | 04F08 Heavy Chain CDR₂ |
| 17 | 04F08 Heavy Chain CDR₃ |
| 23 | 04F08 Light (kappa) Chain CDR₁ |
| 28 | 04F08 Light (kappa) Chain CDR₂ |
| 34 | 04F08 Light (kappa) Chain CDR₃ |
| 45 | 06C11 Heavy Chain Variable Region-nucleic acid |
| 46 | 06C11 Heavy Chain Variable Region-protein |
| 81 | 06C11 Light (kappa) Chain Variable Region-nucleic acid |
| 82 | 06C11 Light (kappa) Chain Variable Region-protein |
| 4 | 06C11 Heavy Chain CDR₁ |
| 9 | 06C11 Heavy Chain CDR₂ |
| 18 | 06C11 Heavy Chain CDR₃ |
| 23 | 06C11 Light (kappa) Chain CDR₁ |
| 28 | 06C11 Light (kappa) Chain CDR₂ |
| 35 | 06C11 Light (kappa) Chain CDR₃ |
| 47 | 08G01 Heavy Chain Variable Region-nucleic acid |
| 48 | 08G01 Heavy Chain Variable Region-protein |
| 83 | 08G01 Light (kappa) Chain Variable Region-nucleic acid |
| 84 | 08G01 Light (kappa) Chain Variable Region-protein |
| 1 | 08G01 Heavy Chain CDR₁ |
| 10 | 08G01 Heavy Chain CDR₂ |
| 15 | 08G01 Heavy Chain CDR₃ |
| 24 | 08G01 Light (kappa) Chain CDR₁ |
| 29 | 08G01 Light (kappa) Chain CDR₂ |
| 32 | 08G01 Light (kappa) Chain CDR₃ |
| 49 | 14F11 Heavy Chain Variable Region-nucleic acid |
| 50 | 14F11 Heavy Chain Variable Region-protein |
| 85 | 14F11 Light (kappa) Chain Variable Region-nucleic acid |
| 86 | 14F11 Light (kappa) Chain Variable Region-protein |
| 5 | 14F11 Heavy Chain CDR₁ |
| 11 | 14F11 Heavy Chain CDR₂ |
| 19 | 14F11 Heavy Chain CDR₃ |
| 23 | 14F11 Light (kappa) Chain CDR₁ |
| 30 | 14F11 Light (kappa) Chain CDR₂ |
| 36 | 14F11 Light (kappa) Chain CDR₃ |
| 51 | 17B11 Heavy Chain Variable Region-nucleic acid |
| 52 | 17B11 Heavy Chain Variable Region-protein |
| 87 | 17B11 Light (kappa) Chain Variable Region-nucleic acid |
| 88 | 17B11 Light (kappa) Chain Variable Region-protein |
| 6 | 17B11 Heavy Chain CDR₁ |
| 12 | 17B11 Heavy Chain CDR₂ |
| 20 | 17B11 Heavy Chain CDR₃ |
| 25 | 17B11 Light (kappa) Chain CDR₁ |
| 31 | 17B11 Light (kappa) Chain CDR₂ |
| 37 | 17B11 Light (kappa) Chain CDR₃ |

Mouse monoclonal antibody heavy chain CDR sequences (Kabat, Chothia, and IMGT definitions) are shown in Table 3.

TABLE 3
Kabat

| | | | | Variable Region |
|---|---|---|---|---|
| | CDR1 | CDR2 | CDR3 | SEQ ID NO |
| 01G06 | DYNMD | QINPNNGGIFFNQKFKG | EAITTVGAMDY | 40 |
| | (SEQ ID NO: 1) | (SEQ ID NO: 7) | (SEQ ID NO: 15) | |
| 03G05 | SYWIH | DINPSNGRSKYNEKFKN | EVLDGAMDY | 42 |
| | (SEQ ID NO: 2) | (SEQ ID NO: 8) | (SEQ ID NO: 16) | |
| 04F08 | TYGMGVT | HIYWDDDKRYNPSLKS | TGYSNLFAY | 44 |
| | (SEQ ID NO: 3) | (SEQ ID NO: 9) | (SEQ ID NO: 17) | |
| 06C11 | TYGMGVS | HIYWDDDKRYNPSLKS | RGYDDYWGY | 46 |
| | (SEQ ID NO: 4) | (SEQ ID NO: 9) | (SEQ ID NO: 18) | |
| 08G01 | DYNMD | EINPNNGGTFYNQKFKG | EAITTVGAMDY | 48 |
| | (SEQ ID NO: 1) | (SEQ ID NO: 10) | (SEQ ID NO: 15) | |
| 14F11 | TYGMGVG | DIWWDDDKYYNPSLKS | RGHYSAMDY | 50 |
| | (SEQ ID NO: 5) | (SEQ ID NO: 11) | (SEQ ID NO: 19) | |
| 17B11 | TSGMGVS | HNDWDDDKRYKSSLKS | RVGGLEGYFDY | 52 |
| | (SEQ ID NO: 6) | (SEQ ID NO: 12) | (SEQ ID NO: 20) | |

Chothia

| | | | | Variable Region |
|---|---|---|---|---|
| | CDR1 | CDR2 | CDR3 | SEQ ID NO |
| 01G06 | GYTFTDY | NPNNGG | EAITTVGAMDY | 40 |
| | (SEQ ID NO: 38) | (SEQ ID NO: 143) | (SEQ ID NO: 15) | |
| 03G05 | GYTFTSY | NPSNGR | EVLDGAMDY | 42 |
| | (SEQ ID NO: 128) | (SEQ ID NO: 144) | (SEQ ID NO: 16) | |
| 04F08 | GFSLSTYGM | YWDDD | TGYSNLFAY | 44 |
| | (SEQ ID NO: 130) | (SEQ ID NO: 145) | (SEQ ID NO: 17) | |
| 06C11 | GFSLNTYGM | YWDDD | RGYDDYWGY | 46 |
| | (SEQ ID NO: 132) | (SEQ ID NO: 145) | (SEQ ID NO: 18) | |
| 08G01 | GYTFTDY | NPNNGG | EAITTVGAMDY | 48 |
| | (SEQ ID NO: 38) | (SEQ ID NO: 143) | (SEQ ID NO: 15) | |
| 14F11 | GFSLSTYGM | WWDDD | RGHYSAMDY | 50 |
| | (SEQ ID NO: 130) | (SEQ ID NO: 146) | (SEQ ID NO: 19) | |
| 17B11 | GFSLSTSGM | DWDDD | RVGGLEGYFDY | 52 |
| | (SEQ ID NO: 134) | (SEQ ID NO: 147) | (SEQ ID NO: 20) | |

IMGT

| | | | | Variable Region |
|---|---|---|---|---|
| | CDR1 | CDR2 | CDR3 | SEQ ID NO |
| 01G06 | GYTFTDYN | INPNNGGI | AREAITTVGAMDY | 40 |
| | (SEQ ID NO: 136) | (SEQ ID NO: 148) | (SEQ ID NO: 154) | |
| 03G05 | GYTFTSYW | INPSNGRS | AREVLDGAMDY | 42 |
| | (SEQ ID NO: 138) | (SEQ ID NO: 149) | (SEQ ID NO: 155) | |
| 04F08 | GFSLSTYGMG | IYWDDDK | AQTGYSNLFAY | 44 |
| | (SEQ ID NO: 140) | (SEQ ID NO: 150) | (SEQ ID NO: 156) | |
| 06C11 | GFSLNTYGMG | IYWDDDK | AQRGYDDYWGY | 46 |
| | (SEQ ID NO: 141) | (SEQ ID NO: 150) | (SEQ ID NO: 157) | |
| 08G01 | GYTFTDYN | INPNNGGT | AREAITTVGAMDY | 48 |
| | (SEQ ID NO: 136) | (SEQ ID NO: 151) | (SEQ ID NO: 154) | |
| 14F11 | GFSLSTYGMG | IWWDDDK | ARRGHYSAMDY | 50 |
| | (SEQ ID NO: 140) | (SEQ ID NO: 152) | (SEQ ID NO: 158) | |
| 17B11 | GFSLSTSGMG | NDWDDDK | ARRVGGLEGYFDY | 52 |
| | (SEQ ID NO: 142) | (SEQ ID NO: 153) | (SEQ ID NO: 159) | |

Mouse monoclonal antibody Kappa light chain CDR sequences (Kabat, Chothia, and IMGT definitions) are shown in Table 4.

TABLE 4
Kabat/Chothia

| | | | | Variable Region |
|---|---|---|---|---|
| | CDR1 | CDR2 | CDR3 | SEQ ID NO |
| 01G06 | RTSENLHNYLA | DAKTLAD | QHFWSSPYT | 76 |
| | (SEQ ID NO: 21) | (SEQ ID NO: 26) | (SEQ ID NO: 32) | |
| 03G05 | RASESVDNYGISFMN | AASNQGS | QQSKEVPWT | 78 |
| | (SEQ ID NO: 22) | (SEQ ID NO: 27) | (SEQ ID NO: 33) | |
| 04F08 | KASQNVGTNVA | SASYRYS | QQYNSYPYT | 80 |
| | (SEQ ID NO: 23) | (SEQ ID NO: 28) | (SEQ ID NO: 34) | |
| 06C11 | KASQNVGTNVA | SASYRYS | QQYNNYPLT | 82 |
| | (SEQ ID NO: 23) | (SEQ ID NO: 28) | (SEQ ID NO: 35) | |
| 08G01 | RASGNIHNYLA | NAKTLAD | QHFWSSPYT | 84 |
| | (SEQ ID NO: 24) | (SEQ ID NO: 29) | (SEQ ID NO: 32) | |
| 14F11 | KASQNVGTNVA | SPSYRYS | QQYNSYPHT | 86 |
| | (SEQ ID NO: 23) | (SEQ ID NO: 30) | (SEQ ID NO: 36) | |
| 17B11 | RASQSVSTSRFSYMH | YASNLES | QHSWEIPYT | 88 |
| | (SEQ ID NO: 25) | (SEQ ID NO: 31) | (SEQ ID NO: 37) | |

IMGT

| | | | | Variable Region |
|---|---|---|---|---|
| | CDR1 | CDR2 | CDR3 | SEQ ID NO |
| 01G06 | ENLHNY | DAK | QHFWSSPYT | 76 |
| | (SEQ ID NO: 160) | | (SEQ ID NO: 32) | |
| 03G05 | ESVDNYGISF | AAS | QQSKEVPWT | 78 |
| | (SEQ ID NO: 161) | | (SEQ ID NO: 33) | |
| 04F08 | QNVGTN | SAS | QQYNSYPYT | 80 |
| | (SEQ ID NO: 162) | | (SEQ ID NO: 34) | |
| 06C11 | QNVGTN | SAS | QQYNNYPLT | 82 |
| | (SEQ ID NO: 162) | | (SEQ ID NO: 35) | |
| 08G01 | GNIHNY | NAK | QHFWSSPYT | 84 |
| | (SEQ ID NO: 163) | | (SEQ ID NO: 32) | |
| 14F11 | QNVGTN | SPS | QQYNSYPHT | 86 |
| | (SEQ ID NO: 162) | | (SEQ ID NO: 36) | |
| 17B11 | QSVSTSRFSY | YAS | QHSWEIPYT | 88 |
| | (SEQ ID NO: 164) | | (SEQ ID NO: 37) | |

To create the complete heavy or kappa chain antibody sequences, each variable sequence above is combined with its respective constant region. For example, a complete heavy chain comprises a heavy variable sequence followed by the murine IgG1 or IgG2b heavy chain constant sequence, and a complete kappa chain comprises a kappa variable sequence followed by the murine kappa light chain constant sequence.

Nucleic Acid Sequence Encoding the Murine IgG1 Heavy Chain Constant Region (SEQ ID NO: 165)

| | |
|---|---|
| 1 | gccaaaacga cacccccatc tgtctatcca ctggcccctg gatctgctgc ccaaactaac |
| 61 | tccatggtga ccctgggatg cctggtcaag ggctatttcc ctgagccagt gacagtgacc |
| 121 | tggaactctg gatccctgtc cagcggtgtg cacaccttcc cagctgtcct gcagtctgac |
| 181 | ctctacactc tgagcagctc agtgactgtc ccctccagca cctggcccag cgagaccgtc |
| 241 | acctgcaacg ttgcccaccc ggccagcagc accaaggtgg acaagaaaat tgtgcccagg |
| 301 | gattgtggtt gtaagccttg catatgtaca gtcccagaag tatcatctgt cttcatcttc |
| 361 | cccccaaagc ccaaggatgt gctcaccatt actctgactc ctaaggtcac gtgtgttgtg |
| 421 | gtagacatca gcaaggatga tcccgaggtc cagttcagct ggtttgtaga tgatgtggag |
| 481 | gtgcacacag ctcagacgca accccgggag gagcagttca acagcacttt ccgctcagtc |
| 541 | agtgaacttc ccatcatgca ccaggactgg ctcaatggca aggagttcaa atgcagggtc |
| 601 | aacagtgcag ctttccctgc ccccatcgag aaaaccatct ccaaaaccaa aggcagaccg |
| 661 | aaggctccac aggtgtacac cattccacct cccaaggagc agatggccaa ggataaagtc |
| 721 | agtctgacct gcatgataac agacttcttc cctgaagaca ttactgtgga gtggcagtgg |
| 781 | aatgggcagc cagcggagaa ctacaagaac actcagccca tcatggacac agatggctct |
| 841 | tacttcgtct acagcaagct caatgtgcag aagagcaact gggaggcagg aaatactttc |
| 901 | acctgctctg tgttacatga gggcctgcac aaccaccata ctgagaagag cctctcccac |
| 961 | tctcctggta aa |

Protein Sequence Defining the Murine IgG1 Heavy Chain Constant Region (SEQ ID NO:166)

| | |
|---|---|
| 1 | akttppsvyp lapgsaaqtn smvtlgclvk gyfpepvtvt wnsgslssgv htfpavlqsd |
| 61 | lytlsssvtv psstwpsetv tcnvahpass tkvdkkivpr dcgckpcict vpevssvfif |
| 121 | ppkpkdvlti tltpkvtcvv vdiskddpev qfswfvddve vhtaqtqpre eqfnstfrsv |
| 181 | selpimhqdw lngkefkcrv nsaafpapie ktisktkgrp kapqvytipp pkeqmakdkv |
| 241 | sltcmitdff peditvewqw ngqpaenykn tqpimdtdgs yfvysklnvq ksnweagntf |
| 301 | tcsvlheglh nhhtekslsh spgk |

Nucleic Acid Sequence Encoding the Murine IgG2b Heavy Chain Constant Region (SEQ ID NO:167)

| | |
|---|---|
| 1 | gccaaaacaa cacccccatc agtctatcca ctggcccctg ggtgtggaga tacaactggt |
| 61 | tcctccgtga ctctgggatg cctggtcaag ggctacttcc ctgagtcagt gactgtgact |
| 121 | tggaactctg gatccctgtc cagcagtgtg cacaccttcc cagctctcct gcagtctgga |
| 181 | ctctacacta tgagcagctc agtgactgtc ccctccagca cctggccaag tcagaccgtc |
| 241 | acctgcagcg ttgctcaccc agccagcagc accacggtgg acaaaaaact tgagcccagc |
| 301 | gggcccattt caacaatcaa cccctgtcct ccatgcaagg agtgtcacaa atgcccagct |
| 361 | cctaacctcg agggtggacc atccgtcttc atcttccctc caaatatcaa ggatgtactc |
| 421 | atgatctccc tgacacccaa ggtcacgtgt gtggtggtgg atgtgagcga ggatgaccca |
| 481 | gacgtccaga tcagctggtt tgtgaacaac gtggaagtac acacagctca gacacaaacc |
| 541 | catagagagg attacaacag tactatccgg gtggtcagca ccctccccat ccagcaccag |
| 601 | gactggatga gtggcaagga gttcaaatgc aaggtcaaca acaaagacct cccatcaccc |
| 661 | atcgagagaa ccatctcaaa aattaaaggg ctagtcagag ctccacaagt atacatcttg |
| 721 | ccgccaccag cagagcagtt gtccaggaaa gatgtcagtc tcacttgcct ggtcgtgggc |
| 781 | ttcaaccctg gagacatcag tgtggagtgg accagcaatg ggcatacaga ggagaactac |
| 841 | aaggacaccg caccagtcct agactctgac ggttcttact tcatatatag caagctcaat |
| 901 | atgaaaacaa gcaagtggga gaaaacagat tccttctcat gcaacgtgag acacgagggt |
| 961 | ctgaaaaatt actacctgaa gaagaccatc tcccggtctc cgggtaaa |

Protein Sequence Defining the Murine IgG2b Heavy Chain Constant Region (SEQ ID NO:168)

| | |
|---|---|
| 1 | akttppsvyp lapgcgdttg ssvtlgclvk gyfpesvtvt wnsgslsssv htfpallqsg |
| 61 | lytmsssvtv psstwpsqtv tcsvahpass ttvdkkleps gpistinpcp pckechkcpa |
| 121 | pnleggpsvf ifppnikdvl misltpkvtc vvvdvseddp dvqiswfvnn vevhtaqtqt |
| 181 | hredynstir vvstlpiqhq dwmsgkefkc kvnnkdlpsp iertiskikg lvrapqvyil |
| 241 | pppaeqlsrk dvsltclvvg fnpgdisvew tsnghteeny kdtapvldsd gsyfiyskln |
| 301 | mktskwektd sfscnvrheg lknyylkkti srspgk |

Nucleic Acid Sequence Encoding the Murine Kappa Light Chain Constant Region (SEQ ID NO:169)

| | |
|---|---|
| 1 | cgggctgatg ctgcaccaac tgtatccatc ttcccaccat ccagtgagca gttaacatct |
| 61 | ggaggtgcct cagtcgtgtg cttcttgaac aacttctacc ccaaagacat caatgtcaag |
| 121 | tggaagattg atggcagtga acgacaaaat ggcgtcctga acagttggac tgatcaggac |
| 181 | agcaaagaca gcacctacag catgagcagc accctcacgt tgaccaagga cgagtatgaa |
| 241 | cgacataaca gctatacctg tgaggccact cacaagacat caacttcacc cattgtcaag |
| 301 | agcttcaaca ggaatgagtg t |

Protein Sequence Defining the Murine Kappa Light Chain Constant Region (SEQ ID NO:170)

| | |
|---|---|
| 1 | radaaptvsi fppsseqlts ggasvvcfln nfypkdinvk wkidgserqn gvlnswtdqd |
| 61 | skdstysmss tltltkdeye rhnsytceat hktstspivk sfnrnec |

The following sequences represent the actual or contemplated full length heavy and light chain sequence (i.e., containing both the variable and constant regions sequences) for each antibody described in this Example. Signal sequences for proper secretion of the antibodies (e.g., signal sequences at the 5′ end of the DNA sequences or the amino terminal end of the protein sequences) are not shown in the full length heavy and light chain sequences disclosed herein and are not included in the final secreted protein. Also not shown are stop codons for termination of translation required at the 3′ end of the DNA sequences. It is within ordinary skill in the art to select a signal sequence and/or a stop codon for expression of the disclosed full length immunoglobulin heavy chain and light chain sequences. It is also contemplated that the variable region sequences can be ligated to other constant region sequences to produce active full length immunoglobulin heavy and light chains.

Nucleic Acid Sequence Encoding the Full Length Heavy Chain Sequence (Heavy Chain Variable Region and IgG1 Constant Region) of 01G06 (SEQ ID NO:99)

| | |
|---|---|
| 1 | gaggtcctgc tgcaacagtc tggacctgag ctggtgaagc ctggggcttc agtgaagata |
| 61 | ccctgcaagg cttctggata cacattcact gactacaaca tggactgggt gaagcagagc |
| 121 | catggaaaga gccttgagtg gattggacaa attaatccta acaatggtgg tattttcttc |
| 181 | aaccagaagt tcaagggcaa ggccacattg actgtagaca agtcctccaa tacagccttc |
| 241 | atggaggtcc gcagcctgac atctgaggac actgcagtct attactgtgc aagagaggca |
| 301 | attactacgg taggcgctat ggactactgg ggtcaaggaa cctcagtcac cgtctcctca |
| 361 | gccaaaacga cacccccatc tgtctatcca ctggcccctg gatctgctgc ccaaactaac |
| 421 | tccatggtga ccctgggatg cctggtcaag ggctatttcc ctgagccagt gacagtgacc |
| 481 | tggaactctg gatccctgtc cagcggtgtg cacaccttcc cagctgtcct gcagtctgac |
| 541 | ctctacactc tgagcagctc agtgactgtc ccctccagca cctggcccag cgagaccgtc |
| 601 | acctgcaacg ttgcccaccc ggccagcagc accaaggtgg acaagaaaat tgtgcccagg |
| 661 | gattgtggtt gtaagccttg catatgtaca gtcccagaag tatcatctgt cttcatcttc |
| 721 | cccccaaagc ccaaggatgt gctcaccatt actctgactc ctaaggtcac gtgtgttgtg |
| 781 | gtagacatca gcaaggatga tcccgaggtc cagttcagct ggtttgtaga tgatgtggag |
| 841 | gtgcacacag ctcagacgca accccgggag gagcagttca acagcacttt ccgctcagtc |
| 901 | agtgaacttc ccatcatgca ccaggactgg ctcaatggca aggagttcaa atgcagggtc |
| 961 | aacagtgcag ctttccctgc ccccatcgag aaaaccatct ccaaaaccaa aggcagaccg |
| 1021 | aaggctccac aggtgtacac cattccacct cccaaggagc agatggccaa ggataaagtc |
| 1081 | agtctgacct gcatgataac agacttcttc cctgaagaca ttactgtgga gtggcagtgg |
| 1141 | aatgggcagc cagcggagaa ctacaagaac actcagccca tcatggacac agatggctct |
| 1201 | tacttcgtct acagcaagct caatgtgcag aagagcaact gggaggcagg aaatactttc |
| 1261 | acctgctctg tgttacatga gggcctgcac aaccaccata ctgagaagag cctctcccac |
| 1321 | tctcctggta aa |

Protein Sequence Defining the Full Length Heavy Chain Sequence (Heavy Chain Variable Region and IgG1 Constant Region) of 01G06 (SEQ ID NO:100)

| | |
|---|---|
| 1 | evllqqsgpe lvkpgasvki pckasgytft dynmdwvkqs hgkslewigq inpnnggiff |
| 61 | nqkfkgkatl tvdkssntaf mevrsltsed tavyycarea ittvgamdyw gqgtsvtvss |
| 121 | akttppsvyp lapgsaaqtn smvtlgclvk gyfpepvtvt wnsgslssgv htfpavlqsd |
| 181 | lytlsssvtv psstwpsetv tcnvahpass tkvdkkivpr dcgckpcict vpevssvfif |
| 241 | ppkpkdvlti tltpkvtcvv vdiskddpev qfswfvddve vhtaqtqpre eqfnstfrsv |
| 301 | selpimhqdw lngkefkcrv nsaafpapie ktisktkgrp kapqvytipp pkeqmakdkv |
| 361 | sltcmitdff peditvewqw ngqpaenykn tqpimdtdgs yfvysklnvq ksnweagntf |
| 421 | tcsvlheglh nhhtekslsh spgk |

Nucleic Acid Sequence Encoding the Full Length Light Chain Sequence (Kappa Chain Variable Region and Constant Region) of 01G06 (SEQ ID NO:101)

| | |
|---|---|
| 1 | gacatccaga tgactcagtc tccagcctcc ctatctgcat ctgtgggaga aactgtcacc |
| 61 | atcacatgtc gaacaagtga gaatcttcac aattatttag catggtatca gcagaaacag |
| 121 | ggaaaatctc ctcagctcct ggtctatgat gcaaaaacct tagcagatgg tgtgccatca |
| 181 | aggttcagtg gcagtggatc aggaacacaa tattctctca agatcaacag cctgcagcct |
| 241 | gaagattttg ggagttatta ctgtcaacat ttttggagta gtccttacac gttcggaggg |
| 301 | gggaccaagc tggaaataaa acgggctgat gctgcaccaa ctgtatccat cttcccacca |
| 361 | tccagtgagc agttaacatc tggaggtgcc tcagtcgtgt gcttcttgaa caacttctac |
| 421 | cccaaagaca tcaatgtcaa gtggaagatt gatggcagtg aacgacaaaa tggcgtcctg |
| 481 | aacagttgga ctgatcagga cagcaaagac agcacctaca gcatgagcag caccctcacg |
| 541 | ttgaccaagg acgagtatga acgacataac agctatacct gtgaggccac tcacaagaca |
| 601 | tcaacttcac ccattgtcaa gagcttcaac aggaatgagt gt |

Protein Sequence Defining the Full Length Light Chain Sequence (Kappa Chain Variable Region and Constant Region) of 01G06 (SEQ ID NO:102)

| | |
|---|---|
| 1 | diqmtqspas lsasvgetvt itcrtsenlh nylawyqqkq gkspqllvyd aktladgvps |
| 61 | rfsgsgsgtq yslkinslqp edfgsyycqh fwsspytfgg gtkleikrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Nucleic Acid Sequence Encoding the Full Length Heavy Chain Sequence (Heavy Chain Variable Region and 12G1 Constant Region) of 03G05 (SEQ ID NO:103)

| | |
|---|---|
| 1 | caggtccaac tgcagcagcc tggggctgaa ctggtgaagc ctggggcttc agtgaagctg |
| 61 | tcctgcaagg cttctggcta caccttcacc agctactgga ttcactgggt gaaccagagg |
| 121 | cctggacaag gccttgagtg gattggagac attaatccta gcaacggccg tagtaagtat |
| 181 | aatgagaagt tcaagaacaa ggccacaatg actgcagaca aatcctccaa cacagcctac |
| 241 | atgcaactca gcagcctgac atctgaggac tctgcggtct attactgtgc aagagaggtt |
| 301 | ctggatggtg ctatggacta ctggggtcaa ggaacctcag tcaccgtctc ctcagccaaa |
| 361 | acgacacccc catctgtcta tccactggcc cctggatctg ctgcccaaac taactccatg |
| 421 | gtgaccctgg gatgcctggt caagggctat ttccctgagc cagtgacagt gacctggaac |
| 481 | tctggatccc tgtccagcgg tgtgcacacc ttcccagctg tcctgcagtc tgacctctac |
| 541 | actctgagca gctcagtgac tgtcccctcc agcacctggc ccagcgagac cgtcacctgc |
| 601 | aacgttgccc acccggccag cagcaccaag gtggacaaga aaattgtgcc cagggattgt |
| 661 | ggttgtaagc cttgcatatg tacagtccca gaagtatcat ctgtcttcat cttcccccca |
| 721 | aagcccaagg atgtgctcac cattactctg actcctaagg tcacgtgtgt tgtggtagac |
| 781 | atcagcaagg atgatcccga ggtccagttc agctggtttg tagatgatgt ggaggtgcac |
| 841 | acagctcaga cgcaaccccg ggaggagcag ttcaacagca ctttccgctc agtcagtgaa |
| 901 | cttcccatca tgcaccagga ctggctcaat ggcaaggagt tcaaatgcag ggtcaacagt |
| 961 | gcagctttcc ctgcccccat cgagaaaacc atctccaaaa ccaaaggcag accgaaggct |
| 1021 | ccacaggtgt acaccattcc acctcccaag gagcagatgg ccaaggataa agtcagtctg |
| 1081 | acctgcatga taacagactt cttccctgaa gacattactg tggagtggca gtggaatggg |
| 1141 | cagccagcgg agaactacaa gaacactcag cccatcatgg acacagatgg ctcttacttc |
| 1201 | gtctacagca agctcaatgt gcagaagagc aactgggagg caggaaatac tttcacctgc |
| 1261 | tctgtgttac atgagggcct gcacaaccac catactgaga agagcctctc ccactctcct |
| 1321 | ggtaaa |

Protein Sequence Defining the Full Length Heavy Chain Sequence (Heavy Chain Variable Region and IgG1 Constant Region) of 03G05 (SEQ ID NO:104)

| | |
|---|---|
| 1 | qvqlqqpgae lvkpgasvkl sckasgytft sywihwvnqr pgqglewigd inpsngrsky |
| 61 | nekfknkatm tadkssntay mqlssltsed savyycarev ldgamdywgq gtsvtvssak |
| 121 | ttppsvypla pgsaaqtnsm vtlgclvkgy fpepvtvtwn sgslssgvht fpavlqsdly |
| 181 | tlsssvtvps stwpsetvtc nvahpasstk vdkkivprdc gckpcictvp evssvfifpp |
| 241 | kpkdvltitl tpkvtcvvvd iskddpevqf swfvddvevh taqtqpreeq fnstfrsvse |
| 301 | lpimhqdwln gkefkcrvns aafpapiekt isktkgrpka pqvytipppk eqmakdkvsl |
| 361 | tcmitdffpe ditvewqwng qpaenykntq pimdtdgsyf vysklnvqks nweagntftc |
| 421 | svlheglhnh htekslshsp gk |

Nucleic Acid Sequence Encoding the Full Length Light Chain Sequence (Kappa Chain Variable Region and Constant Region) of 03G05 (SEQ ID NO:105)

| | |
|---|---|
| 1 | gacattgtgt tgacccaatc tccagcttct ttggctgtgt ctctagggca gagggccacc |
| 61 | atctcctgca gagccagcga aagtgttgat aattatggca ttagttttat gaactggttc |
| 121 | caacagaaac caggacagcc acccaaactc ctcatctatg ctgcatccaa ccaaggctcc |
| 181 | ggggtccctg ccaggtttag tggcagtggg tctgggacag acttcagcct caacatccat |
| 241 | cctatggagg aggatgatac tgcaatgtat ttctgtcagc aaagtaagga ggttccgtgg |
| 301 | acgttcggtg gaggctccaa gctggaaatc aaacgggctg atgctgcacc aactgtatcc |
| 361 | atcttcccac catccagtga gcagttaaca tctggaggtg cctcagtcgt gtgcttcttg |
| 421 | aacaacttct accccaaaga catcaatgtc aagtggaaga ttgatggcag tgaacgacaa |
| 481 | aatggcgtcc tgaacagttg gactgatcag gacagcaaag acagcaccta cagcatgagc |
| 541 | agcaccctca cgttgaccaa ggacgagtat gaacgacata acagctatac ctgtgaggcc |
| 601 | actcacaaga catcaacttc acccattgtc aagagcttca acaggaatga gtgt |

Protein Sequence Defining the Full Length Light Chain Sequence (Kappa Chain Variable Region and Constant Region) of 03G05 (SEQ ID NO:106)

| | |
|---|---|
| 1 | divltqspas lavslgqrat iscrasesvd nygisfmnwf qqkpgqppkl liyaasnqgs |
| 61 | gvparfsgsg sgtdfslnih pmeeddtamy fcqqskevpw tfgggsklei kradaaptvs |
| 121 | ifppsseqlt sggasvvcfl nnfypkdinv kwkidgserq ngvlnswtdq dskdstysms |
| 181 | stltltkdey erhnsytcea thktstspiv ksfnrnec |

Nucleic Acid Sequence Encoding the Full Length Heavy Chain Sequence (Heavy Chain Variable Region and IgG1 Constant Region) of 04F08 (SEQ ID NO:107)

| | |
|---|---|
| 1 | caggttactc tgaaagagtc tggccctggg atattgcagc cctcccagac cctcagtctg |
| 61 | acttgttctt tctctgggtt ttcactgagc acttatggta tgggtgtgac ctggattcgt |
| 121 | cagccttcag gaaagggtct ggagtggctg gcacacattt actgggatga tgacaagcgc |
| 181 | tataacccat ccctgaagag ccggctcaca atctccaagg atacctccaa caaccaggta |
| 241 | ttcctcaaga tcaccagtgt ggacactgca gatactgcca catactactg tgctcaaacg |
| 301 | gggtatagta acttgtttgc ttactggggc caagggactc tggtcactgt ctctgcagcc |
| 361 | aaaacgacac ccccatctgt ctatccactg gcccctggat ctgctgccca aactaactcc |
| 421 | atggtgaccc tgggatgcct ggtcaagggc tatttccctg agccagtgac agtgacctgg |
| 481 | aactctggat ccctgtccag cggtgtgcac accttcccag ctgtcctgca gtctgacctc |
| 541 | tacactctga gcagctcagt gactgtcccc tccagcacct ggcccagcga gaccgtcacc |
| 601 | tgcaacgttg cccacccggc cagcagcacc aaggtggaca agaaaattgt gcccagggat |
| 661 | tgtggttgta agccttgcat atgtacagtc ccagaagtat catctgtctt catcttcccc |
| 721 | ccaaagccca aggatgtgct caccattact ctgactccta aggtcacgtg tgttgtggta |
| 781 | gacatcagca aggatgatcc cgaggtccag ttcagctggt ttgtagatga tgtggaggtg |
| 841 | cacacagctc agacgcaacc ccgggaggag cagttcaaca gcactttccg ctcagtcagt |
| 901 | gaacttccca tcatgcacca ggactggctc aatggcaagg agttcaaatg cagggtcaac |
| 961 | agtgcagctt tccctgcccc catcgagaaa accatctcca aaaccaaagg cagaccgaag |
| 1021 | gctccacagg tgtacaccat tccacctccc aaggagcaga tggccaagga taaagtcagt |
| 1081 | ctgacctgca tgataacaga cttcttccct gaagacatta ctgtggagtg gcagtggaat |
| 1141 | gggcagccag cggagaacta caagaacact cagcccatca tggacacaga tggctcttac |
| 1201 | ttcgtctaca gcaagctcaa tgtgcagaag agcaactggg aggcaggaaa tactttcacc |
| 1261 | tgctctgtgt tacatgaggg cctgcacaac caccatactg agaagagcct ctcccactct |
| 1321 | cctggtaaa |

Protein Sequence Defining the Full Length Heavy Chain Sequence (Heavy Chain Variable Region and IgG1 Constant Region) of 04F08 (SEQ ID NO:108)

| | |
|---|---|
| 1 | qvtlkesgpg ilqpsqtlsl tcsfsgfsls tygmgvtwir qpsgkglewl ahiywdddkr |
| 61 | ynpslksrlt iskdtsnnqv flkitsvdta dtatyycaqt gysnlfaywg qgtlvtvsaa |
| 121 | kttppsvypl apgsaaqtns mvtlgclvkg yfpepvtvtw nsgslssgvh tfpavlqsdl |
| 181 | ytlsssvtvp sstwpsetvt cnvahpasst kvdkkivprd cgckpcictv pevssvfifp |
| 241 | pkpkdvltit ltpkvtcvvv diskddpevq fswfvddvev htaqtqpree qfnstfrsvs |
| 301 | elpimhqdwl ngkefkcrvn saafpapiek tisktkgrpk apqvytippp keqmakdkvs |
| 361 | ltcmitdffp editvewqwn gqpaenyknt qpimdtdgsy fvysklnvqk snweagntft |
| 421 | csvlheglhn hhtekslshs pgk |

Nucleic Acid Sequence Encoding the Full Length Light Chain Sequence (Kappa Chain Variable Region and Constant Region) of 04F08 (SEQ ID NO: 109)

| | |
|---|---|
| 1 | gacattgtga tgacccagtc tcaaaaattc atgtccacat cagtaggaga cagggtcagc |
| 61 | gtcacctgca aggccagtca gaatgtgggt actaatgtag cctggtatca acagaaatta |
| 121 | ggacaatctc ctaaaacact gatttactcg gcatcctacc ggtacagtgg agtccctgat |
| 181 | cgcttcacag gcagtggatc tgggacagat ttcactctca ccatcagcaa tgtgcagtct |
| 241 | gaagacttgg cagagtattt ctgtcagcaa tataacagct atccgtacac gttcggaggg |
| 301 | gggaccaagc tggaaataaa acgggctgat gctgcaccaa ctgtatccat cttcccacca |
| 361 | tccagtgagc agttaacatc tggaggtgcc tcagtcgtgt gcttcttgaa caacttctac |
| 421 | cccaaagaca tcaatgtcaa gtggaagatt gatggcagtg aacgacaaaa tggcgtcctg |
| 481 | aacagttgga ctgatcagga cagcaaagac agcacctaca gcatgagcag caccctcacg |
| 541 | ttgaccaagg acgagtatga acgacataac agctatacct gtgaggccac tcacaagaca |
| 601 | tcaacttcac ccattgtcaa gagcttcaac aggaatgagt gt |

Protein Sequence Defining the Full Length Light Chain Sequence (Kappa Chain Variable Region and Constant Region) of 04F08 (SEQ ID NO:110)

| | |
|---|---|
| 1 | divmtqsqkf mstsvgdrvs vtckasqnvg tnvawyqqkl gqspktliys asyrysgvpd |
| 61 | rftgsgsgtd ftltisnvqs edlaeyfcqq ynsypytfgg gtkleikrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Nucleic Acid Sequence Encoding the Full Length Heavy Chain Sequence (Heavy Chain Variable Region and IgG1 Constant Region) of 06C11 (SEQ ID NO:111)

| | |
|---|---|
| 1 | caggttactc tgaaagagtc tggccctggg atattgcagc cctcccagac cctcagtctg |
| 61 | acttgttctt tctctgggtt ttcactgaac acttatggta tgggtgtgag ctggattcgt |
| 121 | cagccttcag gaaagggtct ggagtggctg gcacacattt actgggatga tgacaagcgc |
| 181 | tataacccat ccctgaagag ccggctcaca atctccaagg atgcctccaa caaccgggtc |
| 241 | ttcctcaaga tcaccagtgt ggacactgca gatactgcca catactactg tgctcaaaga |
| 301 | ggttatgatg attactgggg ttactggggc caagggactc tggtcactat ctctgcagcc |
| 361 | aaaacgacac ccccatctgt ctatccactg gcccctggat ctgctgccca aactaactcc |
| 421 | atggtgaccc tgggatgcct ggtcaagggc tatttccctg agccagtgac agtgacctgg |
| 481 | aactctggat ccctgtccag cggtgtgcac accttcccag ctgtcctgca gtctgacctc |
| 541 | tacactctga gcagctcagt gactgtcccc tccagcacct ggcccagcga gaccgtcacc |
| 601 | tgcaacgttg cccacccggc cagcagcacc aaggtggaca agaaaattgt gcccagggat |
| 661 | tgtggttgta agccttgcat atgtacagtc ccagaagtat catctgtctt catcttcccc |
| 721 | ccaaagccca aggatgtgct caccattact ctgactccta aggtcacgtg tgttgtggta |
| 781 | gacatcagca aggatgatcc cgaggtccag ttcagctggt ttgtagatga tgtggaggtg |
| 841 | cacacagctc agacgcaacc ccgggaggag cagttcaaca gcactttccg ctcagtcagt |
| 901 | gaacttccca tcatgcacca ggactggctc aatggcaagg agttcaaatg cagggtcaac |
| 961 | agtgcagctt tccctgcccc catcgagaaa accatctcca aaaccaaagg cagaccgaag |
| 1021 | gctccacagg tgtacaccat tccacctccc aaggagcaga tggccaagga taaagtcagt |
| 1081 | ctgacctgca tgataacaga cttcttccct gaagacatta ctgtggagtg gcagtggaat |
| 1141 | gggcagccag cggagaacta caagaacact cagcccatca tggacacaga tggctcttac |
| 1201 | ttcgtctaca gcaagctcaa tgtgcagaag agcaactggg aggcaggaaa tactttcacc |
| 1261 | tgctctgtgt tacatgaggg cctgcacaac caccatactg agaagagcct ctcccactct |
| 1321 | cctggtaaa |

Protein Sequence Defining the Full Length Heavy Chain Sequence (Heavy Chain Variable Region and IgG1 Constant Region) of 06C11 (SEQ ID NO:112)

| | |
|---|---|
| 1 | qvtlkesgpg ilqpsqtlsl tcsfsgfsln tygmgvswir qpsgkglewl ahiywdddkr |
| 61 | ynpslksrlt iskdasnnrv flkitsvdta dtatyycaqr gyddywgywg qgtlvtisaa |
| 121 | kttppsvypl apgsaaqtns mvtlgclvkg yfpepvtvtw nsgslssgvh tfpavlqsdl |
| 181 | ytlsssvtvp sstwpsetvt cnvahpasst kvdkkivprd cgckpcictv pevssvfifp |
| 241 | pkpkdvltit ltpkvtcvvv diskddpevq fswfvddvev htaqtqpree qfnstfrsvs |
| 301 | elpimhqdwl ngkefkcrvn saafpapiek tisktkgrpk apqvytippp keqmakdkvs |
| 361 | ltcmitdffp editvewqwn gqpaenyknt qpimdtdgsy fvysklnvqk snweagntft |
| 421 | csvlheglhn hhtekslshs pgk |

Nucleic Acid Sequence Encoding the Full Length Light Chain Sequence (Kappa Chain Variable Region and Constant Region) of 06C11 (SEQ ID NO:113)

| | |
|---|---|
| 1 | gacattgtga tgacccagtc tcaaaaattc atgtccacat cagtaggaga cagggtcagc |
| 61 | gtcacctgca aggccagtca gaatgtgggt actaatgtag cctggtttca acagaaacca |
| 121 | ggtcaatctc ctaaagcact gatttactcg gcatcttacc ggtacagtgg agtccctgat |
| 181 | cgcttcacag gcagtggatc tgggacagat ttcattctca ccatcagcaa tgtgcagtct |
| 241 | gaagacctgg cagagtattt ctgtcagcaa tataacaact atcctctcac gttcggtgct |
| 301 | gggaccaagc tggagctgaa acgggctgat gctgcaccaa ctgtatccat cttcccacca |
| 361 | tccagtgagc agttaacatc tggaggtgcc tcagtcgtgt gcttcttgaa caacttctac |
| 421 | cccaaagaca tcaatgtcaa gtggaagatt gatggcagtg aacgacaaaa tggcgtcctg |
| 481 | aacagttgga ctgatcagga cagcaaagac agcacctaca gcatgagcag caccctcacg |
| 541 | ttgaccaagg acgagtatga acgacataac agctatacct gtgaggccac tcacaagaca |
| 601 | tcaacttcac ccattgtcaa gagcttcaac aggaatgagt gt |

Protein Sequence Defining the Full Length Light Chain Sequence (Kappa Chain Variable Region and Constant Region) of 06C11 (SEQ ID NO:114)

| | |
|---|---|
| 1 | divmtqsqkf mstsvgdrvs vtckasqnvg tnvawfqqkp gqspkaliys asyrysgvpd |
| 61 | rftgsgsgtd filtisnvqs edlaeyfcqq ynnypltfga gtklelkrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Nucleic Acid Sequence Encoding the Full Length Heavy Chain Sequence (Heavy Chain Variable Region and IgG2b Constant Region) of 08G01 (SEQ ID NO:115)

| | |
|---|---|
| 1 | gaggtcctgc tgcaacagtc tggacctgag gtggtgaagc ctggggcttc agtgaagata |
| 61 | ccctgcaagg cttctggata cacattcact gactacaaca tggactgggt gaagcagagc |
| 121 | catggaaaga gccttgagtg gattggagag attaatccta acaatggtgg tactttctac |
| 181 | aaccagaagt tcaagggcaa ggccacattg actgtagaca agtcctccag cacagcctac |
| 241 | atggagctcc gcagcctgac atctgaggac actgcagtct attactgtgc aagagaggca |
| 301 | attactacgg taggcgctat ggactactgg ggtcaaggaa cctcagtcac cgtctcctca |
| 361 | gccaaaacaa cacccccatc agtctatcca ctggcccctg ggtgtggaga tacaactggt |
| 421 | tcctccgtga ctctgggatg cctggtcaag ggctacttcc ctgagtcagt gactgtgact |
| 481 | tggaactctg gatccctgtc cagcagtgtg cacaccttcc cagctctcct gcagtctgga |
| 541 | ctctacacta tgagcagctc agtgactgtc ccctccagca cctggccaag tcagaccgtc |
| 601 | acctgcagcg ttgctcaccc agccagcagc accacggtgg acaaaaaact tgagcccagc |
| 661 | gggcccattt caacaatcaa cccctgtcct ccatgcaagg agtgtcacaa atgcccagct |
| 721 | cctaacctcg agggtggacc atccgtcttc atcttccctc caaatatcaa ggatgtactc |
| 781 | atgatctccc tgacacccaa ggtcacgtgt gtggtggtgg atgtgagcga ggatgaccca |
| 841 | gacgtccaga tcagctggtt tgtgaacaac gtggaagtac acacagctca gacacaaacc |
| 901 | catagagagg attacaacag tactatccgg gtggtcagca ccctccccat ccagcaccag |
| 961 | gactggatga gtggcaagga gttcaaatgc aaggtcaaca acaaagacct cccatcaccc |
| 1021 | atcgagagaa ccatctcaaa aattaaaggg ctagtcagag ctccacaagt atacatcttg |
| 1081 | ccgccaccag cagagcagtt gtccaggaaa gatgtcagtc tcacttgcct ggtcgtgggc |
| 1141 | ttcaaccctg gagacatcag tgtggagtgg accagcaatg ggcatacaga ggagaactac |
| 1201 | aaggacaccg caccagtcct agactctgac ggttcttact tcatatatag caagctcaat |
| 1261 | atgaaaacaa gcaagtggga gaaaacagat tccttctcat gcaacgtgag acacgagggt |
| 1321 | ctgaaaaatt actacctgaa gaagaccatc tcccggtctc cgggtaaa |

Protein Sequence Defining the Full Length Heavy Chain Sequence (Heavy Chain Variable Region and IgG2b Constant Region) of 08G01 (SEQ ID NO:116)

| | |
|---|---|
| 1 | evllqqsgpe vvkpgasvki pckasgytft dynmdwvkqs hgkslewige inpnnggtfy |
| 61 | nqkfkgkatl tvdkssstay melrsltsed tavyycarea ittvgamdyw gqgtsvtvss |
| 121 | akttppsvyp lapgcgdttg ssvtlgclvk gyfpesvtvt wnsgslsssv htfpallqsg |
| 181 | lytmsssvtv psstwpsqtv tcsvahpass ttvdkkleps gpistinpcp pckechkcpa |
| 241 | pnleggpsvf ifppnikdvl misltpkvtc vvvdvseddp dvqiswfvnn vevhtaqtqt |
| 301 | hredynstir vvstlpiqhq dwmsgkefkc kvnnkdlpsp iertiskikg lvrapqvyil |
| 361 | pppaeqlsrk dvsltclvvg fnpgdisvew tsnghteeny kdtapvldsd gsyfiyskln |
| 421 | mktskwektd sfscnvrheg lknyylkkti srspgk |

Nucleic Acid Sequence Encoding the Full Length Light Chain Sequence (Kappa Chain Variable Region and Constant Region) of 08G01 (SEQ ID NO:117)

| | |
|---|---|
| 1 | gacatccaga tgactcagtc tccagcctcc ctatctgcat ctgtgggaga aactgtcacc |
| 61 | atcacatgtc gagcaagtgg gaatattcac aattatttag catggtatca gcagaaacag |
| 121 | ggaaaatctc ctcagctcct ggtctataat gcaaaaacct tagcagatgg tgtgccatca |
| 181 | aggttcagtg gcagtggatc aggaacacaa tattctctca agatcaacag cctgcagcct |
| 241 | gaagattttg ggagttatta ctgtcaacat ttttggagtt ctccttacac gttcggaggg |
| 301 | gggaccaagc tggaaataaa acgggctgat gctgcaccaa ctgtatccat cttcccacca |
| 361 | tccagtgagc agttaacatc tggaggtgcc tcagtcgtgt gcttcttgaa caacttctac |
| 421 | cccaaagaca tcaatgtcaa gtggaagatt gatggcagtg aacgacaaaa tggcgtcctg |
| 481 | aacagttgga ctgatcagga cagcaaagac agcacctaca gcatgagcag caccctcacg |
| 541 | ttgaccaagg acgagtatga acgacataac agctatacct gtgaggccac tcacaagaca |
| 601 | tcaacttcac ccattgtcaa gagcttcaac aggaatgagt gt |

Protein Sequence Defining the Full Length Light Chain Sequence (Kappa Chain Variable Region and Constant Region) of 08G01 (SEQ ID NO:118)

| | |
|---|---|
| 1 | diqmtqspas lsasvgetvt itcrasgnih nylawyqqkq gkspqllvyn aktladgvps |
| 61 | rfsgsgsgtq yslkinslqp edfgsyycqh fwsspytfgg gtkleikrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Nucleic Acid Sequence Encoding the Full Length Heavy Chain Sequence (Heavy Chain Variable Region and IgG1 Constant Region) of 14F11 (SEQ ID NO:119)

| | |
|---|---|
| 1 | caggttactc tgaaagagtc tggccctgga atattgcagc cctcccagac cctcagtctg |
| 61 | acttgttctt tctctgggtt ttcactgagc acttatggta tgggtgtagg ctggattcgt |
| 121 | cagccttcag gaaagggtct agagtggctg gcagacattt ggtgggatga cgataagtac |
| 181 | tataacccat ccctgaagag ccggctcaca atctccaagg atacctccag caatgaggta |
| 241 | ttcctcaaga tcgccattgt ggacactgca gatactgcca cttactactg tgctcgaaga |
| 301 | ggtcactact ctgctatgga ctactggggt caaggaacct cagtcaccgt ctcctcagcc |
| 361 | aaaacgacac ccccatctgt ctatccactg gcccctggat ctgctgccca aactaactcc |
| 421 | atggtgaccc tgggatgcct ggtcaagggc tatttccctg agccagtgac agtgacctgg |
| 481 | aactctggat ccctgtccag cggtgtgcac accttcccag ctgtcctgca gtctgacctc |
| 541 | tacactctga gcagctcagt gactgtcccc tccagcacct ggcccagcga gaccgtcacc |
| 601 | tgcaacgttg cccacccggc cagcagcacc aaggtggaca agaaaattgt gcccagggat |
| 661 | tgtggttgta agccttgcat atgtacagtc ccagaagtat catctgtctt catcttcccc |
| 721 | ccaaagccca aggatgtgct caccattact ctgactccta aggtcacgtg tgttgtggta |
| 781 | gacatcagca aggatgatcc cgaggtccag ttcagctggt ttgtagatga tgtggaggtg |
| 841 | cacacagctc agacgcaacc ccgggaggag cagttcaaca gcactttccg ctcagtcagt |
| 901 | gaacttccca tcatgcacca ggactggctc aatggcaagg agttcaaatg cagggtcaac |
| 961 | agtgcagctt tccctgcccc catcgagaaa accatctcca aaaccaaagg cagaccgaag |
| 1021 | gctccacagg tgtacaccat tccacctccc aaggagcaga tggccaagga taaagtcagt |
| 1081 | ctgacctgca tgataacaga cttcttccct gaagacatta ctgtggagtg gcagtggaat |
| 1141 | gggcagccag cggagaacta caagaacact cagcccatca tggacacaga tggctcttac |
| 1201 | ttcgtctaca gcaagctcaa tgtgcagaag agcaactggg aggcaggaaa tactttcacc |
| 1261 | tgctctgtgt tacatgaggg cctgcacaac caccatactg agaagagcct ctcccactct |
| 1321 | cctaataaa |

Protein Sequence Defining the Full Length Heavy Chain Sequence (Heavy Chain Variable Region and IgG1 Constant Region) of 14F11 (SEQ ID NO:120)

| | |
|---|---|
| 1 | qvtlkesgpg ilqpsqtlsl tcsfsgfsls tygmgvgwir qpsgkglewl adiwwdddky |
| 61 | ynpslksrlt iskdtssnev flkiaivdta dtatyycarr ghysamdywg qgtsvtvssa |
| 121 | kttppsvypl apgsaaqtns mvtlgclvkg yfpepvtvtw nsgslssgvh tfpavlqsdl |
| 181 | ytlsssvtvp sstwpsetvt cnvahpasst kvdkkivprd cgckpcictv pevssvfifp |
| 241 | pkpkdvltit ltpkvtcvvv diskddpevq fswfvddvev htaqtqpree qfnstfrsvs |
| 301 | elpimhqdwl ngkefkcrvn saafpapiek tisktkgrpk apqvytippp keqmakdkvs |
| 361 | ltcmitdffp editvewqwn gqpaenyknt qpimdtdgsy fvysklnvqk snweagntft |
| 421 | csvlheglhn hhtekslshs pgk |

Nucleic Acid Sequence Encoding the Full Length Light Chain Sequence (Kappa Chain Variable Region and Constant Region) of 14F11 (SEQ ID NO: 121)

| | |
|---|---|
| 1 | gacattgtaa tgacccagtc tcaaaaattc atgtccacat cagtaggaga cagggtcagc |
| 61 | gtcacctgca aggccagtca gaatgtgggt actaatgtag cctggtatca acagaaacca |
| 121 | gggcaatctc ctaaagcact gatttactcg ccatcctacc ggtacagtgg agtccctgat |
| 181 | cgcttcacag gcagtggatc tgggacagat ttcactctca ccatcagcaa tgtgcagtct |
| 241 | gaagacttgg cagaatattt ctgtcagcaa tataacagct atcctcacac gttcggaggg |
| 301 | gggaccaagc tggaaatgaa acgggctgat gctgcaccaa ctgtatccat cttcccacca |
| 361 | tccagtgagc agttaacatc tggaggtgcc tcagtcgtgt gcttcttgaa caacttctac |
| 421 | cccaaagaca tcaatgtcaa gtggaagatt gatggcagtg aacgacaaaa tggcgtcctg |
| 481 | aacagttgga ctgatcagga cagcaaagac agcacctaca gcatgagcag caccctcacg |
| 541 | ttgaccaagg acgagtatga acgacataac agctatacct gtgaggccac tcacaagaca |
| 601 | tcaacttcac ccattgtcaa gagcttcaac aggaatgagt gt |

Protein Sequence Defining the Full Length Light Chain Sequence (Kappa Chain Variable Region and Constant Region) of 14F11 (SEQ ID NO:122)

| | |
|---|---|
| 1 | divmtqsqkf mstsvgdrvs vtckasqnvg tnvawyqqkp gqspkaliys psyrysgvpd |
| 61 | rftgsgsgtd ftltisnvqs edlaeyfcqq ynsyphtfgg gtklemkrad aaptvsifpp |
| 121 | sseqltsgga svvcflnnfy pkdinvkwki dgserqngvl nswtdqdskd stysmsstlt |
| 181 | ltkdeyerhn sytceathkt stspivksfn rnec |

Nucleic Acid Sequence Encoding the Full Length Heavy Chain Sequence (Heavy Chain Variable Region and IgG1 Constant Region) of 17B11 (SEQ ID NO:123)

| | |
|---|---|
| 1 | caggttactc tgaaagagtc tggccctggg atattgcagc cctcccagac cctcagtctg |
| 61 | acttgttctt tctctgggtt ttcactgagc acttctggta tgggtgtgag ttggattcgt |
| 121 | cagccttcag gaaagggtct ggagtggctg gcacacaatg actgggatga tgacaagcgc |
| 181 | tataagtcat ccctgaagag ccggctcaca atatccaagg atacctccag aaaccaggta |
| 241 | ttcctcaaga tcaccagtgt ggacactgca gatactgcca catactactg tgctcgaaga |
| 301 | gttgggggat tagagggcta ttttgattac tggggccaag gcaccactct cacagtctcc |
| 361 | tcagccaaaa cgacaccccc atctgtctat ccactggccc ctggatctgc tgcccaaact |
| 421 | aactccatgg tgaccctggg atgcctggtc aagggctatt tccctgagcc agtgacagtg |
| 481 | acctggaact ctggatccct gtccagcggt gtgcacacct tcccagctgt cctgcagtct |
| 541 | gacctctaca ctctgagcag ctcagtgact gtcccctcca gcacctggcc cagcgagacc |
| 601 | gtcacctgca acgttgccca cccggccagc agcaccaagg tggacaagaa aattgtgccc |
| 661 | agggattgtg gttgtaagcc ttgcatatgt acagtcccag aagtatcatc tgtcttcatc |
| 721 | ttccccccaa agcccaagga tgtgctcacc attactctga ctcctaaggt cacgtgtgtt |
| 781 | gtggtagaca tcagcaagga tgatcccgag gtccagttca gctggtttgt agatgatgtg |
| 841 | gaggtgcaca cagctcagac gcaaccccgg gaggagcagt tcaacagcac tttccgctca |
| 901 | gtcagtgaac ttcccatcat gcaccaggac tggctcaatg gcaaggagtt caaatgcagg |
| 961 | gtcaacagtg cagctttccc tgcccccatc gagaaaacca tctccaaaac caaaggcaga |
| 1021 | ccgaaggctc cacaggtgta caccattcca cctcccaagg agcagatggc caaggataaa |
| 1081 | gtcagtctga cctgcatgat aacagacttc ttccctgaag acattactgt ggagtggcag |
| 1141 | tggaatgggc agccagcgga gaactacaag aacactcagc ccatcatgga cacagatggc |
| 1201 | tcttacttcg tctacagcaa gctcaatgtg cagaagagca actgggaggc aggaaatact |
| 1261 | ttcacctgct ctgtgttaca tgagggcctg cacaaccacc atactgagaa gagcctctcc |
| 1321 | cactctcctg gtaaa |

Protein Sequence Defining the Full Length Heavy Chain Sequence (Heavy Chain Variable Region and IgG1 Constant Region) of 17B11 (SEQ ID NO:124)

| | |
|---|---|
| 1 | qvtlkesgpg ilqpsqtlsl tcsfsgfsls tsgmgvswir qpsgkglewl ahndwdddkr |
| 61 | yksslksrlt iskdtsrnqv flkitsvdta dtatyycarr vgglegyfdy wgqgttltvs |
| 121 | sakttppsvy plapgsaaqt nsmvtlgclv kgyfpepvtv twnsgslssg vhtfpavlqs |
| 181 | dlytlsssvt vpsstwpset vtcnvahpas stkvdkkivp rdcgckpcic tvpevssvfi |
| 241 | fppkpkdvlt itltpkvtcv vvdiskddpe vqfswfvddv evhtaqtqpr eeqfnstfrs |
| 301 | vselpimhqd wlngkefkcr vnsaafpapi ektisktkgr pkapqvytip ppkeqmakdk |
| 361 | vsltcmitdf fpeditvewq wngqpaenyk ntqpimdtdg syfvysklnv qksnweagnt |
| 421 | ftcsvlhegl hnhhteksls hspgk |

Nucleic Acid Sequence Encoding the Full Length Light Chain Sequence (Kappa Chain Variable Region and Constant Region) of 17B11 (SEQ ID NO:125)

| | |
|---|---|
| 1 | gacattgtgc tgacacagtc tcctgcttcc ttagctgtat ctctggggca gagggccacc |
| 61 | atctcatgca gggccagcca aagtgtcagt acatctaggt ttagttatat gcactggttc |
| 121 | caacagaaac caggacaggc acccaaactc ctcatcaagt atgcatccaa cctagaatct |
| 181 | ggggtccctg ccaggttcag tggcagtggg tctgggacag acttcaccct caacatccat |
| 241 | cctgtggagg gggaggatac tgcaacatat tactgtcagc acagttggga gattccgtac |
| 301 | acgttcggag gggggaccaa gctggaaata aaacgggctg atgctgcacc aactgtatcc |
| 361 | atcttcccac catccagtga gcagttaaca tctggaggtg cctcagtcgt gtgcttcttg |
| 421 | aacaacttct accccaaaga catcaatgtc aagtggaaga ttgatggcag tgaacgacaa |
| 481 | aatggcgtcc tgaacagttg gactgatcag gacagcaaag acagcaccta cagcatgagc |
| 541 | agcaccctca cgttgaccaa ggacgagtat gaacgacata acagctatac ctgtgaggcc |
| 601 | actcacaaga catcaacttc acccattgtc aagagcttca acaggaatga gtgt |

Protein Sequence Defining the Full Length Light Chain Sequence (Kappa Chain Variable Region and Constant Region) of 17B11 (SEQ ID NO: 126)

| | |
|---|---|
| 1 | divltqspas lavslgqrat iscrasqsvs tsrfsymhwf qqkpgqapkl likyasnles |
| 61 | gvparfsgsg sgtdftlnih pvegedtaty ycqhsweipy tfgggtklei kradaaptvs |
| 121 | ifppsseqlt sggasvvcfl nnfypkdinv kwkidgserq ngvlnswtdq dskdstysms |
| 181 | stltltkdey erhnsytcea thktstspiv ksfnrnec |

Table 5 shows the correspondence between the full-length sequences of the antibodies discussed in this Example with those presented in the Sequence Listing.

TABLE 5

| SEQ ID NO. | Nucleic Acid or Protein |
|---|---|
| 99 | 01G06_Heavy Variable + IgG1 Constant-nucleic acid |
| 100 | 01G06_Heavy Variable + IgG1 Constant-protein |
| 101 | 01G06_Kappa Variable + Constant-nucleic acid |
| 102 | 01G06_Kappa Variable + Constant-protein |
| 103 | 03G05 Heavy Variable + IgG1 Constant-nucleic acid |
| 104 | 03G05 Heavy Variable + IgG1 Constant-protein |
| 105 | 03G05 Kappa Variable + Constant-nucleic acid |
| 106 | 03G05 Kappa Variable + Constant-protein |
| 107 | 04F08 Heavy Variable + IgG1 Constant-nucleic acid |
| 108 | 04F08 Heavy Variable + IgG1 Constant-protein |
| 109 | 04F08 Kappa Variable + Constant-nucleic acid |
| 110 | 04F08 Kappa Variable + Constant-protein |
| 111 | 06C11 Heavy Variable + IgG1 Constant-nucleic acid |
| 112 | 06C11 Heavy Variable + IgG1 Constant-protein |
| 113 | 06C11 Kappa Variable + Constant-nucleic acid |
| 114 | 06C11 Kappa Variable + Constant-protein |
| 115 | 08G01 Heavy Variable + IgG2b Constant-nucleic acid |
| 116 | 08G01 Heavy Variable + IgG2b Constant-protein |
| 117 | 08G01 Kappa Variable + Constant-nucleic acid |
| 118 | 08G01 Kappa Variable + Constant-protein |
| 119 | 14F11 Heavy Variable + IgG1 Constant-nucleic acid |
| 120 | 14F11 Heavy Variable + IgG1 Constant-protein |
| 121 | 14F11 Kappa Variable + Constant-nucleic acid |
| 122 | 14F11 Kappa Variable + Constant-protein |
| 123 | 17B11 Heavy Variable + IgG1 Constant-nucleic acid |
| 124 | 17B11 Heavy Variable + IgG1 Constant-protein |
| 125 | 17B11 Kappa Variable + Constant-nucleic acid |
| 126 | 17B11 Kappa Variable + Constant-protein |

### Example 8: Binding Affinities

The binding affinities and kinetics of binding of antibodies to 6×His tagged (SEQ ID NO: 266) recombinant human GDF15 (His-rhGDF15 (R&D Systems, Inc.)), untagged recombinant human GDF15 (rhGDF15 (Peprotech, Rocky Hill, NJ), and recombinant human GDF15 produced as either mouse Fc fused to human GDF15 (mFc-rhGDF15) or a version in which the Fc was enzymatically removed (cleaved-rhGDF15) were measured by surface plasmon resonance, using a Biacore® T100 instrument (GE Healthcare, Piscataway, NJ).

Rabbit anti-mouse IgGs (GE Healthcare) were immobilized on carboxymethylated dextran CM4 sensor chips (GE Healthcare) by amine coupling, according to a standard protocol. Analyses were performed at 37° C. using PBS containing 0.05% surfactant P20 as running buffer. The antibodies were captured in individual flow cells at a flow rate of 10 μL/minute. Injection time was varied for each antibody to yield an Rmax between 30 and 60 RU. 250 pg/mL mouse Fc (Jackson ImmunoResearch, West Grove, PA) was injected at 30 μL/minute for 120 seconds to block non-specific binding of capture antibodies to mouse Fc portion of the recombinant GDF15 protein when needed. Buffer, mFc-rhGDF15, cleaved-rhGDF15, His-rhGDF15, or rhGDF15 diluted in running buffer was injected sequentially over a reference surface (no antibody captured) and the active surface (antibody to be tested) for 240 seconds at 60 μL/minute. The dissociation phase was monitored for up to 1500 seconds. The surface was then regenerated with two 60-second injections of 10 mM Glycine-HCl, pH 1.7, at a flow rate of 30 μL/minute. The GDF15 concentration range tested was 30 nM to 0.625 nM.

Kinetic parameters were determined using the kinetic function of the BIAevaluation software (GE Healthcare) with double reference subtraction. Kinetic parameters for each antibody, kₐ (association rate constant), k_{d} (dissociation rate constant), and K_{D} (equilibrium dissociation constant) were determined. Kinetic values of the monoclonal antibodies on mFc-rhGDF15, cleaved rhGDF15, His-rhGDF15, or rhGDF15 are summarized in Tables 6, 7, 8, and 9, respectively.

TABLE 6
Antibody Binding to mFc-rhGDF15

| Antibody | kₐ (1/Ms) | k_{d} (1/s) | K_{D} (M) | n |
|---|---|---|---|---|
| 01G06 | 5.6E+06 | 7.0E−04 | 2.1E−10 | 7 |
| 03G05 | 1.0E+07 | 6.4E−04 | 6.9E−11 | 3 |
| 04F08 | 3.6E+06 | 6.4E−04 | 1.9E−10 | 3 |
| 06C11 | 4.5E+06 | 6.8E−04 | 1.7E−10 | 5 |
| 08G01 | 6.0E+06 | 1.1E−03 | 1.9E−10 | 4 |
| 14F11 | 1.7E+06 | 3.3E−04 | 2.2E−10 | 4 |
| 17B11 | 3.7E+06 | 5.1E−04 | 1.4E−10 | 3 |

The data in Table 6 demonstrate that antibodies bind mFc-rhGDF15 with a K_{D} of about 250 pM or less, 200 pM or less, 150 pM or less, 100 pM or less, 75 pM or less, or 50 pM or less.

Kinetic values of the monoclonal antibodies on cleaved-rhGDF15 are summarized in Table 7.

TABLE 7
Antibody Binding to Cleaved-rhGDF15

| Antibody | kₐ (1/Ms) | k_{d} (1/s) | K_{D} (M) | n |
|---|---|---|---|---|
| 01G06 | 7.5E+06 | 8.6E−04 | 1.1E−10 | 1 |
| 06C11 | 1.2E+07 | 2.0E−03 | 1.7E−10 | 2 |
| 14F11 | 5.7E+06 | 6.0E−04 | 1.1E−10 | 1 |

The data in Table 7 demonstrate that antibodies 01G06, 06C11 and 14F11 bind cleaved-rhGDF15 with a K_{D} of about 200 pM or less, 150 pM or less, or 100 pM or less.

Kinetic values of the monoclonal antibodies on His-rhGDF15 are summarized in Table 8.

TABLE 8
Antibody Binding to His-rhGDF15

| Antibody | kₐ (1/Ms) | k_{d} (1/s) | K_{D} (M) | n |
|---|---|---|---|---|
| 01G06 | 1.4E+07 | 1.1E−03 | 8.1E−11 | 2 |
| 06C11 | 2.9E+07 | 1.5E−03 | 5.1E−11 | 2 |
| 14F11 | 4.4E+06 | 4.2E−04 | 9.6E−11 | 1 |

The data in Table 8 demonstrate that antibodies 01G06, 06C11 and 14F11 bind His-rhGDF15 with a K_{D} of about 150 pM or less, 100 pM or less, 75 pM or less, or 50 pM or less.

Kinetic values of the monoclonal antibodies on rhGDF15 are summarized in Table 9.

TABLE 9
Antibody Binding to rhGDF15

| Antibody | kₐ (1/Ms) | k_{d} (1/s) | K_{D} (M) | n |
|---|---|---|---|---|
| 01G06 | 2.1E+07 | 1.9E−03 | 9.3E−11 | 1 |
| 06C11 | 2.2E+07 | 4.6E−03 | 2.1E−10 | 1 |
| 14F11 | 3.1E+07 | 2.2E−03 | 7.1E−11 | 1 |

The data in Table 9 demonstrate that antibodies 01G06, 06C11 and 14F11 bind rhGDF15 with a K_{D} of about 250 pM or less, 200 pM or less, 150 pM or less, 100 pM or less, 75 pM or less, or 50 pM or less.

### Example 9: Reversal of Cachexia in an mFc-rhGDF15-Induced Model

This Example demonstrates the reversal of cachexia (as indicated by body weight loss) by antibody 01G06, 03G05, 04F08, 06C11, 14F11, or 17B11 in an mFc-rhGDF15-induced cachexia model. mFc-rhGDF15 (2 μg/g) was administered subcutaneously into the flank of 8-week old female ICR-SCID mice. Body weight was measured daily. When body weight reached 93%, the mice were randomized into seven groups of ten mice each. Each group received one of the following treatments: murine IgG control, 01G06, 03G05, 04F08, 06C11, 14F11, or 17B11 at 10 mg/kg. Treatment was administered once by intra-peritoneal injection. Treatment with antibody 01G06, 03G05, 04F08, 06C11, 14F11, or 17B11 resulted in body weight increase relative to initial weight or about 100% (p<0.001) (FIG. 14 and Table 10).

TABLE 10

| | | |
|---|---|---|
| Treatment | % Body | ANOVA Analysis |

| Gr. | Agent | mg/kg | Weight | (compared to mIgG) |
|---|---|---|---|---|
| 1 | mIgG | 10 | 77.1 | NA |
| 2 | 01G06 | 10 | 94.1 | p < 0.001 |
| 3 | 03G05 | 10 | 95.1 | p < 0.001 |
| 4 | 04F08 | 10 | 95.8 | p < 0.001 |
| 5 | 06C11 | 10 | 93.8 | p < 0.001 |
| 7 | 14F11 | 10 | 95.4 | p < 0.001 |
| 8 | 17B11 | 10 | 92.8 | p < 0.001 |

The data in FIG. 14 and Table 10 indicate that the disclosed anti-GDF15 antibodies can reverse cachexia in an mFc-rhGDF15-induced mouse model (i.e., a non-tumor bearing mouse model).

### Example 10: Reversal of Cachexia in an HT-1080 Xenograft Tumor Model

This Example demonstrates the reversal of cachexia (as indicated by body weight loss) by antibody 01G06, 03G05, 04F08, 06C11, 08G01, 14F11 or 17B11 in an HT-1080 fibrosarcoma xenograft model. HT-1080 cells were grown in culture at 37° C. in an atmosphere containing 5% CO₂, using Eagle's Minimum Essential Medium (ATCC, Catalog No. 30-2003) containing 10% FBS. Cells were inoculated subcutaneously into the flank of 8-week old female ICR SCID mice with 5×10⁶ cells per mouse in 50% matrigel. Body weight was measured daily. When body weight reached 93%, the mice were randomized into eight groups of ten mice each. Each group received one of the following treatments: murine IgG control, 01G06, 03G05, 04F08, 06C11, 08G01, 14F11 or 17B11 at 10 mg/kg. Treatment was administered every three days by intra-peritoneal injection. Treatment with antibody 01G06, 03G05, 04F08, 06C11, 08G01, 14F11 or 17B11 resulted in body weight increase relative to initial weight or about 100% (p<0.001) (FIG. 15 and Table 11).

TABLE 11

| | | ANOVA |
|---|---|---|
| | | ANALYSIS |
| TREATMENT | % BODY | (COMPARED |

| GR. | AGENT | MG/KG | WEIGHT | TO MIGG) |
|---|---|---|---|---|
| 1 | MIGG | 10 | 81.4 | NA |
| 2 | 01G06 | 10 | 103.3 | P < 0.001 |
| 3 | 03G05 | 10 | 106.1 | p < 0.001 |
| 4 | 04F08 | 10 | 104.3 | p < 0.001 |
| 5 | 06C11 | 10 | 106.6 | p < 0.001 |
| 6 | 08G01 | 10 | 105.3 | p < 0.001 |
| 7 | 14F11 | 10 | 99.6 | p < 0.001 |
| 8 | 17B11 | 10 | 103.7 | p < 0.001 |

The data in FIG. 15 and Table 11 indicate that the disclosed anti-GDF15 antibodies can reverse cachexia in an HT-1080 fibrosarcoma xenograft model.

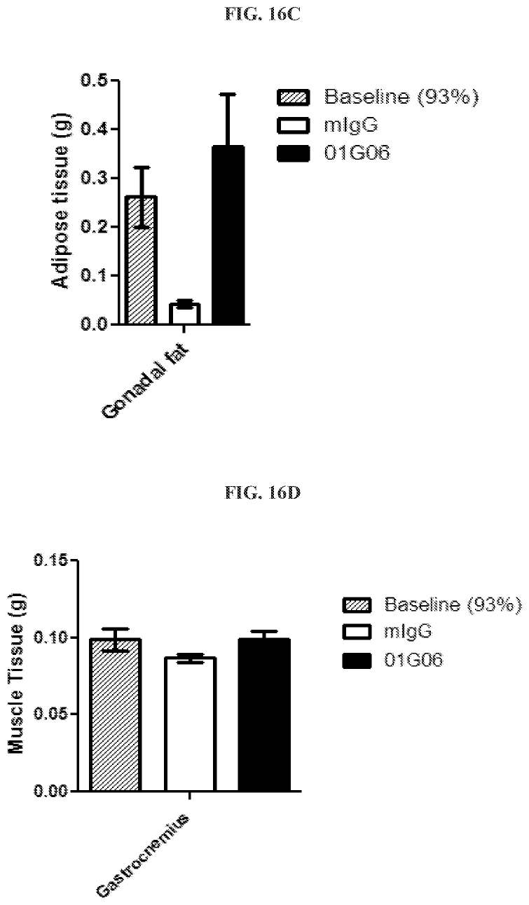

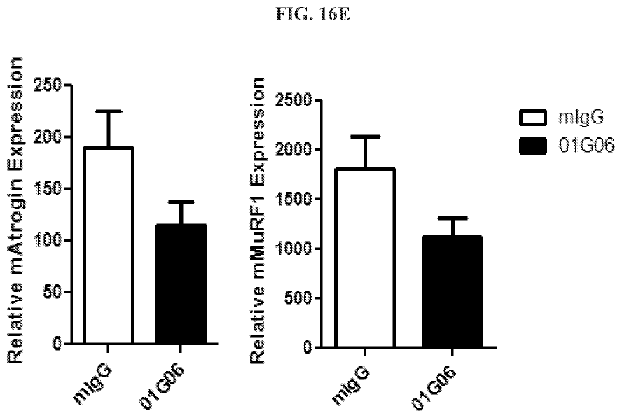

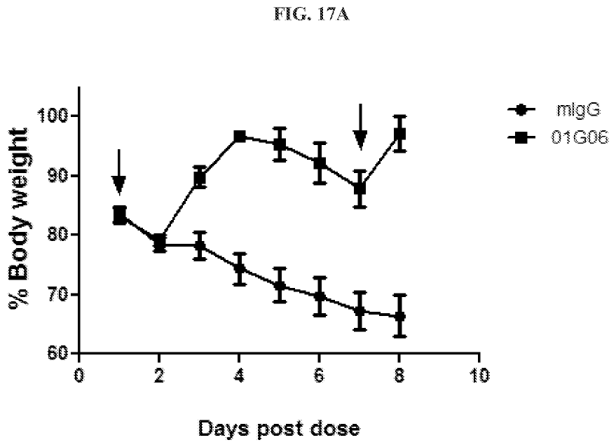

Additional studies were conducted with antibody 01G06 to demonstrate the reversal of cachexia in this mouse model. HT-1080 cells were grown and inoculated subcutaneously into the flank of 8-week old female ICR SCID mice as described above. When body weight reached 93%, the mice were randomized into two groups of ten mice each. Each group received one of the following treatments: murine IgG control or 01G06 at 10 mg/kg. Treatment was administered once by intra-peritoneal injection. As shown in FIG. 16A, treatment with antibody 01G06 resulted in body weight increase to initial weight or 100% (p<0.001) (FIG. 16A).

Food consumption was determined by weighing the food supply given to the mice daily (FIG. 16B). A significant increase in food consumption was observed in the 01G06 treated group for the first three days post treatment. After that time, no significant change was observed compared to the control group (mIgG).

Water consumption was determined by weighing the water supply given to the mice daily. No significant change in water consumption was observed between groups.

In this experiment, a group of ten mice were sacrificed at the time of the dose (baseline or 93% body weight, without treatment) and at the end of study (seven days post dose, either mIgG or 01G06). Gonadal fat and the gastrocnemius muscles were removed surgically and weighed as described above in Example 4 and tissues were snap frozen in liquid nitrogen. RNA was isolated from the gastrocnemius muscle samples to determine the levels of mMuRF1 and mAtrogin mRNA by RT-PCR, as described in Example 4.

As shown in FIG. 16C, a significant reduction in gonadal fat mass was observed seven days post dose with mIgG, but not in the group treated with antibody 01G06. In addition, mice treated with mIgG displayed significant gastrocnemius muscle loss compared to the baseline group, while the group of mice treated with antibody 01G06 did not (FIG. 16D). Further, the levels of muscular degradation markers, mMuRF1 and mAtrogin, were significantly higher in the mIgG group compared to the 01G06 group (FIG. 16E).

These results indicate that the disclosed anti-GDF15 antibodies can reverse cachexia measured by the loss of muscle mass, the loss of fat and involuntary weight loss in an HT-1080 xenograft tumor model.

### Example 11: Reversal of Cachexia in an HT-1080 Xenograft Tumor Model

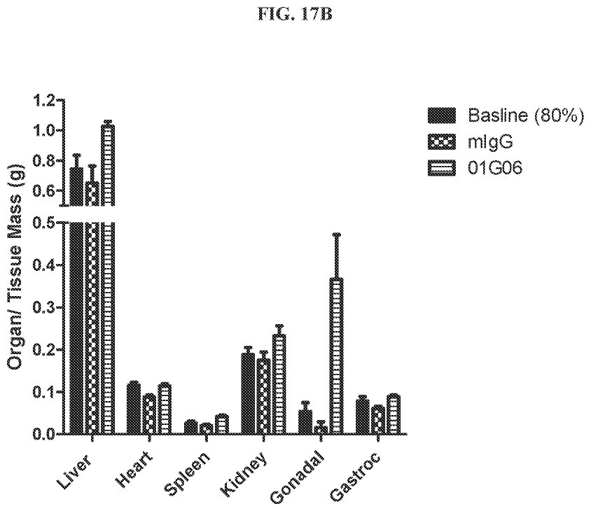

This Example demonstrates the reversal of cachexia (as indicated by body weight loss) by antibody 01G06 in an HT-1080 fibrosarcoma xenograft model. HT-1080 cells were grown in culture at 37° C. in an atmosphere containing 5% CO₂, using Eagle's Minimum Essential Medium (ATCC, Catalog No. 30-2003) containing 10% FBS. Cells were inoculated subcutaneously into the flank of 8-week old female ICR SCID mice with 5×10⁶ cells per mouse in 50% matrigel. Body weight was measured daily. When body weight reached 80%, the mice were randomized into two groups of five mice each. Each group received one of the following treatments: murine IgG control, 01G06 dosed at 2 mg/kg on day 1 and day 7. Treatment was administered by intra-peritoneal injection. Treatment with antibody 01G06 resulted in body weight increase relative to initial weight or about 100% (p<0.001) (FIG. 17A and Table 12).

TABLE 12

| | | |
|---|---|---|
| Treatment | % Body | ANOVA Analysis |

| Gr. | Agent | mg/kg | Weight | (compared to mIgG) |
|---|---|---|---|---|
| 1 | mIgG | 2 | 66.4 | NA |
| 2 | 01G06 | 2 | 97.16 | p < 0.001 |

The data in FIGS. 17A-B and Table 12 indicate that the disclosed anti-GDF15 antibodies can reverse cachexia in an HT-1080 fibrosarcoma xenograft model.

In this experiment, a group of five mice were sacrificed at the time of dosing (baseline or 80% body weight loss, without treatment) and at the end of study (seven days post dose, either mIgG or 01G06). Liver, heart, slpeen, kidney, gonadal fat and the gastrocnemius muscles were removed surgically and weighed. As shown in FIG. 17B, a significant loss in liver, heart, spleen, kidney, gonadal fat and gastrocnemius muscle mass was observed seven days post dose with mIgG, but not in the group treated with antibody 01G06. In addition, mice treated with antibody 01G06 displayed significant liver and gonadal muscle gain compared to the baseline group (FIG. 17B).

These results indicate that the disclosed anti-GDF15 antibodies can reverse cachexia measured by the loss of key organ mass, loss of muscle mass, loss of fat and involuntary weight loss in an HT-1080 xenograft tumor model.

### Example 12: Reversal of Cachexia in a K-562 Xenograft Tumor Model

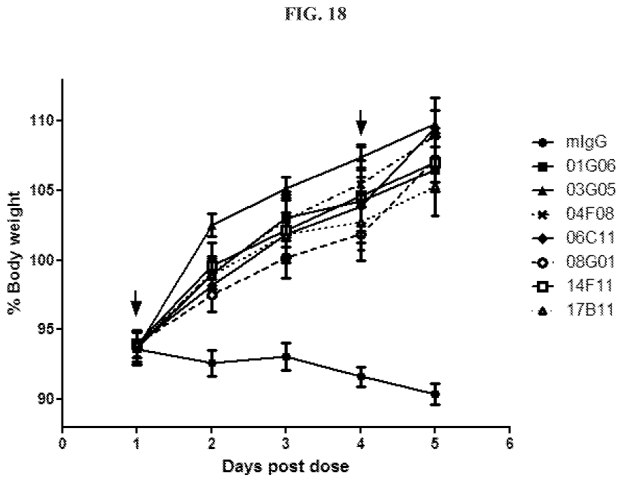

This Example demonstrates the reversal of cachexia (as indicated by body weight loss) by antibody 01G06, 03G05, 04F08, 06C11, 08G01, 14F11 or 17B11 in a K-562 leukemia xenograft model. K-562 cells were grown in culture at 37° C. in an atmosphere containing 5% CO₂, using Iscove's Modified Dulbecco's Medium (ATCC Catalog No. 30-2005) containing 10% FBS. Cells were inoculated subcutaneously into the flank of 8-week old female CB17SCRFMF mice with 2.5×10⁶ cells per mouse in 50% matrigel. Body weight was measured daily. When body weight reached 93%, the mice were randomly distributed into eight groups of ten mice each. Each group received one of the following treatments: murine IgG control, 01G06, 03G05, 04F08, 06C11, 08G01, 14F11 or 17B11 at 10 mg/kg. Treatment was administered every three days by intra-peritoneal injection. Treatment with antibody 01G06, 03G05, 04F08, 06C11, 08G01, 14F11 or 17B11 resulted in body weight increase relative to initial weight or about 100% (p<0.001) (FIG. 18 and Table 13).

TABLE 13

| | | |
|---|---|---|
| Treatment | % Body | ANOVA Analysis |

| Gr. | Agent | mg/kg | Weight | (compared to mIgG) |
|---|---|---|---|---|
| 1 | mIgG | 10 | 90.4 | NA |
| 2 | 01G06 | 10 | 106.5 | p < 0.001 |
| 3 | 03G05 | 10 | 109.8 | p < 0.001 |
| 4 | 04F08 | 10 | 108.9 | p < 0.001 |
| 5 | 06C11 | 10 | 109.5 | p < 0.001 |
| 6 | 08G01 | 10 | 107.2 | p < 0.001 |
| 7 | 14F11 | 10 | 107.0 | p < 0.001 |
| 8 | 17B11 | 10 | 105.3 | p < 0.001 |

The data in FIG. 18 and Table 13 indicate that the disclosed anti-GDF15 antibodies can reverse cachexia in a K-562 xenograft tumor model.

### Example 13: Additional Xenograft Tumor Models

Antibody 01G06 was tested in additional tumor xenograft models including the TOV-21G ovarian xenograft model and the LS1034 colon xenograft model. In each model, antibody 01G06 reversed body weight loss compared to a PBS control (p<0.001 for the TOV-21G model and p<0.01 for the LS1034 model).

### Example 14: Humanization of Anti-GDF15 Antibodies

This Example describes the humanization and chimerization of three murine antibodies, designated 01G06, 06C11, and 14F11, and the characterization of the resulting humanized antibodies. The humanized anti-GDF15 antibodies were designed, affinity matured by targeted CDR mutagenesis, and optimized using methods known in the art. The amino acid sequences were converted to codon-optimized DNA sequences and synthesized to include (in the following order): 5′ HindIII restriction site, Kozak consensus sequence, amino terminal signal sequence, humanized variable region, human IgG1 or Kappa constant region, stop codon, and a 3′ EcoRI restriction site.

Chimeric (murine variable region and human constant region) 01G06, 06C11, and 14F11 heavy (human IgG1) and light (human Kappa) chains were also constructed. To generate chimeric antibodies, the murine variable regions were fused to the human constant region, and codon-optimized DNA sequences were synthesized, including (in the following order): 5′ HindIII restriction site, Kozak consensus sequence, amino terminal signal sequence, mouse variable region, human IgG1 or Kappa constant region, stop codon, and 3′ EcoRI restriction site.

The humanized and chimeric heavy chains were subcloned into pEE6.4 (Lonza, Basel, Switzerland) via HindIII and EcoRI sites using In-Fusion™ PCR cloning (Clontech, Mountain View, CA). The humanized and chimeric Kappa light chains were subcloned into pEE14.4 (Lonza) via HindIII and EcoRI sites using In-Fusion™ PCR cloning.

Humanized antibody chains or chimeric antibody chains were transiently transfected into 293T cells to produce antibody. Antibody was either purified or used in cell culture media supernatant for subsequent in vitro analysis. Binding of the chimeric and humanized antibodies to human GDF15 was measured as described below. The results are summarized in Tables 24-27.

Each of the possible combinations of the chimeric or humanized 01G06 immunoglobulin heavy chain and immunoglobulin light chain variable regions is set forth below in Table 14.

TABLE 14

| Antibody | Light Chain Variable | Heavy Chain Variable |
|---|---|---|
| Name | Region | Region |
| Hu01G06-1 | Ch01G06 Chimeric Kappa | Ch01G06 Chimeric Heavy |
| | (SEQ ID NO: 76) | (SEQ ID NO: 40) |
| Hu01G06-14 | Ch01G06 Chimeric Kappa | Hu01G06 IGHV1-18 Heavy |
| | (SEQ ID NO: 76) | (SEQ ID NO: 54) |
| Hu01G06-15 | Ch01G06 Chimeric Kappa | Hu01G06 IGHV1-69 Heavy |
| | (SEQ ID NO: 76) | (SEQ ID NO: 56) |
| Hu01G06-147 | Ch01G06 Chimeric Kappa | Sh01G06 IGHV1-18 M69L |
| | (SEQ ID NO: 76) | Heavy (SEQ ID NO: 58) |
| Hu01G06-148 | Ch01G06 Chimeric Kappa | Sh01G06 IGHV1-18 M69L K64Q |
| | (SEQ ID NO: 76) | G44S Heavy (SEQ ID NO: 60) |
| Hu01G06-149 | Ch01G06 Chimeric Kappa | Sh01G06 IGHV1-18 M69L K64Q |
| | (SEQ ID NO: 76) | Heavy (SEQ ID NO: 62) |
| Hu01G06-150 | Ch01G06 Chimeric Kappa | Sh01G06 IGHV1-69 T30S I69L |
| | (SEQ ID NO: 76) | Heavy (SEQ ID NO: 64) |
| Hu01G06-151 | Ch01G06 Chimeric Kappa | Sh01G06 IGHV1-69 T30S K64Q |
| | (SEQ ID NO: 76) | I69L Heavy (SEQ ID NO: 66) |
| Hu01G06-4 | Hu01G06 IGKV1-39 Kappa | Ch01G06 Chimeric Heavy |
| | (SEQ ID NO: 90) | (SEQ ID NO: 40) |
| Hu01G06-46 | Hu01G06 IGKV1-39 Kappa | Hu01G06 IGHV1-18 Heavy |
| | (SEQ ID NO: 90) | (SEQ ID NO: 54) |
| Hu01G06-52 | Hu01G06 IGKV1-39 Kappa | Hu01G06 IGHV1-69 Heavy |
| | (SEQ ID NO: 90) | (SEQ ID NO: 56) |
| Hu01G06-100 | Hu01G06 IGKV1-39 Kappa | Sh01G06 IGHV1-18 M69L Heavy |
| | (SEQ ID NO: 90) | (SEQ ID NO: 58) |
| Hu01G06-102 | Hu01G06 IGKV1-39 Kappa | Sh01G06 IGHV1-18 M69L K64Q |
| | (SEQ ID NO: 90) | G44S Heavy (SEQ ID NO: 60) |
| Hu01G06-101 | Hu01G06 IGKV1-39 Kappa | Sh01G06 IGHV1-18 M69L |
| | (SEQ ID NO: 90) | K64Q Heavy (SEQ ID NO: 62) |
| Hu01G06-103 | Hu01G06 IGKV1-39 Kappa | Sh01G06 IGHV1-69 T30S |
| | (SEQ ID NO: 90) | I69L Heavy (SEQ ID NO: 64) |
| Hu01G06-104 | Hu01G06 IGKV1-39 Kappa | Sh01G06 IGHV1-69 T30S K64Q |
| | (SEQ ID NO: 90) | I69L Heavy(SEQ ID NO: 66) |
| Hu01G06-152 | Hu01G06 IGKV1-39 S43A V48I | Ch01G06 Chimeric Heavy |
| | Kappa (SEQ ID NO: 92) | (SEQ ID NO: 40) |
| Hu01G06-71 | Hu01G06 IGKV1-39 S43A V48I | Hu01G06 IGHV1-18 Heavy |
| | Kappa (SEQ ID NO: 92) | (SEQ ID NO: 54) |
| Hu01G06-77 | Hu01G06 IGKV1-39 S43A V48I | Hu01G06 IGHV1-69 Heavy |
| | Kappa (SEQ ID NO: 92) | (SEQ ID NO: 56) |
| Hu01G06-110 | Hu01G06 IGKV1-39 S43A V48I | Sh01G06 IGHV1-18 M69L Heavy |
| | Kappa (SEQ ID NO: 92) | (SEQ ID NO: 58) |
| Hu01G06-112 | Hu01G06 IGKV1-39 S43A V48I | Sh01G06 IGHV1-18 M69L K64Q |
| | Kappa (SEQ ID NO: 92) | G44S Heavy (SEQ ID NO: 60) |
| Hu01G06-111 | Hu01G06 IGKV1-39 S43A V48I | Sh01G06 IGHV1-18 M69L K64Q |
| | Kappa (SEQ ID NO: 92) | Heavy (SEQ ID NO: 62) |
| Hu01G06-113 | Hu01G06 IGKV1-39 S43A V48I | Sh01G06 IGHV1-69 T30S I69L |
| | Kappa (SEQ ID NO: 92) | Heavy (SEQ ID NO: 64) |
| Hu01G06-114 | Hu01G06 IGKV1-39 S43A V48I | Sh01G06 IGHV1-69 T30S K64Q |
| | Kappa (SEQ ID NO: 92) | I69L Heavy (SEQ ID NO: 66) |
| Hu01G06-122 | Hu01G06 IGKV1-39 S43A V48I | Hu01G06 IGHV1-18 F1 Heavy |
| | Kappa(SEQ ID NO: 92) | (SEQ ID NO: 246) |
| Hu01G06-119 | Hu01G06 IGKV1-39 S43A V48I | Hu01G06 IGHV1-18 F2 Heavy |
| | Kappa(SEQ ID NO: 92) | (SEQ ID NO: 248) |
| Hu01G06-135 | Hu01G06 IGKV1-39 S43A V48I | Hu01G06 IGHV1-69 F1 Heavy |
| | Kappa(SEQ ID NO: 92) | (SEQ ID NO: 250) |
| Hu01G06-138 | Hu01G06 IGKV1-39 S43A V48I | Hu01G06 IGHV1-69 F2 Heavy |
| | Kappa(SEQ ID NO: 92) | (SEQ ID NO: 252) |
| Hu01G06-153 | Hu01G06 IGKV1-39 V48I Kappa | Ch01G06 Chimeric Heavy |
| | (SEQ ID NO: 94) | (SEQ ID NO: 40) |
| Hu01G06-69 | Hu01G06 IGKV1-39 V48I Kappa | Hu01G06 IGHV1-18 Heavy |
| | (SEQ ID NO: 94) | (SEQ ID NO: 54) |
| Hu01G06-75 | Hu01G06 IGKV1-39 V48I Kappa | Hu01G06 IGHV1-69 Heavy |
| | (SEQ ID NO: 94) | (SEQ ID NO: 56) |
| HU01G06-105 | Hu01G06 IGKV1-39 V48I Kappa | Sh01G06 IGHV1-18 M69L Heavy |
| | (SEQ ID NO: 94) | (SEQ ID NO: 58) |
| Hu01G06-107 | Hu01G06 IGKV1-39 V48I Kappa | Sh01G06 IGHV1-18 M69L K64Q |
| | (SEQ ID NO: 94) | G44S Heavy (SEQ ID NO: 60) |
| Hu01G06-106 | Hu01G06 IGKV1-39 V48I Kappa | Sh01G06 IGHV1-18 M69L K64Q |
| | (SEQ ID NO: 94) | Heavy (SEQ ID NO: 62) |
| Hu01G06-108 | Hu01G06 IGKV1-39 V48I Kappa | Sh01G06 IGHV1-69 T30S I69L |
| | (SEQ ID NO: 94) | Heavy (SEQ ID NO: 64) |
| Hu01G06-109 | Hu01G06 IGKV1-39 V48I Kappa | Sh01G06 IGHV1-69 T30S K64Q |
| | (SEQ ID NO: 94) | I69L Heavy (SEQ ID NO: 66) |
| Hu01G06-154 | Hu01G06 IGKV1-39 F2 Kappa | Ch01G06 Chimeric Heavy |
| | (SEQ ID NO: 254) | (SEQ ID NO: 40) |
| Hu01G06-155 | Hu01G06 IGKV1-39 F2 Kappa | Hu01G06 IGHV1-18 Heavy |
| | (SEQ ID NO: 254) | (SEQ ID NO: 54) |
| Hu01G06-156 | Hu01G06 IGKV1-39 F2 Kappa | Hu01G06 IGHV1-69 Heavy |
| | (SEQ ID NO: 254) | (SEQ ID NO: 56) |
| Hu01G06-157 | Hu01G06 IGKV1-39 F2 Kappa | Sh01G06 IGHV1-18 M69L Heavy |
| | (SEQ ID NO: 254) | (SEQ ID NO: 58) |
| Hu01G06-158 | Hu01G01 IGKV1-39 F2 Kappa | Sh01G06 IGHV1-18 M69L K64Q |
| | (SEQ ID NO: 254) | G44S Heavy (SEQ ID NO: 60) |
| Hu01G06-159 | Hu01G06 IGKV1-39 F2 Kappa | Sh01G06 IGHV1-18 M69L K64Q |
| | (SEQ ID NO: 254) | Heavy (SEQ ID NO: 62) |
| Hu01G06-160 | Hu01G06 IGKV1-39 F2 Kappa | Sh01G06 IGHV1-69 T30S I69L |
| | (SEQ ID NO: 254) | Heavy (SEQ ID NO: 64) |
| Hu01G06-161 | Hu01G06 IGKV1-39 F2 Kappa | Su01G06 IGHV1-69 T30S K64Q |
| | (SEQ ID NO: 254) | I69L Heavy (SEQ ID NO: 66) |
| Hu01G06-130 | Hu01G06 IGKV1-39 F2 Kappa | Hu01G06 IGHV1-18 F1 Heavy |
| | (SEQ ID NO: 254) | (SEQ ID NO: 246) |
| Hu01G06-127 | Hu01G06 IGKV1-39 F2 Kappa | Hu01G06 IGHV1-18 F2 Heavy |
| | (SEQ ID NO: 254) | (SEQ ID NO: 248) |
| Hu01G06-143 | Hu01G06 IGKV1-39 F2 Kappa | Hu01G06 IGHV1-69 F1 Heavy |
| | (SEQ ID NO: 254) | (SEQ ID NO: 250) |
| Hu01G06-146 | Hu01G06 IGKV1-39 F2 Kappa | Hu01G06 IGHV1-69 F2 Heavy |
| | (SEQ ID NO: 254) | (SEQ ID NO: 252) |

Each of the possible combinations of the chimeric or humanized 06C11 immunoglobulin heavy chain and immunoglobulin light chain variable regions is set forth below in Table 15.

TABLE 15

| Antibody | Light Chain Variable | Heavy Chain Variable |
|---|---|---|
| Name | Region | Region |
| Hu06C11-1 | Ch06C11 Chimeric Kappa | Ch06C11 Chimeric Heavy |
| | (SEQ ID NO: 82) | (SEQ ID NO: 46) |
| Hu06C11-7 | Ch06C11 Chimeric Kappa | HE LM 06C11 IGHV2-70 |
| | (SEQ ID NO: 82) | Heavy (SEQ ID NO: 68) |
| Hu06C11-10 | Ch06C11 Chimeric Kappa | Hu06C11 IGHV2-5 Heavy |
| | (SEQ ID NO: 82) | (SEQ ID NO: 70) |
| Hu06C11-12 | Sh06C11 IGKV1-16 Kappa | Ch06C11 Chimeric Heavy |
| | (SEQ ID NO: 96) | (SEQ ID NO: 46) |
| Hu06C11-27 | Sh06C11 IGKV1-16 Kappa | HE LM 06C11 IGHV2-70 |
| | (SEQ ID NO: 96) | Heavy (SEQ ID NO: 68) |
| Hu06C11-30 | Sh06C11 IGKV1-16 Kappa | Hu06C11 IGHV2-5 Heavy |
| | (SEQ ID NO: 96) | (SEQ ID NO: 70) |

Each of the possible combinations of the chimeric or humanized 14F11 immunoglobulin heavy chain and immunoglobulin light chain variable regions is set forth below in Table 16.

TABLE 16

| Antibody | Light Chain Variable | Heavy Chain Variable |
|---|---|---|
| Name | Region | Region |
| Hu14F11-1 | Ch14F11 Chimeric Kappa | Ch14F11 Chimeric Heavy |
| | (SEQ ID NO: 86) | (SEQ ID NO: 50) |
| Hu14F11-14 | Ch14F11 Chimeric Kappa | Sh14F11 IGHV2-5 Heavy |
| | (SEQ ID NO: 86) | (SEQ ID NO: 72) |
| Hu14F11-15 | Ch14F11 Chimeric Kappa | Sh14F11 IGHV2-70 Heavy |
| | (SEQ ID NO: 86) | (SEQ ID NO: 74) |
| Hu14F11-11 | Hu14F11 IGKV1-16 Kappa | Ch14F11 Chimeric Heavy |
| | (SEQ ID NO: 98) | (SEQ ID NO: 50) |
| Hu14F11-39 | Hu14F11 IGKV1-16 Kappa | Sh14F11 IGHV2-5 Heavy |
| | (SEQ ID NO: 98 | (SEQ ID NO: 72) |
| Hu14F11-47 | Hu14F11 IGKV1-16 Kappa | Sh14F11 IGHV2-70 Heavy |
| | (SEQ ID NO: 98) | (SEQ ID NO: 74) |

Each of the possible combinations of the chimeric 04F08, 06C11, and 14F11 immunoglobulin heavy chain and immunoglobulin light chain variable regions is set forth below in Table 17. Table 17

TABLE 17

| Light Chain Variable Region | Heavy Chain Variable Region |
|---|---|
| 04F08 Chimeric Kappa | Ch06C11 Chimeric Heavy |
| (SEQ ID NO: 80) | (SEQ ID NO: 46) |
| 04F08 Chimeric Kappa | Ch14F11 Chimeric Heavy |
| (SEQ ID NO: 80) | (SEQ ID NO: 50) |
| Ch06C11 Chimeric Kappa | 04F08 Chimeric Heavy |
| (SEQ ID NO: 82) | (SEQ ID NO: 44) |
| Ch06C11 Chimeric Kappa | Ch14F11 Chimeric Heavy |
| (SEQ ID NO: 82) | (SEQ ID NO: 50) |
| Ch14F11 Chimeric Kappa | 04F08 Chimeric Heavy |
| (SEQ ID NO: 86) | (SEQ ID NO: 44) |
| Ch14F11 Chimeric Kappa | Ch06C11 Chimeric Heavy |
| Light Chain Variable Region | Heavy Chain Variable Region |
| (SEQ ID NO: 86) | (SEQ ID NO: 46) |

Each of the possible combinations of the chimeric 01G06 and chimeric 08G01 immunoglobulin heavy chain and immunoglobulin light chain variable regions is set forth below in Table 18.

TABLE 18

| Light Chain Variable Region | Heavy Chain Variable Region |
|---|---|
| Ch01G06 Chimeric Kappa | 08G01 Chimeric Heavy |
| (SEQ ID NO: 76) | (SEQ ID NO: 48) |
| 08G01 Chimeric Kappa | Ch01G06 Chimeric Heavy |
| (SEQ ID NO: 84) | (SEQ ID NO: 40) |

The nucleic acid sequences and the encoded protein sequences defining variable regions of the chimeric and humanized 01G06, 06C11, and 14F11 antibodies are summarized below (amino terminal signal peptide sequences are not shown). CDR sequences (Kabat definition) are shown in bold and are underlined in the amino acid sequences.

Nucleic Acid Sequence Encoding the Ch01G06 Chimeric Heavy Chain Variable Region (SEQ ID NO: 127)

| | |
|---|---|
| 1 | gaagtgttgt tgcagcagtc agggccggag ttggtaaaac cgggagcgtc ggtgaaaatc |
| 61 | ccgtgcaaag cgtcggggta tacgtttacg gactataaca tggattgggt gaaacagtcg |
| 121 | catgggaaat cgcttgaatg gattggtcag atcaatccga ataatggagg aatcttcttt |
| 181 | aatcagaagt ttaaaggaaa agcgacgctt acagtcgata agtcgtcgaa cacggcgttc |
| 241 | atggaagtac ggtcgcttac gtcggaagat acggcggtct attactgtgc gagggaggcg |
| 301 | attacgacgg tgggagcgat ggactattgg ggacaaggga cgtcggtcac ggtatcgtcg |

Protein Sequence Defining the Ch01G06 Chimeric Heavy Chain Variable Region (SEQ ID NO:40)

| | |
|---|---|
| 1 | evllqqsgpe lvkpgasvki pckasgytft dynmdwvkqs hgkslewigq inpnnggiff |
| 61 | nqkfkgkatl tvdkssntaf mevrsltsed tavyycarea ittvgamdyw gqgtsvtvss |

Nucleic Acid Sequence Encoding the Hu01G06 IGHV1-18 Heavy Chain Variable Region (SEQ ID NO:53)

| | |
|---|---|
| 1 | caagtgcaac ttgtgcagtc gggtgcggaa gtcaaaaagc cgggagcgtc ggtgaaagta |
| 61 | tcgtgtaaag cgtcgggata tacgtttacg gactataaca tggactgggt acgacaggca |
| 121 | ccggggaaat cgttggaatg gatcggacag attaatccga acaatggggg aattttcttt |
| 181 | aatcagaaat tcaaaggacg ggcgacgttg acggtcgata catcgacgaa tacggcgtat |
| 241 | atggaattga ggtcgcttcg ctcggacgat acggcggtct attactgcgc cagggaggcg |
| 301 | atcacgacgg taggggcgat ggattattgg ggacagggga cgcttgtgac ggtatcgtcg |

Protein Sequence Defining the Hu01G06 IGHV1-18 Heavy Chain Variable Region (SEQ ID NO:54)

| | |
|---|---|
| 1 | qvqlvqsgae vkkpgasvkv sckasgytft dynmdwvrqa pgkslewigq inpnnggiff |
| 61 | nqkfkgratl tvdtstntay melrslrsdd tavyycarea ittvgamdyw gqgtlvtvss |

Nucleic Acid Sequence Encoding the Hu01G06 IGHV1-69 Heavy Chain Variable Region (SEQ ID NO:55)

| | |
|---|---|
| 1 | caagtccagc ttgtccagtc gggagcggaa gtgaagaaac cggggtcgtc ggtcaaagta |
| 61 | tcgtgtaaag cgtcgggata tacgtttacg gactataaca tggattgggt acgacaggct |
| 121 | ccgggaaaat cattggaatg gattggacag attaatccga ataatggggg tatcttcttt |
| 181 | aatcaaaagt ttaaagggag ggcgacgttg acggtggaca aatcgacaaa tacggcgtat |
| 241 | atggaattgt cgtcgcttcg gtcggaggac acggcggtgt attactgcgc gagggaggcg |
| 301 | atcacgacgg tcggggcgat ggattattgg ggacagggaa cgcttgtgac ggtatcgtcg |

Protein Sequence Defining the Hu01G06 IGHV1-69 Heavy Chain Variable Region (SEQ ID NO:56)

| | |
|---|---|
| 1 | qvqlvqsgae vkkpgssvkv sckasgytft dynmdwvrqa pgkslewigq inpnnggiff |
| 61 | nqkfkgratl tvdkstntay melsslrsed tavyycarea ittvgamdyw gqgtlvtvss |

Nucleic Acid Sequence Encoding the Sh01G06 IGHV1-18 M69L Heavy Chain Variable Region (SEQ ID NO:57)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacagg gtcttgaatg gatggggcag attaatccga ataatggagg gatcttcttt |
| 181 | aatcagaaat tcaaaggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |

Protein Sequence Defining the Sh01G06 IGHV1-18 M69L Heavy Chain Variable Region (SEQ ID NO:58)

| | |
|---|---|
| 1 | qvglvqsgae vkkpgasvkv sckasgytft dynmdwvrqa pgqglewmgq inpnnggiff |
| 61 | nqkfkgrvtl ttdtststay melrslrsdd tavyycarea ittvgamdyw gqgtlvtvss |

Nucleic Acid Sequence Encoding the Sh01G06 IGHV1-18 M69L K64Q G44S Heavy Chain Variable Region (SEQ ID NO:59)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacaga gccttgaatg gatggggcag attaatccga ataatggagg gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |

Protein Sequence Defining the Sh01G06 IGHV1-18 M69L K64Q G44S Heavy Chain Variable Region (SEQ ID NO:60)

| | |
|---|---|
| 1 | qvqlvqsgae vkkpgasvkv sckasgytft dynmdwvrqa pgqslewmgq inpnnggiff |
| 61 | nqkfqgrvtl ttdtststay melrslrsdd tavyycarea ittvgamdyw gqgtlvtvss |

Nucleic Acid Sequence Encoding the Sh01G06 IGHV1-18 M69L K64Q Heavy Chain Variable Region (SEQ ID NO:61)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacagg gtcttgaatg gatggggcag attaatccga ataatggagg gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |

Protein Sequence Defining the Sh01G06 IGHV1-18 M69L K64Q Heavy Chain Variable Region (SEQ ID NO:62)

| | |
|---|---|
| 1 | qvqlvqsgae vkkpgasvkv sckasgytft dynmdwvrqa pgqglewmgq inpnnggiff |
| 61 | nqkfqgrvtl ttdtststay melrslrsdd tavyycarea ittvgamdyw gqgtlvtvss |

Nucleic Acid Sequence Encoding the Sh01G06 IGHV1-69 T30S I69L Heavy Chain Variable Region (SEQ ID NO:63)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatggggg aatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |

Protein Sequence Defining the Sh01G06 IGHV1-69 T30S I69L Heavy Chain Variable Region (SEQ ID NO:64)

| | |
|---|---|
| 1 | qvqlvqsgae vkkpgssvkv sckasgytfs dynmdwvrqa pgqglewmgq inpnnggiff |
| 61 | nqkfkgrvtl tadkststay melsslrsed tavyycarea ittvgamdyw gqgtlvtvss |

Nucleic Acid Sequence Encoding the Sh01G06 IGHV1-69 T30S K64Q I69L Heavy Chain Variable Region (SEQ ID NO:65)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatggggg aatctttttc |
| 181 | aatcagaagt ttcaggggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |

Protein Sequence Defining the Sh01G06 IGHV1-69 T30S K64Q I69L Heavy Chain Variable Region (SEQ ID NO:66)

| | |
|---|---|
| 1 | qvqlvqsgae vkkpgssvkv sckasgytfs dynmdwvrqa pgqglewmgq inpnnggiff |
| 61 | nqkfqgrvtl tadkststay melsslrsed tavyycarea ittvgamdyw gqgtlvtvss |

Nucleic Acid Sequence Encoding the Hu01G06 IGHV1-18 F1 Heavy Chain Variable Region (SEQ ID NO:245)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacaga gccttgaatg gatggggcag attaatccgt acaatcacct gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |

Protein Sequence Defining the Hu01G06 IGHV1-18 F1 Heavy Chain Variable Region (SEQ ID NO:246)

| | |
|---|---|
| 1 | qvqlvqsgae vkkpgasvkv sckasgytft dynmdwvrqa pgqslewmgq inpynhliff |
| 61 | nqkfqgrvtl ttdtststay melrslrsdd tavyycarea ittvgamdyw gqgtlvtvss |

Nucleic Acid Sequence Encoding the Hu01G06 IGHV1-18 F2 Heavy Chain Variable Region (SEQ ID NO:247)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacaga gccttgaatg gatggggcag attaatccga ataatggact gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |

Protein Sequence Defining the Hu01G06 IGHV1-18 F2 Heavy Chain Variable Region (SEQ ID NO:248)

| | |
|---|---|
| 1 | qvqlvqsgae vkkpgasvkv sckasgytft dynmdwvrqa pgqslewmgq inpnngliff |
| 61 | nqkfqgrvtl ttdtststay melrslrsdd tavyycarea ittvgamdyw gqgtlvtvss |

Nucleic Acid Sequence Encoding the Hu01G06 IGHV1-69 F1 Heavy Chain Variable Region (SEQ ID NO:249)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatgggct gatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |

Protein Sequence Defining the Hu01G06 IGHV1-69 F1 Heavy Chain Variable Region (SEQ ID NO:250)

| | |
|---|---|
| 1 | qvglvqsgae vkkpgssvkv sckasgytfs dynmdwvrqa pgqglewmgq inpnngliff |
| 61 | nqkfkgrvtl tadkststay melsslrsed tavyycarea ittvgamdyw gqgtlvtvss |

Nucleic Acid Sequence Encoding the Hu01G06 IGHV1-69 F2 Heavy Chain Variable Region (SEQ ID NO:251)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccgt acaatcacct gatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |

Protein Sequence Defining the Hu01G06 IGHV1-69 F2 Heavy Chain Variable Region (SEQ ID NO:252)

| | |
|---|---|
| 1 | qvqlvqsgae vkkpgssvkv sckasgytfs dynmdwvrqa pgqglewmgq inpynhliff |
| 61 | nqkfkgrvtl tadkststay melsslrsed tavyycarea ittvgamdyw gqgtlvtvss |

Nucleic Acid Sequence Encoding the Ch06C11 Chimeric Heavy Chain Variable Region (SEQ ID NO: 129)

| | |
|---|---|
| 1 | caggtgacac tcaaagaatc aggacccgga atccttcagc ccagccagac cttgtcgctg |
| 61 | acttgttcgt tctccggttt cagcctgaat acttatggga tgggtgtgtc atggatcagg |
| 121 | caaccgtccg ggaaaggatt ggagtggctc gcgcacatct actgggacga tgacaaacgc |
| 181 | tacaatcctt cgctgaagag ccgattgacg atttccaagg atgcctcgaa caaccgggta |
| 241 | tttcttaaga tcacgtcggt cgatacggca gacacggcga cctattactg cgcccaaaga |
| 301 | gggtacgatg actattgggg atattggggc caggggacac tcgtcacaat ttcagct |

Protein Sequence Defining the Ch06C11 Chimeric Heavy Chain Variable Region (SEQ ID NO:46)

| | |
|---|---|
| 1 | qvtlkesgpg ilqpsqtlsl tcsfsgfsln tygmgvswir qpsgkglewl ahiywdddkr |
| 61 | ynpslksrlt iskdasnnrv flkitsvdta dtatyycaqr gyddywgywg qgtlvtisa |

Nucleic Acid Sequence Encoding the HE LM 06C11 IGHV2-70 Heavy Chain Variable Region (SEQ ID NO:67)

| | |
|---|---|
| 1 | caggtgactt tgaaagaatc cggtcccgca ttggtaaagc caacccagac acttacgctc |
| 61 | acatgtacat tttccggatt cagcttgaac acttacggga tgggagtgtc gtggattcgg |
| 121 | caacctccgg ggaaggctct ggagtggctg gcgcacatct actgggatga tgacaaaagg |
| 181 | tataacccct cacttaaaac gagactgacg atctcgaagg acacaagcaa gaatcaggtc |
| 241 | gtcctcacga ttacgaatgt agacccggtg gatactgccg tctattactg cgcgcaacgc |
| 301 | gggtatgatg actactgggg atattggggt cagggcaccc tcgtgaccat ctcgtca |

Protein Sequence Defining the HE LM 06C11 IGHV2-70 Heavy Chain Variable Region (SEQ ID NO:68)

| | |
|---|---|
| 1 | qvtlkesgpa lvkptqtltl tctfsgfsln tygmgvswir qppgkalewl ahiywdddkr |
| 61 | ynpslktrlt iskdtsknqv vltitnvdpv dtavyycaqr gyddywgywg qgtlvtiss |

Nucleic Acid Sequence Encoding the Hu06C11 IGHV2-5 Heavy Chain Variable Region (SEQ ID NO:69)

| | |
|---|---|
| 1 | caagtaacgc tcaaggagtc cggacccacc ttggtgaagc |
| | cgacgcagac cttgactctt |
| 61 | acgtgcactt tctcggggtt ttcactgaat acgtacggga |
| | tgggtgtctc atggatcagg |
| 121 | caacctccgg ggaaaggatt ggaatggctg gcgcacatct |
| | actgggatga cgataagaga |
| 181 | tataacccaa gcctcaagtc gcggctcacc attacaaaag |
| | atacatcgaa aaatcaggtc |
| 241 | gtacttacta tcacgaacat ggaccccgtg gacacagcaa |
| | catattactg tgcccagcgc |
| 301 | ggctatgacg attattgggg ttactgggga cagggaacac |
| | tggtcacggt gtccagc |

Protein Sequence Defining the Hu06C11 IGHV-2-5 Heavy Chain Variable Region (SEQ ID NO:70)

| | |
|---|---|
| 1 | qvtlkesgpt lvkptqtltl tctfsgfsln tygmgvswir |
| | qppgkglewl ahiywdddkr |
| 61 | ynpslksrlt itkdtsknqv vltitnmdpv dtatyycaqr |
| | gyddywgywg qgtlvtvss |

Nucleic Acid Sequence Encoding the Ch14F11 Chimeric Heavy Chain Variable Region (SEQ ID NO:131)

| | |
|---|---|
| 1 | caggtcacgc tgaaagagtc aggtcccgga atccttcaac |
| | cttcgcagac attgtcactc |
| 61 | acatgttcct tctccgggtt ctcgctctcg acttatggca |
| | tgggtgtagg atggattcgg |
| 121 | cagcccagcg ggaaggggct tgagtggttg gcggatatct |
| | ggtgggacga cgacaaatac |
| 181 | tacaatccga gcctgaagtc ccgcctcacc atttcgaaag |
| | atacgtcatc aaacgaagtc |
| 241 | tttttgaaga tcgccatcgt ggacacggcg gatacagcga |
| | cgtattactg cgccagaagg |
| 301 | ggacactaca gcgcaatgga ttattgggga caggggacct |
| | cggtgactgt gtcgtcc |

Protein Sequence Defining the Ch14F11 Chimeric Heavy Chain Variable Region (SEQ ID NO:50)

| | |
|---|---|
| 1 | qvtlkesgpg ilqpsqtlsl tcsfsgfsls tygmgvgwir |
| | qpsgkglewl adiwwdddky |
| 61 | ynpslksrlt iskdtssnev flkiaivdta dtatyycarr |
| | ghysamdywg qgtsvtvss |

Nucleic Acid Sequence Encoding the Sh14F11 IGHV2-5 Heavy Chain Variable Region (SEQ ID NO:71)

| | |
|---|---|
| 1 | cagatcactt tgaaagaaag cggaccgacc ttggtcaagc |
| | ccacacaaac cctcacgctc |
| 61 | acgtgtacat tttcggggtt ctcgctttca acttacggga |
| | tgggagtagg gtggattcgc |
| 121 | cagccgcctg gtaaagcgtt ggagtggctt gcagacatct |
| | ggtgggacga cgataagtac |
| 181 | tataatccct cgctcaagtc cagactgacc atcacgaaag |
| | atacgagcaa gaaccaggtc |
| 241 | gtgctgacaa tgactaacat ggacccagtg gatacggcta |
| | catattactg cgccaggcgg |
| 301 | ggtcactact cagcgatgga ttattggggc cagggaacac |
| | tggtaacggt gtcgtcc |

Protein Sequence Defining the Sh14F11 IGHV2-5 Heavy Chain Variable Region (SEQ ID NO:72)

| | |
|---|---|
| 1 | qitlkesgpt lvkptqtltl tctfsgfsls tygmgvgwir |
| | qppgkalewl adiwwdddky |
| 61 | ynpslksrlt itkdtsknqv vltmtnmdpv dtatyycarr |
| | ghysamdywg qgtlvtvss |

Nucleic Acid Sequence Encoding the Sh14F11 IGHV2-70 Heavy Chain Variable Region (SEQ ID NO:73)

| | |
|---|---|
| 1 | caagtgactc tcaaggagtc cggacccgcc ctggtcaaac |
| | caacgcagac actgacgctc |
| 61 | acatgcacct tcagcggatt ttcgttgtca acgtacggca |
| | tgggtgtggg gtggattcgc |
| 121 | cagcctccgg ggaaagccct tgaatggttg gcggacatct |
| | ggtgggatga tgacaagtac |
| 181 | tataatccct cacttaagtc acggttgacg atctcgaaag |
| | acaccagcaa gaaccaggta |
| 241 | gtgctgacaa tgactaacat ggacccggtc gatacagcgg |
| | tctactattg tgctagaagg |
| 301 | ggacactact ccgcaatgga ttattggggt caggggacgc |
| | tcgtaaccgt gtcgtcg |

Protein Sequence Defining the Sh14F11 IGHV2-70 Heavy Chain Variable Region (SEQ ID NO:74)

| | |
|---|---|
| 1 | qvtlkesgpa lvkptqtltl tctfsgfsls tygmgvgwir |
| | qppgkalewl adiwwdddky |
| 61 | ynpslksrlt iskdtsknqv vltmtnmdpv dtavyycarr |
| | ghysamdywg qgtlvtvss |

Nucleic Acid Sequence Encoding the Ch01G06 Chimeric Kappa Chain Variable Region (SEQ ID NO:133)

| | |
|---|---|
| 1 | gacatccaaa tgacccagtc acccgcgagc ctttcggcgt |
| | cggtcggaga aacggtcacg |
| 61 | atcacgtgcc ggacatcaga gaatctccat aactacctcg |
| | cgtggtatca acagaagcag |
| 121 | gggaagtcgc cccagttgct tgtatacgat gcgaaaacgt |
| | tggcggatgg ggtgccgtcc |
| 181 | agattctcgg gatcgggctc ggggacgcag tactcgctca |
| | agatcaattc gctgcagccg |
| 241 | gaggactttg ggtcgtacta ttgtcagcat ttttggtcat |
| | caccgtatac atttggaggt |
| 301 | ggaacgaaac ttgagattaa g |

Protein Sequence Defining the Ch01G06 Chimeric Kappa Chain Variable Region (SEQ ID NO:76)

| | |
|---|---|
| 1 | diqmtqspas lsasvgetvt itcrtsenlh nylawyqqkq |
| | gkspqllvyd aktladgvps |
| 61 | rfsgsgsgtq yslkinslqp edfgsyycqh fwsspytfgg |
| | gtkleik |

Nucleic Acid Sequence Encoding the Hu01G06 IGKV1-39 Kappa Chain Variable Region (SEQ ID NO:89)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaagtcac cgaaactcct tgtctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa g |

Protein Sequence Defining the Hu01G06 IGKV1-39 Kappa Chain Variable Region (SEQ ID NO:90)

| | |
|---|---|
| 1 | diqmtqspss lsasvgdrvt itcrtsenlh nylawyqqkp |
| | gkspkllvyd aktladgvps |
| 61 | rfsgsgsgtd ytltisslqp edfatyycqh fwsspytfgq |
| | gtkleik |

Nucleic Acid Sequence Encoding the Hu01G06 IGKV1-39 S43A V48I Kappa Chain Variable Region (Also Referred to Herein as Hu01G06 IGKV1-39 F1 Kappa Chain Variable Region; SEQ ID NO:91)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaaggccc cgaaactcct tatctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa g |

Protein Sequence Defining the Hu01G06 IGKV1-39 S43A V48I Kappa Chain Variable Region (Also Referred to Herein as Hu01G06 IGKV1-39 F1 Kappa Chain Variable Region; SEQ ID NO:92)

| | |
|---|---|
| 1 | diqmtqspss lsasvgdrvt itcrtsenlh nylawyqqkp |
| | gkapklliyd aktladgvps |
| 61 | rfsgsgsgtd ytltisslqp edfatyycqh fwsspytfgq |
| | gtkleik |

Nucleic Acid Sequence Encoding the Hu01G06 IGKV1-39 V48I Kappa Chain Variable Region (SEQ ID NO:93)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaagtcac cgaaactcct tatctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa g |

Protein Sequence Defining the Hu01G06 IGKV1-39 V48I Kappa Chain Variable Region (SEQ ID NO:94)

| | |
|---|---|
| 1 | diqmtqspss lsasvgdrvt itcrtsenlh nylawyqqkp |
| | gkspklliyd aktladgvps |
| 61 | rfsgsgsgtd ytltisslqp edfatyycqh fwsspytfgq |
| | gtkleik |

Nucleic Acid Sequence Encoding the Hu01G06 IGKV1-39 F1 Kappa Chain Variable Region (Also Referred to Herein as Hu01G06 IGKV1-39 S43A V48I Kappa Chain Variable Region; SEQ ID NO:91)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaaggccc cgaaactcct tatctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa g |

Protein Sequence Defining the Hu01G06 IGKV1-39 F1 Kappa Chain Variable Region (Also Referred to Herein as Hu01G06 IGKV1-39 S43A V48I Kappa Chain Variable Region; SEQ ID NO:92)

| | |
|---|---|
| 1 | diqmtqspss lsasvgdrvt itcrtsenlh nylawyqqkp |
| | gkapklliyd aktladgvps |
| 61 | rfsgsgsgtd ytltisslqp edfatyycqh fwsspytfgq |
| | gtkleik |

Nucleic Acid Sequence Encoding the Hu01G06 IGKV1-39 F2 Kappa Chain Variable Region (SEQ ID NO:253)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaagtcac cgaaactcct tatctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgg |
| | acccctacac atttgggcag |
| 301 | gggaccaagt tggaaatcaa g |

Protein Sequence Defining the Hu01G06 IGKV1-39 F2 Kappa Chain Variable Region (SEQ ID NO:254)

| | |
|---|---|
| 1 | diqmtqspss lsasvgdrvt itcrtsenlh nylawyqqkp |
| | gkspklliyd aktladgvps |
| 61 | rfsgsgsgtd ytltisslqp edfatyycqh fwsdpytfgq |
| | gtkleik |

Nucleic Acid Sequence Encoding the Ch06C11 Chimeric Kappa Chain Variable Region (SEQ ID NO:135)

| | |
|---|---|
| 1 | gatatcgtca tgacccagtc ccagaagttc atgtcaactt |
| | cagtgggaga cagagtgtcc |
| 61 | gtcacatgta aagcctcgca aaatgtggga accaacgtag |
| | cgtggttcca gcagaaacct |
| 121 | ggccaatcac cgaaggcact gatctactcg gccagctata |
| | ggtactcggg agtaccagat |
| 181 | cggtttacgg ggtcggggag cgggacggac tttatcctca |
| | ctatttccaa tgtccagtcg |
| 241 | gaggaccttg cggaatactt ctgccagcag tataacaact |
| | atcccctcac gtttggtgct |
| 301 | ggtacaaaat tggagttgaa g |

Protein Sequence Defining the Ch06C11 Chimeric Kappa Chain Variable Region (SEQ ID NO:82)

| | |
|---|---|
| 1 | divmtqsqkf mstsvgdrvs vtckasqnvg tnvawfqqkp |
| | gqspkaliys asyrysgvpd |
| 61 | rftgsgsgtd filtisnvqs edlaeyfcqq ynnypltfga |
| | gtklelk |

Nucleic Acid Sequence Encoding the Sh06C11 IGKV1-16 Kappa Chain Variable Region (SEQ ID NO:95)

| | |
|---|---|
| 1 | gacatccaaa tgacccaatc gccctcctcc ctctccgcat |
| | cagtagggga ccgcgtcaca |
| 61 | attacttgca aagcgtcgca gaacgtcgga acgaatgtgg |
| | cgtggtttca gcagaagccc |
| 121 | ggaaaagctc cgaagagctt gatctactcg gcctcatata |
| | ggtattcggg tgtgccgagc |
| 181 | cggtttagcg ggtcggggtc aggtactgat ttcacgctca |
| | caatttcatc gttgcagcca |
| 241 | gaagatttcg ccacatatta ctgtcagcag tacaacaatt |
| | accctctgac gttcggccag |
| 301 | ggaaccaaac ttgagatcaa g |

Protein Sequence Defining the Sh06C11 IGKV1-16 Kappa Chain Variable Region (SEQ ID NO:96)

| | |
|---|---|
| 1 | diqmtqspss lsasvgdrvt itckasqnvg tnvawfqqkp |
| | gkapksliys asyrysgvps |
| 61 | rfsgsgsgtd ftltisslqp edfatyycqq ynnypltfgq |
| | gtkleik |

Nucleic Acid Sequence Encoding the Ch14F11 Chimeric Kappa Chain Variable Region (SEQ ID NO: 137)

| | |
|---|---|
| 1 | gacatcgtga tgacacagtc acagaaattc atgtccacat |
| | ccgtcggtga tagagtatcc |
| 61 | gtcacgtgta aggcctcgca aaacgtagga actaatgtgg |
| | cgtggtatca acagaagcca |
| 121 | ggacagtcac ccaaagcact catctacagc ccctcatatc |
| | ggtacagcgg ggtgccggac |
| 181 | aggttcacgg gatcggggag cgggaccgat tttacactga |
| | ccatttcgaa tgtccagtcg |
| 241 | gaggaccttg cggaatactt ctgccagcag tataactcgt |
| | accctcacac gtttggaggt |
| 301 | ggcactaagt tggagatgaa a |

Protein Sequence Defining the Ch14F11 Chimeric Kappa Chain Variable Region (SEQ ID NO:86)

| | |
|---|---|
| 1 | divmtqsqkf mstsvgdrvs vtckasqnvg tnvawyqqkp |
| | gqspkaliys psyrysgvpd |
| 61 | rftgsgsgtd ftltisnvqs edlaeyfcqq ynsyphtfgg |
| | gtklemk |

Nucleic Acid Sequence Encoding the Hu14F11 IGKV1-16 Kappa Chain Variable Region (SEQ ID NO:97)

| | |
|---|---|
| 1 | gatatccaga tgacacagtc accctcgtcg ctctcagctt |
| | ccgtaggcga cagggtcact |
| 61 | attacgtgta aagcatcaca gaacgtcgga acgaatgtgg |
| | cgtggtttca gcagaagccc |
| 121 | gggaagagcc ccaaagcgct tatctactcc ccgtcgtatc |
| | ggtattccgg tgtgccaagc |
| 181 | agattttcgg ggtcaggttc gggaactgac tttaccctga |
| | ccatctcgtc cctccaaccg |
| 241 | gaagatttcg ccacgtactt ctgccagcag tacaacagct |
| | atcctcacac attcggacaa |
| 301 | gggacaaagt tggagattaa a |

Protein Sequence Defining the Hu14F11 IGKV1-16 Kappa Chain Variable Region (SEQ ID NO:98)

| | |
|---|---|
| 1 | diqmtqspss lsasvgdrvt itckasqnvg tnvawfqqkp |
| | gkspkaliys psyrysgvps |
| 61 | rfsgsgsgtd ftltisslqp edfatyfcqq ynsyphtfgq |
| | gtkleik |

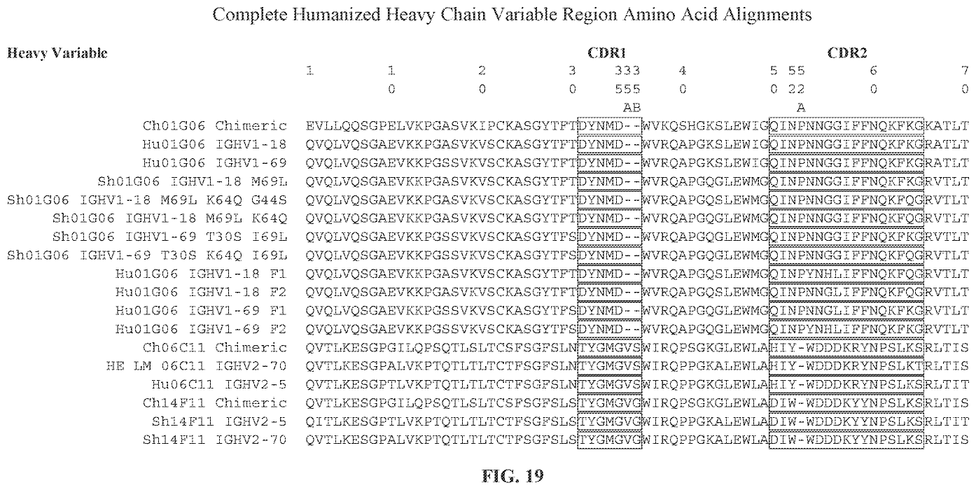

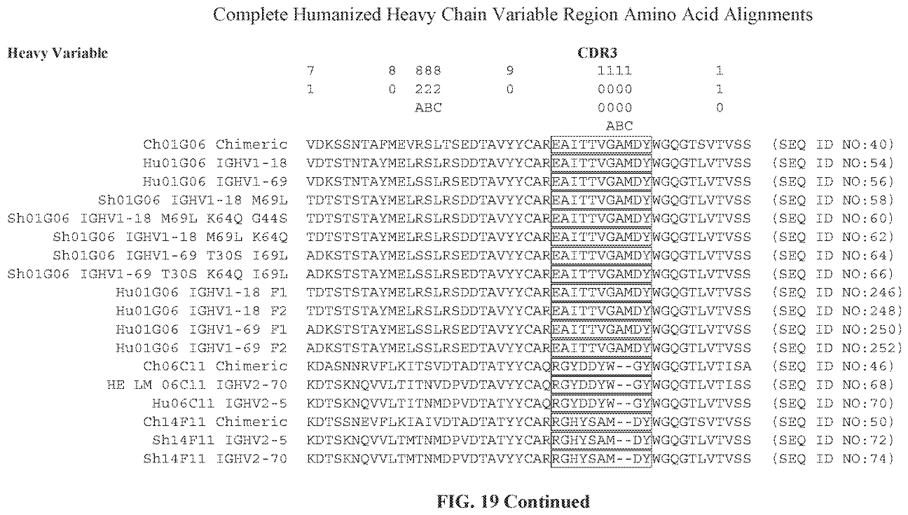

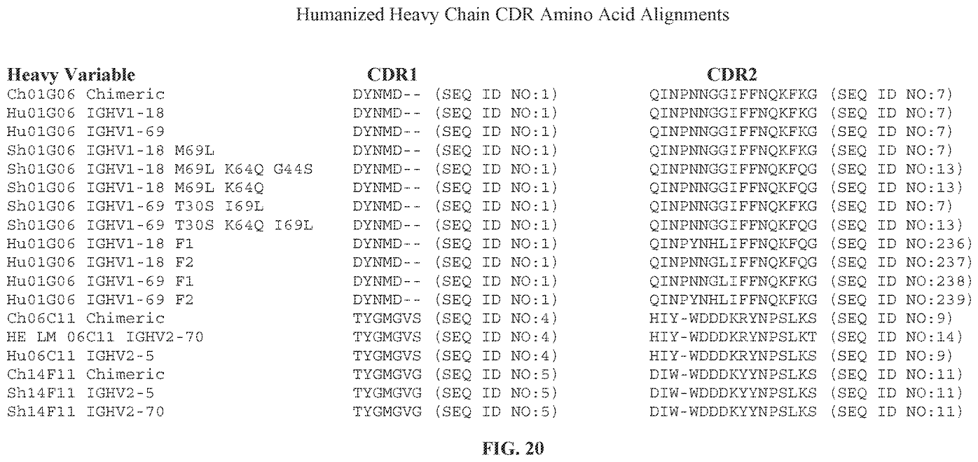

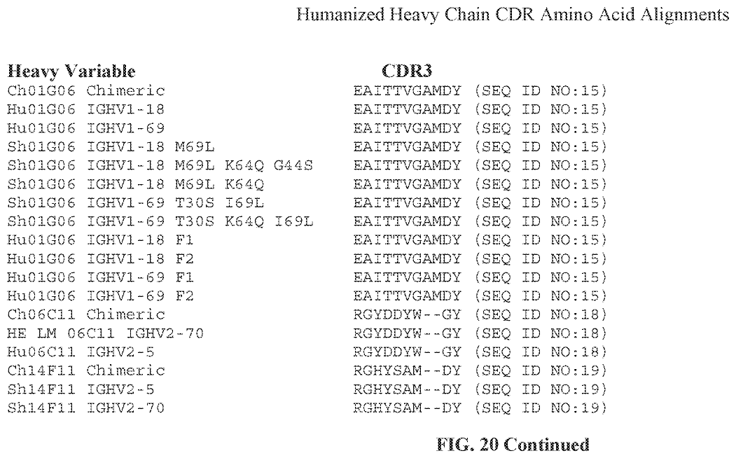

The amino acid sequences defining the immunoglobulin heavy chain variable regions for the antibodies produced in Example 13 are aligned in FIG. 19. Amino terminal signal peptide sequences (for proper expression/secretion) are not shown. CDR₁, CDR₂, and CDR₃ (Kabat definition) are identified by boxes. FIG. 20 show an alignment of the separate CDR₁, CDR₂, and CDR₃ sequences for each of the variable region sequences shown in FIG. 19.

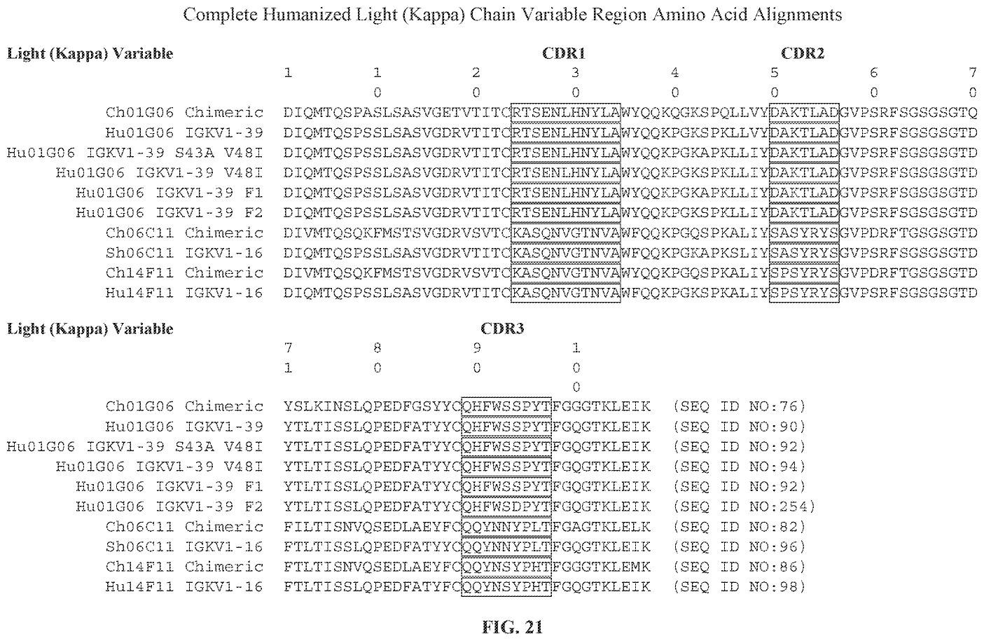

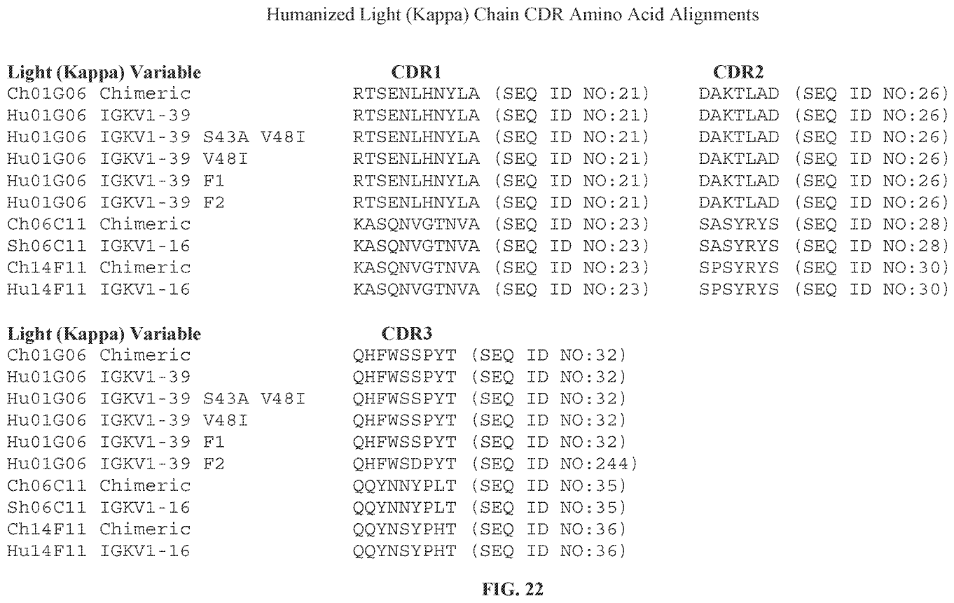

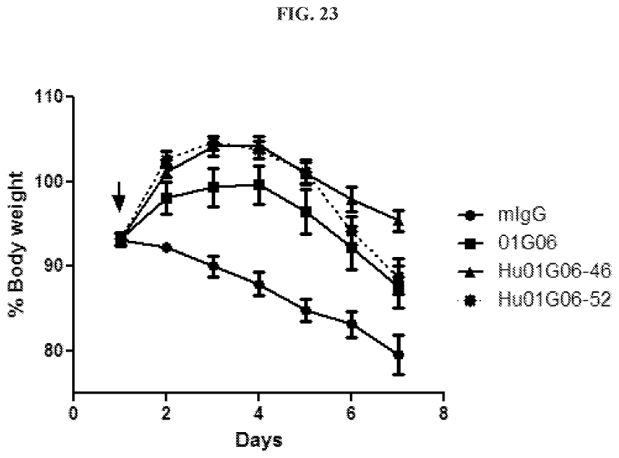

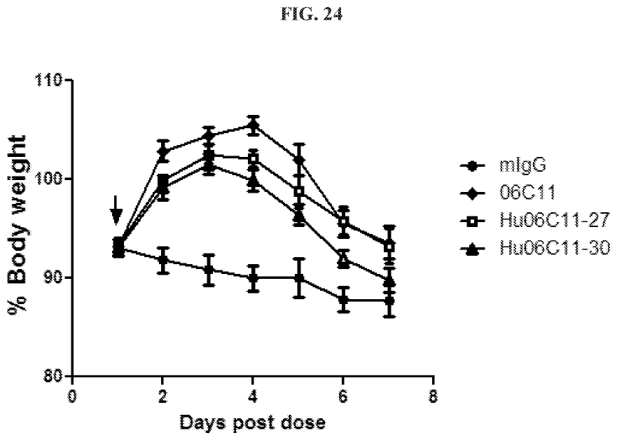

The amino acid sequences defining the immunoglobulin light chain variable regions for the antibodies in Example 13 are aligned in FIG. 21. Amino terminal signal peptide sequences (for proper expression/secretion) are not shown. CDR₁, CDR₂ and CDR₃ are identified by boxes. FIG. 22 shows an alignment of the separate CDR₁, CDR₂, and CDR₃ sequences for each of the variable region sequences shown in FIG. 21.

Table 19 is a concordance chart showing the SEQ ID NO. of each sequence discussed in this Example.

TABLE 19

| SEQ. ID NO. | Nucleic Acid or Protein |
|---|---|
| 127 | Ch01G06 Chimeric Heavy Chain Variable Region-nucleic acid |
| 40 | Ch01G06 Chimeric Heavy Chain Variable Region-protein |
| 1 | Ch01G06 Chimeric Heavy Chain CDR₁ |
| 7 | Ch01G06 Chimeric Heavy Chain CDR₂ |
| 15 | Ch01G06 Chimeric Heavy Chain CDR₃ |
| 53 | Hu01G06 IGHV1-18 Heavy Chain Variable Region-nucleic acid |
| 54 | Hu01G06 IGHV1-18 Heavy Chain Variable Region-protein |
| 1 | Hu01G06 IGHV1-18 Heavy Chain CDR₁ |
| 7 | Hu01G06 IGHV1-18 Heavy Chain CDR₂ |
| 15 | Hu01G06 IGHV1-18 Heavy Chain CDR₃ |
| 55 | Hu01G06 IGHV1-69 Heavy Chain Variable Region-nucleic acid |
| 56 | Hu01G06 IGHV1-69 Heavy Chain Variable Region-protein |
| 1 | Hu01G06 IGHV1-69 Heavy Chain CDR₁ |
| 7 | Hu01G06 IGHV1-69 Heavy Chain CDR₂ |
| 15 | Hu01G06 IGHV1-69 Heavy Chain CDR₃ |
| 57 | Sh01G06 IGHV1-18 M69L Heavy Chain Variable Region-nucleic acid |
| 58 | Sh01G06 IGHV1-18 M69L Heavy Chain Variable Region-protein |
| 1 | Sh01G06 IGHV1-18 M69L Heavy Chain CDR₁ |
| 7 | Sh01G06 IGHV1-18 M69L Heavy Chain CDR₂ |
| 15 | Sh01G06 IGHV1-18 M69L Heavy Chain CDR₃ |
| 59 | Sh01G06 IGHV1-18 M69L K64Q G44S Heavy Chain Variable Region-nucleic acid |
| 60 | Sh01G06 IGHV1-18 M69L K64Q G44S Heavy Chain Variable Region-protein |
| 1 | Sh01G06 IGHV1-18 M69L K64Q G44S Heavy Chain CDR₁ |
| 13 | Sh01G06 IGHV1-18 M69L K64Q G44S Heavy Chain CDR₂ |
| 15 | Sh01G06 IGHV1-18 M69L K64Q G44S Heavy Chain CDR₃ |
| 61 | Sh01G06 IGHV1-18 M69L K64Q Heavy Chain Variable Region-nucleic acid |
| 62 | Sh01G06 IGHV1-18 M69L K64Q Heavy Chain Variable Region-protein |
| 1 | Sh01G06 IGHV1-18 M69L K64Q Heavy Chain CDR₁ |
| 13 | Sh01G06 IGHV1-18 M69L K64Q Heavy Chain CDR₂ |
| 15 | Sh01G06 IGHV1-18 M69L K64Q Heavy Chain CDR₃ |
| 63 | Sh01G06 IGHV1-69 T30S I69L Heavy Chain Variable Region-nucleic acid |
| 64 | Sh01G06 IGHV1-69 T30S I69L Heavy Chain Variable Region-protein |
| 1 | Sh01G06 IGHV1-69 T30S I69L Heavy Chain CDR₁ |
| 7 | Sh01G06 IGHV1-69 T30S I69L Heavy Chain CDR₂ |
| 15 | Sh01G06 IGHV1-69 T30S I69L Heavy Chain CDR₃ |
| 65 | Sh01G06 IGHV1-69 T30S K64Q I69L Heavy Chain Variable Region-nucleic acid |
| 66 | Sh01G06 IGHV1-69 T30S K64Q I69L Heavy Chain Variable Region-protein |
| 1 | Sh01G06 IGHV1-69 T30S K64Q I69L Heavy Chain CDR₁ |
| 13 | Sh01G06 IGHV1-69 T30S K64Q I69L Heavy Chain CDR₂ |
| 15 | Sh01G06 IGHV1-69 T30S K64Q I69L Heavy Chain CDR₃ |
| 245 | Hu01G06 IGHV1-18 F1 Heavy Chain Variable Region-nucleic acid |
| 246 | Hu01G06 IGHV1-18 F1 Heavy Chain Variable Region-protein |
| 1 | Hu01G06 IGHV1-18 F1 Heavy Chain CDR₁ |
| 236 | Hu01G06 IGHV1-18 F1 Heavy Chain CDR₂ |
| 15 | Hu01G06 IGHV1-18 F1 Heavy Chain CDR₃ |
| 247 | Hu01G06 IGHV1-18 F2 Heavy Chain Variable Region-nucleic acid |
| 248 | Hu01G06 IGHV1-18 F2 Heavy Chain Variable Region-protein |
| 1 | Hu01G06 IGHV1-18 F2 Heavy Chain CDR₁ |
| 237 | Hu01G06 IGHV1-18 F2 Heavy Chain CDR₂ |
| 15 | Hu01G06 IGHV1-18 F2 Heavy Chain CDR₃ |
| 259 | Hu01G06 IGHV1-69 F1 Heavy Chain Variable Region-nucleic acid |
| 250 | Hu01G06 IGHV1-69 F1 Heavy Chain Variable Region-protein |
| 1 | Hu01G06 IGHV1-69 F1 Heavy Chain CDR₁ |
| 238 | Hu01G06 IGHV1-69 F1 Heavy Chain CDR₂ |
| 15 | Hu01G06 IGHV1-69 F1 Heavy Chain CDR₃ |
| 251 | Hu01G06 IGHV1-69 F2 Heavy Chain Variable Region-nucleic acid |
| 252 | Hu01G06 IGHV1-69 F2 Heavy Chain Variable Region-protein |
| 1 | Hu01G06 IGHV1-69 F2 Heavy Chain CDR₁ |
| 239 | Hu01G06 IGHV1-69 F2 Heavy Chain CDR₂ |
| 15 | Hu01G06 IGHV1-69 F2 Heavy Chain CDR₃ |
| 129 | Ch06C11 Chimeric Heavy Chain Variable Region-nucleic acid |
| 46 | Ch06C11 Chimeric Heavy Chain Variable Region-protein |
| 4 | Ch06C11 Chimeric Heavy Chain CDR₁ |
| 9 | Ch06C11 Chimeric Heavy Chain CDR₂ |
| 18 | Ch06C11 Chimeric Heavy Chain CDR₃ |
| 67 | HE LM 06C11 IGHV2-70 Heavy Chain Variable Region-nucleic acid |
| 68 | HE LM 06C11 IGHV2-70 Heavy Chain Variable Region-protein |
| 4 | HE LM 06C11 IGHV2-70 Heavy Chain CDR₁ |
| 14 | HE LM 06C11 IGHV2-70 Heavy Chain CDR₂ |
| 18 | HE LM 06C11 IGHV2-70 Heavy Chain CDR₃ |
| 69 | Hu06C11 IGHV2-5 Heavy Chain Variable Region-nucleic acid |
| 70 | Hu06C11 IGHV2-5 Heavy Chain Variable Region-protein |
| 4 | Hu06C11 IGHV2-5 Heavy Chain CDR₁ |
| 9 | Hu06C11 IGHV2-5 Heavy Chain CDR₂ |
| 18 | Hu06C11 IGHV2-5 Heavy Chain CDR₃ |
| 131 | Ch14F11 Chimeric Heavy Chain Variable Region-nucleic acid |
| 50 | Ch14F11 Chimeric Heavy Chain Variable Region-protein |
| 5 | Ch14F11 Chimeric Heavy Chain CDR₁ |
| 11 | Ch14F11 Chimeric Heavy Chain CDR₂ |
| 19 | Ch14F11 Chimeric Heavy Chain CDR₃ |
| 71 | Sh14F11 IGHV2-5 Heavy Chain Variable Region-nucleic acid |
| 72 | Sh14F11 IGHV2-5 Heavy Chain Variable Region-protein |
| 5 | Sh14F11 IGHV2-5 Heavy Chain CDR₁ |
| 11 | Sh14F11 IGHV2-5 Heavy Chain CDR₂ |
| 19 | Sh14F11 IGHV2-5 Heavy Chain CDR₃ |
| 73 | Sh14F11 IGHV2-70 Heavy Chain Variable Region-nucleic acid |
| 74 | Sh14F11 IGHV2-70 Heavy Chain Variable Region-protein |
| 5 | Sh14F11 IGHV2-70 Heavy Chain CDR₁ |
| 11 | Sh14F11 IGHV2-70 Heavy Chain CDR₂ |
| 19 | Sh14F11 IGHV2-70 Heavy Chain CDR₃ |
| 133 | Ch01G06 Chimeric Light (kappa) Chain Variable Region-nucleic acid |
| 76 | Ch01G06 Chimeric Light (kappa) Chain Variable Region-protein |
| 21 | Ch01G06 Chimeric Light (kappa) Chain CDR₁ |
| 26 | Ch01G06 Chimeric Light (kappa) Chain CDR₂ |
| 32 | Ch01G06 Chimeric Light (kappa) Chain CDR₃ |
| 89 | Hu01G06 IGKV1-39 Light (kappa) Chain Variable Region-nucleic acid |
| 90 | Hu01G06 IGKV1-39 Light (kappa) Chain Variable Region-protein |
| 21 | Hu01G06 IGKV1-39 Light (kappa) Chain CDR₁ |
| 26 | Hu01G06 IGKV1-39 Light (kappa) Chain CDR₂ |
| 32 | Hu01G06 IGKV1-39 Light (kappa) Chain CDR₃ |
| 91 | Hu01G06 IGKV1-39 S43A V48I Light (kappa) Chain Variable Region-nucleic acid |
| 92 | Hu01G06 IGKV1-39 S43A V48I Light (kappa) Chain Variable Region-protein |
| 21 | Hu01G06 IGKV1-39 S43A V48I Light (kappa) Chain CDR₁ |
| 26 | Hu01G06 IGKV1-39 S43A V48I Light (kappa) Chain CDR₂ |
| 32 | Hu01G06 IGKV1-39 S43A V48I Light (kappa) Chain CDR₃ |
| 93 | Hu01G06 IGKV1-39 V48I Light (kappa) Chain Variable Region-nucleic acid |
| 94 | Hu01G06 IGKV1-39 V48I Light (kappa) Chain Variable Region-protein |
| 21 | Hu01G06 IGKV1-39 V48I Light (kappa) Chain CDR₁ |
| 26 | Hu01G06 IGKV1-39 V48I Light (kappa) Chain CDR₂ |
| 32 | Hu01G06 IGKV1-39 V48I Light (kappa) Chain CDR₃ |
| 91 | Hu01G06 IGKV1-39 F1 Light (kappa) Chain Variable Region-nucleic acid |
| 92 | Hu01G06 IGKV1-39 F1 Light (kappa) Chain Variable Region-protein |
| 21 | Hu01G06 IGKV1-39 F1 Light (kappa) Chain CDR₁ |
| 26 | Hu01G06 IGKV1-39 F1 Light (kappa) Chain CDR₂ |
| 32 | Hu01G06 IGKV1-39 F1 Light (kappa) Chain CDR₃ |
| 253 | Hu01G06 IGKV1-39 F2 Light (kappa) Chain Variable Region-nucleic acid |
| 254 | Hu01G06 IGKV1-39 F2 Light (kappa) Chain Variable Region-protein |
| 21 | Hu01G06 IGKV1-39 F2 Light (kappa) Chain CDR₁ |
| 26 | Hu01G06 IGKV1-39 F2 Light (kappa) Chain CDR₂ |
| 244 | Hu01G06 IGKV1-39 F2 Light (kappa) Chain CDR₃ |
| 135 | Ch06C11 Chimeric Light (kappa) Chain Variable Region-nucleic acid |
| 82 | Ch06C11 Chimeric Light (kappa) Chain Variable Region-protein |
| 23 | Ch06C11 Chimeric Light (kappa) Chain CDR₁ |
| 28 | Ch06C11 Chimeric Light (kappa) Chain CDR₂ |
| 35 | Ch06C11 Chimeric Light (kappa) Chain CDR₃ |
| 95 | Sh06C11 IGKV1-16 Light (kappa) Chain Variable Region-nucleic acid |
| 96 | Sh06C11 IGKV1-16 Light (kappa) Chain Variable Region-protein |
| 23 | Sh06C11 IGKV1-16 Light (kappa) Chain CDR₁ |
| 28 | Sh06C11 IGKV1-16 Light (kappa) Chain CDR₂ |
| 35 | Sh06C11 IGKV1-16 Light (kappa) Chain CDR₃ |
| 137 | Ch14F11 Chimeric Light (kappa) Chain Variable Region-nucleic acid |
| 86 | Ch14F11 Chimeric Light (kappa) Chain Variable Region-protein |
| 23 | Ch14F11 Chimeric Light (kappa) Chain CDR₁ |
| 30 | Ch14F11 Chimeric Light (kappa) Chain CDR₂ |
| 36 | Ch14F11 Chimeric Light (kappa) Chain CDR₃ |
| 97 | Hu14F11 IGKV1-16 Light (kappa) Chain Variable Region-nucleic acid |
| 98 | Hu14F11 IGKV1-16 Light (kappa) Chain Variable Region-protein |
| 23 | Hu14F11 IGKV1-16 Light (kappa) Chain CDR₁ |
| 30 | Hu14F11 IGKV1-16 Light (kappa) Chain CDR₂ |
| 36 | Hu14F11 IGKV1-16 Light (kappa) Chain CDR₃ |

Humanized monoclonal antibody heavy chain CDR sequences (Kabat, Chothia, and IMGT definitions) are shown in Table 20.

TABLE 20

| | | | |
|---|---|---|---|
| | | | Variable Region |
| CDR1 | CDR2 | CDR3 | SEQ ID NO: |

Kabat

| | | | | |
|---|---|---|---|---|
| Ch01G06 Chimeric | DYNMD | QINPNNGGIFFNQKFKG | EAITTVGAMDY | 40 |
| | (SEQ ID NO: 1) | (SEQ ID NO: 7) | (SEQ ID NO: 15) | |
| Hu01G06 IGHV1- | DYNMD | QINPNNGGIFFNQKFKG | EAITTVGAMDY | 54 |
| 18 | (SEQ ID NO: 1) | (SEQ ID NO: 7) | (SEQ ID NO: 15) | |
| Hu01G06 IGHV1- | DYNMD | QINPNNGGIFFNQKFKG | EAITTVGAMDY | 56 |
| 69 | (SEQ ID NO: 1) | (SEQ ID NO: 7) | (SEQ ID NO: 15) | |
| Sh01G06 IGHV1- | DYNMD | QINPNNGGIFFNQKFKG | EAITTVGAMDY | 58 |
| 18 M69L | (SEQ ID NO: 1) | (SEQ ID NO: 7) | (SEQ ID NO: 15) | |
| Sh01G06 IGHV1- | DYNMD | QINPNNGGIFFNQKFQG | EAITTVGAMDY | 60 |
| 18 M69L K64Q | (SEQ ID NO: 1) | (SEQ ID NO: 13) | (SEQ ID NO: 15) | |
| G44S | | | | |
| Sh01G06 IGHV1- | DYNMD | QINPNNGGIFFNQKFQG | EAITTVGAMDY | 62 |
| 18 M69L K64Q | (SEQ ID NO: 1) | (SEQ ID NO: 13) | (SEQ ID NO: 15) | |
| Sh01G06 IGHV1- | DYNMD | QINPNNGGIFFNQKFKG | EAITTVGAMDY | 64 |
| 69 T30S I69L | (SEQ ID NO: 1) | (SEQ ID NO: 7) | (SEQ ID NO: 15) | |
| Sh01G06 IGHV1- | DYNMD | QINPNNGGIFFNQKFQG | EAITTVGAMDY | 66 |
| 69 T30S K64Q | (SEQ ID NO: 1) | (SEQ ID NO: 13) | (SEQ ID NO: 15) | |
| I69L | | | | |
| Hu01G06 IGHV1- | DYNMD | QINPYNHLIFFNQKFQG | EAITTVGAMDY | 246 |
| 18F1 | (SEQ ID NO: 1) | (SEQ ID NO: 236) | (SEQ ID NO: 15) | |
| Hu01G06 IGHV1- | DYNMD | QINPNNGLIFFNQKFQG | EAITTVGAMDY | 248 |
| 18F2 | (SEQ ID NO: 1) | (SEQ ID NO: 237) | (SEQ ID NO: 15) | |
| Hu01G06 IGHV1- | DYNMD | QINPNNGLIFFNQKFKG | EAITTVGAMDY | 250 |
| 69F1 | (SEQ ID NO: 1) | (SEQ ID NO: 238) | (SEQ ID NO: 15) | |
| Hu01G06 IGHV1- | DYNMD | QINPYNHLIFFNQKFKG | EAITTVGAMDY | 252 |
| 69F2 | (SEQ ID NO: 1) | (SEQ ID NO: 239) | (SEQ ID NO: 15) | |
| Ch06C11 Chimeric | TYGMGVS | HIYWDDDKRYNPSLKS | RGYDDYWGY | 46 |
| | (SEQ ID NO: 4) | (SEQ ID NO: 9) | (SEQ ID NO: 18) | |
| HE LM 06C11 | TYGMGVS | HIYWDDDKRYNPSLKT | RGYDDYWGY | 68 |
| IGHV2-70 | (SEQ ID NO: 4) | (SEQ ID NO: 14) | (SEQ ID NO: 18) | |
| Hu06C11 IGHV2- | TYGMGVS | HIYWDDDKRYNPSLKS | RGYDDYWGY | 70 |
| 5 | (SEQ ID NO: 4) | (SEQ ID NO: 9) | (SEQ ID NO: 18) | |
| Ch14F11 Chimeric | TYGMGVG | DIWWDDDKYYNPSLKS | RGHYSAMDY | 50 |
| | (SEQ ID NO: 5) | (SEQ ID NO: 11) | (SEQ ID NO: 19) | |
| Sh14F11 IGHV2-5 | TYGMGVG | DIWWDDDKYYNPSLKS | RGHYSAMDY | 72 |
| | (SEQ ID NO: 5) | (SEQ ID NO: 11) | (SEQ ID NO: 19) | |
| Sh14F11 IGHV2- | TYGMGVG | DIWWDDDKYYNPSLKS | RGHYSAMDY | 74 |
| 70 | (SEQ ID NO: 5) | (SEQ ID NO: 11) | (SEQ ID NO: 19) | |

Chothia

| | | | | |
|---|---|---|---|---|
| Ch01G06 | GYTFTDY | NPNNGG | EAITTVGAMDY | 40 |
| Chimeric | (SEQ ID NO: 38) | (SEQ ID NO: 143) | (SEQ ID NO: 15) | |
| Hu0106 IGHV1- | GYTFTDY | NPNNGG | EAITTVGAMDY | 54 |
| 18 | (SEQ ID NO: 38) | (SEQ ID NO: 143) | (SEQ ID NO: 15) | |
| Hu01G06 IGHV1- | GYTFTDY | NPNNGG | EAITTVGAMDY | 56 |
| 69 | (SEQ ID NO: 38) | (SEQ ID NO: 143) | (SEQ ID NO: 15) | |
| Sh01G06 IGHV1- | GYTFTDY | NPNNGG | EAITTVGAMDY | 58 |
| 18 M69L | (SEQ ID NO: 38) | (SEQ ID NO: 143) | (SEQ ID NO: 15) | |
| Sh01G06 IGHV1- | GYTFTDY | NPNNGG | EAITTVGAMDY | 60 |
| 18 M69L K64Q | (SEQ ID NO: 38) | (SEQ ID NO: 143) | (SEQ ID NO: 15) | |
| G44S | | | | |
| Sh01G06 IGHV1- | GYTFTDY | NPNNGG | EAITTVGAMDY | 62 |
| 18 M69L K64Q | (SEQ ID NO: 38) | (SEQ ID NO: 143) | (SEQ ID NO: 15) | |
| Sh01G06 IGHV1- | GYTFSDY | NPNNGG | EAITTVGAMDY | 64 |
| 69 T30S I69L | (SEQ ID NO: 234) | (SEQ ID NO: 143) | (SEQ ID NO: 15) | |
| Sh01G06 IGHV1- | GYTFSDY | NPNNGG | EAITTVGAMDY | 66 |
| 69 T30S K64Q | (SEQ ID NO: 234) | (SEQ ID NO: 143) | (SEQ ID NO: 15) | |
| I69L | | | | |
| Hu01G06 IGHV1- | GYTFTDY | NPYNHL | EAITTVGAMDY | 246 |
| 18 F1 | (SEQ ID NO: 38) | (SEQ ID NO: 240) | (SEQ ID NO: 15) | |
| Hu01G06 IGHV1- | GYTFTDY | NPNNGL | EAITTVGAMDY | 248 |
| 18 F2 | (SEQ ID NO: 38) | (SEQ ID NO: 241) | (SEQ ID NO: 15) | |
| Hu01G06 IGHV1- | GYTFSDY | NPNNGL | EAITTVGAMDY | 250 |
| 69 Fl | (SEQ ID NO: 234) | (SEQ ID NO: 241) | (SEQ ID NO: 15) | |
| Hu01G06 IGHV1- | GYTFSDY | NPYNHL | EAITTVGAMDY | 252 |
| 69 F2 | (SEQ ID NO: 234) | (SEQ ID NO: 240) | (SEQ ID NO: 15) | |
| Ch06C11 | GFSLNTYGM | YWDDD | RGYDDYWGY | 46 |
| Chimeric | (SEQ ID NO: 132) | (SEQ ID NO: 145) | (SEQ ID NO: 18) | |
| HE LM 06C11 | GFSLNTYGM | YWDDD | RGYDDYWGY | 68 |
| IGHV2-70 | (SEQ ID NO: 132) | (SEQ ID NO: 145) | (SEQ ID NO: 18) | |
| Hu06C11 IGHV2- | GFSLNTYGM | YWDDD | RGYDDYWGY | 70 |
| 5 | (SEQ ID NO: 132) | (SEQ ID NO: 145) | (SEQ ID NO: 18) | |
| Ch14F11 Chimeric | GFSLSTYGM | WWDDD | RGHYSAMDY | 50 |
| | (SEQ ID NO: 130) | (SEQ ID NO: 146) | (SEQ ID NO: 19) | |
| | GFSLSTYGM | WWDDD | RGHYSAMDY | 72 |
| Sh14F11 IGHV2-5 | (SEQ ID NO: 130) | (SEQ ID NO: 146) | (SEQ ID NO: 19) | |
| Sh14F11 IGHV2- | GFSLSTYGM | WWDDD | RGHYSAMDY | 74 |
| 70 | (SEQ ID NO: 130) | (SEQ ID NO: 146) | (SEQ ID NO: 19) | |

IMGT

| | | | | |
|---|---|---|---|---|
| Ch01G06 | GYTFTDYN | INPNNGGI | AREAITTVGAMDY | 40 |
| Chimeric | (SEQ ID NO: 136) | (SEQ ID NO: 148) | (SEQ ID NO: 154) | |
| Hu01G06 | GYTFTDYN | INPNNGGI | AREAITTVGAMDY | 54 |
| IGHV1-18 | (SEQ ID NO: 136) | (SEQ ID NO: 148) | (SEQ ID NO: 154) | |
| Hu01G06 | GYTFTDYN | INPNNGGI | AREAITTVGAMDY | 56 |
| IGHV1-69 | (SEQ ID NO: 136) | (SEQ ID NO: 148) | (SEQ ID NO: 154) | |
| Sh01G06 | GYTFTDYN | INPNNGGI | AREAITTVGAMDY | 58 |
| IGHV1-18 M69L | (SEQ ID NO: 136) | (SEQ ID NO: 148) | (SEQ ID NO: 154) | |
| Sh01G06 | GYTFTDYN | INPNNGGI | AREAITTVGAMDY | 60 |
| IGHV1-18 M69L | (SEQ ID NO: 136) | (SEQ ID NO: 148) | (SEQ ID NO: 154) | |
| K64Q G44S | | | | |
| Sh01G06 | GYTFTDYN | INPNNGGI | AREAITTVGAMDY | 62 |
| IGHV1-18 M69L | (SEQ ID NO: 136) | (SEQ ID NO: 148) | (SEQ ID NO: 154) | |
| K64Q | | | | |
| Sh01G06 | GYTFSDYN | INPNNGGI | AREAITTVGAMDY | 64 |
| IGHV1-69 T30S | (SEQ ID NO: 235) | (SEQ ID NO: 148) | (SEQ ID NO: 154) | |
| I69L | | | | |
| Sh01G06 | GYTFSDYN | INPNNGGI | AREAITTVGAMDY | 66 |
| IGHV1-69 T30S | (SEQ ID NO: 235) | (SEQ ID NO: 148) | (SEQ ID NO: 154) | |
| K64Q I69L | | | | |
| Hu01G06 | GYTFTDYN | INPYNHLI | AREAITTVGAMDY | 246 |
| IGHV1-18 F1 | (SEQ ID NO: 136) | (SEQ ID NO: 242) | (SEQ ID NO: 154) | |
| Hu01G06 | GYTFTDYN | INPNNGLI | AREAITTVGAMDY | 248 |
| IGHV1-18 F2 | (SEQ ID NO: 136) | (SEQ ID NO: 243) | (SEQ ID NO: 154) | |
| Hu01G06 | GYTFSDYN | INPNNGLI | AREAITTVGAMDY | 250 |
| IGHV1-69 F1 | (SEQ ID NO: 235) | (SEQ ID NO: 243) | (SEQ ID NO: 154) | |
| Hu01G06 | GYTFSDYN | INPYNHLI | AREAITTVGAMDY | 252 |
| IGHV1-69 F2 | (SEQ ID NO: 235) | (SEQ ID NO: 242) | (SEQ ID NO: 154) | |
| Ch06C11 | GFSLNTYGMG | IYWDDDK | AQRGYDDYWGY | 46 |
| Chimeric | (SEQ ID NO: 141) | (SEQ ID NO: 150) | (SEQ ID NO: 157) | |
| HE LM 06C11 | GFSLNTYGMG | IYWDDDK | AQRGYDDYWGY | 68 |
| IGHV2-70 | (SEQ ID NO: 141) | (SEQ ID NO: 150) | (SEQ ID NO: 157) | |
| Hu06C11 | GFSLNTYGMG | IYWDDDK | AQRGYDDYWGY | 70 |
| IGHV2-5 | (SEQ ID NO: 141) | (SEQ ID NO: 150) | (SEQ ID NO: 157) | |
| Ch14F11 | GFSLSTYGMG | IWWDDDK | ARRGHYSAMDY | 50 |
| Chimeric | (SEQ ID NO: 140) | (SEQ ID NO: 152) | (SEQ ID NO: 158) | |
| Sh14F11 IGHV2- | GFSLSTYGMG | IWWDDDK | ARRGHYSAMDY | 72 |
| 5 | (SEQ ID NO: 140) | (SEQ ID NO: 152) | (SEQ ID NO: 158) | |
| Sh14F11 IGHV2- | GFSLSTYGMG | IWWDDDK | ARRGHYSAMDY | 74 |
| 70 | (SEQ ID NO: 140) | (SEQ ID NO: 152) | (SEQ ID NO: 158) | |

Humanized monoclonal antibody Kappa light chain CDR sequences (Kabat, Chothia, and IMGT definitions) are shown in Table 21.

TABLE 21

| | | | |
|---|---|---|---|
| | | | Variable Region |
| CDR1 | CDR2 | CDR3 | SEQ ID NO: |

Kabat/Chothia

| | | | | |
|---|---|---|---|---|
| Ch01G06 Chimeric | RTSENLHNYLA | DAKTLAD | QHFWSSPYT | 76 |
| | (SEQ ID NO: 21) | (SEQ ID NO: 26) | (SEQ ID NO: 32) | |
| Hu01G06 IGKV1-39 | RTSENLHNYLA | DAKTLAD | QHFWSSPYT | 90 |
| | (SEQ ID NO: 21) | (SEQ ID NO: 26) | (SEQ ID NO: 32) | |
| Hu01G06 IGKV1-39 | RTSENLHNYLA | DAKTLAD | QHFWSSPYT | 92 |
| S43A V48I (also | (SEQ ID NO: 21) | (SEQ ID NO: 26) | (SEQ ID NO: 32) | |
| known as Hu0106 | | | | |
| IGKV1-39 F1) | | | | |
| Hu01G06 IGKV1-39 | RTSENLHNYLA | DAKTLAD | QHFWSSPYT | 94 |
| V48I | (SEQ ID NO: 21) | (SEQ ID NO: 26) | (SEQ ID NO: 32) | |
| Hu01G06 IGKV1-39 | RTSENLHNYLA | DAKTLAD | QHFWSSPYT | 92 |
| F1 (also known as | (SEQ ID NO: 21) | (SEQ ID NO: 26) | (SEQ ID NO: 32) | |
| Hu01G06 IGKV1-39 | | | | |
| S43A V481) | | | | |
| Hu01G06 IGKV1-39 | RTSENLHNYLA | DAKTLAD | QHFWSDPYT | 254 |
| F2 | (SEQ ID NO: 21) | (SEQ ID NO: 26) | (SEQ ID NO: 244) | |
| Ch06C11 Chimeric | KASQNVGTNVA | SASYRYS | QQYNNYPLT | 82 |
| | (SEQ ID NO: 23) | (SEQ ID NO: 28) | (SEQ ID NO: 35) | |
| Sh06C11 IGKV1-16 | KASQNVGTNVA | SASYRYS | QQYNNYPLT | 96 |
| | (SEQ ID NO: 23) | (SEQ ID NO: 28) | (SEQ ID NO: 35) | |
| Ch14F11 Chimeric | KASQNVGTNVA | SPSYRYS | QQYNSYPHT | 86 |
| | (SEQ ID NO: 23) | (SEQ ID NO: 30) | (SEQ ID NO: 36) | |
| Hu14F11 IGKV1-16 | KASQNVGTNVA | SPSYRYS | QQYNSYPHT | 98 |
| | (SEQ ID NO: 23) | (SEQ ID NO: 30) | (SEQ ID NO: 36) | |

IMGT

| | | | | |
|---|---|---|---|---|
| Ch01G06 Chimeric | ENLHNY | DAK | QHFWSSPYT | 76 |
| | (SEQ ID NO: 160) | | (SEQ ID NO: 32) | |
| Hu01G06 IGKV1-39 | ENLHNY | DAK | QHFWSSPYT | 90 |
| | (SEQ ID NO: 160) | | (SEQ ID NO: 32) | |
| Hu01G06 IGKV1-39 | ENLHNY | DAK | QHFWSSPYT | 92 |
| S43A V48I (also | (SEQ ID NO: 160) | | (SEQ ID NO: 32) | |
| known as Hu01G06 | | | | |
| IGKV1-39 Fl) | | | | |
| Hu01G06 IGKV1-39 | ENLHNY | DAK | QHFWSSPYT | 94 |
| V48I | (SEQ ID NO: 160) | DAK | (SEQ ID NO: 32) | |
| Hu01G06 IGKV1-39 | ENLHNY | | QHFWSSPYT | 92 |
| F1 (also known as | (SEQ ID NO: 160) | | (SEQ ID NO: 32) | |
| Hu01G06 IGKV1-39 | | | | |
| S43A V48I) | | | | |
| Hu01G06 IGKV1-39 | ENLHNY | DAK | QHFWSDPYT | 254 |
| F2 | (SEQ ID NO: 160) | | (SEQ ID NO: 244) | |
| Ch06C11 Chimeric | QNVGTN | SAS | QQYNNYPLT | 82 |
| | (SEQ ID NO: 162) | | (SEQ ID NO: 35) | |
| Sh06C11 IGKV1-16 | QNVGTN | SAS | QQYNNYPLT | 96 |
| | (SEQ ID NO: 162) | | (SEQ ID NO: 35) | |
| Ch14F11 Chimeric | QNVGTN | SPS | QQYNSYPHT | 86 |
| | (SEQ ID NO: 162) | | (SEQ ID NO: 36) | |
| Hu14F11 IGKV1-16 | QNVGTN | SPS | QQYNSYPHT | 98 |
| | (SEQ ID NO: 162) | | (SEQ ID NO: 36) | |

To create the complete chimeric and humanized heavy or kappa chain antibody sequences, each variable sequence above is combined with its respective human constant region. For example, a complete heavy chain comprises a heavy variable sequence followed by a human IgG1 heavy chain constant sequence. A complete kappa chain comprises a kappa variable sequence followed by the human kappa light chain constant sequence.

Nucleic Acid Sequence Encoding the Human IgG1 Heavy Chain Constant Region (SEQ ID NO:171)

| | |
|---|---|
| 1 | gcctcaacaa aaggaccaag tgtgttccca ctcgccccta |
| | gcagcaagag tacatccggg |
| 61 | ggcactgcag cactcggctg cctcgtcaag gattattttc |
| | cagagccagt aaccgtgagc |
| 121 | tggaacagtg gagcactcac ttctggtgtc catacttttc |
| | ctgctgtcct gcaaagctct |
| 181 | ggcctgtact cactcagctc cgtcgtgacc gtgccatctt |
| | catctctggg cactcagacc |
| 241 | tacatctgta atgtaaacca caagcctagc aatactaagg |
| | tcgataagcg ggtggaaccc |
| 301 | aagagctgcg acaagactca cacttgtccc ccatgccctg |
| | cccctgaact tctgggcggt |
| 361 | cccagcgtct ttttgttccc accaaagcct aaagatactc |
| | tgatgataag tagaacaccc |
| 421 | gaggtgacat gtgttgttgt agacgtttcc cacgaggacc |
| | cagaggttaa gttcaactgg |
| 481 | tacgttgatg gagtcgaagt acataatgct aagaccaagc |
| | ctagagagga gcagtataat |
| 541 | agtacatacc gtgtagtcag tgttctcaca gtgctgcacc |
| | aagactggct caacggcaaa |
| 601 | gaatacaaat gcaaagtgtc caacaaagca ctcccagccc |
| | ctatcgagaa gactattagt |
| 661 | aaggcaaagg ggcagcctcg tgaaccacag gtgtacactc |
| | tgccacccag tagagaggaa |
| 721 | atgacaaaga accaagtctc attgacctgc ctggtgaaag |
| | gcttctaccc cagcgacatc |
| 781 | gccgttgagt gggagagtaa cggtcagcct gagaacaatt |
| | acaagacaac ccccccagtg |
| 841 | ctggatagtg acgggtcttt ctttctgtac agtaagctga |
| | ctgtggacaa gtcccgctgg |
| 901 | cagcagggta acgtcttcag ctgttccgtg atgcacgagg |
| | cattgcacaa ccactacacc |
| 961 | cagaagtcac tgagcctgag cccagggaag |

Protein Sequence Defining the Human IgG1 Heavy Chain Constant Region (SEQ ID NO:172)

| | |
|---|---|
| 1 | astkgpsvfp lapsskstsg gtaalgclvk dyfpepvtvs |
| | wnsgaltsgv htfpavlqss |
| 61 | glyslssvvt vpssslgtqt yicnvnhkps ntkvdkrvep |
| | kscdkthtcp pcpapellgg |
| 121 | psvflfppkp kdtlmisrtp evtcvvvdvs hedpevkfnw |
| | yvdgvevhna ktkpreeqyn |
| 181 | styrvvsvlt vlhqdwlngk eykckvsnka lpapiektis |
| | kakgqprepq vytlppsree |
| 241 | mtknqvsltc lvkgfypsdi avewesngqp ennykttppv |
| | ldsdgsffly skltvdksrw |
| 301 | qqgnvfscsv mhealhnhyt qkslslspgk |

Nucleic Acid Sequence Encoding the Human Kappa Light Chain Constant Region (SEQ ID NO:173)

| | |
|---|---|
| 1 | cgcacagttg ctgcccccag cgtgttcatt ttcccaccta |
| | gcgatgagca gctgaaaagc |
| 61 | ggtactgcct ctgtcgtatg cttgctcaac aacttttacc |
| | cacgtgaggc taaggtgcag |
| 121 | tggaaagtgg ataatgcact tcaatctgga aacagtcaag |
| | agtccgtgac agaacaggac |
| 181 | agcaaagact caacttattc actctcttcc accctgactc |
| | tgtccaaggc agactatgaa |
| 241 | aaacacaagg tatacgcctg cgaggttaca caccagggtt |
| | tgtctagtcc tgtcaccaag |
| 301 | tccttcaata ggggcgaatg t |

Protein Sequence Defining the Human Kappa Light Chain Constant Region (SEQ ID NO:174)

| | |
|---|---|
| 1 | rtvaapsvfi fppsdeqlks gtasvvclln nfypreakvq |
| | wkvdnalqsg nsqesvteqd |
| 61 | skdstyslss tltlskadye khkvyacevt hqglsspvtk |
| | sfnrgec |

The following sequences represent the actual or contemplated full length heavy and light chain sequence (i.e., containing both the variable and constant regions sequences) for each antibody described in this Example. Signal sequences for proper secretion of the antibodies (e.g., signal sequences at the 5′ end of the DNA sequences or the amino terminal end of the protein sequences) are not shown in the full length heavy and light chain sequences disclosed herein and are not included in the final secreted protein. Also not shown are stop codons for termination of translation required at the 3′ end of the DNA sequences. It is within ordinary skill in the art to select a signal sequence and/or a stop codon for expression of the disclosed full length immunoglobulin heavy chain and light chain sequences. It is also contemplated that the variable region sequences can be ligated to other constant region sequences to produce active full length immunoglobulin heavy and light chains.

Nucleic Acid Sequence Encoding the Full Length Ch01G06 Chimeric Heavy Chain (Mouse Heavy Chain Variable Region and Human IgG1 Constant Region) (SEQ ID NO:175)

| | |
|---|---|
| 1 | gaagtgttgt tgcagcagtc agggccggag ttggtaaaac |
| | cgggagcgtc ggtgaaaatc |
| 61 | ccgtgcaaag cgtcggggta tacgtttacg gactataaca |
| | tggattgggt gaaacagtcg |
| 121 | catgggaaat cgcttgaatg gattggtcag atcaatccga |
| | ataatggagg aatcttcttt |
| 181 | aatcagaagt ttaaaggaaa agcgacgctt acagtcgata |
| | agtcgtcgaa cacggcgttc |
| 241 | atggaagtac ggtcgcttac gtcggaagat acggcggtct |
| | attactgtgc gagggaggcg |
| 301 | attacgacgg tgggagcgat ggactattgg ggacaaggga |
| | cgtcggtcac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Ch01G06 Chimeric Heavy Chain (Mouse Heavy Chain Variable Region and Human IgG1 Constant Region) (SEQ ID NO:176)

| | |
|---|---|
| 1 | evllqqsgpe lvkpgasvki pckasgytft dynmdwvkqs |
| | hgkslewigq inpnnggiff |
| 61 | nqkfkgkatl tvdkssntaf mevrsltsed tavyycarea |
| | ittvgamdyw gqgtsvtvss |
| 121 | astkgpsvfp lapsskstsg gtaalgclvk dyfpepvtvs |
| | wnsgaltsgv htfpavlqss |
| 181 | glyslssvvt vpssslgtqt yicnvnhkps ntkvdkrvep |
| | kscdkthtcp pcpapellgg |
| 241 | psvflfppkp kdtlmisrtp evtcvvvdvs hedpevkfnw |
| | yvdgvevhna ktkpreeqyn |
| 301 | styrvvsvlt vlhqdwlngk eykckvsnka lpapiektis |
| | kakgqprepq vytlppsree |
| 361 | mtknqvsltc lvkgfypsdi avewesngqp ennykttppv |
| | ldsdgsffly skltvdksrw |
| 421 | qqgnvfscsv mhealhnhyt qkslslspgk |

Nucleic Acid Sequence Encoding the Full Length Hu01G06 IGHV1-18 Heavy Chain (Humanized Heavy Chain Variable Region and Human IgG1 Constant Region) (SEQ ID NO:177)

| | |
|---|---|
| 1 | caagtgcaac ttgtgcagtc gggtgcggaa gtcaaaaagc |
| | cgggagcgtc ggtgaaagta |
| 61 | tcgtgtaaag cgtcgggata tacgtttacg gactataaca |
| | tggactgggt acgacaggca |
| 121 | ccggggaaat cgttggaatg gatcggacag attaatccga |
| | acaatggggg aattttcttt |
| 181 | aatcagaaat tcaaaggacg ggcgacgttg acggtcgata |
| | catcgacgaa tacggcgtat |
| 241 | atggaattga ggtcgcttcg ctcggacgat acggcggtct |
| | attactgcgc cagggaggcg |
| 301 | atcacgacgg taggggcgat ggattattgg ggacagggga |
| | cgcttgtgac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Hu01G06 IGHV1-18 Heavy Chain (Humanized Heavy Chain Variable Region and Human IgG1 Constant Region) (SEQ ID NO:178)

| | |
|---|---|
| 1 | qvqlvqsgae vkkpgasvkv sckasgytft dynmdwvrqa |
| | pgkslewigq inpnnggiff |
| 61 | nqkfkgratl tvdtstntay melrslrsdd tavyycarea |
| | ittvgamdyw gqgtlvtvss |
| 121 | astkgpsvfp lapsskstsg gtaalgclvk dyfpepvtvs |
| | wnsgaltsgv htfpavlqss |
| 181 | glyslssvvt vpssslgtqt yicnvnhkps ntkvdkrvep |
| | kscdkthtcp pcpapellgg |
| 241 | psvflfppkp kdtlmisrtp evtcvvvdvs hedpevkfnw |
| | yvdgvevhna ktkpreeqyn |
| 301 | styrvvsvlt vlhqdwlngk eykckvsnka lpapiektis |
| | kakgqprepq vytlppsree |
| 361 | mtknqvsltc lvkgfypsdi avewesngqp ennykttppv |
| | ldsdgsffly skltvdksrw |
| 421 | qqgnvfscsv mhealhnhyt qkslslspgk |

Nucleic Acid Sequence Encoding the Full Length Hu01G06 IGHV1-69 Heavy Chain (Humanized Heavy Chain Variable Region and Human IgG1 Constant Region) (SEQ ID NO:179)

| | |
|---|---|
| 1 | caagtccagc ttgtccagtc gggagcggaa gtgaagaaac |
| | cggggtcgtc ggtcaaagta |
| 61 | tcgtgtaaag cgtcgggata tacgtttacg gactataaca |
| | tggattgggt acgacaggct |
| 121 | ccgggaaaat cattggaatg gattggacag attaatccga |
| | ataatggggg tatcttcttt |
| 181 | aatcaaaagt ttaaagggag ggcgacgttg acggtggaca |
| | aatcgacaaa tacggcgtat |
| 241 | atggaattgt cgtcgcttcg gtcggaggac acggcggtgt |
| | attactgcgc gagggaggcg |
| 301 | atcacgacgg tcggggcgat ggattattgg ggacagggaa |
| | cgcttgtgac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Hu01G06 IGHV1-69 Heavy Chain (Humanized Heavy Chain Variable Region and Human IgG1 Constant Region) (SEQ ID NO:180)

| | |
|---|---|
| 1 | qvqlvqsgae vkkpgssvkv sckasgytft dynmdwvrqa |
| | pgkslewigq inpnnggiff |
| 61 | nqkfkgratl tvdkstntay melsslrsed tavyycarea |
| | ittvgamdyw gqgtlvtvss |
| 121 | astkgpsvfp lapsskstsg gtaalgclvk dyfpepvtvs |
| | wnsgaltsgv htfpavlqss |
| 181 | glyslssvvt vpssslgtqt yicnvnhkps ntkvdkrvep |
| | kscdkthtcp pcpapellgg |
| 241 | psvflfppkp kdtlmisrtp evtcvvvdvs hedpevkfnw |
| | yvdgvevhna ktkpreeqyn |
| 301 | styrvvsvlt vlhqdwlngk eykckvsnka lpapiektis |
| | kakgqprepq vytlppsree |
| 361 | mtknqvsltc lvkgfypsdi avewesngqp ennykttppv |
| | ldsdgsffly skltvdksrw |
| 421 | qqgnvfscsv mhealhnhyt qkslslspgk |

Nucleic Acid Sequence Encoding the Full Length Sh01G06 IGHV1-18 M69L Heavy Chain (Humanized Heavy Chain Variable Region and Human IgG1 Constant Region) (SEQ ID NO:181)

| | |
|---|---|
| 1 | caggtccagc ttgtgcaatc gggagcggaa gtgaagaaac |
| | cgggagcgtc ggtaaaagtc |
| 61 | tcgtgcaaag cgtcggggta tacgtttacg gactataaca |
| | tggactgggt gcgccaagcg |
| 121 | cctggacagg gtcttgaatg gatggggcag attaatccga |
| | ataatggagg gatcttcttt |
| 181 | aatcagaaat tcaaaggaag ggtaacgctg acgacagaca |
| | cgtcaacatc gacggcctat |
| 241 | atggaattgc ggtcgttgcg atcagatgat acggcggtct |
| | actattgtgc gagggaggcg |
| 301 | attacgacgg tgggagcgat ggattattgg ggacagggga |
| | cgttggtaac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Sh01G06 IGHV1-18 M69L Heavy Chain (Humanized Heavy Chain Variable Region and Human IgG1 Constant Region) (SEQ ID NO:182)

| | |
|---|---|
| 1 | qvqlvqsgae vkkpgasvkv sckasgytft dynmdwvrqa |
| | pgqglewmgq inpnnggiff |
| 61 | nqkfkgrvtl ttdtststay melrslrsdd tavyycarea |
| | ittvgamdyw gqgtlvtvss |
| 121 | astkgpsvfp lapsskstsg gtaalgclvk dyfpepvtvs |
| | wnsgaltsgv htfpavlqss |
| 181 | glyslssvvt vpssslgtqt yicnvnhkps ntkvdkrvep |
| | kscdkthtcp pcpapellgg |
| 241 | psvflfppkp kdtlmisrtp evtcvvvdvs hedpevkfnw |
| | yvdgvevhna ktkpreeqyn |
| 301 | styrvvsvlt vlhqdwlngk eykckvsnka lpapiektis |
| | kakgqprepq vytlppsree |
| 361 | mtknqvsltc lvkgfypsdi avewesngqp ennykttppv |
| | ldsdgsffly skltvdksrw |
| 421 | qqgnvfscsv mhealhnhyt qkslslspgk |

Nucleic Acid Sequence Encoding the Full Length Sh01G06 IGHV1-18 M69L K64Q G44S Heavy Chain (Humanized Heavy Chain Variable Region and Human IgG1 Constant Region) (SEQ ID NO:183)

| | |
|---|---|
| 1 | caggtccagc ttgtgcaatc gggagcggaa gtgaagaaac |
| | cgggagcgtc ggtaaaagtc |
| 61 | tcgtgcaaag cgtcggggta tacgtttacg gactataaca |
| | tggactgggt gcgccaagcg |
| 121 | cctggacaga gccttgaatg gatggggcag attaatccga |
| | ataatggagg gatcttcttt |
| 181 | aatcagaaat tccagggaag ggtaacgctg acgacagaca |
| | cgtcaacatc gacggcctat |
| 241 | atggaattgc ggtcgttgcg atcagatgat acggcggtct |
| | actattgtgc gagggaggcg |
| 301 | attacgacgg tgggagcgat ggattattgg ggacagggga |
| | cgttggtaac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Sh01G06 IGHV1-18 M69L K64Q G44S Heavy Chain (Humanized Heavy Chain Variable Region and Human IgG1 Constant Region) (SEQ ID NO:184)

| | |
|---|---|
| 1 | qvqlvqsgae vkkpgasvkv sckasgytft dynmdwvrqa |
| | pgqslewmgq inpnnggiff |
| 61 | nqkfqgrvtl ttdtststay melrslrsdd tavyycarea |
| | ittvgamdyw gqgtlvtvss |
| 121 | astkgpsvfp lapsskstsg gtaalgclvk dyfpepvtvs |
| | wnsgaltsgv htfpavlqss |
| 181 | glyslssvvt vpssslgtqt yicnvnhkps ntkvdkrvep |
| | kscdkthtcp pcpapellgg |
| 241 | psvflfppkp kdtlmisrtp evtcvvvdvs hedpevkfnw |
| | yvdgvevhna ktkpreeqyn |
| 301 | styrvvsvlt vlhqdwlngk eykckvsnka lpapiektis |
| | kakgqprepq vytlppsree |
| 361 | mtknqvsltc lvkgfypsdi avewesngqp ennykttppv |
| | ldsdgsffly skltvdksrw |
| 421 | qqgnvfscsv mhealhnhyt qkslslspgk |

Nucleic Acid Sequence Encoding the Full Length Sh01G06 IGHV1-18 M69L K64Q Heavy Chain (Humanized Heavy Chain Variable Region and Human IgG1 Constant Region) (SEQ ID NO:185)

| | |
|---|---|
| 1 | caggtccagc ttgtgcaatc gggagcggaa gtgaagaaac |
| | cgggagcgtc ggtaaaagtc |
| 61 | tcgtgcaaag cgtcggggta tacgtttacg gactataaca |
| | tggactgggt gcgccaagcg |
| 121 | cctggacagg gtcttgaatg gatggggcag attaatccga |
| | ataatggagg gatcttcttt |
| 181 | aatcagaaat tccagggaag ggtaacgctg acgacagaca |
| | cgtcaacatc gacggcctat |
| 241 | atggaattgc ggtcgttgcg atcagatgat acggcggtct |
| | actattgtgc gagggaggcg |
| 301 | attacgacgg tgggagcgat ggattattgg ggacagggga |
| | cgttggtaac ggtatcgtcg |
| 361 | gcctcaacaa aaggaccaag tgtgttccca ctcgccccta |
| | gcagcaagag tacatccggg |
| 421 | ggcactgcag cactcggctg cctcgtcaag gattattttc |
| | cagagccagt aaccgtgagc |
| 481 | tggaacagtg gagcactcac ttctggtgtc catacttttc |
| | ctgctgtcct gcaaagctct |
| 541 | ggcctgtact cactcagctc cgtcgtgacc gtgccatctt |
| | catctctggg cactcagacc |
| 601 | tacatctgta atgtaaacca caagcctagc aatactaagg |
| | tcgataagcg ggtggaaccc |
| 661 | aagagctgcg acaagactca cacttgtccc ccatgccctg |
| | cccctgaact tctgggcggt |
| 721 | cccagcgtct ttttgttccc accaaagcct aaagatactc |
| | tgatgataag tagaacaccc |
| 781 | gaggtgacat gtgttgttgt agacgtttcc cacgaggacc |
| | cagaggttaa gttcaactgg |
| 841 | tacgttgatg gagtcgaagt acataatgct aagaccaagc |
| | ctagagagga gcagtataat |
| 901 | agtacatacc gtgtagtcag tgttctcaca gtgctgcacc |
| | aagactggct caacggcaaa |
| 961 | gaatacaaat gcaaagtgtc caacaaagca ctcccagccc |
| | ctatcgagaa gactattagt |
| 1021 | aaggcaaagg ggcagcctcg tgaaccacag gtgtacactc |
| | tgccacccag tagagaggaa |
| 1081 | atgacaaaga accaagtctc attgacctgc ctggtgaaag |
| | gcttctaccc cagcgacatc |
| 1141 | gccgttgagt gggagagtaa cggtcagcct gagaacaatt |
| | acaagacaac ccccccagtg |
| 1201 | ctggatagtg acgggtcttt ctttctgtac agtaagctga |
| | ctgtggacaa gtcccgctgg |
| 1261 | cagcagggta acgtcttcag ctgttccgtg atgcacgagg |
| | cattgcacaa ccactacacc |
| 1321 | cagaagtcac tgagcctgag cccagggaag |

Protein Sequence Defining the Full Length Sh01G06 IGHV1-18 M69L K64Q Heavy Chain (Humanized Heavy Chain Variable Region and Human IgG1 Constant Region) (SEQ ID NO:186)

| | |
|---|---|
| 1 | qvqlvqsgae vkkpgasvkv sckasgytft dynmdwvrqa pgqglewmgq inpnnggiff |
| 61 | nqkfqgrvtl ttdtststay melrslrsdd tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Sh01G06 IGHV1-69 T30S I69L Heavy Chain (Humanized Heavy Chain Variable Region and Human IgG1 Constant Region) (SEQ ID NO:187)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatggggg aatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Sh01G06 IGHV1-69 T30S I69L Heavy Chain (Humanized Heavy Chain Variable Region and Human IgG1 Constant Region) (SEQ ID NO:188)

| | |
|---|---|
| 1 | qvqlvqsgae vkkpgssvkv sckasgytfs dynmdwvrqa pgqglewmgq inpnnggiff |
| 61 | nqkfkgrvtl tadkststay melsslrsed tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Sh01G06 IGHV1-69 T30S K640 I69L Heavy Chain (Humanized Heavy Chain Variable Region and Human IgG1 Constant Region) (SEQ ID NO:189)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatggggg aatctttttc |
| 181 | aatcagaagt ttcaggggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Sh01G06 IGHV1-69 T30S K64Q I69L Heavy Chain (Humanized Heavy Chain Variable Region and Human IgG1 Constant Region) (SEQ ID NO:190)

| | |
|---|---|
| 1 | qvqlvqsgae vkkpgssvkv sckasgytfs dynmdwvrqa pgqglewmgq inpnnggiff |
| 61 | nqkfqgrvtl tadkststay melsslrsed tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Hu01G06 IGHV1-18 F1 Heavy Chain (Humanized Heavy Chain Variable Region and Human IgG1 Constant Region) (SEQ ID NO:255)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacaga gccttgaatg gatggggcag attaatccgt acaatcacct gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Hu01G06 IGHV1-18 F1 Heavy Chain (Humanized Heavy Chain Variable Region and Human IgG1 Constant Region) (SEQ ID NO:256)

| | |
|---|---|
| 1 | qvqlvqsgae vkkpgasvkv sckasgytft dynmdwvrqa pgqslewmgq inpynhliff |
| 61 | nqkfqgrvtl ttdtststay melrslrsdd tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Hu01G06 IGHV1-18 F2 Heavy Chain (Humanized Heavy Chain Variable Region and Human IgG1 Constant Region) (SEQ ID NO:257)

| | |
|---|---|
| 1 | caggtccagc ttgtgcaatc gggagcggaa gtgaagaaac cgggagcgtc ggtaaaagtc |
| 61 | tcgtgcaaag cgtcggggta tacgtttacg gactataaca tggactgggt gcgccaagcg |
| 121 | cctggacaga gccttgaatg gatggggcag attaatccga ataatggact gatcttcttt |
| 181 | aatcagaaat tccagggaag ggtaacgctg acgacagaca cgtcaacatc gacggcctat |
| 241 | atggaattgc ggtcgttgcg atcagatgat acggcggtct actattgtgc gagggaggcg |
| 301 | attacgacgg tgggagcgat ggattattgg ggacagggga cgttggtaac ggtatcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Hu01G06 IGHV1-18 F2 Heavy Chain (Humanized Heavy Chain Variable Region and Human IgG1 Constant Region) (SEQ ID NO:258)

| | |
|---|---|
| 1 | qvqlvqsgae vkkpgasvkv sckasgytft dynmdwvrqa pgqslewmgq inpnngliff |
| 61 | nqkfqgrvtl ttdtststay melrslrsdd tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Hu01G06 IGHV1-69 F1 Heavy Chain (Humanized Heavy Chain Variable Region and Human IgG1 Constant Region) (SEQ ID NO:259)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccga ataatgggct gatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Hu01G06 IGHV1-69 F1 Heavy Chain (Humanized Heavy Chain Variable Region and Human IgG1 Constant Region) (SEQ ID NO:260)

| | |
|---|---|
| 1 | qvqlvqsgae vkkpgssvkv sckasgytfs dynmdwvrqa pgqglewmgq inpnngliff |
| 61 | nqkfkgrvtl tadkststay melsslrsed tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Hu01G06 IGHV1-69 F2 Heavy Chain (Humanized Heavy Chain Variable Region and Human IgG1 Constant Region) (SEQ ID NO:261)

| | |
|---|---|
| 1 | caagtacagc ttgtacagtc gggagcggaa gtcaagaaac cgggatcgtc ggtcaaagtg |
| 61 | tcgtgtaaag cgtcgggata tacgtttagc gactataaca tggattgggt gcgacaagcg |
| 121 | cctgggcagg gacttgaatg gatgggtcag atcaatccgt acaatcacct gatctttttc |
| 181 | aatcagaagt ttaaagggag ggtaacgctg acggcggata aaagcacgtc aacggcgtat |
| 241 | atggagttgt cgtcgttgcg gtcggaggac acggcggtct attactgcgc gagggaagcg |
| 301 | attacgacgg tgggagcgat ggattattgg gggcagggaa cgcttgtaac ggtgtcatcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |

Protein Sequence Defining the Full Length Hu01G06 IGHV1-69 F2 Heavy Chain (Humanized Heavy Chain Variable Region and Human IgG1 Constant Region) (SEQ ID NO:262)

| | |
|---|---|
| 1 | qvqlvqsgae vkkpgssvkv sckasgytfs dynmdwvrqa pgqglewmgq inpynhliff |
| 61 | nqkfkgrvtl tadkststay melsslrsed tavyycarea ittvgamdyw gqgtivtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |

Nucleic Acid Sequence Encoding the Full Length Ch06C11 Chimeric Heavy Chain (Mouse Heavy Chain Variable Region and Human IgG1 Constant Region) (SEQ ID NO:191)

| | |
|---|---|
| 1 | caggtgacac tcaaagaatc aggacccgga atccttcagc ccagccagac cttgtcgctg |
| 61 | acttgttcgt tctccggttt cagcctgaat acttatggga tgggtgtgtc atggatcagg |
| 121 | caaccgtccg ggaaaggatt ggagtggctc gcgcacatct actgggacga tgacaaacgc |
| 181 | tacaatcctt cgctgaagag ccgattgacg atttccaagg atgcctcgaa caaccgggta |
| 241 | tttcttaaga tcacgtcggt cgatacggca gacacggcga cctattactg cgcccaaaga |
| 301 | gggtacgatg actattgggg atattggggc caggggacac tcgtcacaat ttcagctgcc |
| 361 | tcaacaaaag gaccaagtgt gttcccactc gcccctagca gcaagagtac atccgggggc |
| 421 | actgcagcac tcggctgcct cgtcaaggat tattttccag agccagtaac cgtgagctgg |
| 481 | aacagtggag cactcacttc tggtgtccat acttttcctg ctgtcctgca aagctctggc |
| 541 | ctgtactcac tcagctccgt cgtgaccgtg ccatcttcat ctctgggcac tcagacctac |
| 601 | atctgtaatg taaaccacaa gcctagcaat actaaggtcg ataagcgggt ggaacccaag |
| 661 | agctgcgaca agactcacac ttgtccccca tgccctgccc ctgaacttct gggcggtccc |
| 721 | agcgtctttt tgttcccacc aaagcctaaa gatactctga tgataagtag aacacccgag |
| 781 | gtgacatgtg ttgttgtaga cgtttcccac gaggacccag aggttaagtt caactggtac |
| 841 | gttgatggag tcgaagtaca taatgctaag accaagccta gagaggagca gtataatagt |
| 901 | acataccgtg tagtcagtgt tctcacagtg ctgcaccaag actggctcaa cggcaaagaa |
| 961 | tacaaatgca aagtgtccaa caaagcactc ccagccccta tcgagaagac tattagtaag |
| 1021 | gcaaaggggc agcctcgtga accacaggtg tacactctgc cacccagtag agaggaaatg |
| 1081 | acaaagaacc aagtctcatt gacctgcctg gtgaaaggct tctaccccag cgacatcgcc |
| 1141 | gttgagtggg agagtaacgg tcagcctgag aacaattaca agacaacccc cccagtgctg |
| 1201 | gatagtgacg ggtctttctt tctgtacagt aagctgactg tggacaagtc ccgctggcag |
| 1261 | cagggtaacg tcttcagctg ttccgtgatg cacgaggcat tgcacaacca ctacacccag |
| 1321 | aagtcactga gcctgagccc agggaag |

Protein Sequence Defining the Full Length Ch06C11 Chimeric Heavy Chain (Mouse Heavy Chain Variable Region and Human IgG1 Constant Region) (SEQ ID NO:192)

| | |
|---|---|
| 1 | qvtlkesgpg ilqpsqtlsl tcsfsgfsln tygmgvswir qpsgkglewl ahiywdddkr |
| 61 | ynpslksrlt iskdasnnry flkitsvdta dtatyycaqr gyddywgywg qgtivtisaa |
| 121 | stkgpsvfpl apsskstsgg taalgclvkd yfpepvtvsw nsgaltsgvh tfpavlqssg |
| 181 | lyslssvvtv pssslgtqty icnvnhkpsn tkvdkrvepk scdkthtcpp cpapellggp |
| 241 | svflfppkpk dtlmisrtpe vtcvvvdvsh edpevkfnwy vdgvevhnak tkpreeqyns |
| 301 | tyrvvsvltv lhqdwlngke ykckvsnkal papiektisk akgqprepqv ytlppsreem |
| 361 | tknqvsltcl vkgfypsdia vewesngqpe nnykttppvl dsdgsfflys kltvdksrwq |
| 421 | qgnvfscsvm healhnhytq kslslspgk |

Nucleic Acid Sequence Encoding the Full Length HE LM 06C11 IGHV2-70 Heavy Chain (Humanized Heavy Chain Variable Region and Human IgG1 Constant Region) (SEQ ID NO:193)

| | |
|---|---|
| 1 | caggtgactt tgaaagaatc cggtcccgca ttggtaaagc |
| | caacccagac acttacgctc |
| 61 | acatgtacat tttccggatt cagcttgaac acttacggga |
| | tgggagtgtc gtggattcgg |
| 121 | caacctccgg ggaaggctct ggagtggctg gcgcacatct |
| | actgggatga tgacaaaagg |
| 181 | tataacccct cacttaaaac gagactgacg atctcgaagg |
| | acacaagcaa gaatcaggtc |
| 241 | gtcctcacga ttacgaatgt agacccggtg gatactgccg |
| | tctattactg cgcgcaacgc |
| 301 | gggtatgatg actactgggg atattggggt cagggcaccc |
| | tcgtgaccat ctcgtcagcc |
| 361 | tcaacaaaag gaccaagtgt gttcccactc gcccctagca |
| | gcaagagtac atccgggggc |
| 421 | actgcagcac tcggctgcct cgtcaaggat tattttccag |
| | agccagtaac cgtgagctgg |
| 481 | aacagtggag cactcacttc tggtgtccat acttttcctg |
| | ctgtcctgca aagctctggc |
| 541 | ctgtactcac tcagctccgt cgtgaccgtg ccatcttcat |
| | ctctgggcac tcagacctac |
| 601 | atctgtaatg taaaccacaa gcctagcaat actaaggtcg |
| | ataagcgggt ggaacccaag |
| 661 | agctgcgaca agactcacac ttgtccccca tgccctgccc |
| | ctgaacttct gggcggtccc |
| 721 | agcgtctttt tgttcccacc aaagcctaaa gatactctga |
| | tgataagtag aacacccgag |
| 781 | gtgacatgtg ttgttgtaga cgtttcccac gaggacccag |
| | aggttaagtt caactggtac |
| 841 | gttgatggag tcgaagtaca taatgctaag accaagccta |
| | gagaggagca gtataatagt |
| 901 | acataccgtg tagtcagtgt tctcacagtg ctgcaccaag |
| | actggctcaa cggcaaagaa |
| 961 | tacaaatgca aagtgtccaa caaagcactc ccagccccta |
| | tcgagaagac tattagtaag |
| 1021 | gcaaaggggc agcctcgtga accacaggtg tacactctgc |
| | cacccagtag agaggaaatg |
| 1081 | acaaagaacc aagtctcatt gacctgcctg gtgaaaggct |
| | tctaccccag cgacatcgcc |
| 1141 | gttgagtggg agagtaacgg tcagcctgag aacaattaca |
| | agacaacccc cccagtgctg |
| 1201 | gatagtgacg ggtctttctt tctgtacagt aagctgactg |
| | tggacaagtc ccgctggcag |
| 1261 | cagggtaacg tcttcagctg ttccgtgatg cacgaggcat |
| | tgcacaacca ctacacccag |
| 1321 | aagtcactga gcctgagccc agggaag |

Protein Sequence Defining the Full Length HE LM 06C11 IGHV2-70 Heavy Chain (Humanized Heavy Chain Variable Region and Human IgG1 Constant Region) (SEQ ID NO:194)

| | |
|---|---|
| 1 | qvtlkesgpa lvkptqtltl tctfsgfsln tygmgvswir |
| | qppgkalewl ahiywdddkr |
| 61 | ynpslktrlt iskdtsknqv vltitnvdpv dtavyycaqr |
| | gyddywgywg qgtivtissa |
| 121 | stkgpsvfpl apsskstsgg taalgclvkd yfpepvtvsw |
| | nsgaltsgvh tfpavlqssg |
| 181 | lyslssvvtv pssslgtqty icnvnhkpsn tkvdkrvepk |
| | scdkthtcpp cpapellggp |
| 241 | svflfppkpk dtlmisrtpe vtcvvvdvsh edpevkfnwy |
| | vdgvevhnak tkpreeqyns |
| 301 | tyrvvsvltv lhqdwlngke ykckvsnkal papiektisk |
| | akgqprepqv ytlppsreem |
| 361 | tknqvsltcl vkgfypsdia vewesngqpe nnykttppvl |
| | dsdgsfflys kltvdksrwq |
| 421 | qgnvfscsvm healhnhytq kslslspgk |

Nucleic Acid Sequence Encoding the Full Length Hu06C11 IGHV2-5 Heavy Chain (Humanized Heavy Chain Variable Region and Human IgG1 Constant Region) (SEQ ID NO:195)

| | |
|---|---|
| 1 | caagtaacgc tcaaggagtc cggacccacc ttggtgaagc |
| | cgacgcagac cttgactctt |
| 61 | acgtgcactt tctcggggtt ttcactgaat acgtacggga |
| | tgggtgtctc atggatcagg |
| 121 | caacctccgg ggaaaggatt ggaatggctg gcgcacatct |
| | actgggatga cgataagaga |
| 181 | tataacccaa gcctcaagtc gcggctcacc attacaaaag |
| | atacatcgaa aaatcaggtc |
| 241 | gtacttacta tcacgaacat ggaccccgtg gacacagcaa |
| | catattactg tgcccagcgc |
| 301 | ggctatgacg attattgggg ttactgggga cagggaacac |
| | tggtcacggt gtccagcgcc |
| 361 | tcaacaaaag gaccaagtgt gttcccactc gcccctagca |
| | gcaagagtac atccgggggc |
| 421 | actgcagcac tcggctgcct cgtcaaggat tattttccag |
| | agccagtaac cgtgagctgg |
| 481 | aacagtggag cactcacttc tggtgtccat acttttcctg |
| | ctgtcctgca aagctctggc |
| 541 | ctgtactcac tcagctccgt cgtgaccgtg ccatcttcat |
| | ctctgggcac tcagacctac |
| 601 | atctgtaatg taaaccacaa gcctagcaat actaaggtcg |
| | ataagcgggt ggaacccaag |
| 661 | agctgcgaca agactcacac ttgtccccca tgccctgccc |
| | ctgaacttct gggcggtccc |
| 721 | agcgtctttt tgttcccacc aaagcctaaa gatactctga |
| | tgataagtag aacacccgag |
| 781 | gtgacatgtg ttgttgtaga cgtttcccac gaggacccag |
| | aggttaagtt caactggtac |
| 841 | gttgatggag tcgaagtaca taatgctaag accaagccta |
| | gagaggagca gtataatagt |
| 901 | acataccgtg tagtcagtgt tctcacagtg ctgcaccaag |
| | actggctcaa cggcaaagaa |
| 961 | tacaaatgca aagtgtccaa caaagcactc ccagccccta |
| | tcgagaagac tattagtaag |
| 1021 | gcaaaggggc agcctcgtga accacaggtg tacactctgc |
| | cacccagtag agaggaaatg |
| 1081 | acaaagaacc aagtctcatt gacctgcctg gtgaaaggct |
| | tctaccccag cgacatcgcc |
| 1141 | gttgagtggg agagtaacgg tcagcctgag aacaattaca |
| | agacaacccc cccagtgctg |
| 1201 | gatagtgacg ggtctttctt tctgtacagt aagctgactg |
| | tggacaagtc ccgctggcag |
| 1261 | cagggtaacg tcttcagctg ttccgtgatg cacgaggcat |
| | tgcacaacca ctacacccag |
| 1321 | aagtcactga gcctgagccc agggaag |

Protein Sequence Defining the Full Length Hu06C11 IGHV2-5 Heavy Chain (Humanized Heavy Chain Variable Region and Human IgG1 Constant Region) (SEQ ID NO:196)

| | |
|---|---|
| 1 | qvtlkesgpt lvkptqtltl tctfsgfsln tygmgvswir |
| | qppgkglewl ahiywdddkr |
| 61 | ynpslksrlt itkdtsknqv vltitnmdpv dtatyycaqr |
| | gyddywgywg qgtlvtvssa |
| 121 | stkgpsvfpl apsskstsgg taalgclvkd yfpepvtvsw |
| | nsgaltsgvh tfpavlqssg |
| 181 | lyslssvvtv pssslgtqty icnvnhkpsn tkvdkrvepk |
| | scdkthtcpp cpapellggp |
| 241 | svflfppkpk dtlmisrtpe vtcvvvdvsh edpevkfnwy |
| | vdgvevhnak tkpreeqyns |
| 301 | tyrvvsvltv lhqdwlngke ykckvsnkal papiektisk |
| | akgqprepqv ytlppsreem |
| 361 | tknqvsltcl vkgfypsdia vewesngqpe nnykttppvl |
| | dsdgsfflys kltvdksrwq |
| 421 | qgnvfscsvm healhnhytq kslslspgk |

Nucleic Acid Sequence Encoding the Full Length Ch14F11 Chimeric Heavy Chain (Mouse Heavy Chain Variable Region and Human IgG1 Constant Region) (SEQ ID NO:197)

| | |
|---|---|
| 1 | caggtcacgc tgaaagagtc aggtcccgga atccttcaac |
| | cttcgcagac attgtcactc |
| 61 | acatgttcct tctccgggtt ctcgctctcg acttatggca |
| | tgggtgtagg atggattcgg |
| 121 | cagcccagcg ggaaggggct tgagtggttg gcggatatct |
| | ggtgggacga cgacaaatac |
| 181 | tacaatccga gcctgaagtc ccgcctcacc atttcgaaag |
| | atacgtcatc aaacgaagtc |
| 241 | tttttgaaga tcgccatcgt ggacacggcg gatacagcga |
| | cgtattactg cgccagaagg |
| 301 | ggacactaca gcgcaatgga ttattgggga caggggacct |
| | cggtgactgt gtcgtccgcc |
| 361 | tcaacaaaag gaccaagtgt gttcccactc gcccctagca |
| | gcaagagtac atccgggggc |
| 421 | actgcagcac tcggctgcct cgtcaaggat tattttccag |
| | agccagtaac cgtgagctgg |
| 481 | aacagtggag cactcacttc tggtgtccat acttttcctg |
| | ctgtcctgca aagctctggc |
| 541 | ctgtactcac tcagctccgt cgtgaccgtg ccatcttcat |
| | ctctgggcac tcagacctac |
| 601 | atctgtaatg taaaccacaa gcctagcaat actaaggtcg |
| | ataagcgggt ggaacccaag |
| 661 | agctgcgaca agactcacac ttgtccccca tgccctgccc |
| | ctgaacttct gggcggtccc |
| 721 | agcgtctttt tgttcccacc aaagcctaaa gatactctga |
| | tgataagtag aacacccgag |
| 781 | gtgacatgtg ttgttgtaga cgtttcccac gaggacccag |
| | aggttaagtt caactggtac |
| 841 | gttgatggag tcgaagtaca taatgctaag accaagccta |
| | gagaggagca gtataatagt |
| 901 | acataccgtg tagtcagtgt tctcacagtg ctgcaccaag |
| | actggctcaa cggcaaagaa |
| 961 | tacaaatgca aagtgtccaa caaagcactc ccagccccta |
| | tcgagaagac tattagtaag |
| 1021 | gcaaaggggc agcctcgtga accacaggtg tacactctgc |
| | cacccagtag agaggaaatg |
| 1081 | acaaagaacc aagtctcatt gacctgcctg gtgaaaggct |
| | tctaccccag cgacatcgcc |
| 1141 | gttgagtggg agagtaacgg tcagcctgag aacaattaca |
| | agacaacccc cccagtgctg |
| 1201 | gatagtgacg ggtctttctt tctgtacagt aagctgactg |
| | tggacaagtc ccgctggcag |
| 1261 | cagggtaacg tcttcagctg ttccgtgatg cacgaggcat |
| | tgcacaacca ctacacccag |
| 1321 | aagtcactga gcctgagccc agggaag |

Protein Sequence Defining the Full Length Ch14F11 Chimeric Heavy Chain (Mouse Heavy Chain Variable Region and Human IgG1 Constant Region) (SEQ ID NO:198)

| | |
|---|---|
| 1 | qvtlkesgpg ilqpsqtlsl tcsfsgfsls tygmgvgwir |
| | qpsgkglewl adiwwdddky |
| 61 | ynpslksrlt iskdtssnev flkiaivdta dtatyycarr |
| | ghysamdywg qgtsvtvssa |
| 121 | stkgpsvfpl apsskstsgg taalgclvkd yfpepvtvsw |
| | nsgaltsgvh tfpavlqssg |
| 181 | lyslssvvtv pssslgtqty icnvnhkpsn tkvdkrvepk |
| | scdkthtcpp cpapellggp |
| 241 | svflfppkpk dtlmisrtpe vtcvvvdvsh edpevkfnwy |
| | vdgvevhnak tkpreeqyns |
| 301 | tyrvvsvltv lhqdwlngke ykckvsnkal papiektisk |
| | akgqprepqv ytlppsreem |
| 361 | tknqvsltcl vkgfypsdia vewesngqpe nnykttppvl |
| | dsdgsfflys kltvdksrwq |
| 421 | qgnvfscsvm healhnhytq kslslspgk |

Nucleic Acid Sequence Encoding the Full Length Sh14F11 IGHV2-5 Heavy Chain (Humanized Heavy Chain Variable Region and Human IgG1 Constant Region) (SEQ ID NO:199)

| | |
|---|---|
| 1 | cagatcactt tgaaagaaag cggaccgacc ttggtcaagc |
| | ccacacaaac cctcacgctc |
| 61 | acgtgtacat tttcggggtt ctcgctttca acttacggga |
| | tgggagtagg gtggattcgc |
| 121 | cagccgcctg gtaaagcgtt ggagtggctt gcagacatct |
| | ggtgggacga cgataagtac |
| 181 | tataatccct cgctcaagtc cagactgacc atcacgaaag |
| | atacgagcaa gaaccaggtc |
| 241 | gtgctgacaa tgactaacat ggacccagtg gatacggcta |
| | catattactg cgccaggcgg |
| 301 | ggtcactact cagcgatgga ttattggggc cagggaacac |
| | tggtaacggt gtcgtccgcc |
| 361 | tcaacaaaag gaccaagtgt gttcccactc gcccctagca |
| | gcaagagtac atccgggggc |
| 421 | actgcagcac tcggctgcct cgtcaaggat tattttccag |
| | agccagtaac cgtgagctgg |
| 481 | aacagtggag cactcacttc tggtgtccat acttttcctg |
| | ctgtcctgca aagctctggc |
| 541 | ctgtactcac tcagctccgt cgtgaccgtg ccatcttcat |
| | ctctgggcac tcagacctac |
| 601 | atctgtaatg taaaccacaa gcctagcaat actaaggtcg |
| | ataagcgggt ggaacccaag |
| 661 | agctgcgaca agactcacac ttgtccccca tgccctgccc |
| | ctgaacttct gggcggtccc |
| 721 | agcgtctttt tgttcccacc aaagcctaaa gatactctga |
| | tgataagtag aacacccgag |
| 781 | gtgacatgtg ttgttgtaga cgtttcccac gaggacccag |
| | aggttaagtt caactggtac |
| 841 | gttgatggag tcgaagtaca taatgctaag accaagccta |
| | gagaggagca gtataatagt |
| 901 | acataccgtg tagtcagtgt tctcacagtg ctgcaccaag |
| | actggctcaa cggcaaagaa |
| 961 | tacaaatgca aagtgtccaa caaagcactc ccagccccta |
| | tcgagaagac tattagtaag |
| 1021 | gcaaaggggc agcctcgtga accacaggtg tacactctgc |
| | cacccagtag agaggaaatg |
| 1081 | acaaagaacc aagtctcatt gacctgcctg gtgaaaggct |
| | tctaccccag cgacatcgcc |
| 1141 | gttgagtggg agagtaacgg tcagcctgag aacaattaca |
| | agacaacccc cccagtgctg |
| 1201 | gatagtgacg ggtctttctt tctgtacagt aagctgactg |
| | tggacaagtc ccgctggcag |
| 1261 | cagggtaacg tcttcagctg ttccgtgatg cacgaggcat |
| | tgcacaacca ctacacccag |
| 1321 | aagtcactga gcctgagccc agggaag |

Protein Sequence Defining the Full Length Sh14F11 IGHV2-5 Heavy Chain (Humanized Heavy Chain Variable Region and Human IgG1 Constant Region) (SEQ ID NO:200)

| | |
|---|---|
| 1 | qitlkesgpt lvkptqtltl tctfsgfsls tygmgvgwir |
| | qppgkalewl adiwwdddky |
| 61 | ynpslksrlt itkdtsknqv vltmtnmdpv dtatyycarr |
| | ghysamdywg qgtlvtvssa |
| 121 | stkgpsvfpl apsskstsgg taalgclvkd yfpepvtvsw |
| | nsgaltsgvh tfpavlqssg |
| 181 | lyslssvvtv pssslgtqty icnvnhkpsn tkvdkrvepk |
| | scdkthtcpp cpapellggp |
| 241 | svflfppkpk dtlmisrtpe vtcvvvdvsh edpevkfnwy |
| | vdgvevhnak tkpreeqyns |
| 301 | tyrvvsvltv lhqdwlngke ykckvsnkal papiektisk |
| | akgqprepqv ytlppsreem |
| 361 | tknqvsltcl vkgfypsdia vewesngqpe nnykttppvl |
| | dsdgsfflys kltvdksrwq |
| 421 | qgnvfscsvm healhnhytq kslslspgk |

Nucleic Acid Sequence Encoding the Full Length Sh14F11 IGHV2-70 Heavy Chain (Humanized Heavy Chain Variable Region and Human IgG1 Constant Region) (SEQ ID NO:201)

| | |
|---|---|
| 1 | caagtgactc tcaaggagtc cggacccgcc ctggtcaaac |
| | caacgcagac actgacgctc |
| 61 | acatgcacct tcagcggatt ttcgttgtca acgtacggca |
| | tgggtgtggg gtggattcgc |
| 121 | cagcctccgg ggaaagccct tgaatggttg gcggacatct |
| | ggtgggatga tgacaagtac |
| 181 | tataatccct cacttaagtc acggttgacg atctcgaaag |
| | acaccagcaa gaaccaggta |
| 241 | gtgctgacaa tgactaacat ggacccggtc gatacagcgg |
| | tctactattg tgctagaagg |
| 301 | ggacactact ccgcaatgga ttattggggt caggggacgc |
| | tcgtaaccgt gtcgtcggcc |
| 361 | tcaacaaaag gaccaagtgt gttcccactc gcccctagca |
| | gcaagagtac atccgggggc |
| 421 | actgcagcac tcggctgcct cgtcaaggat tattttccag |
| | agccagtaac cgtgagctgg |
| 481 | aacagtggag cactcacttc tggtgtccat acttttcctg |
| | ctgtcctgca aagctctggc |
| 541 | ctgtactcac tcagctccgt cgtgaccgtg ccatcttcat |
| | ctctgggcac tcagacctac |
| 601 | atctgtaatg taaaccacaa gcctagcaat actaaggtcg |
| | ataagcgggt ggaacccaag |
| 661 | agctgcgaca agactcacac ttgtccccca tgccctgccc |
| | ctgaacttct gggcggtccc |
| 721 | agcgtctttt tgttcccacc aaagcctaaa gatactctga |
| | tgataagtag aacacccgag |
| 781 | gtgacatgtg ttgttgtaga cgtttcccac gaggacccag |
| | aggttaagtt caactggtac |
| 841 | gttgatggag tcgaagtaca taatgctaag accaagccta |
| | gagaggagca gtataatagt |
| 901 | acataccgtg tagtcagtgt tctcacagtg ctgcaccaag |
| | actggctcaa cggcaaagaa |
| 961 | tacaaatgca aagtgtccaa caaagcactc ccagccccta |
| | tcgagaagac tattagtaag |
| 1021 | gcaaaggggc agcctcgtga accacaggtg tacactctgc |
| | cacccagtag agaggaaatg |
| 1081 | acaaagaacc aagtctcatt gacctgcctg gtgaaaggct |
| | tctaccccag cgacatcgcc |
| 1141 | gttgagtggg agagtaacgg tcagcctgag aacaattaca |
| | agacaacccc cccagtgctg |
| 1201 | gatagtgacg ggtctttctt tctgtacagt aagctgactg |
| | tggacaagtc ccgctggcag |
| 1261 | cagggtaacg tcttcagctg ttccgtgatg cacgaggcat |
| | tgcacaacca ctacacccag |
| 1321 | aagtcactga gcctgagccc agggaag |

Protein Sequence Defining the Full Length Sh14F11 IGHV2-70 Heavy Chain (Humanized Heavy Chain Variable Region and Human IgG1 Constant Region) (SEQ ID NO:202)

| | |
|---|---|
| 1 | qvtlkesgpa lvkptqtltl tctfsgfsls tygmgvgwir |
| | qppgkalewl adiwwdddky |
| 61 | ynpslksrlt iskdtsknqv vltmtnmdpv dtavyycarr |
| | ghysamdywg qgtlvtvssa |
| 121 | stkgpsvfpl apsskstsgg taalgclvkd yfpepvtvsw |
| | nsgaltsgvh tfpavlqssg |
| 181 | lyslssvvtv pssslgtqty icnvnhkpsn tkvdkrvepk |
| | scdkthtcpp cpapellggp |
| 241 | svflfppkpk dtlmisrtpe vtcvvvdvsh edpevkfnwy |
| | vdgvevhnak tkpreeqyns |
| 301 | tyrvvsvltv lhqdwlngke ykckvsnkal papiektisk |
| | akgqprepqv ytlppsreem |
| 361 | tknqvsltcl vkgfypsdia vewesngqpe nnykttppvl |
| | dsdgsfflys kltvdksrwq |
| 421 | qgnvfscsvm healhnhytq kslslspgk |

Nucleic Acid Sequence Encoding the Full Length Ch01G06 Chimeric Light Chain (Mouse Kappa Chain Variable Region and Human Kappa Constant Region) (SEQ ID NO:203)

| | |
|---|---|
| 1 | gacatccaaa tgacccagtc acccgcgagc ctttcggcgt |
| | cggtcggaga aacggtcacg |
| 61 | atcacgtgcc ggacatcaga gaatctccat aactacctcg |
| | cgtggtatca acagaagcag |
| 121 | gggaagtcgc cccagttgct tgtatacgat gcgaaaacgt |
| | tggcggatgg ggtgccgtcc |
| 181 | agattctcgg gatcgggctc ggggacgcag tactcgctca |
| | agatcaattc gctgcagccg |
| 241 | gaggactttg ggtcgtacta ttgtcagcat ttttggtcat |
| | caccgtatac atttggaggt |
| 301 | ggaacgaaac ttgagattaa gcgcacagtt gctgccccca |
| | gcgtgttcat tttcccacct |
| 361 | agcgatgagc agctgaaaag cggtactgcc tctgtcgtat |
| | gcttgctcaa caacttttac |
| 421 | ccacgtgagg ctaaggtgca gtggaaagtg gataatgcac |
| | ttcaatctgg aaacagtcaa |
| 481 | gagtccgtga cagaacagga cagcaaagac tcaacttatt |
| | cactctcttc caccctgact |
| 541 | ctgtccaagg cagactatga aaaacacaag gtatacgcct |
| | gcgaggttac acaccagggt |
| 601 | ttgtctagtc ctgtcaccaa gtccttcaat aggggcgaat gt |

Protein Sequence Defining the Full Length Ch01G06 Chimeric Light Chain (Mouse Kappa Chain Variable Region and Human Kappa Constant Region) (SEQ ID NO:204)

| | |
|---|---|
| 1 | diqmtqspas lsasvgetvt itcrtsenlh nylawyqqkq |
| | gkspqllvyd aktladgvps |
| 61 | rfsgsgsgtq yslkinslqp edfgsyycqh fwsspytfgg |
| | gtkleikrtv aapsvfifpp |
| 121 | sdeqlksgta svvcllnnfy preakvqwkv dnalqsgnsq |
| | esvteqdskd styslsstlt |
| 181 | lskadyekhk vyacevthqg lsspvtksfn rgec |

Nucleic Acid Sequence Encoding the Full Length Hu01G06 IGKV1-39 Light Chain (Humanized Kappa Chain Variable Region and Human Kappa Constant Region) (SEQ ID NO:205)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaagtcac cgaaactcct tgtctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa gcgcacagtt gctgccccca |
| | gcgtgttcat tttcccacct |
| 361 | agcgatgagc agctgaaaag cggtactgcc tctgtcgtat |
| | gcttgctcaa caacttttac |
| 421 | ccacgtgagg ctaaggtgca gtggaaagtg gataatgcac |
| | ttcaatctgg aaacagtcaa |
| 481 | gagtccgtga cagaacagga cagcaaagac tcaacttatt |
| | cactctcttc caccctgact |
| 541 | ctgtccaagg cagactatga aaaacacaag gtatacgcct |
| | gcgaggttac acaccagggt |
| 601 | ttgtctagtc ctgtcaccaa gtccttcaat aggggcgaat gt |

Protein Sequence Defining the Full Length Hu01G06 IGKV1-39 Light Chain (Humanized Kappa Chain Variable Region and Human Kappa Constant Region) (SEQ ID NO:206)

| | |
|---|---|
| 1 | diqmtqspss lsasvgdrvt itcrtsenlh nylawyqqkp |
| | gkspkllvyd aktladgvps |
| 61 | rfsgsgsgtd ytltisslqp edfatyycqh fwsspytfgq |
| | gtkleikrtv aapsvfifpp |
| 121 | sdeqlksgta svvcllnnfy preakvqwkv dnalqsgnsq |
| | esvteqdskd styslsstlt |
| 181 | lskadyekhk vyacevthqg lsspvtksfn rgec |

Nucleic Acid Sequence Encoding the Full Length Hu01G06 IGKV1-39 S43A V48I Light Chain (Humanized Kappa Chain Variable Region and Human Kappa Constant Region) (Also Referred to Herein as the Full Length Hu01G06 IGFV1-39 F1 Light Chain; SEQ ID NO:207)

| | |
|---|---|
| 1 | gacatccaaa tgacccagtc gccgtcgtcg ctttcagcgt |
| | cggtagggga tcgggtcaca |
| 61 | attacgtgcc gaacgtcaga gaatttgcat aactacctcg |
| | cgtggtatca gcagaagccc |
| 121 | gggaaggccc cgaaactcct tatctacgat gcgaaaacgc |
| | tggcggatgg agtgccgtcg |
| 181 | agattctcgg gaagcggatc cggtacggac tatacgctta |
| | cgatctcatc gctccagccc |
| 241 | gaggactttg cgacgtacta ttgtcagcat ttttggtcgt |
| | cgccctacac atttgggcag |
| 301 | gggaccaagt tggaaatcaa gcgcacagtt gctgccccca |
| | gcgtgttcat tttcccacct |
| 361 | agcgatgagc agctgaaaag cggtactgcc tctgtcgtat |
| | gcttgctcaa caacttttac |
| 421 | ccacgtgagg ctaaggtgca gtggaaagtg gataatgcac |
| | ttcaatctgg aaacagtcaa |
| 481 | gagtccgtga cagaacagga cagcaaagac tcaacttatt |
| | cactctcttc caccctgact |
| 541 | ctgtccaagg cagactatga aaaacacaag gtatacgcct |
| | gcgaggttac acaccagggt |
| 601 | ttgtctagtc ctgtcaccaa gtccttcaat aggggcgaat gt |

Protein Sequence Defining the Full Length Hu01G06 IGKV1-39 S43A V48I Light Chain (Humanized Kappa Chain Variable Region and Human Kappa Constant Region) (Also Referred to Herein as the Full Length Hu01G06 IGFV1-39 F1 Light Chain; SEQ ID NO:208)

| | |
|---|---|
| 1 | diqmtqspss lsasvgdrvt itcrtsenlh nylawyqqkp gkapklliyd aktladgvps |
| 61 | rfsgsgsgtd ytltisslqp edfatyycqh fwsspytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Hu01G06 IGKV1-39 V48I Light Chain (Humanized Kappa Chain Variable Region and Human Kappa Constant Region) (SEQ ID NO:209)

| | |
|---|---|
| 1 | gacatccaaa tgacccagtc gccgtcgtcg ctttcagcgt cggtagggga tcgggtcaca |
| 61 | attacgtgcc gaacgtcaga gaatttgcat aactacctcg cgtggtatca gcagaagccc |
| 121 | gggaagtcac cgaaactcct tatctacgat gcgaaaacgc tggcggatgg agtgccgtcg |
| 181 | agattctcgg gaagcggatc cggtacggac tatacgctta cgatctcatc gctccagccc |
| 241 | gaggactttg cgacgtacta ttgtcagcat ttttggtcgt cgccctacac atttgggcag |
| 301 | gggaccaagt tggaaatcaa gcgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Hu01G06 IGKV1-39 V48I Light Chain (Humanized Kappa Chain Variable Region and Human Kappa Constant Region) (SEQ ID NO:210)

| | |
|---|---|
| 1 | diqmtqspss lsasvgdrvt itcrtsenlh nylawyqqkp gkspklliyd aktladgvps |
| 61 | rfsgsgsgtd ytltisslqp edfatyycqh fwsspytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Hu01G06 IGKV1-39 F1 Light Chain (Humanized Kappa Chain Variable Region and Human Kappa Constant Region) (Also Referred to Herein as the Full Length Hu01G06 IGKV1-39 S43A V48I Light Chain; SEQ ID NO:207)

| | |
|---|---|
| 1 | gacatccaaa tgacccagtc gccgtcgtcg ctttcagcgt cggtagggga tcgggtcaca |
| 61 | attacgtgcc gaacgtcaga gaatttgcat aactacctcg cgtggtatca gcagaagccc |
| 121 | gggaaggccc cgaaactcct tatctacgat gcgaaaacgc tggcggatgg agtgccgtcg |
| 181 | agattctcgg gaagcggatc cggtacggac tatacgctta cgatctcatc gctccagccc |
| 241 | gaggactttg cgacgtacta ttgtcagcat ttttggtcgt cgccctacac atttgggcag |
| 301 | gggaccaagt tggaaatcaa gcgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Hu01G06 IGKV1-39 F1 Light Chain (Humanized Kappa Chain Variable Region and Human Kappa Constant Region) (Also Referred to Herein as the Full Length Hu01G06 IGKV1-39 S43A V48I Light Chain; SEQ ID NO:208)

| | |
|---|---|
| 1 | diqmtqspss lsasvgdrvt itcrtsenlh nylawyqqkp gkapklliyd aktladgvps |
| 61 | rfsgsgsgtd ytltisslqp edfatyycqh fwsspytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Hu01G06 IGKV1-39 F2 Light Chain (Humanized Kappa Chain Variable Region and Human Kappa Constant Region) (SEQ ID NO:263)

| | |
|---|---|
| 1 | gacatccaaa tgacccagtc gccgtcgtcg ctttcagcgt cggtagggga tcgggtcaca |
| 61 | attacgtgcc gaacgtcaga gaatttgcat aactacctcg cgtggtatca gcagaagccc |
| 121 | gggaagtcac cgaaactcct tatctacgat gcgaaaacgc tggcggatgg agtgccgtcg |
| 181 | agattctcgg gaagcggatc cggtacggac tatacgctta cgatctcatc gctccagccc |
| 241 | gaggactttg cgacgtacta ttgtcagcat ttttggtcgg acccctacac atttgggcag |
| 301 | gggaccaagt tggaaatcaa gcgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Hu01G06 IGKV1-39 F2 Light Chain (Humanized Kappa Chain Variable Region and Human Kappa Constant Region) (SEQ ID NO:264)

| | |
|---|---|
| 1 | diqmtqspss lsasvgdrvt itcrtsenlh nylawyqqkp gkspklliyd aktladgvps |
| 61 | rfsgsgsgtd ytltisslqp edfatyycqh fwsdpytfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Ch06C11 Chimeric Light Chain (Mouse Kappa Chain Variable Region and Human Kappa Constant Region) (SEQ ID NO:211)

| | |
|---|---|
| 1 | gatatcgtca tgacccagtc ccagaagttc atgtcaactt cagtgggaga cagagtgtcc |
| 61 | gtcacatgta aagcctcgca aaatgtggga accaacgtag cgtggttcca gcagaaacct |
| 121 | ggccaatcac cgaaggcact gatctactcg gccagctata ggtactcggg agtaccagat |
| 181 | cggtttacgg ggtcggggag cgggacggac tttatcctca ctatttccaa tgtccagtcg |
| 241 | gaggaccttg cggaatactt ctgccagcag tataacaact atcccctcac gtttggtgct |
| 301 | ggtacaaaat tggagttgaa gcgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Ch06C11 Chimeric Light Chain (Mouse Kappa Chain Variable Region and Human Kappa Constant Region) (SEQ ID NO:212)

| | |
|---|---|
| 1 | divmtqsqkf mstsvgdrvs vtckasqnvg tnvawfqqkp gqspkaliys asyrysgvpd |
| 61 | rftgsgsgtd filtisnvqs edlaeyfcqq ynnypltfga gtklelkrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Sh06C11 IGKV1-16 Light Chain (Humanized Kappa Chain Variable Region and Human Kappa Constant Region) (SEQ ID NO:213)

| | |
|---|---|
| 1 | gacatccaaa tgacccaatc gccctcctcc ctctccgcat cagtagggga ccgcgtcaca |
| 61 | attacttgca aagcgtcgca gaacgtcgga acgaatgtgg cgtggtttca gcagaagccc |
| 121 | ggaaaagctc cgaagagctt gatctactcg gcctcatata ggtattcggg tgtgccgagc |
| 181 | cggtttagcg ggtcggggtc aggtactgat ttcacgctca caatttcatc gttgcagcca |
| 241 | gaagatttcg ccacatatta ctgtcagcag tacaacaatt accctctgac gttcggccag |
| 301 | ggaaccaaac ttgagatcaa gcgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Sh06C11 IGKV1-16 Light Chain (Humanized Kappa Chain Variable Region and Human Kappa Constant Region) (SEQ ID NO:214)

| | |
|---|---|
| 1 | diqmtqspss lsasvgdrvt itckasqnvg tnvawfqqkp gkapksliys asyrysgvps |
| 61 | rfsgsgsgtd ftltisslqp edfatyycqq ynnypltfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Ch14F11 Chimeric Light Chain (Mouse Kappa Chain Variable Region and Human Kappa Constant Region) (SEQ ID NO:215)

| | |
|---|---|
| 1 | gacatcgtga tgacacagtc acagaaattc atgtccacat ccgtcggtga tagagtatcc |
| 61 | gtcacgtgta aggcctcgca aaacgtagga actaatgtgg cgtggtatca acagaagcca |
| 121 | ggacagtcac ccaaagcact catctacagc ccctcatatc ggtacagcgg ggtgccggac |
| 181 | aggttcacgg gatcggggag cgggaccgat tttacactga ccatttcgaa tgtccagtcg |
| 241 | gaggaccttg cggaatactt ctgccagcag tataactcgt accctcacac gtttggaggt |
| 301 | ggcactaagt tggagatgaa acgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Ch14F11 Chimeric Light Chain (Mouse Kappa Chain Variable Region and Human Kappa Constant Region) (SEQ ID NO:216)

| | |
|---|---|
| 1 | divmtqsqkf mstsvgdrvs vtckasqnvg tnvawyqqkp gqspkaliys psyrysgvpd |
| 61 | rftgsgsgtd ftltisnvqs edlaeyfcqq ynsyphtfgg gtklemkrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Nucleic Acid Sequence Encoding the Full Length Hu14F11 IGKV1-16 Light Chain (Humanized Kappa Chain Variable Region and Human Kappa Constant Region) (SEQ ID NO:217)

| | |
|---|---|
| 1 | gatatccaga tgacacagtc accctcgtcg ctctcagctt ccgtaggcga cagggtcact |
| 61 | attacgtgta aagcatcaca gaacgtcgga acgaatgtgg cgtggtttca gcagaagccc |
| 121 | gggaagagcc ccaaagcgct tatctactcc ccgtcgtatc ggtattccgg tgtgccaagc |
| 181 | agattttcgg ggtcaggttc gggaactgac tttaccctga ccatctcgtc cctccaaccg |
| 241 | gaagatttcg ccacgtactt ctgccagcag tacaacagct atcctcacac attcggacaa |
| 301 | gggacaaagt tggagattaa acgcacagtt gctgccccca gcgtgttcat tttcccacct |
| 361 | agcgatgagc agctgaaaag cggtactgcc tctgtcgtat gcttgctcaa caacttttac |
| 421 | ccacgtgagg ctaaggtgca gtggaaagtg gataatgcac ttcaatctgg aaacagtcaa |
| 481 | gagtccgtga cagaacagga cagcaaagac tcaacttatt cactctcttc caccctgact |
| 541 | ctgtccaagg cagactatga aaaacacaag gtatacgcct gcgaggttac acaccagggt |
| 601 | ttgtctagtc ctgtcaccaa gtccttcaat aggggcgaat gt |

Protein Sequence Defining the Full Length Hu14F11 IGKV1-16 Light Chain (Humanized Kappa Chain Variable Region and Human Kappa Constant Region) (SEQ ID NO:218)

| | |
|---|---|
| 1 | diqmtqspss lsasvgdrvt itckasqnvg tnvawfqqkp gkspkaliys psyrysgvps |
| 61 | rfsgsgsgtd ftltisslqp edfatyfcqq ynsyphtfgq gtkleikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

Table 22 is a concordance chart showing the SEQ ID NO. of each sequence discussed in this Example.

TABLE 22

| SEQ ID NO. | Nucleic Acid or Protein |
|---|---|
| 171 | Human IgG1 constant-nucleic acid |
| 172 | Human IgG1 constant-protein |
| 173 | Human Kappa constant-nucleic acid |
| 174 | Human Kappa constant-protein |
| 175 | Humanized Ch01G06 Chimeric Heavy Human Variable + Human IgG1 constant-nucleic acid |
| 176 | Humanized Ch01G06 Chimeric Heavy Human Variable + Human IgG1 constant-protein |
| 177 | Humanized Hu01G06 IGHV1-18 Heavy Human Variable + Human IgG1 constant-nucleic acid |
| 178 | Humanized Hu01G06 IGHV1-18 Heavy Human Variable + Human IgG1 constant-protein |
| 179 | Humanized Hu01G06 IGHV1-69 Heavy Human Variable + Human IgG1 constant-nucleic acid |
| 180 | Humanized Hu01G06 IGHV1-69 Heavy Human Variable + Human IgG1 constant-protein |
| 181 | Humanized Sh01G06 IGHV1-18 M69L Heavy Human Variable + Human IgG1 constant-nucleic acid |
| 182 | Humanized Sh01G06 IGHV1-18 M69L Heavy Human Variable + Human IgG1 constant-protein |
| 183 | Humanized Sh01G06 IGHV1-18 M69L K64Q G44S Heavy Human Variable + Human IgG1 constant-nucleic acid |
| 184 | Humanized Sh01G06 IGHV1-18 M69L K64Q G44S Heavy Human Variable + Human IgG1 constant-protein |
| 185 | Humanized Sh01G06 IGHV1-18 M69L K64Q Heavy Human Variable + Human IgG1 constant-nucleic acid |
| 186 | Humanized Sh01G06 IGHV1-18 M69L K64Q Heavy Human Variable + Human IgG1 constant-protein |
| 187 | Humanized Sh01G06 IGHV1-69 T30S I69L Heavy Human Variable + Human IgG1 constant-nucleic acid |
| 188 | Humanized Sh01G06 IGHV1-69 T30S I69L Heavy Human Variable + Human IgG1 constant-protein |
| 189 | Humanized Sh01G06 IGHV1-69 T30S K64Q I69L Heavy Human Variable + Human IgG1 constant-nucleic acid |
| 190 | Humanized Sh01G06 IGHV1-69 T30S K64Q I69L Heavy Human Variable + Human IgG1 constant-protein |
| 255 | Humanized Hu01G06 IGHV1-18 F1 Heavy Human Variable + Human IgG1 constant-nucleic acid |
| 256 | Humanized Hu01G06 IGHV1-18 F1 Heavy Human Variable + Human IgG1 constant-protein |
| 257 | Humanized Hu01G06 IGHV1-18 F2 Heavy Human Variable + Human IgG1 constant-nucleic acid |
| 258 | Humanized Hu01G06 IGHV1-18 F2 Heavy Human Variable + Human IgG1 constant-protein |
| 259 | Humanized Hu01G06 IGHV1-69 F1 Heavy Human Variable + Human IgG1 constant-nucleic acid |
| 260 | Humanized Hu01G06 IGHV1-69 F1 Heavy Human Variable + Human IgG1 constant-protein |
| 261 | Humanized Hu01G06 IGHV1-69 F2 Heavy Human Variable + Human IgG1 constant-nucleic acid |
| 262 | Humanized Hu01G06 IGHV1-69 F2 Heavy Human Variable + Human IgG1 constant-protein |
| 191 | Humanized Ch06C11 Chimeric Heavy Human Variable + Human IgG1 constant-nucleic acid |
| 192 | Humanized Ch06C11 Chimeric Heavy Human Variable + Human IgG1 constant-protein |
| 193 | Humanized HE LM 06C11 IGHV2-70 Heavy Human Variable + Human IgG1 constant-nucleic acid |
| 194 | Humanized HE LM 06C11 IGHV2-70 Heavy Human Variable + Human IgG1 constant-protein |
| 195 | Humanized Hu06C11 IGHV2-5 Heavy Human Variable + Human IgG1 constant-nucleic acid |
| 196 | Humanized Hu06C11 IGHV2-5 Heavy Human Variable + Human IgG1 constant-protein |
| 197 | Humanized Ch14F11 Chimeric Heavy Human Variable + Human IgG1 constant-nucleic acid |
| 198 | Humanized Ch14F11 Chimeric Heavy Human Variable + Human IgG1 constant-protein |
| 199 | Humanized Sh14F11 IGHV2-5 Heavy Human Variable + Human IgG1 constant-nucleic acid |
| 200 | Humanized Sh14F11 IGHV2-5 Heavy Human Variable + Human IgG1 constant-protein |
| 201 | Humanized Sh14F11-IGHV2-70 Heavy Human Variable + Human IgG1 constant-nucleic acid |
| 202 | Humanized Sh14F11-IGHV2-70 Heavy Human Variable + Human IgG1 constant-protein |
| 203 | Humanized Ch01G06 Chimeric Human Variable + Human Kappa constant-nucleic acid |
| 204 | Humanized Ch01G06 Chimeric Human Variable + Human Kappa constant-protein |
| 205 | Humanized Hu01G06 IGKV1-39 Human Variable + Human Kappa constant-nucleic acid |
| 206 | Humanized Hu01G06 IGKV1-39 Human Variable + Human Kappa constant-protein |
| 207 | Humanized Hu01G06 IGKV1-39 S43A V48I Human Variable + Human Kappa constant-nucleic acid |
| 208 | Humanized Hu01G06 IGKV1-39 S43A V48I Human Variable + Human Kappa constant-protein |
| 209 | Humanized Hu01G06 IGKV1-39 V48I Human Variable + Human Kappa constant-nucleic acid |
| 210 | Humanized Hu01G06 IGKV1-39 V48I Human Variable + Human Kappa constant-protein |
| 207 | Humanized Hu01G06 IGKV1-39 F1 Human Variable + Human Kappa constant-nucleic acid |
| 208 | Humanized Hu01G06 IGKV1-39 F1 Human Variable + Human Kappa constant-protein |
| 263 | Humanized Hu01G06 IGKV1-39 F2 Human Variable + Human Kappa constant-nucleic acid |
| 264 | Humanized Hu01G06 IGKV1-39 F2 Human Variable + Human Kappa constant-protein |
| 211 | Humanized Ch06C11 Chimeric Human Variable + Human Kappa constant-nucleic acid |
| 212 | Humanized Ch06C11 Chimeric Human Variable + Human Kappa constant-protein |
| 213 | Humanized Sh06C11 IGKV1-16 Human Variable + Human Kappa constant-nucleic acid |
| 214 | Humanized Sh06C11 IGKV1-16 Human Variable + Human Kappa constant-protein |
| 215 | Humanized Ch14F11 Chimeric Human Variable + Human Kappa constant-nucleic acid |
| 216 | Humanized Ch14F11 Chimeric Human Variable + Human Kappa constant-protein |
| 217 | Humanized Hu14F11 IGKV1-16 Human Variable + Human Kappa constant-nucleic acid |
| 218 | Humanized Hu14F11 IGKV1-16 Human Variable + Human Kappa constant-protein |

Table 23 below shows antibodies containing chimeric immunoglobulin heavy and light chains and exemplary combinations of the full-length chimeric or humanized immunoglobulin heavy and light chains.

TABLE 23

| Antibody Name | Light Chain | Heavy Chain |
|---|---|---|
| Hu01G06-1 | Humanized Ch01G06 Chimeric | Humanized Ch01G06 Chimeric Heavy |
| | Human Variable + Human Kappa | Human Variable + Human IgG1 |
| | constant (SEQ ID NO: 204) | constant (SEQ ID NO: 176) |
| Hu01G06-46 | Humanized Hu01G06 IGKV1-39 | Humanized Hu01G06 IGHV1-18 Heavy |
| | Human Variable + Human Kappa | Human Variable + Human IgG1 |
| | constant (SEQ ID NO: 206) | constant (SEQ ID NO: 178) |
| Hu01G06-52 | Humanized Hu01G06 IGKV1-39 | Humanized Hu01G06 IGHV1-69 Heavy |
| | Human Variable + Human Kappa | Human Variable + Human IgG1 |
| | constant (SEQ ID NO: 206) | constant (SEQ ID NO: 180) |
| Hu01G06-100 | Humanized Hu01G06 IGKV1-39 | Humanized Sh01G06 IGHV1-18 M69L |
| | Human Variable + Human Kappa | Heavy Human Variable + Human IgG1 |
| | constant (SEQ ID NO: 206) | constant (SEQ ID NO: 182) |
| Hu01G06-101 | Humanized Hu01G06 IGKV1-39 | Humanized Sh01G06 IGHV1-18 M69L |
| | Human Variable + Human Kappa | K64Q Heavy Human Variable + Human |
| | constant (SEQ ID NO: 206) | IgG1 constant (SEQ ID NO: 186) |
| Hu01G06-102 | Humanized Hu01G06 IGKV1-39 | Humanized Sh01G06 IGHV1-18 M69L K64Q |
| | Human Variable + Human Kappa | G44S Heavy Human Variable + Human |
| | constant (SEQ ID NO: 206) | IgG1 constant (SEQ ID NO: 184) |
| Hu01G06-103 | Humanized Hu01G06 IGKV1-39 | Humanized Sh01G06 IGHV1-69 T30S |
| | Human Variable + Human Kappa | I69L Heavy Human Variable + Human |
| | constant (SEQ ID NO: 206) | IgG1 constant (SEQ ID NO: 188) |
| Hu01G06-104 | Humanized Hu01G06 IGKV1-39 | Humanized Sh01G06 IGHV1-69 T30S K64Q |
| | Human Variable + Human Kappa | I69L Heavy Human Variable + Human |
| | constant (SEQ ID NO: 206) | IgG1 constant (SEQ ID NO: 190) |
| Hu01G06-105 | Humanized Hu01G06 IGKV1-39 V48I | Humanized Sh01G06 IGHV1-18 M69L |
| | Human Variable + Human Kappa | Heavy Human Variable + Human IgG1 |
| | constant (SEQ ID NO: 210) | constant (SEQ ID NO: 182) |
| Hu01G06-106 | Humanized Hu01G06 IGKV1-39 V48I | Humanized Sh01G06 IGHV1-18 M69L |
| | Human Variable + Human Kappa | K64Q Heavy Human Variable + Human |
| | constant (SEQ ID NO: 210) | IgG1 constant (SEQ ID NO: 186) |
| Hu01G06-107 | Humanized Hu01G06 IGKV1-39 V48I | Humanized Sh01G06 IGHV1-18 M69L K64Q |
| | Human Variable + Human Kappa | G44S Heavy Human Variable + Human |
| | constant (SEQ ID NO: 210) | IgG1 constant (SEQ ID NO: 184) |
| Hu01G06-108 | Humanized Hu01G06 IGKV1-39 V48I | Humanized Sh01G06 IGHV1-69 T30S |
| | Human Variable + Human Kappa | I69L Heavy Human Variable + Human |
| | constant (SEQ ID NO: 210) | IgG1 constant (SEQ ID NO: 188) |
| Hu01G06-109 | Humanized Hu01G06 IGKV1-39 V48I | Humanized Sh01G06 IGHV1-69 T30S K64Q |
| | Human Variable + Human Kappa | I69L Heavy Human Variable + Human |
| | constant (SEQ ID NO: 210) | IgG1 constant (SEQ ID NO: 190) |
| Hu01G06-110 | Humanized Hu01G06 IGKV1-39 S43A | Humanized Sh01G06 IGHV1-18 M69L |
| | V48I Human Variable + Human Kappa | Heavy Human Variable + Human |
| | constant (SEQ ID NO: 208) | IgG1 constant (SEQ ID NO: 182) |
| Hu01G06-111 | Humanized Hu01G06 IGKV1-39 S43A | Humanized Sh01G06 IGHV1-18 M69L |
| | V48I Human Variable + Human Kappa | K64Q Heavy Human Variable + Human |
| | constant (SEQ ID NO: 208) | IgG1 constant (SEQ ID NO: 186) |
| Hu01G06-112 | Humanized Hu01G06 IGKV1-39 S43A | Humanized Sh01G06 IGHV1-18 M69L |
| | V48I Human Variable + Human Kappa | K64Q G44S Heavy Human Variable + |
| | constant (SEQ ID NO: 208) | Human IgG1 constant (SEQ ID NO: 184) |
| Hu01G06-113 | Humanized Hu01G06 IGKV1-39 S43A | Humanized Sh01G06 IGHV1-69 T30S |
| | V48I Human Variable + Human Kappa | I69L Heavy Human Variable + Human |
| | constant (SEQ ID NO: 208) | IgG1 constant (SEQ ID NO: 188) |
| Hu01G06-114 | Humanized Hu01G06 IGKV1-39 S43A | Humanized Sh01G06 IGHV1-69 T30S K64Q |
| | V48I Human Variable + Human Kappa | I69L Heavy Human Variable + Human |
| | constant (SEQ ID NO: 208) | IgG1 constant (SEQ ID NO: 190) |
| Hu01G06-122 | Humanized Hu01G06 IGKV1-39 F1 | Humanized Hu01G06 IGHV1-18 F1 |
| | Human Variable + Human Kappa | Heavy Human Variable + Human IgG1 |
| | constant (SEQ ID NO: 208) | constant (SEQ ID NO: 256) |
| Hu01G06-127 | Humanized Hu01G06 IGKV1-39 F2 | Humanized Hu01G06 IGHV1-18 F2 |
| | Human Variable + Human Kappa | Heavy Human Variable + Human IgG1 |
| | constant (SEQ ID NO: 264) | constant (SEQ ID NO: 258) |
| Hu01G06-135 | Humanized Hu01G06 IGKV1-39 F1 | Humanized Hu01G06 IGHV1-69 F1 |
| | Human Variable + Human Kappa | Heavy Human Variable + Human IgG1 |
| | constant (SEQ ID NO: 208) | constant (SEQ ID NO: 260) |
| Hu01G06-138 | Humanized Hu01G06 IGKV1-39 F1 | Humanized Hu01G06 IGHV1-69 F2 |
| | Human Variable + Human Kappa | Heavy Human Variable + Human IgG1 |
| | constant (SEQ ID NO: 208) | constant (SEQ ID NO: 262) |
| Hu01G06-146 | Humanized Hu01G06 IGKV1-39 F2 | Humanized Hu01G06 IGHV1-69 F2 |
| | Human Variable + Human Kappa | Heavy Human Variable + Human IgG1 |
| | constant (SEQ ID NO: 264) | constant (SEQ ID NO: 262) |
| Hu06C11-1 | Humanized Ch06C11 Chimeric | Humanized Ch06C11 Chimeric Heavy |
| | Human Variable + Human Kappa | Human Variable + Human IgG1 |
| | constant (SEQ ID NO: 212) | constant (SEQ ID NO: 176) |
| Hu06C11-27 | Humanized Sh06C11 IGKV1-16 | Humanized HE LM 06C11 IGHV2-70 |
| | Human Variable + Human Kappa | Heavy Human Variable + Human IgG1 |
| | constant (SEQ ID NO: 214) | constant (SEQ ID NO: 194) |
| Hu06C11-30 | Humanized Sh06C11 IGKV1-16 | Humanized Hu06C11 IGHV2-5 Heavy |
| | Human Variable + Human Kappa | Human Variable + Human IgG1 |
| | constant (SEQ ID NO: 214) | constant (SEQ ID NO: 196) |
| Hu14F11-1 | Humanized Ch14F11 Chimeric | Humanized Ch14F11 Chimeric Heavy |
| | Human Variable + Human Kappa | Human Variable + Human IgG1 |
| | constant (SEQ ID NO: 216) | constant (SEQ ID NO: 198) |
| Hu14F11-23 | Humanized Ch14F11 Chimeric | Humanized Ch06C11 Chimeric Heavy |
| | Human Variable + Human Kappa | Human Variable + Human IgG1 |
| | constant (SEQ ID NO: 216) | constant (SEQ ID NO: 192) |
| Hu14F11-24 | Humanized Ch06C11 Chimeric | Humanized Ch14F11 Chimeric Heavy |
| | Human Variable + Human Kappa | Human Variable + Human IgG1 |
| | constant (SEQ ID NO: 212) | constant (SEQ ID NO: 198) |
| Hu14F11-39 | Humanized Hu14F11 IGKV1-16 | Humanized Sh14F11 IGHV2-5 Heavy |
| | Human Variable + Human Kappa | Human Variable + Human IgG1 |
| | constant (SEQ ID NO: 218) | constant (SEQ ID NO: 200) |
| Hu14F11-47 | Humanized Hu14F11 IGKV1-16 | Humanized Sh14F11-IGHV2-70 Heavy |
| | Human Variable + Human Kappa | Human Variable + Human IgG1 |
| | constant (SEQ ID NO: 218) | constant (SEQ ID NO: 202) |

The antibody constructs containing the full length chimeric heavy and light chains are designated below:
Chimeric 01G06 (Hu01G06-1)=Full Length Ch01G06 Chimeric Heavy Chain (Mouse Heavy Chain Variable Region and Human IgG1 Constant Region) (SEQ ID NO:176) plus Full Length Ch01G06 Chimeric Light Chain (Mouse Kappa Chain Variable Region and Human Kappa Constant Region) (SEQ ID NO:204)
Chimeric 06C11 (Hu06C11-1)=Full Length Ch06C11 Chimeric Heavy Chain (Mouse Heavy Chain Variable Region and Human IgG1 Constant Region) (SEQ ID NO:192) plus Full Length Ch06C11 Chimeric Light Chain (Mouse Kappa Chain Variable Region and Human Kappa Constant Region) (SEQ ID NO:212)
Chimeric 14F11 (Hu14F11-1)=Full Length Ch14F11 Chimeric Heavy Chain (Mouse Heavy Chain Variable Region and Human IgG1 Constant Region) (SEQ ID NO:198) plus Full Length Ch14F11 Chimeric Light Chain (Mouse Kappa Chain Variable Region and Human Kappa Constant Region) (SEQ ID NO:216)

Fifteen of the possible antibody constructs containing the full length immunoglobulin heavy and light chains containing humanized variable regions are designated below:
Hu01G06-46=Full Length Hu01G06 IGHV1-18 Heavy Chain (Humanized Heavy Chain Variable Region and Human IgG1 Constant Region) (SEQ ID NO:178) plus Full Length Hu01G06 IGKV1-39 Light Chain (Humanized Kappa Chain Variable Region and Human Kappa Constant Region) (SEQ ID NO:206)
Hu01G06-52=Full Length Hu01G06 IGHV1-69 Heavy Chain (Humanized Heavy Chain Variable Region and Human IgG1 Constant Region) (SEQ ID NO:180) plus Full Length Hu01G06 IGKV1-39 Light Chain (Humanized Kappa Chain Variable Region and Human Kappa Constant Region) (SEQ ID NO:206)
Hu01G06-107=Full Length Sh01G06 IGHV1-18 M69L K64Q G44S Heavy Chain (Humanized Heavy Chain Variable Region and Human IgG1 Constant Region) (SEQ ID NO:184) plus Full Length Hu01G06 IGKV1-39 V48I Light Chain (Humanized Kappa Chain Variable Region and Human Kappa Constant Region) (SEQ ID NO:210)
Hu01G06-108=Full Length Sh01G06 IGHV1-69 T30S I69L Heavy Chain (Humanized Heavy Chain Variable Region and Human IgG1 Constant Region) (SEQ ID NO:188) plus Full Length Hu01G06 IGKV1-39 V48I Light Chain (Humanized Kappa Chain Variable Region and Human Kappa Constant Region) (SEQ ID NO:210)
Hu01G06-112=Full Length Sh01G06 IGHV1-18 M69L K64Q G44S Heavy Chain (Humanized Heavy Chain Variable Region and Human IgG1 Constant Region) (SEQ ID NO:184) plus Full Length Hu01G06 IGKV1-39 S43A V48I Light Chain (Humanized Kappa Chain Variable Region and Human Kappa Constant Region) (SEQ ID NO:208)
Hu01G06-113=Full Length Sh01G06 IGHV1-69 T30S I69L Heavy Chain (Humanized Heavy Chain Variable Region and Human IgG1 Constant Region) (SEQ ID NO:188) plus Full Length Hu01G06 IGKV1-39 S43A V48I Light Chain (Humanized Kappa Chain Variable Region and Human Kappa Constant Region) (SEQ ID NO:208)
Hu01G06-122=Full Length Hu01G06 IGHV1-18 F1 Heavy Chain (Humanized Heavy Chain Variable Region and Human IgG1 Constant Region) (SEQ ID NO:256) plus Full Length Hu01G06 IGKV1-39 F1 Light Chain (Humanized Kappa Chain Variable Region and Human Kappa Constant Region) (SEQ ID NO:208)
Hu01G06-127=Full Length Hu01G06 IGHV1-18 F2 Heavy Chain (Humanized Heavy Chain Variable Region and Human IgG1 Constant Region) (SEQ ID NO:258) plus Full Length Hu01G06 IGKV1-39 F2 Light Chain (Humanized Kappa Chain Variable Region and Human Kappa Constant Region) (SEQ ID NO:264)
Hu01G06-135=Full Length Hu01G06 IGHV1-69 F1 Heavy Chain (Humanized Heavy Chain Variable Region and Human IgG1 Constant Region) (SEQ ID NO:260) plus Full Length Hu01G06 IGKV1-39 F1 Light Chain (Humanized Kappa Chain Variable Region and Human Kappa Constant Region) (SEQ ID NO:208)
Hu01G06-138=Full Length Hu01G06 IGHV1-69 F2 Heavy Chain (Humanized Heavy Chain Variable Region and Human IgG1 Constant Region) (SEQ ID NO:262) plus Full Length Hu01G06 IGKV1-39 F1 Light Chain (Humanized Kappa Chain Variable Region and Human Kappa Constant Region) (SEQ ID NO:208)
Hu01G06-146=Full Length Hu01G06 IGHV1-69 F2 Heavy Chain (Humanized Heavy Chain Variable Region and Human IgG1 Constant Region) (SEQ ID NO:262) plus Full Length Hu01G06 IGKV1-39 F2 Light Chain (Humanized Kappa Chain Variable Region and Human Kappa Constant Region) (SEQ ID NO:264)
Hu06C11-27=Full Length HE LM 06C11 IGHV2-70 Heavy Chain (Humanized Heavy Chain Variable Region and Human IgG1 Constant Region) (SEQ ID NO:194) plus Full Length Sh06C11 IGKV1-16 Light Chain (Humanized Kappa Chain Variable Region and Human Kappa Constant Region) (SEQ ID NO:214)
Hu06C11-30=Full Length Hu06C11 IGHV2-5 Heavy Chain (Humanized Heavy Chain Variable Region and Human IgG1 Constant Region) (SEQ ID NO:196) plus Full Length Sh06C11 IGKV1-16 Light Chain (Humanized Kappa Chain Variable Region and Human Kappa Constant Region) (SEQ ID NO:214)
Hu14F11-39=Full Length Sh14F11 IGHV2-5 Heavy Chain (Humanized Heavy Chain Variable Region and Human IgG1 Constant Region) (SEQ ID NO:200) plus Full Length Hu14F11 IGKV1-16 Light Chain (Humanized Kappa Chain Variable Region and Human Kappa Constant Region) (SEQ ID NO:218)
Hu14F11-47=Full Length Sh14F11-IGHV2-70 Heavy Chain (Humanized Heavy Chain Variable Region and Human IgG1 Constant Region) (SEQ ID NO:202) plus Full Length Hu14F11 IGKV1-16 Light Chain (Humanized Kappa Chain Variable Region and Human Kappa Constant Region) (SEQ ID NO:218)

### Example 15: Binding Affinities of Humanized and Chimeric Anti-GDF15 Monoclonal Antibodies

The binding affinities and kinetics of binding of chimeric and humanized antibodies to mFc-rhGDF15 were measured by surface plasmon resonance, using a BIAcore® T100 instrument (GE Healthcare, Piscataway, NJ).

Goat anti-human IgGs (Fc fragment specific, Jackson ImmunoResearch, West Grove, PA) were immobilized on carboxymethylated dextran CM4 sensor chips by amine coupling, according to a standard protocol. Analyses were performed at 37° C. using PBS containing 0.05% surfactant P20 as running buffer. The antibodies were captured in individual flow cells at a flow rate of 10 μL/minute. Injection time was varied for each antibody to yield an Rmax between 30 and 60 RU. Buffer or mFc-rhGDF15 diluted in running buffer was injected sequentially over a reference surface (no antibody captured) and the active surface (antibody to be tested) for 240 seconds at 60 μL/minute. The dissociation phase was monitored for up to 1200 seconds. The surface was then regenerated with two 60-second injections of 10 mM Glycine-HCl, pH 2.25, at a flow rate of 30 μL/minute. The GDF15 concentration range tested was 20 nM to 0.625 nM.

Kinetic parameters were determined using the kinetic function of the BIAevaluation software (GE Healthcare) with double reference subtraction. Kinetic parameters for each antibody, kₐ (association rate constant), k_{d} (dissociation rate constant), and K_{D} (equilibrium dissociation constant) were determined. Kinetic values of purified monoclonal antibodies on mFc-rhGDF15 are summarized in Table 24.

TABLE 24

| Antibody | kₐ (1/Ms) | k_{d} (1/s) | K_{D} (M) | n |
|---|---|---|---|---|
| Hu01G06-1 | 4.2E+06 | 6.4E−04 | 1.6E−10 | 8 |
| Hu01G06-46 | 3.6E+06 | 3.8E−04 | 1.1E−10 | 11 |
| Hu01G06-52 | 3.6E+06 | 3.6E−04 | 9.9E−11 | 10 |
| Hu06C11-1 | 5.3E+06 | 8.4E−04 | 1.8E−10 | 2 |
| Hu06C11-27 | 4.7E+06 | 8.2E−04 | 1.8E−10 | 2 |
| Hu06C11-30 | 4.8E+06 | 8.7E−04 | 1.8E−10 | 2 |
| Hu14F11-1 | 3.0E+06 | 4.6E−04 | 1.6E−10 | 2 |
| Hu14F11-39 | 3.0E+06 | 1.9E−04 | 6.6E−11 | 2 |
| Hu14F11-47 | 3.3E+06 | 1.8E−04 | 6.5E−11 | 2 |

The results in Table 24 demonstrate that the chimeric and each of the humanized antibodies, have fast association rates (kₐ), very slow disassociation rates (k_{d}) and very high affinities (K_{D}). In particular, the antibodies have affinities ranging from about 65 pM to about 200 pM.

Kinetic values of chimeric 01G06 (Hu01G06-1), two initial lead humanized 01G06 monoclonal antibodies (Hu01G06-46 and -52), and sequence optimized humanized 01G06 monoclonal antibody variants Hu01G06-100 through -114 (in supernatant) on mFc-rhGDF15 are summarized in Table 25.

TABLE 25

| Antibody | kₐ (1/Ms) | k_{d} (1/s) | K_{D} (M) | n |
|---|---|---|---|---|
| Hu01G06-1 | 4.9E+06 | 7.1E−04 | 1.4E−10 | 3 |
| Hu01G06-46 | 4.1E+06 | 4.3E−04 | 1.0E−10 | 3 |
| Hu01G06-52 | 5.0E+06 | 4.4E−04 | 8.9E−11 | 3 |
| Hu01G06-100 | 4.1E+06 | 6.2E−04 | 1.5E−10 | 3 |
| Hu01G06-101 | 4.4E+06 | 6.3E−04 | 1.4E−10 | 3 |
| Hu01G06-102 | 4.4E+06 | 4.6E−04 | 1.1E−10 | 3 |
| Hu01G06-103 | 4.4E+06 | 4.7E−04 | 1.1E−10 | 3 |
| Hu01G06-104 | 4.5E+06 | 5.2E−04 | 1.2E−10 | 3 |
| Hu01G06-105 | 4.3E+06 | 5.6E−04 | 1.3E−10 | 3 |
| Hu01G06-106 | 4.3E+06 | 7.0E−04 | 1.6E−10 | 3 |
| Hu01G06-107 | 4.1E+06 | 4.7E−04 | 1.2E−10 | 3 |
| Hu01G06-108 | 4.2E+06 | 4.6E−04 | 1.2E−10 | 3 |
| Hu01G06-109 | 4.6E+06 | 5.6E−04 | 1.3E−10 | 4 |
| Hu01G06-110 | 4.3E+06 | 5.8E−04 | 1.4E−10 | 4 |
| Hu01G06-111 | 4.3E+06 | 6.6E−04 | 1.6E−10 | 3 |
| Hu01G06-112 | 4.7E+06 | 5.3E−04 | 1.2E−10 | 3 |
| Hu01G06-113 | 4.5E+06 | 4.8E−04 | 1.1E−10 | 3 |
| Hu01G06-114 | 4.5E+06 | 5.4E−04 | 1.3E−10 | 3 |

The results in Table 25 demonstrate that the sequence optimized antibodies, Hu01G06-100 through -114, have binding affinities ranging from about 89 pM to about 160 pM.

Binding affinities and binding kinetics of mFc-rhGDF15 with chimeric 14F11 (Hu14F11-1), chimeric light 14F11 with chimeric heavy 06C11 (Hu14F11-23), and chimeric light 06C11 with chimeric heavy 14F11 (Hu14F11-24) heavy monoclonal antibody variants (in supernatant) were measured using biolayer interferometry (BLI) on an Octet™ QK instrument (ForteBio, Inc., Menlo Park, CA). The Octet analysis was performed at 30° C. using 1× Kinetics Buffer (ForteBio, Inc.) as assay buffer. Anti-human IgG Fc Capture (AHC) biosensors (ForteBio, Inc.) were used to capture human antibodies onto the sensors. Sensors were saturated in assay buffer for at 300 seconds before the assay. Antibodies were loaded onto sensors by dipping the sensors into antibody supernatant solution for 220 seconds, which typically resulted in capture levels of 1.5-2 nm. Baseline was established by dipping the sensors into 1× assay buffer for 200 seconds. Next, association was monitored for 220 seconds in 400 nM mFc-rhGDF15 protein, and dissociation was followed for 600 seconds in buffer alone.

Kinetic parameters for Hu14F11-1, Hu14F11-23, and Hu14F11-24 were determined using the kinetic function of the ForteBio Analysis Software Version 7.0. Kinetic parameters of the antibody, kₐ, k_{d}, and K_{D} were determined.

Kinetic values of Hu14F11-1, Hu14F11-23, and Hu14F11-24 heavy monoclonal antibody variants (in supernatant) on mFc-rhGDF15 are summarized in Table 26.

TABLE 26

| Antibody | kₐ (1/Ms) | k_{d} (1/s) | K_{D} (M) | n |
|---|---|---|---|---|
| Hu14F11-1 | 6.3E+05 | 1.9E−05 | 3.2E−11 | 3 |
| Hu14F11-23 | 3.4E+05 | 6.2E−05 | 1.8E−10 | 1 |
| Hu14F11-24 | 7.1E+05 | 2.2E−04 | 3.1E−10 | 1 |

The results in Table 26 demonstrate that Hu14F11-23 and Hu14F11-24, (i.e., antibodies that consist of one chimeric 06C11 chain (heavy or light) mixed with one chimeric 14F11 chain (heavy or light)), retain binding to GDF15. In particular, these antibodies have high affinities ranging from about 180 pM to about 310 pM.

### Example 16: Binding Affinities of Affinity Matured Humanized Anti-GDF15 Monoclonal Antibodies

The binding affinities and kinetics of binding of chimeric and humanized antibodies to mFc-rhGDF15, cleaved-rhGDF15, rabbit Fc mature recombinant mouse GDF15 (rFc-rmGDF15), and mouse Fc mature recombinant cynomolgus monkey GDF15 (mFc-rcGDF15) were measured by surface plasmon resonance, using a BIAcore® T100 instrument (GE Healthcare, Piscataway, NJ).

Goat anti-human IgGs (Fc fragment specific, Jackson ImmunoResearch, West Grove, PA) were immobilized on carboxymethylated dextran CM4 sensor chips by amine coupling, according to a standard protocol. Analyses were performed at 37° C. using PBS containing 0.05% surfactant P20 as running buffer. The antibodies were captured in individual flow cells at a flow rate of 10 μL/minute. Injection time was varied for each antibody to yield an Rmax between 30 and 60 RU. Buffer, mFc-rhGDF15, cleaved-rhGDF15, rFc-rmGDF15, or mFc-rcGDF15 diluted in running buffer was injected sequentially over a reference surface (no antibody captured) and the active surface (antibody to be tested) for 240 seconds at 60 μL/minute. The dissociation phase was monitored for up to 1500 seconds. The surface was then regenerated with two 60-second injections of 10 mM Glycine-HCl, pH 2.25, at a flow rate of 30 μL/minute. The GDF15 concentration range tested for each GDF15 protein was 5 nM to 0.3125 nM (two-fold dilutions).

Kinetic parameters were determined using the kinetic function of the BIAevaluation software (GE Healthcare) with double reference subtraction. Kinetic parameters for each antibody, kₐ (association rate constant), k_{d} (dissociation rate constant), and K_{D} (equilibrium dissociation constant) were determined. Kinetic values of purified monoclonal antibodies on mFc-rhGDF15, mature human GDF15, rFc-rmGDF15, and mFc-rcGDF15 are summarized in Table 27.

TABLE 27

| Protein | Antibody | kₐ (1/Ms) | k_{d} (1/s) | K_{D} (M) | n |
|---|---|---|---|---|---|
| mFc- | Hu01G06-122 | 5.9E+06 | 2.1E−05 | 6.5E−12 | 5 |
| rhGDF15 | Hu01G06-127 | 4.6E+06 | 4.2E−05 | 1.8E−11 | 4 |
| | Hu01G06-135 | 5.3E+06 | 4.4E−05 | 1.4E−11 | 5 |
| | Hu01G06-138 | 5.9E+06 | 4.1E−05 | 1.1E−11 | 5 |
| | Hu01G06-146 | 5.3E+06 | 2.6E−05 | 9.3E−12 | 5 |
| Cleaved- | Hu01G06-122 | 7.9E+06 | 3.4E−05 | 7.9E−12 | 4 |
| rhGDF15 | Hu01G06-127 | 6.1E+06 | 3.6E−05 | 1.0E−11 | 4 |
| | Hu01G06-135 | 7.3E+06 | 6.2E−05 | 1.0E−11 | 4 |
| | Hu01G06-138 | 7.9E+06 | 2.5E−05 | 4.5E−12 | 4 |
| | Hu01G06-146 | 6.5E+06 | 5.2E−05 | 1.1E−11 | 4 |
| mFc- | Hu01G06-122 | 2.3E+06 | 2.4E−05 | 1.0E−11 | 4 |
| rcGDF15 | Hu01G06-127 | 1.8E+06 | 1.6E−05 | 9.5E−12 | 4 |
| | Hu01G06-135 | 2.2E+06 | 7.9E−05 | 3.8E−11 | 4 |
| | Hu01G06-138 | 2.3E+06 | 5.3E−05 | 2.5E−11 | 4 |
| | Hu01G06-146 | 2.0E+06 | 1.5E−05 | 8.0E−12 | 4 |
| rFc- | Hu01G06-122 | 2.2E+07 | 1.4E−03 | 6.3E−11 | 2 |
| rmGDF15 | Hu01G06-127 | 3.9E+07 | 2.1E−03 | 5.1E−11 | 2 |
| | Hu01G06-135 | 3.7E+07 | 1.9E−03 | 5.5E−11 | 2 |
| | Hu01G06-138 | 1.9E+07 | 8.0E−04 | 4.4E−11 | 2 |
| | Hu01G06-146 | 1.1E+07 | 7.2E−04 | 6.3E−11 | 2 |

The results in Table 27 demonstrate that the chimeric and each of the humanized antibodies, have fast association rates (kₐ), very slow disassociation rates (k_{d}) and very high affinities (K_{D}). In particular, the antibodies have affinities ranging from less than 5 pM (e.g., about 4.5 pM) to about 65 pM.

### Example 17: Reversal of Cachexia in an HT-1080 Fibroscarcoma Xenograft Model

This Example demonstrates the reversal of cachexia (as indicated by body weight loss) by humanized 01G06, 06C11, 14F11 antibodies in an HT-1080 fibrosarcoma xenograft model. HT-1080 cells were grown in culture at 37° C. and inoculated subcutaneously into the flank of 8-week old female ICR-SCID mice as described above in Example 10. Body weight was measured daily. When body weight reached 93%, the mice were randomly divided into groups of ten mice each. Each group received one of the following treatments: murine IgG control, 01G06, 06C11, 14F11, and their respective humanized versions at 2 mg/kg. Treatment was administered once a day by intra-peritoneal injection. Antibody treatment with 01G06, Hu01G06-46 and Hu01G06-52 resulted in body weight increase to initial weight or 100% (p<0.001) (FIG. 23). Statistical analysis was performed using ANOVA. Results for reversal of body weight on day in the HT-1080 model are shown in FIG. 23 and Table 28, respectively.

TABLE 28

| | | |
|---|---|---|
| Treatment | % Body | ANOVA Analysis |

| Gr. | Agent | mg/kg | weight | (compared to mIgG) |
|---|---|---|---|---|
| 1 | mIgG | 2 | 79.5 | NA |
| 2 | 01G06 | 2 | 87.6 | p < 0.001 |
| 3 | Hu01G06-46 | 2 | 95.4 | p < 0.001 |
| 4 | Hu01G06-52 | 2 | 87.8 | p < 0.001 |

The data in FIG. 23 and Table 28 indicated that antibodies 01G06, Hu01G06-46 and Hu01G06-52 can reverse cachexia in an HT-1080 fibrosarcoma xenograft model.

Antibody treatment with 06C11, Hu06C11-27, and Hu06C11-30 resulted in body weight increase relative to initial weight or about 100% (p<0.001) (FIG. 24). Statistical analysis was performed using ANOVA. Results for reversal of body weight in the HT-1080 model are shown in FIG. 24 and Table 29.

TABLE 29

| | | |
|---|---|---|
| Treatment | % Body | ANOVA Analysis |

| Gr. | Agent | mg/kg | weight | (compared to mIgG) |
|---|---|---|---|---|
| 1 | mIgG | 2 | 87.7 | NA |
| 2 | 01G06 | 2 | 93.6 | p < 0.001 |
| 3 | Hu06C11-27 | 2 | 93.2 | p < 0.001 |
| 4 | Hu06C11-30 | 2 | 89.8 | p < 0.001 |

The data in FIG. 24 and Table 29 indicate that antibodies 06C11, Hu06C11-27, and Hu06C11-30 can reverse cachexia in an HT-1080 fibrosarcoma xenograft model.

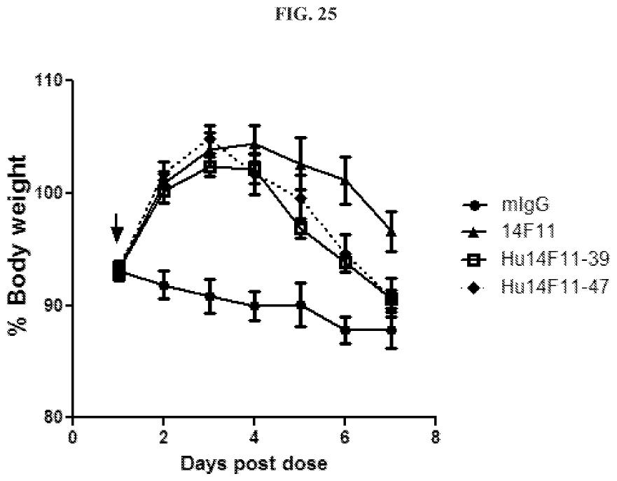

Antibody treatment with 14F11, Hu14F11-39, and Hu14F11-47 resulted in body weight increase relative to initial weight or about 100% (p<0.001) (FIG. 25). Statistical analysis was performed using ANOVA. Results for reversal of body weight in the HT-1080 model are shown in FIG. 25 and Table 30.

TABLE 30

| | | |
|---|---|---|
| Treatment | % Body | ANOVA Analysis |

| Gr. | Agent | mg/kg | weight | (compared to mIgG) |
|---|---|---|---|---|
| 1 | mIgG | 2 | 87.7 | NA |
| 2 | 14F11 | 2 | 96.6 | p < 0.001 |
| 3 | Hu14F11-39 | 2 | 90.5 | p < 0.001 |
| 4 | Hu14F11-47 | 2 | 90.7 | p < 0.001 |

The data in FIG. 25 and Table 30 indicated that antibodies 14F11, Hu14F11-39, and Hu14F11-47 can reverse cachexia in an HT-1080 fibrosarcoma xenograft model.

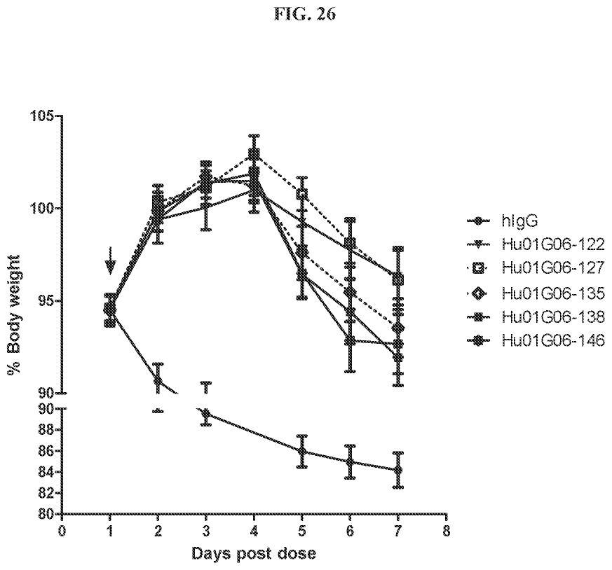

Antibody treatment with humanized 01G06 antibodies (i.e., antibodies Hu01G06-122, Hu01G06-127, Hu01G06-135, Hu01G06-138 and Hu01G06-146) resulted in body weight increase relative to initial weight or about 100% (p<0.001) (FIG. 26). Statistical analysis was performed using ANOVA. Treatment with human IgG (hIgG) was used as a control. Results for reversal of body weights in the HT-1080 model are shown in FIG. 26 and Table 31.

TABLE 31

| | Treatment | | % Body | ANOVA Analysis |
|---|---|---|---|---|
| Gr. | Agent | mg/kg | weight | (compared to mIgG) |
| 1 | mIgG | 2 | 84.2 | NA |
| 2 | Hu01G06-122 | 2 | 96.3 | p < 0.001 |
| 3 | Hu01G06-127 | 2 | 96.1 | p < 0.001 |
| 4 | Hu01G06-135 | 2 | 93.5 | p < 0.001 |
| 5 | Hu01G06-138 | 2 | 91.9 | p < 0.001 |
| 6 | Hu01G06-146 | 2 | 92.7 | p < 0.001 |

The data in FIG. 26 and Table 31 indicated that humanized anti-GDF15 antibodies Hu01G06-122, Hu01G06-127, Hu01G06-135, Hu01G06-138 and Hu01G06-146 can reverse cachexia in an HT-1080 fibrosarcoma xenograft model.

### Example 18: Reversal of Cachexia in an mFc-rhGDF15-Induced Model

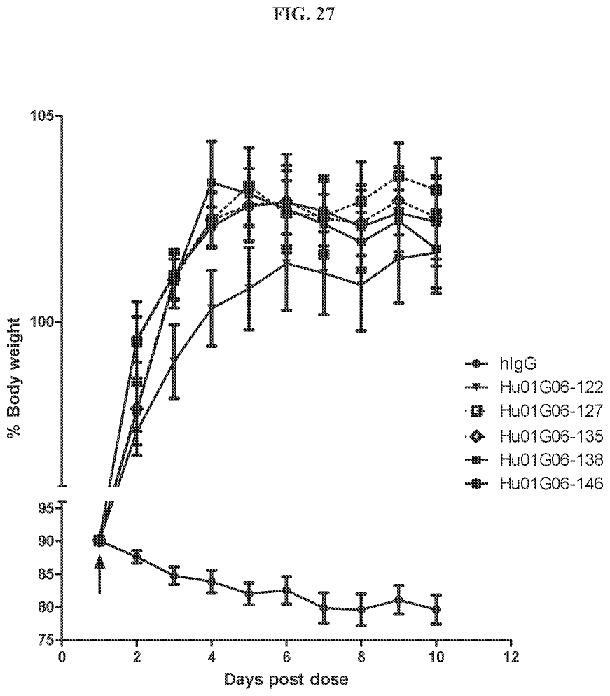

This Example demonstrates the reversal of cachexia (as indicated by body weight loss) by humanized 01G06 antibodies (i.e., antibody Hu01G06-122, Hu01G06-127, Hu01G06-135, Hu01G06-138, or Hu01G06-146) in an mFc-rhGDF15-induced cachexia model. mFc-rhGDF15 (1 μg/g) was administered subcutaneously into the flank of 8-week old female ICR-SCID mice. Body weight was measured daily. When body weight reached 93%, the mice were randomly divided into six groups of ten mice each. Each group received one of the following treatments: human IgG control (hIgG), Hu01G06-122, Hu01G06-127, Hu01G06-135, Hu01G06-138 or Hu01G06-146 at 2 mg/kg. Treatment was administered once by intra-peritoneal injection. Treatment with antibody Hu01G06-122, Hu01G06-127, Hu01G06-135, Hu01G06-138 or Hu01G06-146 resulted in body weight increase relative to initial weight or about 100% (p<0.001) (FIG. 27 and Table 32).

TABLE 32

| | Treatment | | % Body | ANOVA Analysis |
|---|---|---|---|---|
| Gr. | Agent | mg/kg | weight | (compared to mIgG) |
| 1 | mIgG | 2 | 70.6 | NA |
| 2 | Hu01G06-122 | 2 | 101.7 | p < 0.001 |
| 3 | Hu01G06-127 | 2 | 103.2 | p < 0.001 |
| 4 | Hu01G06-135 | 2 | 102.5 | p < 0.001 |
| 5 | Hu01G06-138 | 2 | 101.8 | p < 0.001 |
| 6 | Hu01G06-146 | 2 | 102.5 | p < 0.001 |

The data in FIG. 27 and Table 32 indicate that the disclosed anti-GDF15 antibodies can reverse cachexia in an mFc-rhGDF15-induced mouse model (i.e., a non-tumor bearing mouse model).

These results indicate that humanized anti-GDF15 antibodies can reverse cachexia in an mFc-rhGDF15-induced cachexia model.

### Example 19: Dose Response Reversal of Cachexia in an HT-1080 Fibroscarcoma Xenograft Model

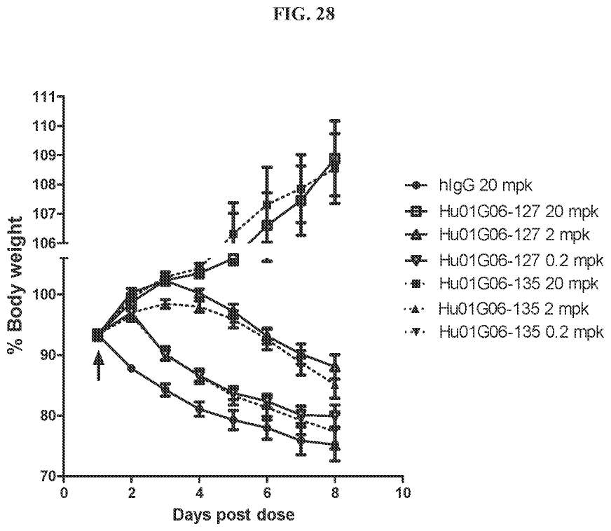

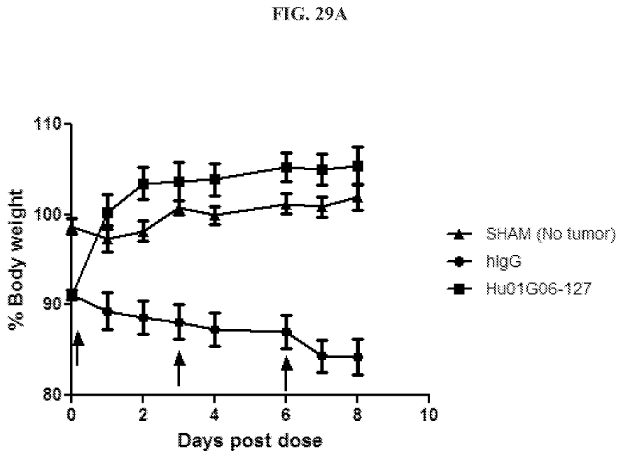

This Example demonstrates the dose response reversal of cachexia (as indicated by body weight loss) by humanized Hu01G06-127 and Hu01G06-135 antibodies in an HT-1080 fibrosarcoma xenograft model. HT-1080 cells were grown in culture at 37° C. and inoculated subcutaneously into the flank of 8-week old female ICR-SCID mice as described above in Example 10. Body weight was measured daily. When body weight reached 93%, the mice were randomly divided into groups of ten mice each. Each group received one of the following treatments: human IgG control (hIgG; 20 mg/kg), Hu01G06-127 (20 mg/kg, 2 mg/kg, or 0.2 mg/kg) and Hu01G06-135 (20 mg/kg, 2 mg/kg, or 0.2 mg/kg). Treatment was administered once a day by intravenous injection. Antibody treatment with Hu01G06-127 and Hu01G06-135 at 20 mg/kg resulted in body weight increase above the initial weight or 108% (p<0.001) (FIG. 28). Antibody treatment with Hu01G06-127 and Hu01G06-135 at 2 mg/kg resulted in limited body weight decrease compare to control (hIgG) from the initial weight or 88-85% (p<0.001) (FIG. 28). Statistical analysis was performed using ANOVA. Results for changes of body weight at the end of the study in the HT-1080 model are shown in FIG. 28 and Table 33.

TABLE 33

| | Treatment | | % Body | ANOVA Analysis |
|---|---|---|---|---|
| Gr. | Agent | mg/kg | weight | (compared to mIgG) |
| 1 | mIgG | 20 | 75.2 | NA |
| 2 | Hu01G06-127 | 20 | 108.9 | p < 0.001 |
| 3 | Hu01G06-127 | 2.0 | 88.1 | p < 0.001 |
| 4 | Hu01G06-127 | 0.2 | 80.0 | NS |
| 5 | Hu01G06-135 | 20 | 108.6 | p < 0.001 |
| 6 | Hu01G06-135 | 2.0 | 85.2 | p < 0.001 |
| 7 | Hu01G06-135 | 0.2 | 77.3 | NS |

The data in FIG. 28 and Table 33 indicated that antibodies Hu01G06-127 and Hu01G06-135 can reverse cachexia in an HT-1080 fibrosarcoma xenograft model in a dose-dependent manner.

### Example 20: Reversal of Muscle and Fat Loss in an HT-1080 Xenograft Tumor Model

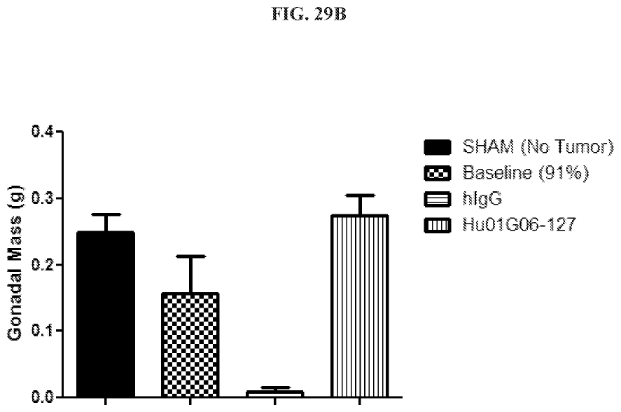

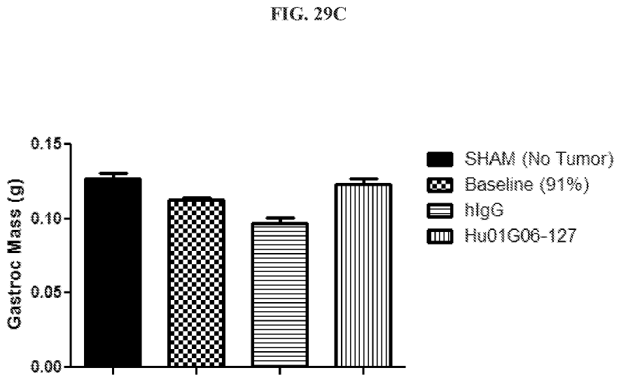

This Example demonstrates the reversal of cachexia (as indicated by body weight loss, muscle mass loss and fat mass loss) by antibody 01G06 in an HT-1080 fibrosarcoma xenograft model. HT-1080 cells were grown in culture at 37° C. in an atmosphere containing 5% CO₂, using Eagle's Minimum Essential Medium (ATCC, Catalog No. 30-2003) containing 10% FBS. Cells were inoculated subcutaneously into the flank of 8-week old female ICR SCID mice with 5×10⁶ cells per mouse in 50% matrigel. A cohort of ten 8-week old female ICR SCID mice with the same body weight was selected for subcutaneous inoculation into the flank with matrigel, as a non tumor (SHAM) control arm. Body weight was measured daily. When body weight reached 91% in the tumor bearing mice, the mice were randomly divided into two groups of ten mice each. Each group received one of the following treatments: human IgG control (hIgG) or Hu01G06-127 10 mg/kg on day 1, day 3 and day 6. Treatment was administered by intra-peritoneal injection. Treatment with antibody Hu01G06-127 resulted in body weight increase to 105% of initial weight compared to non-tumor bearing control mice (SHAM; p<0.001) (FIG. 29A and Table 34).

TABLE 34

| | Treatment | | % Body | ANOVA Analysis |
|---|---|---|---|---|
| Gr. | Agent | mg/kg | weight | (compared to hIgG) |
| 1 | hIgG | 10 | 84.3 | NA |
| 2 | Hu01G06-127 | 10 | 105.4 | p < 0.001 |
| 2 | SHAM no tumor control | none | 101.9 | p < 0.001 |

The data in FIG. 29A and Table 34 indicate that the disclosed anti-GDF15 antibody can completely reverse cachexia in an HT-1080 fibrosarcoma xenograft model.

In this experiment, a group of ten mice were sacrificed at the time of dosing (baseline or 91% body weight loss, without treatment) and at the end of the study (eight days post dose, either hIgG or Hu01G06-127 as well SHAM non tumor control mice). Gonadal fat and the gastrocnemius muscles were removed surgically and weighed. As shown in FIG. 29B, significant gonadal fat mass loss was observed seven days post dose with hIgG, but not in the group treated with antibody Hu01G06-127 compared to baseline control (91% body weight loss). Moreover, treatment with Hu01G06-127 not only prevented further fat loss (compared to baseline group), but also, was able to restore the normal levels of gonadal fat (compared to SHAM non-tumor control) (FIG. 29B). In addition, significant gastrocnemius muscle mass loss was observed seven days post dose with hIgG, but not in the group treated with antibody Hu01G06-127 compared to baseline control (91% body weight loss) (FIG. 29C). Treatment with Hu01G06-127 not only prevented further muscle loss (compare to baseline group), but also was able to restore the normal levels of gastrocnemius muscle (compared to SHAM non tumor control) (FIG. 29C).

These results indicate that the disclosed anti-GDF15 antibodies can completely reverse cachexia measured by the loss of muscle mass, loss of fat and involuntary weight loss in an HT-1080 xenograft tumor model.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1-61. (canceled)

62. A method of treating cachexia in a mammal comprising administering an effective amount of an anti-GDF15 antibody or antigen-binding fragment thereof that specifically binds to the same epitope on GDF15 as an antibody comprising an immunoglobulin heavy chain variable region and an immunoglobulin light chain variable region selected from the group consisting of:
(a) (i) an immunoglobulin heavy chain variable region comprising a CDR H1 comprising an amino acid sequence selected from the group consisting of SEQ ID NO: 1, SEQ ID NO:38 and SEQ ID NO:136 (Hu01G06 IGHV1-18 F2), a CDR H2 comprising an amino acid sequence selected from the group consisting of SEQ ID NO:237, SEQ ID NO:241 and SEQ ID NO:243 (Hu01G06 IGHV1-18 F2), and a CDR H3 comprising an amino acid sequence selected from the group consisting of SEQ ID NO:15 and SEQ ID NO:154 (Hu01G06 IGHV1-18 F2); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2);
(b) (i) an immunoglobulin heavy chain variable region comprising a CDR H1 comprising an amino acid sequence selected from the group consisting of SEQ ID NO: 1, SEQ ID NO:234 and SEQ ID NO:235 (Hu01G06 IGHV1-69 F1), a CDR H2 comprising an amino acid sequence selected from the group consisting of SEQ ID NO:238, SEQ ID NO:241 and SEQ ID NO:243 (Hu01G06 IGHV1-69 F1), and a CDR H3 comprising an amino acid sequence selected from the group consisting of SEQ ID NO:15 and SEQ ID NO:154 (Hu01G06 IGHV1-69 F1); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F1), a CDR L2 comprising an amino acid sequence selected from the group consisting of SEQ ID NO:26 and DAK (Hu01G06 IGKV1-39 F1), and a CDR L3 comprising the amino acid sequence of SEQ ID NO:32 (Hu01G06 IGKV1-39 F1);
(c) (i) an immunoglobulin heavy chain variable region comprising a CDR H1 comprising an amino acid sequence selected from the group consisting of SEQ ID NO:1, SEQ ID NO:38 and SEQ ID NO:136 (01G06, Ch01G06 Chimeric, Hu01G06 IGHV1-18, Hu01G06 IGHV1-69, Sh01G06 IGHV1-18 M69L), a CDR H2 comprising an amino acid sequence selected from the group consisting of SEQ ID NO:7, SEQ ID NO:143 and SEQ ID NO:148 (01G06, Ch01G06 Chimeric, Hu01G06 IGHV1-18, Hu01G06 IGHV1-69, Sh01G06 IGHV1-18 M69L), and a CDR H3 comprising an amino acid sequence selected from the group consisting of SEQ ID NO:15 and SEQ ID NO:154 (01G06, Ch01G06 Chimeric, Hu01G06 IGHV1-18, Hu01G06 IGHV1-69, Sh01G06 IGHV1-18 M69L); and
(ii) an immunoglobulin light chain variable region comprising a CDR L1 comprising an amino acid sequence selected from the group consisting of SEQ ID NO:21 and SEQ ID NO:160 (01G06, Ch01G06 Chimeric, Hu01G06 IGKV1-39, Hu01G06 IGKV1-39 S43A V48I, Hu01G06 IGKV1-39 V48I), a CDR L2 comprising an amino acid sequence selected from the group consisting of SEQ ID NO:26 and DAK (01G06, Ch01G06 Chimeric, Hu01G06 IGKV1-39, Hu01G06 IGKV1-39 S43A V48I, Hu01G06 IGKV1-39 V48I), and a CDR L3 comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I);
(d) (i) an immunoglobulin heavy chain variable region comprising a CDR H1 comprising the amino acid sequence of SEQ ID NO:2 (03G05), a CDR H2 comprising the amino acid sequence of SEQ ID NO:8 (03G05), and a CDR H3 comprising the amino acid sequence of SEQ ID NO:16 (03G05); and
(ii) an immunoglobulin light chain variable region comprising a CDR L1 comprising the amino acid sequence of SEQ ID NO:22 (03G05), a CDR L2 comprising the amino acid sequence of SEQ ID NO:27 (03G05), and a CDR L3 comprising the amino acid sequence of SEQ ID NO: 33 (03G05);
(e) (i) an immunoglobulin heavy chain variable region comprising a CDR H1 comprising the amino acid sequence of SEQ ID NO:3 (04F08), a CDR H2 comprising the amino acid sequence of SEQ ID NO:9 (04F08), and a CDR H3 comprising the amino acid sequence of SEQ ID NO:17 (04F08); and
(ii) an immunoglobulin light chain variable region comprising a CDR L1 comprising the amino acid sequence of SEQ ID NO:23 (04F08), a CDR L2 comprising the amino acid sequence of SEQ ID NO:28 (04F08), and a CDR L3 comprising the amino acid sequence of SEQ ID NO:34 (04F08);
(f) (i) an immunoglobulin heavy chain variable region comprising a CDR H1 comprising the amino acid sequence of SEQ ID NO:4 (06C11, Ch06C11 Chimeric, Hu06C11 IGHV2-5), a CDR H2 comprising the amino acid sequence of SEQ ID NO:9 (06C11, Ch06C11 Chimeric, Hu06C11 IGHV2-5), and a CDR H3 comprising the amino acid sequence of SEQ ID NO:18 (06C11, Ch06C11 Chimeric, Hu06C11 IGHV2-5); and
(ii) an immunoglobulin light chain variable region comprising a CDR L1 comprising the amino acid sequence of SEQ ID NO:23 (06C11, Ch06C11 Chimeric, Sh06C11 IGKV1-16), a CDR L2 comprising the amino acid sequence of SEQ ID NO:28 (06C11, Ch06C11 Chimeric, Sh06C11 IGKV1-16), and a CDR L3 comprising the amino acid sequence of SEQ ID NO:35 (06C11, Ch06C11 Chimeric, Sh06C11 IGKV1-16);
(g) (i) an immunoglobulin heavy chain variable region comprising a CDR H1 comprising the amino acid sequence of SEQ ID NO:1 (08G01), a CDR H2 comprising the amino acid sequence of SEQ ID NO:10 (08G01), and a CDR H3 comprising the amino acid sequence of SEQ ID NO:15 (08G01); and
(ii) an immunoglobulin light chain variable region comprising a CDR L1 comprising the amino acid sequence of SEQ ID NO:24 (08G01), a CDR L2 comprising the amino acid sequence of SEQ ID NO:29 (08G01), and a CDR L3 comprising the amino acid sequence of SEQ ID NO:32 (08G01);
(h) (i) an immunoglobulin heavy chain variable region comprising a CDR H1 comprising the amino acid sequence of SEQ ID NO:5 (14F11, Ch14F11 Chimeric, Sh14F11 IGHV2-5, Sh14F11 IGHV2-70), a CDR H2 comprising the amino acid sequence of SEQ ID NO:11 (14F11, Ch14F11 Chimeric, Sh14F11 IGHV2-5, Sh14F11 IGHV2-70), and a CDR H3 comprising the amino acid sequence of SEQ ID NO:19 (14F11, Ch14F11 Chimeric, Sh14F11 IGHV2-5, Sh14F11 IGHV2-70); and
(ii) an immunoglobulin light chain variable region comprising a CDR L1 comprising the amino acid sequence of SEQ ID NO:23 (14F11, Ch14F11 Chimeric, Hu14F11 IGKV1-16), a CDR L2 comprising the amino acid sequence of SEQ ID NO:30 (14F11, Ch14F11 Chimeric, Hu14F11 IGKV1-16), and a CDR L3 comprising the amino acid sequence of SEQ ID NO:36 (14F11, Ch14F11 Chimeric, Hu14F11 IGKV1-16);
(i) (i) an immunoglobulin heavy chain variable region comprising a CDR H1 comprising the amino acid sequence of SEQ ID NO:6 (17B11), a CDR H2 comprising the amino acid sequence of SEQ ID NO:12 (17B11), and a CDR H3 comprising the amino acid sequence of SEQ ID NO:20 (17B11); and
(ii) an immunoglobulin light chain variable region comprising a CDR L1 comprising the amino acid sequence of SEQ ID NO:25 (17B11), a CDR L2 comprising the amino acid sequence of SEQ ID NO:31 (17B11), and a CDR L3 comprising the amino acid sequence of SEQ ID NO:37 (17B11);
(j) (i) an immunoglobulin heavy chain variable region comprising a CDR H1 comprising an amino acid sequence selected from the group consisting of SEQ ID NO:1, SEQ ID NO:38 and SEQ ID NO:136 (Sh01G06 IGHV1-18 M69L K64Q G44S, Sh01G06 IGHV1-18 M69L K64Q), a CDR H2 comprising an amino acid sequence selected from the group consisting of SEQ ID NO:13, SEQ ID NO:143 and SEQ ID NO:148 (Sh01G06 IGHV1-18 M69L K64Q G44S, Sh01G06 IGHV1-18 M69L K64Q), and a CDR H3 comprising an amino acid sequence selected from the group consisting of SEQ ID NO:15 and SEQ ID NO:154 (Sh01G06 IGHV1-18 M69L K64Q G44S, Sh01G06 IGHV1-18 M69L K64Q); and
(ii) an immunoglobulin light chain variable region comprising a CDR L1 comprising an amino acid sequence selected from the group consisting of SEQ ID NO:21 and SEQ ID NO:160 (01G06, Ch01G06 Chimeric, Hu01G06 IGKV1-39, Hu01G06 IGKV1-39 S43A V48I, Hu01G06 IGKV1-39 V48I), a CDR L2 comprising an amino acid sequence selected from the group consisting of SEQ ID NO:26 and DAK (01G06, Ch01G06 Chimeric, Hu01G06 IGKV1-39, Hu01G06 IGKV1-39 S43A V48I, Hu01G06 IGKV1-39 V48I), and a CDR L3 comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I);
(k) (i) an immunoglobulin heavy chain variable region comprising a CDR H1 comprising an amino acid sequence selected from the group consisting of SEQ ID NO: 1, SEQ ID NO:38 and SEQ ID NO:136 (Hu01G06 IGHV1-18 F1), a CDR H2 comprising an amino acid sequence selected from the group consisting of SEQ ID NO:236, SEQ ID NO:240 and SEQ ID NO:242 (Hu01G06 IGHV1-18 F1), and a CDR H3 comprising an amino acid sequence selected from the group consisting of SEQ ID NO:15 and SEQ ID NO:154 (Hu01G06 IGHV1-18 F1); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F1), a CDR L2 comprising an amino acid sequence selected from the group consisting of SEQ ID NO:26 and DAK (Hu01G06 IGKV1-39 F1), and a CDR L3 comprising the amino acid sequence of SEQ ID NO:32 (Hu01G06 IGKV1-39 F1);
(l) (i) an immunoglobulin heavy chain variable region comprising a CDR H1 comprising an amino acid sequence selected from the group consisting of SEQ ID NO: 1, SEQ ID NO:234, and SEQ ID NO:235 (Hu01G06 IGHV1-69 F2), a CDR H2 comprising an amino acid sequence selected from the group consisting of SEQ ID NO:239, SEQ ID NO:240 and SEQ ID NO:242 (Hu01G06 IGHV1-69 F2), and a CDR H3 comprising an amino acid sequence selected from the group consisting of SEQ ID NO:15 and SEQ ID NO:154 (Hu01G06 IGHV1-69 F2); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F1), a CDR L2 comprising an amino acid sequence selected from the group consisting of SEQ ID NO:26 and DAK (Hu01G06 IGKV1-39 F1), and a CDR L3 comprising the amino acid sequence of SEQ ID NO:32 (Hu01G06 IGKV1-39 F1);
(m) (i) an immunoglobulin heavy chain variable region comprising a CDR H1 comprising an amino acid sequence selected from the group consisting of SEQ ID NO: 1, SEQ ID NO:234 and SEQ ID NO:235 (Hu01G06 IGHV1-69 F2), a CDR H2 comprising an amino acid sequence selected from the group consisting of SEQ ID NO:239, SEQ ID NO:240 and SEQ ID NO:242 (Hu01G06 IGHV1-69 F2), and a CDR H3 comprising an amino acid sequence selected from the group consisting of SEQ ID NO:15 and SEQ ID NO:154 (Hu01G06 IGHV1-69 F2); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2);
(n) (i) an immunoglobulin heavy chain variable region comprising a CDR H1 comprising the amino acid sequence of SEQ ID NO:4 (HE LM 06C11 IGHV2-70), a CDR H2 comprising the amino acid sequence of SEQ ID NO:14 (HE LM 06C11 IGHV2-70), and a CDR H3 comprising the amino acid sequence of SEQ ID NO:18 (HE LM 06C11 IGHV2-70); and
(ii) an immunoglobulin light chain variable region comprising a CDR L1 comprising the amino acid sequence of SEQ ID NO:23 (Ch06C11 Chimeric, Sh06C11 IGKV1-16), a CDR L2 comprising the amino acid sequence of SEQ ID NO:28 (Ch06C11 Chimeric, Sh06C11 IGKV1-16), and a CDR L3 comprising the amino acid sequence of SEQ ID NO:35 (Ch06C11 Chimeric, Sh06C11 IGKV1-16);
(o) (i) an immunoglobulin heavy chain variable region comprising a CDR H1 comprising the amino acid sequence of SEQ ID NO:4 (Ch06C11 Chimeric), a CDR H2 comprising the amino acid sequence of SEQ ID NO:9 (Ch06C11 Chimeric), and a CDR H3 comprising the amino acid sequence of SEQ ID NO:18 (Ch06C11 Chimeric); and
(ii) an immunoglobulin light chain variable region comprising a CDR L1 comprising the amino acid sequence of SEQ ID NO:23 (Ch14F11 Chimeric), a CDR L2 comprising the amino acid sequence of SEQ ID NO:30 (Ch14F11 Chimeric), and a CDR L3 comprising the amino acid sequence of SEQ ID NO:36 (Ch14F11 Chimeric);
(p) (i) an immunoglobulin heavy chain variable region comprising a CDR H1 comprising the amino acid sequence of SEQ ID NO:5 (Ch14F11 Chimeric), a CDR H2 comprising the amino acid sequence of SEQ ID NO:11 (Ch14F11 Chimeric), and a CDR H3 comprising the amino acid sequence of SEQ ID NO:19 (Ch14F11 Chimeric); and
(ii) an immunoglobulin light chain variable region comprising a CDR L1 comprising the amino acid sequence of SEQ ID NO:23 (Ch06C11 Chimeric), a CDR L2 comprising the amino acid sequence of SEQ ID NO:28 (Ch06C11 Chimeric), and a CDR L3 comprising the amino acid sequence of SEQ ID NO:35 (Ch06C11 Chimeric);
(q) (i) an immunoglobulin heavy chain variable region comprising a CDR H1 comprising an amino acid sequence selected from the group consisting of SEQ ID NO:1, SEQ ID NO:234 and SEQ ID NO:235 (Sh01G06 IGHV1-69 T30S I69L), a CDR H2 comprising an amino acid sequence selected from the group consisting of SEQ ID NO:7, SEQ ID NO:143 and SEQ ID NO:148 (Sh01G06 IGHV1-69 T30S I69L), and a CDR H3 comprising an amino acid sequence selected from the group consisting of SEQ ID NO:15 and SEQ ID NO:154 (Sh01G06 IGHV1-69 T30S I69L); and
(ii) an immunoglobulin light chain variable region comprising a CDR L1 comprising an amino acid sequence selected from the group consisting of SEQ ID NO:21 and SEQ ID NO:160 (01G06, Ch01G06 Chimeric, Hu01G06 IGKV1-39, Hu01G06 IGKV1-39 S43A V48I, Hu01G06 IGKV1-39 V48I), a CDR L2 comprising an amino acid sequence selected from the group consisting of SEQ ID NO:26 and DAK (01G06, Ch01G06 Chimeric, Hu01G06 IGKV1-39, Hu01G06 IGKV1-39 S43A V48I, Hu01G06 IGKV1-39 V48I), and a CDR L3 comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I);
(r) (i) an immunoglobulin heavy chain variable region comprising a CDR H1 comprising an amino acid sequence selected from the group consisting of SEQ ID NO:1, SEQ ID NO:234 and SEQ ID NO:235 (Sh01G06 IGHV1-69 T30S K64Q I69L), a CDR H2 comprising an amino acid sequence selected from the group consisting of SEQ ID NO:13, SEQ ID NO:143 and SEQ ID NO:148 (Sh01G06 IGHV1-69 T30S K64Q I69L), and a CDR H3 comprising an amino acid sequence selected from the group consisting of SEQ ID NO:15 and SEQ ID NO:154 (Sh01G06 IGHV1-69 T30S K64Q I69L); and
(ii) an immunoglobulin light chain variable region comprising a CDR L1 comprising an amino acid sequence selected from the group consisting of SEQ ID NO:21 and SEQ ID NO:160 (01G06, Ch01G06 Chimeric, Hu01G06 IGKV1-39, Hu01G06 IGKV1-39 S43A V48I, Hu01G06 IGKV1-39 V48I), a CDR L2 comprising an amino acid sequence selected from the group consisting of SEQ ID NO:26 and DAK (01G06, Ch01G06 Chimeric, Hu01G06 IGKV1-39, Hu01G06 IGKV1-39 S43A V48I, Hu01G06 IGKV1-39 V48I), and a CDR L3 comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I);
(s) (i) an immunoglobulin heavy chain variable region comprising a CDR H1 comprising an amino acid sequence selected from the group consisting of SEQ ID NO:1, SEQ ID NO:38 and SEQ ID NO:136 (Hu01G06 IGHV1-18 F2), a CDR H2 comprising an amino acid sequence selected from the group consisting of SEQ ID NO:237, SEQ ID NO:241 and SEQ ID NO:243 (Hu01G06 IGHV1-18 F2), and a CDR H3 comprising an amino acid sequence selected from the group consisting of SEQ ID NO:15 and SEQ ID NO:154 (Hu01G06 IGHV1-18 F2); and
(ii) an immunoglobulin light chain variable region comprising a CDR L1 comprising an amino acid sequence selected from the group consisting of SEQ ID NO:21 and SEQ ID NO:160 (01G06, Ch01G06 Chimeric, Hu01G06 IGKV1-39, Hu01G06 IGKV1-39 S43A V48I, Hu01G06 IGKV1-39 V48I), a CDR L2 comprising an amino acid sequence selected from the group consisting of SEQ ID NO:26 and DAK (01G06, Ch01G06 Chimeric, Hu01G06 IGKV1-39, Hu01G06 IGKV1-39 S43A V48I, Hu01G06 IGKV1-39 V48I), and a CDR L3 comprising the amino acid sequence of SEQ ID NO:32 (01G06, Ch01G06 Chimeric, Hu01G06 IGKV1-39, Hu01G06 IGKV1-39 S43A V48I, Hu01G06 IGKV1-39 V48I);
(t) (i) an immunoglobulin heavy chain variable region comprising a CDR H1 comprising an amino acid sequence selected from the group consisting of SEQ ID NO:1, SEQ ID NO:38 and SEQ ID NO:136 (01G06, Ch01G06 Chimeric, Hu01G06 IGHV1-18, Hu01G06 IGHV1-69, Sh01G06 IGHV1-18 M69L), a CDR H2 comprising an amino acid sequence selected from the group consisting of SEQ ID NO:7, SEQ ID NO:143 and SEQ ID NO:148 (01G06, Ch01G06 Chimeric, Hu01G06 IGHV1-18, Hu01G06 IGHV1-69, Sh01G06 IGHV1-18 M69L), and a CDR H3 comprising an amino acid sequence selected from the group consisting of SEQ ID NO:15 and SEQ ID NO:154 (01G06, Ch01G06 Chimeric, Hu01G06 IGHV1-18, Hu01G06 IGHV1-69, Sh01G06 IGHV1-18 M69L); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2);
(u) (i) an immunoglobulin heavy chain variable region comprising a CDR H1 comprising an amino acid sequence selected from the group consisting of SEQ ID NO:1, SEQ ID NO:38 and SEQ ID NO:136 (Sh01G06 IGHV1-18 M69L K64Q G44S, Sh01G06 IGHV1-18 M69L K64Q), a CDR H2 comprising an amino acid sequence selected from the group consisting of SEQ ID NO:13, SEQ ID NO:143 and SEQ ID NO:148 (Sh01G06 IGHV1-18 M69L K64Q G44S, Sh01G06 IGHV1-18 M69L K64Q), and a CDR H3 comprising an amino acid sequence selected from the group consisting of SEQ ID NO:15 and SEQ ID NO:154 (Sh01G06 IGHV1-18 M69L K64Q G44S, Sh01G06 IGHV1-18 M69L K64Q); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2);
(v) (i) an immunoglobulin heavy chain variable region comprising a CDR H1 comprising an amino acid sequence selected from the group consisting of SEQ ID NO:1, SEQ ID NO:234 and SEQ ID NO:235 (Sh01G06 IGHV1-69 T30S I69L), a CDR H2 comprising an amino acid sequence selected from the group consisting of SEQ ID NO:7, SEQ ID NO:143 and SEQ ID NO:148 (Sh01G06 IGHV1-69 T30S I69L), and a CDR H3 comprising an amino acid sequence selected from the group consisting of SEQ ID NO:15 and SEQ ID NO:154 (Sh01G06 IGHV1-69 T30S I69L); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2); and a CDR L3 comprising the amino acid sequence of SEQ ID NO:244 (Hu01G06 IGKV1-39 F2);
(w) (i) an immunoglobulin heavy chain variable region comprising a CDR H1 comprising an amino acid sequence selected from the group consisting of SEQ ID NO:1, SEQ ID NO:234 and SEQ ID NO:235 (Sh01G06 IGHV1-69 T30S K64Q I69L), a CDR H2 comprising an amino acid sequence selected from the group consisting of SEQ ID NO:13, SEQ ID NO:143 and SEQ ID NO:148 (Sh01G06 IGHV1-69 T30S K64Q I69L), and a CDR H3 comprising an amino acid sequence selected from the group consisting of SEQ ID NO:15 and SEQ ID NO:154 (Sh01G06 IGHV1-69 T30S K64Q 169L); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2);
(x) (i) an immunoglobulin heavy chain variable region comprising a CDR H1 comprising an amino acid sequence selected from the group consisting of SEQ ID NO:1, SEQ ID NO:38 and SEQ ID NO:136 (Hu01G06 IGHV1-18 F1), a CDR H2 comprising an amino acid sequence selected from the group consisting of SEQ ID NO:236, SEQ ID NO:240 and SEQ ID NO:242 (Hu01G06 IGHV1-18 F1), and a CDR H3 comprising an amino acid sequence selected from the group consisting of SEQ ID NO:15 and SEQ ID NO:154 (Hu01G06 IGHV1-18 F1); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2); and
(y) (i) an immunoglobulin heavy chain variable region comprising a CDR H1 comprising an amino acid sequence selected from the group consisting of SEQ ID NO:1, SEQ ID NO:234 and SEQ ID NO:235 (Hu01G06 IGHV1-69 F1), a CDR H2 comprising an amino acid sequence selected from the group consisting of SEQ ID NO:238, SEQ ID NO:241 and SEQ ID NO:243 (Hu01G06 IGHV1-69 F1), and a CDR H3 comprising an amino acid sequence selected from the group consisting of SEQ ID NO:15 and SEQ ID NO:154 (Hu01G06 IGHV1-69 F1); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F2), a CDR L2 comprising an amino acid sequence selected from the group consisting of SEQ ID NO:26 and DAK (Hu01G06 IGKV1-39 F2), and a CDR L3 comprising the amino acid sequence of SEQ ID NO:244 (Hu01G06 IGKV1-39 F2) to a mammal in need thereof.

63. The method of claim 62, wherein loss of muscle mass associated with cachexia is inhibited.

64. The method of claim 63, wherein the cachexia is associated with an underlying disease selected from the group consisting of cancer, chronic heart failure, chronic kidney disease, COPD, AIDS, multiple sclerosis, rheumatoid arthritis, sepsis, and tuberculosis.

65. The method of claim 63, wherein the loss of muscle mass is accompanied by a loss of fat mass.

66. The method of claim 62, wherein involuntary weight loss associated with cachexia is inhibited or reduced.

67. The method of claim 62, wherein loss of organ mass associated with cachexia is inhibited.

68. The method of claim 67, wherein the cachexia is associated with an underlying disease selected from the group consisting of cancer, chronic heart failure, chronic kidney disease, COPD, AIDS, multiple sclerosis, rheumatoid arthritis, sepsis, and tuberculosis.

69. The method of claim 67, wherein the organ is kidney, liver, heart or spleen.

70. The method of claim 67, wherein the loss of organ mass is accompanied by a loss of muscle mass, a loss of fat mass or involuntary weight loss.

71. The method of claim 62 further comprising administering a second agent to the mammal in need thereof, wherein the second agent is selected from the group consisting of an inhibitor of Activin-A, an inhibitor of ActRIIB, an inhibitor of IL-6, an inhibitor of IL-6R, a melanocortin peptide inhibitor, a melanocortin receptor inhibitor, a ghrelin, a ghrelin mimetic, a GHS-R1a agonist, a SARM, a TNFα inhibitor, an IL-1α inhibitor, a myostatin inhibitor, a beta-blocker and an anti-cancer agent.

72. The method of claim 62, wherein sarcopenia associated with cachexia is treated.

73. The method of claim 62, wherein the incidence and/or severity of cachexia is decreased.

74. The method of claim 71, wherein the antibody or antigen-binding fragment thereof is administered in combination with the anti-cancer agent.

75. The method of claim 62, wherein the anti-GDF15 antibody or antigen-binding fragment thereof specifically binds to the same epitope on GDF15 as an antibody comprising an immunoglobulin heavy chain variable region and an immunoglobulin light chain variable region selected from the group consisting of:
(a) an immunoglobulin heavy chain variable region comprising the amino acid sequence of SEQ ID NO:248 (Hu01G06 IGHV1-18 F2), and an immunoglobulin light chain variable region comprising the amino acid sequence of SEQ ID NO:254 (Hu01G06 IGKV1-39 F2);
(b) an immunoglobulin heavy chain variable region comprising the amino acid sequence of SEQ ID NO:250 (Hu01G06 IGHV1-69 F1), and an immunoglobulin light chain variable region comprising the amino acid sequence of SEQ ID NO:92 (Hu01G06 IGKV1-39 F1);
(c) an immunoglobulin heavy chain variable region comprising the amino acid sequence of SEQ ID NO:40 (01G06, Ch01G06 Chimeric), and an immunoglobulin light chain variable region comprising the amino acid sequence of SEQ ID NO:76 (01G06, Ch01G06 Chimeric);
(d) an immunoglobulin heavy chain variable region comprising the amino acid sequence of SEQ ID NO:42 (03G05), and an immunoglobulin light chain variable region comprising the amino acid sequence of SEQ ID NO:78 (03G05);
(e) an immunoglobulin heavy chain variable region comprising the amino acid sequence of SEQ ID NO:44 (04F08), and an immunoglobulin light chain variable region comprising the amino acid sequence of SEQ ID NO:80 (04F08);
(f) an immunoglobulin heavy chain variable region comprising the amino acid sequence of SEQ ID NO:46 (06C11), and an immunoglobulin light chain variable region comprising the amino acid sequence of SEQ ID NO:82 (06C11);
(g) an immunoglobulin heavy chain variable region comprising the amino acid sequence of SEQ ID NO:48 (08G01), and an immunoglobulin light chain variable region comprising the amino acid sequence of SEQ ID NO:84 (08G01);
(h) an immunoglobulin heavy chain variable region comprising the amino acid sequence of SEQ ID NO:50 (14F11), and an immunoglobulin light chain variable region comprising the amino acid sequence of SEQ ID NO:86 (14F11);
(i) an immunoglobulin heavy chain variable region comprising the amino acid sequence of SEQ ID NO:52 (17B11), and an immunoglobulin light chain variable region comprising the amino acid sequence of SEQ ID NO:88 (17B11);
(j) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:76 (Ch01G06 Chimeric);
(k) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:76 (Ch01G06 Chimeric);
(l) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:76 (Ch01G06 Chimeric);
(m) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:76 (Ch01G06 Chimeric);
(n) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:76 (Ch01G06 Chimeric);
(o) an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:76 (Ch01G06 Chimeric);
(p) an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:76 (Ch01G06 Chimeric);
(q) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:90 (Hu01G06 IGKV1-39);
(r) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:90 (Hu01G06 IGKV1-39);
(s) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:90 (Hu01G06 IGKV1-39);
(t) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:90 (Hu01G06 IGKV1-39);
(u) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:90 (Hu01G06 IGKV1-39);
(v) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:90 (Hu01G06 IGKV1-39);
(w) an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:90 (Hu01G06 IGKV1-39);
(x) an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:90 (Hu01G06 IGKV1-39);
(y) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:92 (Hu01G06 IGKV1-39 S43A V48I);
(z) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:92 (Hu01G06 IGKV1-39 S43A V48I);
(aa) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:92 (Hu01G06 IGKV1-39 S43A V48I);
(bb) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:92 (Hu01G06 IGKV1-39 S43A V48I);
(cc) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:92 (Hu01G06 IGKV1-39 S43A V48I);
(dd) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:92 (Hu01G06 IGKV1-39 S43A V48I);
(ee) an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:92 (Hu01G06 IGKV1-39 S43A V48I);
(ff) an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:92 (Hu01G06 IGKV1-39 S43A V48I);
(gg) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:94 (Hu01G06 IGKV1-39 V48I);
(hh) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:94 (Hu01G06 IGKV1-39 V48I);
(ii) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:94 (Hu01G06 IGKV1-39 V48I);
(jj) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:94 (Hu01G06 IGKV1-39 V48I);
(kk) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:94 (Hu01G06 IGKV1-39 V48I);
(ll) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:94 (Hu01G06 IGKV1-39 V48I);
(mm) an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO:94 (Hu01G06 IGKV1-39 V48I);
(nn) an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO:94 (Hu01G06 IGKV1-39 V48I);
(oo) an immunoglobulin heavy chain variable region comprising the amino acid sequence of SEQ ID NO:246 (Hu01G06 IGHV1-18 F1), and an immunoglobulin light chain variable region comprising the amino acid sequence of SEQ ID NO:92 (Hu01G06 IGKV1-39 F1);
(pp) an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:92 (Hu01G06 IGKV1-39 F1);
(qq) an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:254 (Hu01G06 IGKV1-39 F2);
(rr) an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:82 (Ch06C11 Chimeric);
(ss) an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:82 (Ch06C11 Chimeric);
(tt) an immunoglobulin heavy chain variable region comprising the amino acid sequence of SEQ ID NO:46 (Ch06C11 Chimeric), and an immunoglobulin light chain variable region comprising the amino acid sequence of SEQ ID NO:96 (Sh06C11 IGKV1-16);
(uu) an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:96 (Sh06C11 IGKV1-16);
(vv) an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:96 (Sh06C11 IGKV1-16);
(ww) an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:86 (Ch14F11 Chimeric);
(xx) an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:86 (Ch14F11 Chimeric);
(yy) an immunoglobulin heavy chain variable region comprising the amino acid sequence of SEQ ID NO:50 (Ch14F11 Chimeric), and an immunoglobulin light chain variable region comprising the amino acid sequence of SEQ ID NO:98 (Hu14F11 IGKV1-16);
(zz) an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:98 (Hu14F11 IGKV1-16);
(aaa) an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:98 (Hu14F11 IGKV1-16);
(bbb) an immunoglobulin heavy chain variable region comprising the amino acid sequence of SEQ ID NO:248 (Hu01G06 IGHV1-18 F2), and an immunoglobulin light chain variable region comprising the amino acid sequence of SEQ ID NO:92 (Hu01G06 IGKV1-39 F1);
(ccc) an immunoglobulin heavy chain variable region comprising the amino acid sequence of SEQ ID NO:40 (01G06, Ch01G06 Chimeric), and an immunoglobulin light chain variable region comprising the amino acid sequence of SEQ ID NO:254 (Hu01G06 IGKV1-39 F2);
(ddd) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO: 254 (Hu01G06 IGKV1-39 F2);
(eee) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO: 254 (Hu01G06 IGKV1-39 F2);
(fff) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO: 254 (Hu01G06 IGKV1-39 F2);
(ggg) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO: 254 (Hu01G06 IGKV1-39 F2);
(hhh) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO: 254 (Hu01G06 IGKV1-39 F2);
(iii) an immunoglobulin heavy chain variable region comprising the amino acid sequence of SEQ ID NO:64 (Sh01G06 IGHV1-69 T30S I69L), and an immunoglobulin light chain variable region comprising the amino acid sequence of SEQ ID NO: 254 (Hu01G06 IGKV1-39 F2);
(jjj) an immunoglobulin heavy chain variable region comprising the amino acid sequence of SEQ ID NO:66 (Sh01G06 IGHV1-69 T30S K64Q I69L), and an immunoglobulin light chain variable region comprising the amino acid sequence of SEQ ID NO: 254 (Hu01G06 IGKV1-39 F2);
(kkk) an immunoglobulin heavy chain variable region comprising the amino acid sequence of SEQ ID NO:246 (Hu01G06 IGHV1-18 F1), and an immunoglobulin light chain variable region comprising the amino acid sequence of SEQ ID NO: 254 (Hu01G06 IGKV1-39 F2); and
(lll) an immunoglobulin heavy chain variable region comprising the amino acid sequence of SEQ ID NO:250 (Hu01G06 IGHV1-69 F1), and an immunoglobulin light chain variable region comprising the amino acid sequence of SEQ ID NO: 254 (Hu01G06 IGKV1-39 F2).

76. The method of claim 62, wherein the anti-GDF15 antibody or antigen-binding fragment thereof specifically binds to the same epitope on GDF15 as an antibody comprising an immunoglobulin heavy chain and an immunoglobulin light chain selected from the group consisting of:
(a) an immunoglobulin heavy chain comprising the amino acid sequence of SEQ ID NO:258 (Hu01G06 IGHV1-18 F2), and an immunoglobulin light chain comprising the amino acid sequence of SEQ ID NO:264 (Hu01G06 IGKV1-39 F2);
(b) an immunoglobulin heavy chain comprising the amino acid sequence of SEQ ID NO:260 (Hu01G06 IGHV1-69 F1), and an immunoglobulin light chain comprising the amino acid sequence of SEQ ID NO:208 (Hu01G06 IGKV1-39 F1);
(c) an immunoglobulin heavy chain comprising the amino acid sequence of SEQ ID NO:176 (Ch01G06 Chimeric), and an immunoglobulin light chain comprising the amino acid sequence of SEQ ID NO:204 (Ch01G06 Chimeric);
(d) an immunoglobulin heavy chain comprising the amino acid sequence of SEQ ID NO:192 (Ch06C11 Chimeric), and an immunoglobulin light chain comprising the amino acid sequence of SEQ ID NO:212 (Ch06C11 Chimeric);
(e) an immunoglobulin heavy chain comprising the amino acid sequence of SEQ ID NO:198 (Ch14F11 Chimeric), and an immunoglobulin light chain comprising the amino acid sequence of SEQ ID NO:216 (Ch14F11 Chimeric);
(f) an immunoglobulin heavy chain comprising the amino acid sequence of SEQ ID NO:178 (Hu01G06 IGHV1-18), and an immunoglobulin light chain comprising the amino acid sequence of SEQ ID NO:206 (Hu01G06 IGKV1-39);
(g) an immunoglobulin heavy chain comprising the amino acid sequence of SEQ ID NO:180 (Hu01G06 IGHV1-69), and an immunoglobulin light chain comprising the amino acid sequence of SEQ ID NO:206 (Hu01G06 IGKV1-39);
(h) an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:210 (Hu01G06 IGKV1-39 V48I);
(i) an immunoglobulin heavy chain comprising the amino acid sequence of SEQ ID NO:188 (Sh01G06 IGHV1-69 T30S I69L), and an immunoglobulin light chain comprising the amino acid sequence of SEQ ID NO:210 (Hu01G06 IGKV1-39 V48I);
(j) an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:208 (Hu01G06 IGKV1-39 S43A V48I);
(k) an immunoglobulin heavy chain comprising the amino acid sequence of SEQ ID NO:188 (Sh01G06 IGHV1-69 T30S I69L), and an immunoglobulin light chain comprising the amino acid sequence of SEQ ID NO:208 (Hu01G06 IGKV1-39 S43A V48I);
(l) an immunoglobulin heavy chain comprising the amino acid sequence of SEQ ID NO:256 (Hu01G06 IGHV1-18 F1), and an immunoglobulin light chain comprising the amino acid sequence of SEQ ID NO:208 (Hu01G06 IGKV1-39 F1);
(m) an immunoglobulin heavy chain comprising the amino acid sequence of SEQ ID NO:262 (Hu01G06 IGHV1-69 F2), and an immunoglobulin light chain comprising the amino acid sequence of SEQ ID NO:208 (Hu01G06 IGKV1-39 F1);
(n) an immunoglobulin heavy chain comprising the amino acid sequence of SEQ ID NO:262 (Hu01G06 IGHV1-69 F2), and an immunoglobulin light chain comprising the amino acid sequence of SEQ ID NO:264 (Hu01G06 IGKV1-39 F2);
(o) an immunoglobulin heavy chain comprising the amino acid sequence of SEQ ID NO:194 (HE LM 06C11 IGHV2-70), and an immunoglobulin light chain comprising the amino acid sequence of SEQ ID NO:214 (Sh06C11 IGKV1-16);
(p) an immunoglobulin heavy chain comprising the amino acid sequence of SEQ ID NO:196 (Hu06C11 IGHV2-5), and an immunoglobulin light chain comprising the amino acid sequence of SEQ ID NO:214 (Sh06C11 IGKV1-16);
(q) an immunoglobulin heavy chain comprising the amino acid sequence of SEQ ID NO:200 (Sh14F11 IGHV2-5), and an immunoglobulin light chain comprising the amino acid sequence of SEQ ID NO:218 (Hu14F11 IGKV1-16);
(r) an immunoglobulin heavy chain comprising the amino acid sequence of SEQ ID NO:202 (Sh14F11 IGHV2-70), and an immunoglobulin light chain comprising the amino acid sequence of SEQ ID NO:218 (Hu14F11 IGKV1-16);
(s) an immunoglobulin heavy chain comprising the amino acid sequence of SEQ ID NO:182 (Sh01G06 IGHV1-18 M69L), and an immunoglobulin light chain comprising the amino acid sequence of SEQ ID NO:206 (Hu01G06 IGKV1-39);
(t) an immunoglobulin heavy chain comprising the amino acid sequence of SEQ ID NO:186 (Sh01G06 IGHV1-18 M69L K64Q), and an immunoglobulin light chain comprising the amino acid sequence of SEQ ID NO:206 (Hu01G06 IGKV1-39);
(u) an immunoglobulin heavy chain comprising the amino acid sequence of SEQ ID NO: 184 (Sh01G06 IGHV1-18 M69L K64Q G44S), and an immunoglobulin light chain comprising the amino acid sequence of SEQ ID NO:206 (Hu01G06 IGKV1-39);
(v) an immunoglobulin heavy chain comprising the amino acid sequence of SEQ ID NO:188 (Sh01G06 IGHV1-69 T30S I69L), and an immunoglobulin light chain comprising the amino acid sequence of SEQ ID NO:206 (Hu01G06 IGKV1-39);
(w) an immunoglobulin heavy chain comprising the amino acid sequence of SEQ ID NO:190 (Sh01G06 IGHV1-69 T30S K64Q I69L), and an immunoglobulin light chain comprising the amino acid sequence of SEQ ID NO:206 (Hu01G06 IGKV1-39);
(x) an immunoglobulin heavy chain comprising the amino acid sequence of SEQ ID NO:182 (Sh01G06 IGHV1-18 M69L), and an immunoglobulin light chain comprising the amino acid sequence of SEQ ID NO:210 (Hu01G06 IGKV1-39 V48I);
(y) an immunoglobulin heavy chain comprising the amino acid sequence of SEQ ID NO:186 (Sh01G06 IGHV1-18 M69L K64Q), and an immunoglobulin light chain comprising the amino acid sequence of SEQ ID NO:210 (Hu01G06 IGKV1-39 V48I);
(z) an immunoglobulin heavy chain comprising the amino acid sequence of SEQ ID NO:190 (Sh01G06 IGHV1-69 T30S K64Q I69L), and an immunoglobulin light chain comprising the amino acid sequence of SEQ ID NO:210 (Hu01G06 IGKV1-39 V48I);
(aa) an immunoglobulin heavy chain comprising the amino acid sequence of SEQ ID NO:182 (Sh01G06 IGHV1-18 M69L), and an immunoglobulin light chain comprising the amino acid sequence of SEQ ID NO:208 (Hu01G06 IGKV1-39 S43A V48I);
(bb) an immunoglobulin heavy chain comprising the amino acid sequence of SEQ ID NO:186 (Sh01G06 IGHV1-18 M69L K64Q), and an immunoglobulin light chain comprising the amino acid sequence of SEQ ID NO:208 (Hu01G06 IGKV1-39 S43A V48I);
(cc) an immunoglobulin heavy chain comprising the amino acid sequence of SEQ ID NO:190 (Sh01G06 IGHV1-69 T30S K64Q I69L), and an immunoglobulin light chain comprising the amino acid sequence of SEQ ID NO:208 (Hu01G06 IGKV1-39 S43A V48I);
(dd) an immunoglobulin light chain comprising the amino acid sequence of SEQ ID NO: 204 and an immunoglobulin heavy chain comprising the amino acid sequence of SEQ ID NO:178, 180, 182, 184, 186, 188, or 190;
(ee) an immunoglobulin light chain comprising the amino acid sequence of SEQ ID NO: 208 and an immunoglobulin heavy chain comprising the amino acid sequence of SEQ ID NO: 176, 178, 180, or 258;
(ff) an immunoglobulin light chain comprising the amino acid sequence of SEQ ID NO: 210 and an immunoglobulin heavy chain comprising the amino acid sequence of SEQ ID NO: 176, 178, or 180;
(gg) an immunoglobulin light chain comprising the amino acid sequence of SEQ ID NO: 264 and an immunoglobulin heavy chain comprising the amino acid sequence of SEQ ID NO: 176, 178, 180, 182, 184, 186, 188, 190, 256, or 260; and
(hh) an immunoglobulin light chain comprising the amino acid sequence of SEQ ID NO: 206 and an immunoglobulin heavy chain comprising the amino acid sequence of SEQ ID NO: 176.

77. The method of claim 62, wherein the cachexia is associated with an underlying disease selected from the group consisting of cancer, chronic heart failure, chronic kidney disease, COPD, AIDS, multiple sclerosis, rheumatoid arthritis, sepsis, and tuberculosis.

78. The method of claim 62, wherein the anti-GDF15 antibody or antigen-binding fragment thereof specifically binds to the same epitope on GDF15 as an antibody comprising
(i) an immunoglobulin heavy chain variable region comprising a CDR H1 comprising an amino acid sequence selected from the group consisting of SEQ ID NO:1, SEQ ID NO:234, and SEQ ID NO:235 (Hu01G06 IGHV1-69 F1), a CDR H2 comprising an amino acid sequence selected from the group consisting of SEQ ID NO:238, SEQ ID NO:241, and SEQ ID NO:243, (Hu01G06 IGHV1-69 F1), and a CDR H3 comprising an amino acid sequence selected from the group consisting of SEQ ID NO:15 and SEQ ID NO:154 (Hu01G06 IGHV1-69 F1); and
(ii) an immunoglobulin light chain variable region comprising a CDR L1 comprising an amino acid sequence selected from the group consisting of SEQ ID NO:21 and SEQ ID NO:160 (Hu01G06 IGKV1-39 F1), CDR L2 comprising an amino acid sequence selected from the group consisting of SEQ ID NO:26 and DAK (Hu01G06 IGKV1-39 F1), and a CDR L3 comprising the amino acid sequence of SEQ ID NO:32 (Hu01G06 IGKV1-39 F1).

79. The method of claim 78, further comprising administering a second agent to the mammal in need thereof, wherein the second agent is selected from the group consisting of an inhibitor of Activin-A, an inhibitor of ActRIIB, an inhibitor of IL-6, an inhibitor of IL-6R, a melanocortin peptide inhibitor, a melanocortin receptor inhibitor, a ghrelin, a ghrelin mimetic, a GHS-R1a agonist, a SARM, a TNFα inhibitor, an IL-1α inhibitor, a myostatin inhibitor, a beta-blocker and an anti-cancer agent.

80. The method of claim 78, wherein the cachexia is associated with an underlying disease selected from the group consisting of cancer, chronic heart failure, chronic kidney disease, COPD, AIDS, multiple sclerosis, rheumatoid arthritis, sepsis, and tuberculosis.

81. The method of claim 62, wherein appetite in a mammal suffering from cachexia is increased.
